(19) United States
(12) Patent Application Publication
(10) Pub. No.: US 2025/0170098 A1
(43) Pub. Date: May 29, 2025

(54) MOLECULAR JACKHAMMER FOR MECHANICAL DESTRUCTION OF CELLULAR STRUCTURE

(71) Applicants: William Marsh Rice University, Houston, TX (US); Nanorobotics, LTD, Hod Hasharon (IL)

(72) Inventors: Ciceron AYALA-OROZCO, Houston, TX (US); James M. TOUR, Houston, TX (US); Gang LI, Houston, TX (US); Bowen LI, Houston, TX (US)

(73) Assignees: William Marsh Rice University, Houston, TX (US); Nanorobotics, LTD, Hod Hasharon (IL)

(21) Appl. No.: 18/841,126

(22) PCT Filed: Feb. 24, 2023
(86) PCT No.: PCT/US2023/063288

§ 371 Date: Aug. 23, 2024

Related U.S. Application Data
(60) Provisional application No. 63/314,094, filed on Feb. 25, 2022.

(51) Int. Cl. A61K 31/4045; A61K 45/06; A61P 35/00; C07D 403/08

(52) U.S. Cl. CPC A61K 31/4045; A61K 45/06; A61P 35/00; C07D 403/08

(57) ABSTRACT

In one aspect, the present disclosure describes methods which may be used to disrupt cellular membranes. These compounds may generate a vibronic-driven action when exposed to a sufficient energy source and that action may be used to disrupt the membrane of a cell or other organism. Also described are compounds and uses thereof.

This application claims the benefit of priority to U.S. Provisional Application No. 63/314,094, filed on Feb. 25, 2022, the entire contents of which are hereby incorporated by reference.

### BACKGROUND

### 1. Field

This disclosure relates to the fields of biology, biochemistry, chemistry, pharmacology and medicine. In particular, new methods, compounds, and methods of treatment related to vibronic-driven action are disclosed.

### 2. Related Art

While UV and visible light have only hundreds of microns to 1 mm of light penetration through human tissue (skin, muscle, fat), the near-infrared (NIR) window of 650 nm to 900 nm, also known as the optical therapeutic window, is ideally suited for in vivo applications because of minimal light absorption by hemoglobin and water with significant penetration through human tissue reaching ˜10 cm (Weissleder, 2001). Two-photon NIR activation of Feringa-type motors has been previously exploited for inducing rapid cellular necrosis, but the technique requires enormous laser-generated fluxes of photons and hence the depth of penetration is shallow, ˜0.5 mm, and the area of coverage is restricted to smaller-sized domains (Liu et al., 2019). Single photo NIR light is insufficient in energy to activate Feringa-type motors, which require excitation in the UV (˜360 nm) or blue visible light region (˜405 nm).

In a typical molecular absorbance of photons, an individual bond or small part of the molecule starts vibrating (FIG. 1A). In a thermal activation of a molecule, many bonds vibrate in a disconcerted manner (FIG. 1). But there is another way to excite a molecule wherein a whole-molecule-vibration or collective vibration is achieved that is much longer-range and concerted, spreading through the entire length or width of the molecule (FIG. 1C). When vibrational and electronic modes, sometimes called the plasmonic and phonon modes, respectively, are coupled, the two modes together result in vibronic coupling (Qian et al., 2020; Orlandi and Siebrand, 2003) or molecular plasmon-phonon coupling (Cui et al., 2016). More specifically, by absorbance of a suitable energy of light, the vibrational modes of a molecule hybridize with the electronic transitions of the molecule to induce the vibronic mode. The vibronic mode is analogous to an ultrafast breathing mode of a molecule where the entire molecule is vibrating in unison throughout its length and/or its width because one can have a longitudinal or transverse collective vibration, respectively (Cui et al., 2016; Chapkin et al, 2018). Cyanine dyes have been used in photothermal and photodynamic therapies and they are readily accepted in biological and medicinal studies (Mishra et al., 2000; Li et al., 2021; Shi et al., 2016; Lange et al., 2021; Bilici et al., 2021). Heating a molecule through photothermal therapy can cause many vibrations in a molecule, but those vibrations are not coordinated, as shown in FIG. 1B, hence there is no concerted longitudinal or transverse vibration that is sufficient to rapidly open a cell membrane. Hence, high powers and extended times are need in photothermal therapy to cause slow apoptotic death. In addition, photodynamic therapy generates reactive oxygen species (ROS), and is therefore susceptible to the action of ROS-inhibitors.

Additionally, US Patent Application No. 2020/0289676 relates to the use of near-infrared dye with conjugates for treating tumors. WO 2020/020905 relates to the use of near-infrared containg N-triazole chromophores that may be used in treatments such as photodynamtic therapy. WO 1997/040829 relates to the use of compounds for neuroendocrine resetting therapy or photodynamic therapy. U.S. Pat. No. 7,229,447 relates to the use of methylene blue in photodynamic disruption of cells. Finally, Yan et al. relates to the use of water-soluble cyanine dye for photodynamic therapy of cancer cells. Furthermore, WO 2022023496 relates to the preparation of isonitrile containing compound including fluorophores. US 2008/0233050 relates to cyanine and indocyanine dye conjugates that may be used to visualize and detect a tumor. WO 2011152046 relate to compositions of indocyanine dye and liposomes. Finally, WO 2005/082423 relates to methods of imaging the lympthatic or circulatory system using near IR dyes.

Therefore, there remains a need to find new and unique ways to achieve rapid cellular death that are distinct from photothermal therapy and ROS-based photodynamic therapies.

### SUMMARY

As provided herein, the present disclosure relates to methods of disrupting cell membranes using vibronic-driven actions.

In one aspect, the present disclosure provides methods of disrupting a membrane comprising:
(A) contacting the membrane with a compound, wherein the compound is capable of generating a vibronic-driven action and optionally further comprising a targeting moiety; and
(B) exposing the compound to an energy source sufficient to generate the vibronic-driven action, wherein the vibronic-driven action is sufficient to disrupt the membrane.

In one aspect, the present disclosure provides compounds for use in disrupting a membrane comprising a compound is capable of generating a vibronic-driven action and optionally further comprising a targeting moiety; wherein the vibronic-driven action is sufficient to disrupt the membrane.

In one aspect, the present disclosure provides uses of a compound of disrupting a membrane comprising:
(A) contacting the membrane with the compound, wherein the compound is capable of generating a vibronic-driven action and optionally further comprising a targeting moiety; and
(B) exposing the compound to an energy source sufficient to generate the vibronic-driven action, wherein the vibronic-driven action is sufficient to disrupt the membrane.

In another aspect, the present disclosure provides methods of disrupting a membrane comprising:
(A) contacting the membrane with a compound, wherein the compound optionally further comprises a targeting moiety; and
(B) exposing the compound to an energy source, wherein the compound generates motion sufficient to disrupt the membrane and the energy source has an intensity of less than 250 mW/cm².

In another aspect, the present disclosure provides compound for use in disrupting a membrane comprising a compound, wherein the compound optionally further comprises a targeting moiety; and wherein the compound generates motion sufficient to disrupt the membrane and the energy source has an intensity of less than 250 mW/cm².

In another aspect, the present disclosure provides uses of a compound for disrupting a membrane comprising:
(A) contacting the membrane with the compound, wherein the compound optionally further comprises a targeting moiety; and
(B) exposing the compound to an energy source, wherein the compound generates motion sufficient to disrupt the membrane and the energy source has an intensity of less than 250 mW/cm².

In some embodiments, the membrane is the outer membrane of a cell. In other embodiments, the membrane is the inner membrane of a cell. In some embodiments, the inner membrane is the membrane of an organelle such as a mitochondria, a nucleus, an endoplasmic reticulum, or a golgi apparatus. In some embodiments, the membrane is the membrane of prokaryotic cell. In other embodiments, the membrane is the membrane of eukaryotic cell. In further embodiments, the membrane is the membrane of a human cell. In some embodiments, the human cell is a cancer cell. In other embodiments, the human cell is a healthy cell. In some embodiments, the human cell is an adipose cell.

In some embodiments, the membrane is a bacterial membrane, a viral membrane, a fungal membrane, or a protozoal membrane. In some embodiments, the membrane is a bacterial membrane. In other embodiments, the membrane is a viral membrane. In other embodiments, the membrane is a fungal membrane. In still other embodiments, the membrane is a protozoal membrane. In some embodiments, the membrane is a membrane of a parasite.

In some embodiments, the disruption creates a pore in the membrane. In some embodiments, the methods result in necrosis of the cell. In other embodiments, the methods result in death through the disruption of an organelle of the cell. In other embodiments, the methods result in death through the disruption of an nucleus of the cell.

In some embodiments, the compound comprises:
(i) has a net dipole via a charge (cation or anion or radical cation or radical anion) or radical (single unpaired electron);
(ii) has a high degree of symmetry across the longitudinal and/or transverse axis; and
(iii) has a resonance structure through a pi-bonded system whereby the charge or radical can oscillate between the near-symmetric two ends via resonance.

In some embodiments, the compound is an organomettalic compound. In further embodiments, the organometallic compound is not a nanoparticle. In some embodiments, the organometallic compound is an organic ligand bound individually to one or more metal atoms. In some embodiments, the organic ligand is bound to one metal atom. In other embodiments, the organic ligand is bound to two or more metal atoms. In some embodiments, the metal atom is bound to the organic ligand via a covalent bond. In other embodiments, the metal atom is bound to the organic ligand via an ionic bond. In some embodiments, the compound is an organic molecule. In further embodiments, the organic molecule exhibits either a longitudinal molecular plasmon or a transverse molecular plasmon. In still further embodiments, the organic molecule exhibits both a longitudinal molecular plasmon and a transverse molecular plasmon. In some embodiments, the compound is an organic dye.

In some embodiments, the present disclosure provides methods wherein the compound is further defined by the formula:

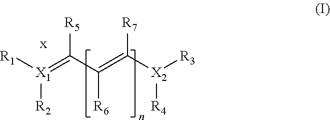

wherein:
   x is a positive or negative charge;
   n is an integer from 0 to 100;
   X₁ and X₂ are each independently a heteroatom selected from O, N, S, B, P, Ge, As, or Se; and
   R₁, R₂, R₃, R₄, R₅, R₆, and R₇ are each independently alkyl_{(C≤18)}, alkenyl_{(C≤18)}, alkynyl_{(C≤18)}, aryl_{(C≤18)}, aralkyl_{(C≤18)}, heteroaryl_{(C≤18)}, heterocycloalkyl_{(C≤18)}, or a substituted version of any of these groups; or
   R₁ and R₂, R₁ and R₅, R₂ and R₅, R₃ and R₄, R₃ and R₇, and R₄ and R₇ are taken together to form one, two, three, four, five, or six aliphatic or aromatic rings; comprising at least three carbon atoms and no more than 36 carbon atoms; optionally comprising one, two, three, four, or five nitrogen, sulfur, or oxygen atom.

In some embodiments, the compound is further defined as:

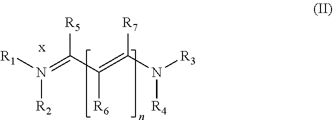

wherein:
   x is a positive charge;
   n is an integer from 0 to 100;
   each R₁, R₂, R₃, R₄, R₅, R₆, and R₇ are each independently hydrogen, alkyl_{(C≤18)}, alkenyl_{(C≤18)}, alkynyl_{(C≤18)}, aryl_{(C≤18)}, aralkyl_{(C≤18)}, heteroaryl_{(C≤18)}, heterocycloalkyl_{(C≤18)}, or a substituted version of any of these groups; or
   each R₁, R₂, R₃, R₄, R₅, R₆, and R₇ are each independently a cell membrane targeting moiety, wherein the cell targeting moiety optionally comprises a linker; or
   each R₁ and R₂, R₁ and R₅, R₂ and R₅, R₃ and R₄, R₃ and R₇, R₄ and R₇, and R₅ and R₇ are taken together and each independently form one, two, three, four, five, or six aliphatic or aromatic rings; comprising at least three carbon atoms and no more than 36 carbon atoms; optionally comprising one, two, three, four, or five nitrogen, sulfur, or oxygen atom.

In some embodiments, X₁ and X₂ are identical. In some embodiments, X₁ is N. In some embodiments, X₂ is N. In some embodiments, X₁ and X₂ are N.

In some embodiments, R₁ or R₂ are symmetric with R₃ or R₄. In some embodiments, R₁ is taken together with R₅ to form one, two, three, four, or five rings. In further embodiments, R₁ is taken together with R₅ to form two, three, or four rings. In still further embodiments, R₁ is taken together with R₅ to form three rings. In yet further embodiments, R₁ is taken together with R₅ to form three rings, wherein one ring is aliphatic and two rings are aromatic.

In some embodiments, R₂ is alkyl_{(C≤18)} or substituted alkyl_{(C≤18)}. In further embodiments, R₂ is alkyl_{(C≤18)}. In further embodiments, R₂ is alkyl_{(C≤8)}, such as methyl.

In some embodiments, R₃ is taken together with R₇ to form one, two, three, four, or five rings. In further embodiments, R₃ is taken together with R₇ to form two, three, or four rings. In still further embodiments, R₃ is taken together with R₇ to form three rings. In even further embodiments, R₃ is taken together with R₇ to form three rings, wherein one ring is aliphatic and two rings are aromatic.

In some embodiments, R₄ is alkyl_{(C≤8)} or substituted alkyl_{(C≤8)}. In some embodiments, R₄ is alkyl_{(C≤18)}. In further embodiments, R₄ is alkyl_{(C≤8)}, such as methyl.

In some embodiments, R₆ is hydrogen.

In some embodiments, R₅ and R₇ are taken together and form one, two, or three rings. In some embodiments, R₅ and R₇ are taken together and form a single ring, such as a five, six, or seven membered ring.

In some embodiments, n is an integer from 1 to 10. In further embodiments, n is an integer selected from 2, 3, or 4. In some embodiments, n is 3.

In some embodiments, R₄ is a cell targeting moiety with a linker. In some embodiments the linker is an alkyl chain, an alkenyl chain, an aryl chain, a peptide chain, a polyethylene glycol chain, or a polypropylene chain. In some embodiments, the linker further comprises one or more joining functional group selected from ether, amide, disulfide, ester, amine, or thioether. In further embodiments, the linker is two alkyl chains with an amide joining functional group.

In some embodiments, the the cell targeting moiety is a functional group that associates with the membrane, a carbohydrate or polysaccharide that binds to one or more markers on the membrane, a lipid that binds to one or more markers on the cell membrane, a small molecule that binds to one or more markers on the cell membrane, an aptamer that binds to one or more markers on the membrane, or a peptide or an antibody that binds to one or more markers on the membrane. In further embodiments, the cell targeting moiety is a functional group that associates with the cell membrane. In some embodiments, the functional group is an amine. In some embodiments, the amine is protonated. In other embodiments, the functional group is a natural product. In other embodiments, the functional group is a non-natural product small molecule.

In some embodiments, methods of the present disclosure are provided wherein the compound is further defined as:

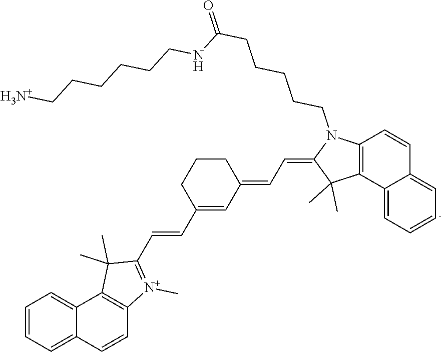 [SMILES: C[N+]1=C(/C=C/C2=CC(=C/C=C3/N(CCCCCC(=O)NCCCCCC[NH3+])c4ccc5ccccc5c4C3(C)C)/CCC2)C(C)(C)c2c1ccc1ccccc21]

In some embodiments, the compound is further defined as:

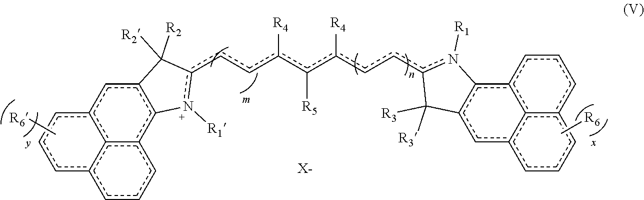

wherein:
   R₁ and R₁′ are each independently alkyl_{(C≤8)}, substituted alkyl_{(C≤8)}, or a group of the formula:

-A-NRₐRₐ′Rₐ″
wherein:
   A is an alkanediyl_{(C≤12)} or substituted alkanediyl_{(C≤12)};
   Rₐ and Rₐ′ are each independently hydrogen or alkyl_{(C≤8)}; and
   Rₐ″ is absent, hydrogen, or alkyl_{(C≤8)};
provided at least one of R₁ and R₁′ is a group of the formula: -A-NRₐRₐ′Rₐ″ R₂, R₂′, R₃, and R₃′ are each independently hydrogen, alkyl_{(C≤8)}, or substituted alkyl_{(C≤8)};
R₄ and R₄′ are each independently hydrogen, alkyl_{(C≤8)}, substituted alkyl_{(C≤8)}, or R₄ and R₄′ are taken together as a cycloalkyl group;
R₅ is hydrogen, halo, carboxy, alkyl_{(C≤8)}, substituted alkyl_{(C≤8)}, or —C(O)OR_{b}, wherein R_{b} is alkyl_{(C≤6)} or substituted alkyl_{(C≤6)};
R₆ is hydrogen, amino, halo, hydroxy, or alkyl_{(C≤12)}, alkoxy_{(C≤12)}, alkylamino_{(C≤8)}, dialkylamino_{(C≤8)}, acyl_{(C≤8)}, acyl_{(C≤8)}, acyl_{(C≤8)}, or a substituted version thereof,
m and n are each 0, 1, 2, or 3;
x and y are each independently 0, 1, 2, 3, 4, or 5;
X is a monovalent anion; and
each of the rings is optionally present as either an aromatic or aliphatic ring.

In some embodiments, the compound is at least one compound shown below:

Structure

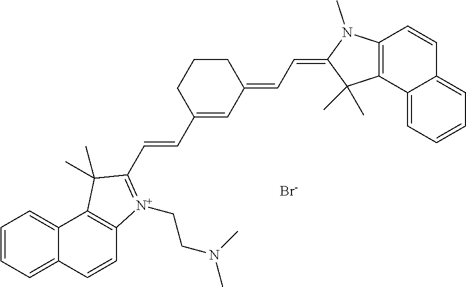 [SMILES: CN(C)CC[N+]1=C(/C=C/C2=CC(=C/C=C3/N(C)c4ccc5ccccc5c4C3(C)C)/CCC2)C(C)(C)c2c1ccc1ccccc21 ; [Br-]]

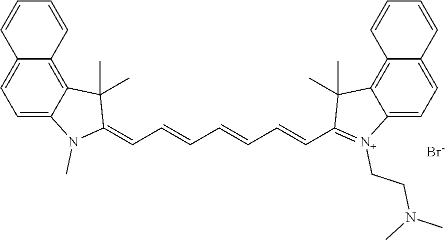 [SMILES: CN(C)CC[N+]1=C(/C=C/C=C/C=C/C=C2/N(C)c3ccc4ccccc4c3C2(C)C)C(C)(C)c2c1ccc1ccccc21 ; [Br-]]

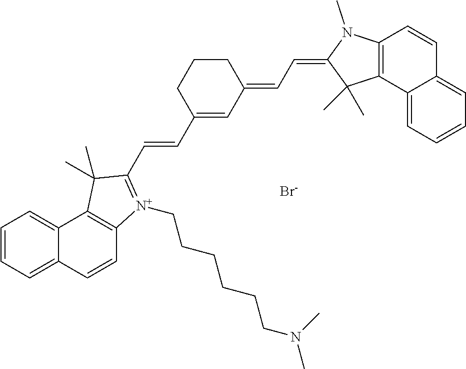 [SMILES: CN(C)CCCCCC[N+]1=C(/C=C/C2=CC(=C/C=C3/N(C)c4ccc5ccccc5c4C3(C)C)/CCC2)C(C)(C)c2c1ccc1ccccc21 ; [Br-]]

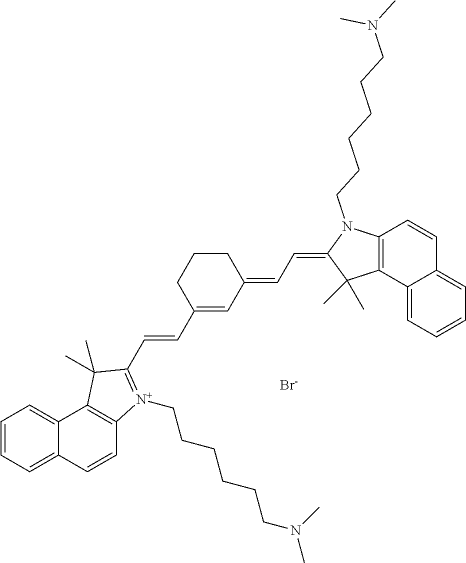 [SMILES: CN(C)CCCCCCN1/C(=C/C=C2C=C(/C=C/C3=[N+](CCCCCCN(C)C)c4ccc5ccccc5c4C3(C)C)CCC/2)C(C)(C)c2c1ccc1ccccc21 ; [Br-]]

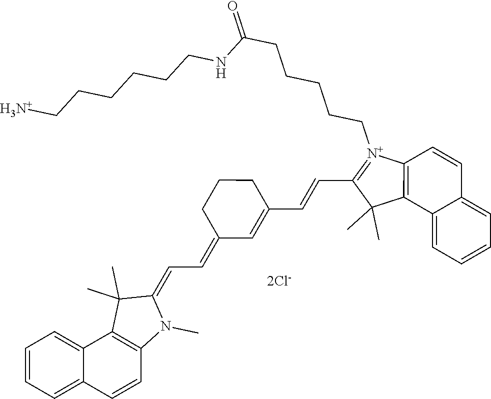 [SMILES: CN1/C(=C\C=C2C=C(/C=C/C3=[N+](CCCCCC(=O)NCCCCCC[NH3+])c4ccc5ccccc5c4C3(C)C)CCC\2)C(C)(C)c2c1ccc1ccccc21]

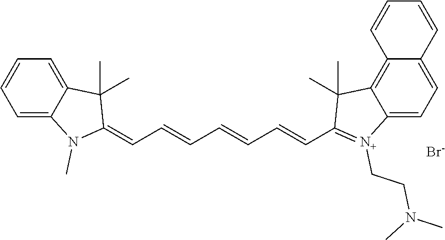 [SMILES: CN(C)CC[N+]1=C(/C=C/C=C/C=C/C=C2/N(C)c3ccccc3C2(C)C)C(C)(C)c2c1ccc1ccccc21 ; [Br-]]

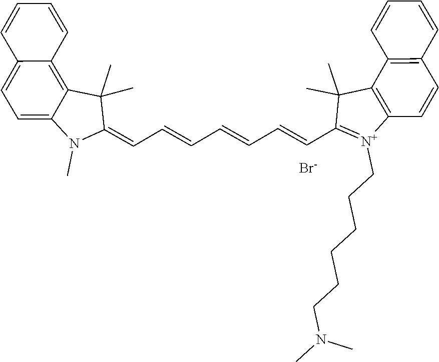 [SMILES: CN(C)CCCCCC[N+]1=C(/C=C/C=C/C=C/C=C2/N(C)c3ccc4ccccc4c3C2(C)C)C(C)(C)c2c1ccc1ccccc21 ; [Br-]]

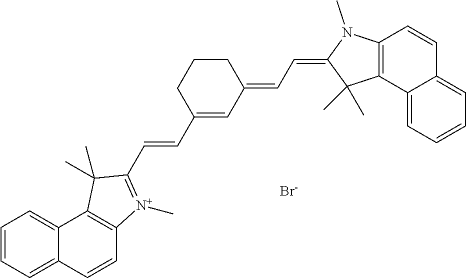 [SMILES: CN1/C(=C/C=C2C=C(/C=C/C3=[N+](C)c4ccc5ccccc5c4C3(C)C)CCC/2)C(C)(C)c2c1ccc1ccccc21 ; [Br-]]

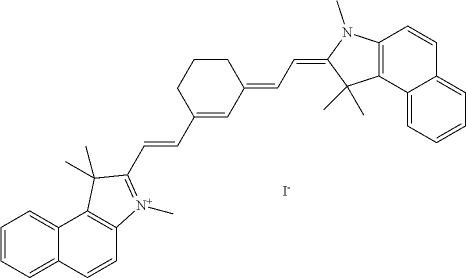 [SMILES: CN1/C(=C/C=C2C=C(/C=C/C3=[N+](C)c4ccc5ccccc5c4C3(C)C)CCC/2)C(C)(C)c2c1ccc1ccccc21 ; [I-]]

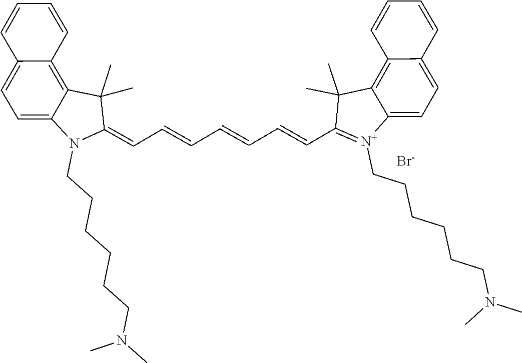 [SMILES: CN(C)CCCCCCN1/C(=C/C=C/C=C/C=C/C2=[N+](CCCCCCN(C)C)c3ccc4ccccc4c3C2(C)C)C(C)(C)c2c1ccc1ccccc21 ; [Br-]]

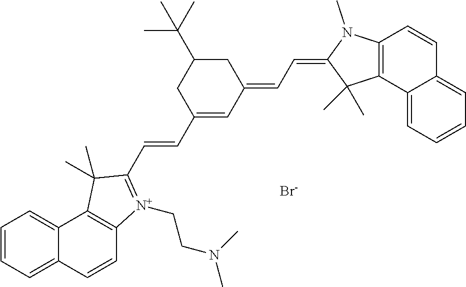 [SMILES: CN(C)CC[N+]1=C(/C=C/C2=CC(=C/C=C3/N(C)c4ccc5ccccc5c4C3(C)C)/CC(C(C)(C)C)C2)C(C)(C)c2c1ccc1ccccc21 ; [Br-]]

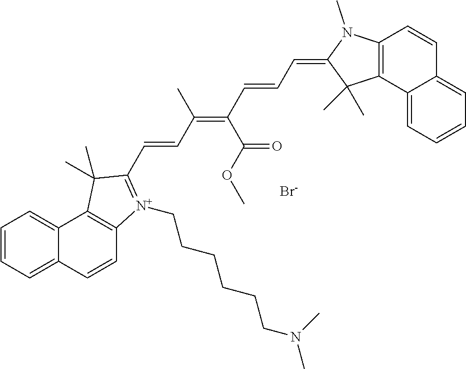 [SMILES: COC(=O)C(/C=C/C=C1/N(C)c2ccc3ccccc3c2C1(C)C)=C(C)\C=C\C1=[N+](CCCCCCN(C)C)c2ccc3ccccc3c2C1(C)C ; [Br-]]

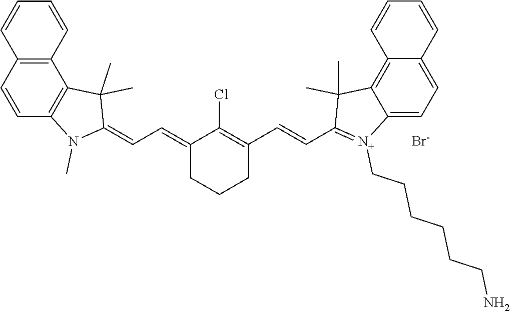 [SMILES: CN1/C(=C/C=C2\CCCC(/C=C/C3=[N+](CCCCCCN)c4ccc5ccccc5c4C3(C)C)=C2Cl)C(C)(C)c2c1ccc1ccccc21 ; [Br-]]

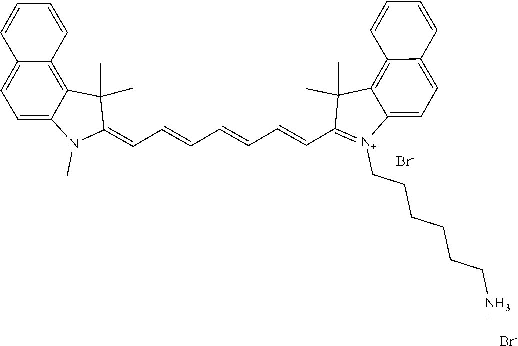 [SMILES: CN1/C(=C/C=C/C=C/C=C/C2=[N+](CCCCCCN)c3ccc4ccccc4c3C2(C)C)C(C)(C)c2c1ccc1ccccc21 ; [Br-] ; [Br-]]

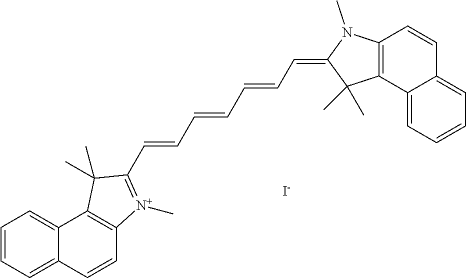 [SMILES: CN1/C(=C/C=C/C=C/C=C/C2=[N+](C)c3ccc4ccccc4c3C2(C)C)C(C)(C)c2c1ccc1ccccc21 ; [I-]]

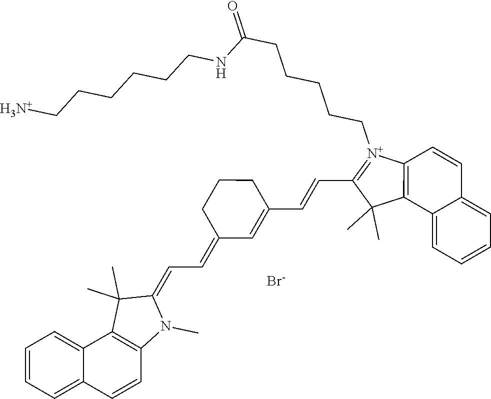 [SMILES: CN1/C(=C\C=C2C=C(/C=C/C3=[N+](CCCCCC(=O)NCCCCCC[NH3+])c4ccc5ccccc5c4C3(C)C)CCC\2)C(C)(C)c2c1ccc1ccccc21 ; [Br-]]

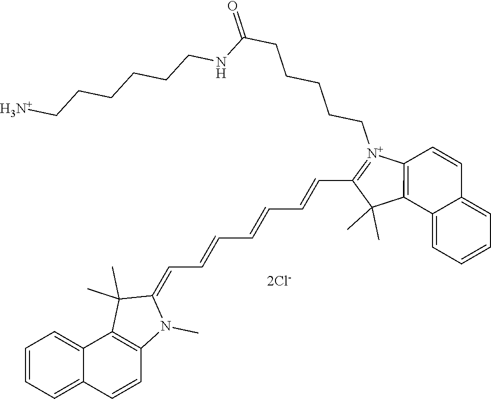 [SMILES: CN1\C(=C/C=C/C=C/C=C/C2=[N+](CCCCCC(=O)NCCCCCC[NH3+])c3ccc4ccccc4c3C2(C)C)C(C)(C)c2c1ccc1ccccc21]

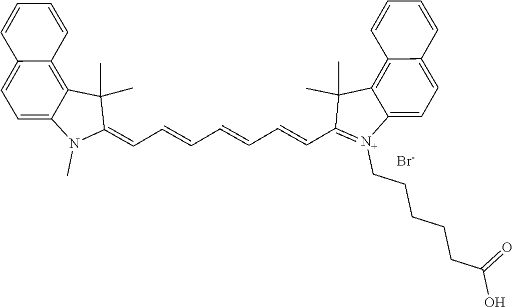 [SMILES: CN1/C(=C/C=C/C=C/C=C/C2=[N+](CCCCCC(=O)O)c3ccc4ccccc4c3C2(C)C)C(C)(C)c2c1ccc1ccccc21 ; [Br-]]

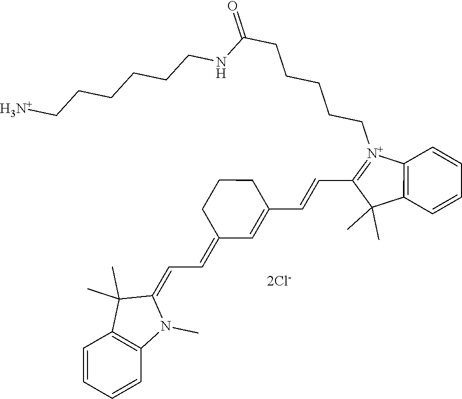 [SMILES: CN1/C(=C\C=C2C=C(/C=C/C3=[N+](CCCCCC(=O)NCCCCCC[NH3+])c4ccccc4C3(C)C)CCC\2)C(C)(C)c2ccccc21]

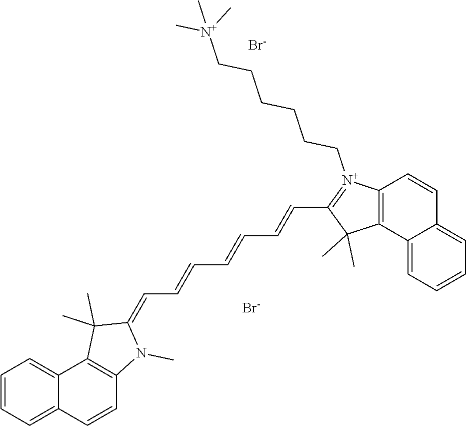 [SMILES: CN1\C(=C/C=C/C=C/C=C/C2=[N+](CCCCCC[N+](C)(C)C)c3ccc4ccccc4c3C2(C)C)C(C)(C)c2c1ccc1ccccc21 ; [Br-] ; [Br-]]

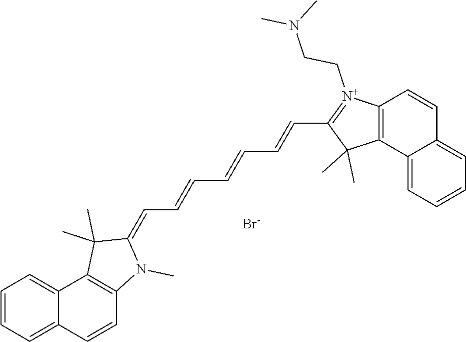 [SMILES: CN(C)CC[N+]1=C(/C=C/C=C/C=C/C=C2\N(C)c3ccc4ccccc4c3C2(C)C)C(C)(C)c2c1ccc1ccccc21 ; [Br-]]

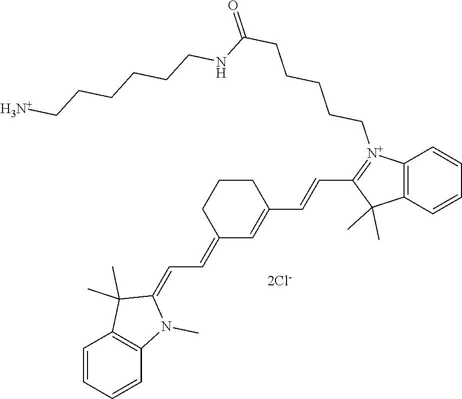 [SMILES: CN1/C(=C\C=C2C=C(/C=C/C3=[N+](CCCCCC(=O)NCCCCCC[NH3+])c4ccccc4C3(C)C)CCC\2)C(C)(C)c2ccccc21]

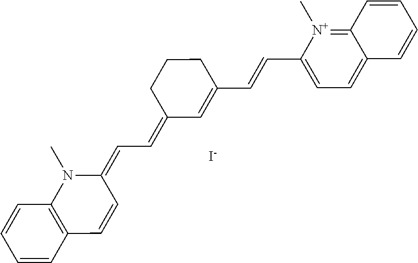 [SMILES: CN1/C(=C/C=C2C=C(/C=C/c3ccc4ccccc4[n+]3C)CCC/2)C=Cc2ccccc21 ; [I-]]

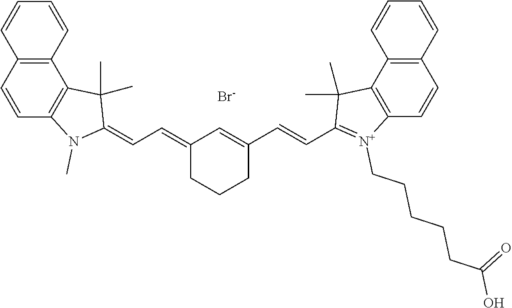 [SMILES: CN1/C(=C/C=C2C=C(/C=C/C3=[N+](CCCCCC(=O)O)c4ccc5ccccc5c4C3(C)C)CCC/2)C(C)(C)c2c1ccc1ccccc21 ; [Br-]]

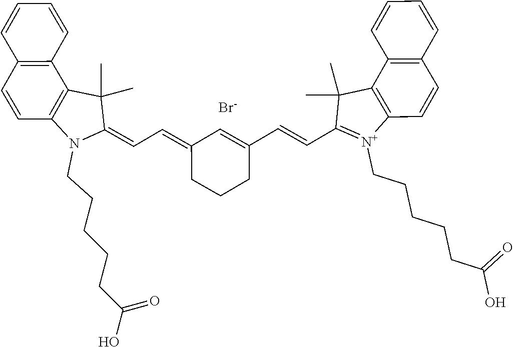 [SMILES: CC1(C)C(/C=C/C2=CC(=C/C=C3/N(CCCCCC(=O)O)c4ccc5ccccc5c4C3(C)C)/CCC2)=[N+](CCCCCC(=O)O)c2ccc3ccccc3c21 ; [Br-]]

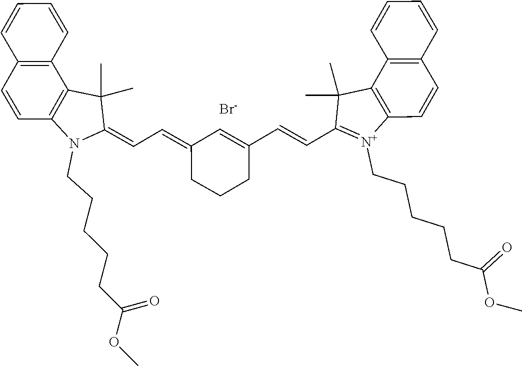 [SMILES: COC(=O)CCCCCN1/C(=C/C=C2C=C(/C=C/C3=[N+](CCCCCC(=O)OC)c4ccc5ccccc5c4C3(C)C)CCC/2)C(C)(C)c2c1ccc1ccccc21 ; [Br-]]

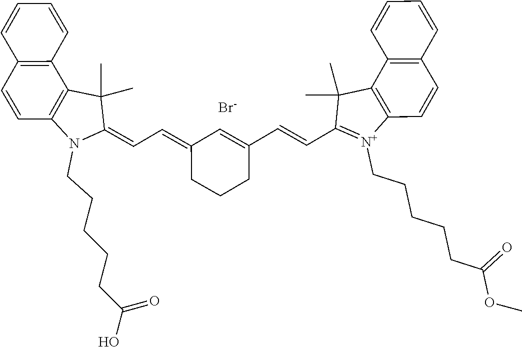 [SMILES: COC(=O)CCCCC[N+]1=C(/C=C/C2=CC(=C/C=C3/N(CCCCCC(=O)O)c4ccc5ccccc5c4C3(C)C)/CCC2)C(C)(C)c2c1ccc1ccccc21 ; [Br-]]

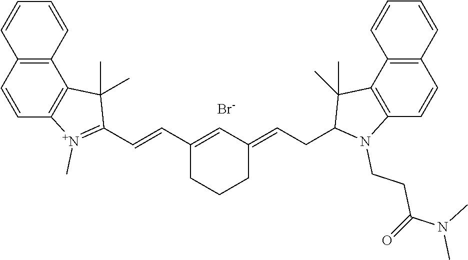 [SMILES: CN(C)C(=O)CCN1c2ccc3ccccc3c2C(C)(C)C1C/C=C1C=C(/C=C/C2=[N+](C)c3ccc4ccccc4c3C2(C)C)CCC/1 ; [Br-]]

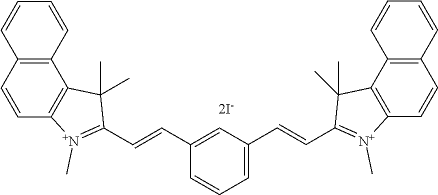

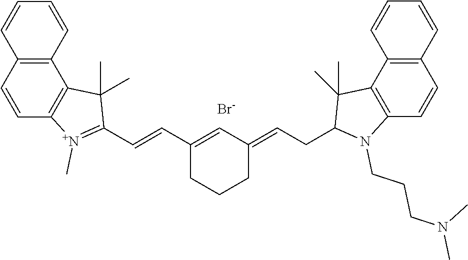 [SMILES: CN(C)CCCN1c2ccc3ccccc3c2C(C)(C)C1C/C=C1C=C(/C=C/C2=[N+](C)c3ccc4ccccc4c3C2(C)C)CCC/1 ; [Br-]]

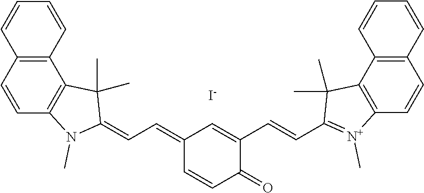 [SMILES: CN1/C(=C/C=C2\C=CC(=O)C(/C=C/C3=[N+](C)c4ccc5ccccc5c4C3(C)C)=C2)C(C)(C)c2c1ccc1ccccc21 ; [I-]]

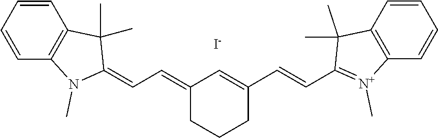 [SMILES: CN1/C(=C/C=C2C=C(/C=C/C3=[N+](C)c4ccccc4C3(C)C)CCC/2)C(C)(C)c2ccccc21 ; [I-]]

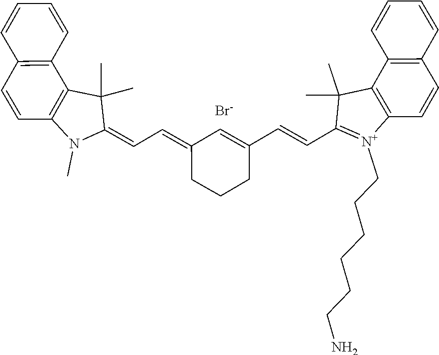 [SMILES: CN1/C(=C/C=C2C=C(/C=C/C3=[N+](CCCCCCN)c4ccc5ccccc5c4C3(C)C)CCC/2)C(C)(C)c2c1ccc1ccccc21 ; [Br-]]

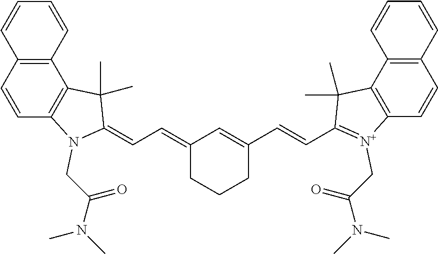 [SMILES: CN(C)C(=O)CN1/C(=C/C=C2C=C(/C=C/C3=[N+](CC(=O)N(C)C)c4ccc5ccccc5c4C3(C)C)CCC/2)C(C)(C)c2c1ccc1ccccc21]

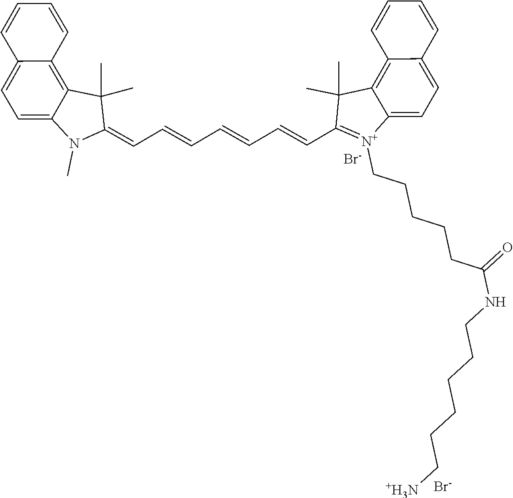 [SMILES: CN1/C(=C/C=C/C=C/C=C/C2=[N+](CCCCCC(=O)NCCCCCC[NH3+])c3ccc4ccccc4c3C2(C)C)C(C)(C)c2c1ccc1ccccc21 ; [Br-] ; [Br-]]

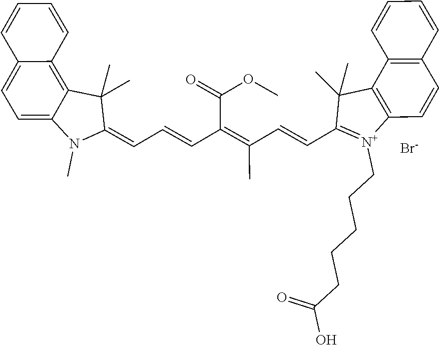 [SMILES: COC(=O)C(/C=C/C=C1/N(C)c2ccc3ccccc3c2C1(C)C)=C(C)\C=C\C1=[N+](CCCCCC(=O)O)c2ccc3ccccc3c2C1(C)C ; [Br-]]

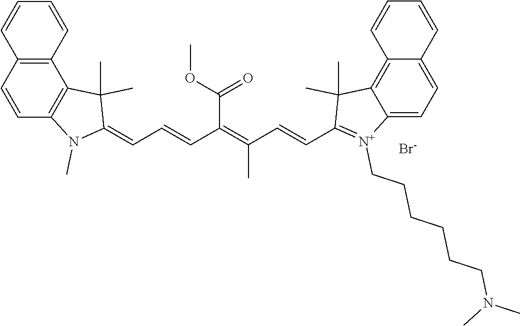 [SMILES: COC(=O)C(/C=C/C=C1/N(C)c2ccc3ccccc3c2C1(C)C)=C(C)\C=C\C1=[N+](CCCCCCN(C)C)c2ccc3ccccc3c2C1(C)C ; [Br-]]

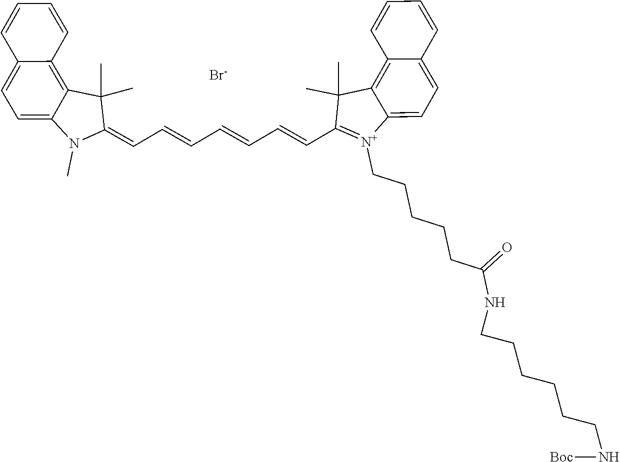 [SMILES: CN1/C(=C/C=C/C=C/C=C/C2=[N+](CCCCCC(=O)NCCCCCCNC(=O)OC(C)(C)C)c3ccc4ccccc4c3C2(C)C)C(C)(C)c2c1ccc1ccccc21 ; [Br-]]

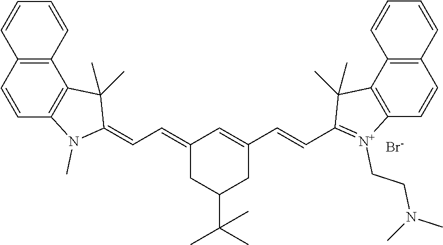 [SMILES: CN(C)CC[N+]1=C(/C=C/C2=CC(=C/C=C3/N(C)c4ccc5ccccc5c4C3(C)C)/CC(C(C)(C)C)C2)C(C)(C)c2c1ccc1ccccc21 ; [Br-]]

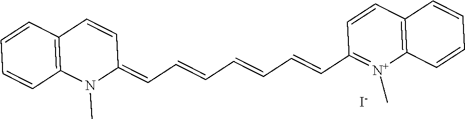 [SMILES: CN1/C(=C/C=C/C=C/C=C/c2ccc3ccccc3[n+]2C)C=Cc2ccccc21 ; [I-]]

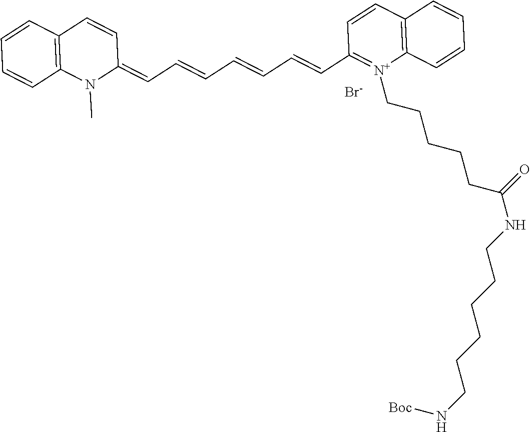 [SMILES: CN1/C(=C/C=C/C=C/C=C/c2ccc3ccccc3[n+]2CCCCCC(=O)NCCCCCCNC(=O)OC(C)(C)C)C=Cc2ccccc21 ; [Br-]]

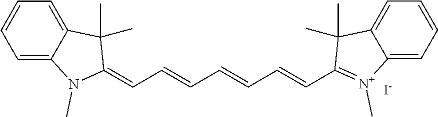 [SMILES: CN1/C(=C/C=C/C=C/C=C/C2=[N+](C)c3ccccc3C2(C)C)C(C)(C)c2ccccc21 ; [I-]]

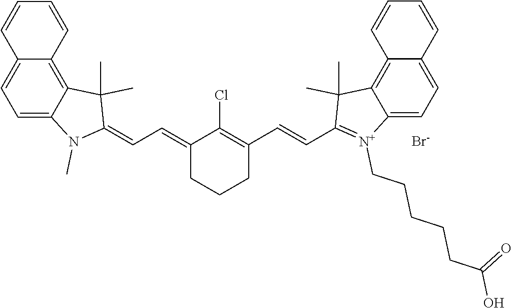 [SMILES: CN1/C(=C/C=C2\CCCC(/C=C/C3=[N+](CCCCCC(=O)O)c4ccc5ccccc5c4C3(C)C)=C2Cl)C(C)(C)c2c1ccc1ccccc21 ; [Br-]]

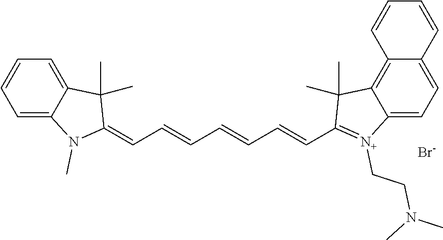 [SMILES: CN(C)CC[N+]1=C(/C=C/C=C/C=C/C=C2/N(C)c3ccccc3C2(C)C)C(C)(C)c2c1ccc1ccccc21 ; [Br-]]

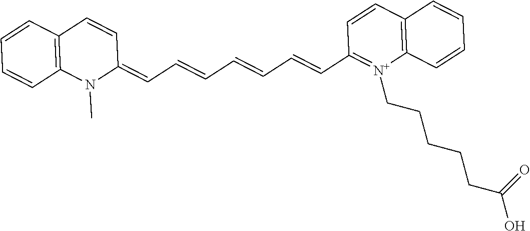 [SMILES: CN1/C(=C/C=C/C=C/C=C/c2ccc3ccccc3[n+]2CCCCCC(=O)O)C=Cc2ccccc21]

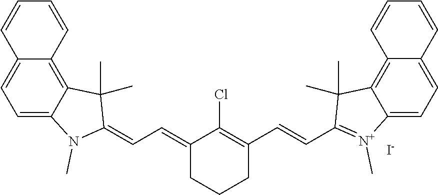 [SMILES: CN1/C(=C/C=C2\CCCC(/C=C/C3=[N+](C)c4ccc5ccccc5c4C3(C)C)=C2Cl)C(C)(C)c2c1ccc1ccccc21 ; [I-]]

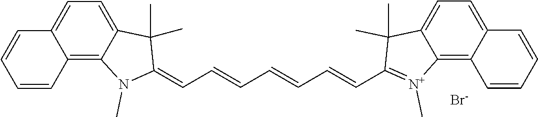 [SMILES: CN1/C(=C/C=C/C=C/C=C/C2=[N+](C)c3c(ccc4ccccc34)C2(C)C)C(C)(C)c2ccc3ccccc3c21 ; [Br-]]

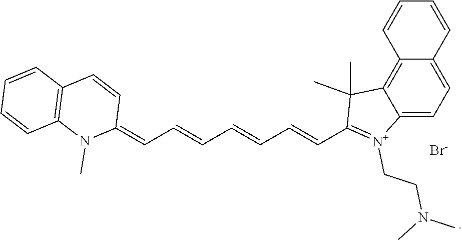 [SMILES: CN(C)CC[N+]1=C(/C=C/C=C/C=C/C=C2\C=Cc3ccccc3N2C)C(C)(C)c2c1ccc1ccccc21 ; [Br-]]

In other embodiments, methods of the present disclosure are provided wherein the compound is further defined as:

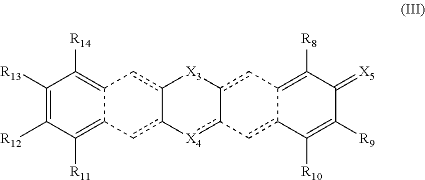

wherein:
   X₃ is —O—, —S—, —C(O)—, —C(S)—, —NRₐ—, or —Si(Rᵤ)(R_{u′})—, wherein:
      Rₐ is hydrogen, alkyl_{(C≤8)}, substituted alkyl_{(C≤8)}, acyl_{(C≤8)}, or substituted acyl_{(C≤8)};
      Rᵤ and R_{u′} are each independently alkyl_{(C≤8)}, substituted alkyl_{(C≤8)}, aryl_{(C≤12)}, or substituted aryl_{(C≤12)};
   X₄ is —N═, —C(R_{b})═, or —C(R_{c})(R_{d})— wherein:
      R_{b} is hydrogen; or
      alkyl_{(C≤8)}, substituted alkyl_{(C≤8)}, aryl_{(C≤12)}, or substituted aryl_{(C≤12)};
      R_{c} and R_{d} are taken together to form a group of the formula:

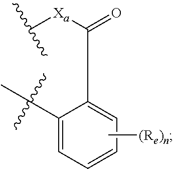

wherein:
   n is 0, 1, 2, 3, or 4;
   Rₑ at each instance is independently hydrogen, halo, hydroxy, or amino; or
   alkyl_{(C≤8)}, alkoxy_{(C≤8)}, alkylamino_{(C≤8)}, dialkylamino_{(C≤8)}, or a substituted version of any of these groups; or
   Xₐ is —O— or —NR_{f}—, wherein:
   R_{f} is hydrogen; or
   alkyl_{(C≤8)}, substituted alkyl_{(C≤8)}, acyl_{(C≤8)}, or substituted acyl_{(C≤8)}; or
   —C(O)NHR_{g}, wherein:
   R_{g} is alkyl_{(C≤8)}, substituted alkyl_{(C≤8)}, alkenyl_{(C≤8)}, or substituted alkenyl_{(C≤8)};
X₅ is NH, O, or S;
R₈ is hydrogen, halo, hydroxy, amino, nitro, or cyano; or
   alkyl_{(C≤8)}, alkoxy_{(C≤8)}, alkylamino_{(C≤8)}, dialkylamino_{(C≤8)}, amido_{(C≤8)}, or a substituted version of any of these groups; or
   —C(O)Rₕ, wherein;
      Rₕ is hydroxy or amino; or
      alkyl_{(C≤8)}, alkoxy_{(C≤8)}, alkylamino_{(C≤8)}, dialkylamino_{(C≤8)}, or a substituted version of any of these groups; and
R₉, R₁₀, R₁₁, R₁₂, R₁₃, and R₁₄ are each independently hydrogen, halo, hydroxy, amino, nitro, or cyano; or
   alkyl_{(C≤8)}, alkoxy_{(C≤8)}, alkylamino_{(C≤8)}, dialkylamino_{(C≤8)}, amido_{(C≤8)}, or a substituted version of any of these groups; or
   —C(O)Rᵢ, wherein;
      Rᵢ is hydroxy or amino; or
      alkyl_{(C≤8)}, alkoxy_{(C≤8)}, alkylamino_{(C≤8)}, dialkylamino_{(C≤8)}, or a substituted version of any of these groups; or
R₉ and R₁₀, R₁₁ and R₁₂, R₁₂ and R₁₃, or R₁₃ and R₁₄ are taken together to form an arene_{(C≤12)}, a substituted arene_{(C≤12)}, a heteroarene_{(C≤12)}, or a substituted heteroarene_{(C≤12)};
   or a pharmaceutically acceptable salt thereof.

In some embodiments, the compound is further defined as:

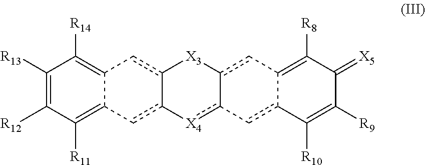

wherein:
   X₃ is —O—, —S—, —C(O)—, —C(S)—, —NRₐ—, or —Si(Rᵤ)(R_{u′})—, wherein:
      Rₐ is hydrogen, alkyl_{(C≤8)}, substituted alkyl_{(C≤8)}, acyl_{(C≤8)}, or substituted acyl_{(C≤8)};
      Rᵤ and R_{u′} are each independently alkyl_{(C≤8)}, substituted alkyl_{(C≤8)}, aryl_{(C≤12)}, or substituted aryl_{(C≤12)};
   X₄ is —N═, —C(R_{b})═, or —C(R_{c})(R_{d})— wherein:
      R_{b} is hydrogen; or
      alkyl_{(C≤8)}, substituted alkyl_{(C≤8)}, aryl_{(C≤12)}, or substituted aryl_{(C≤12)};
      R_{c} and R_{d} are taken together to form a group of the formula:

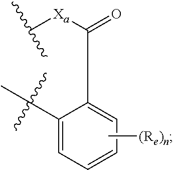

wherein:
   n is 0, 1, 2, 3, or 4;
   Rₑ at each instance is independently hydrogen, halo, hydroxy, or amino; or
   alkyl_{(C≤8)}, alkoxy_{(C≤8)}, alkylamino_{(C≤8)}, dialkylamino_{(C≤8)}, or a substituted version of any of these groups; or
   Xₐ is —O— or —NR_{f}—, wherein:
   R_{f} is hydrogen; or
   alkyl_{(C≤8)}, substituted alkyl_{(C≤8)}, acyl_{(C≤8)}, or substituted acyl_{(C≤8)}; or
   —C(O)NHR_{g}, wherein:
   R_{g} is alkyl_{(C≤8)}, substituted alkyl_{(C≤8)}, alkenyl_{(C≤8)}, or substituted alkenyl_{(C≤8)};
X₅ is NH, ⁺NR′R″, O, or S;
   wherein:
      R′ and R″ are each independently hydrogen, alkyl_{(C≤8)}, or substituted alkyl_{(C≤8)}; or a cell targeting moiety that optionally comprises a linker;
R₈ is hydrogen, halo, hydroxy, amino, nitro, or cyano; or
   alkyl_{(C≤8)}, alkoxy_{(C≤8)}, alkylamino_{(C≤8)}, dialkylamino_{(C≤8)}, amido_{(C≤8)}, or a substituted version of any of these groups; or
   —C(O)Rₕ, wherein;
      Rₕ is hydroxy or amino; or
      alkyl_{(C≤8)}, alkoxy_{(C≤8)}, alkylamino_{(C≤8)}, dialkylamino_{(C≤8)}, or a substituted version of any of these groups; and
R₉, R₁₀, R₁₁, R₁₂, R₁₃, and R₁₄ are each independently hydrogen, halo, hydroxy, amino, nitro, or cyano; or
   alkyl_{(C≤8)}, alkoxy_{(C≤8)}, alkylamino_{(C≤8)}, dialkylamino_{(C≤8)}, amido_{(C≤8)}, or a substituted version of any of these groups; or
   —C(O)Rᵢ, wherein;
      Rᵢ is hydroxy or amino; or
      alkyl_{(C≤8)}, alkoxy_{(C≤8)}, alkylamino_{(C≤8)}, dialkylamino_{(C≤8)}, or a substituted version of any of these groups; or
R₉ and R₁₀, R₁₁ and R₁₂, R₁₂ and R₁₃, or R₁₃ and R₁₄ are taken together to form an arene_{(C≤12)}, a substituted arene_{(C≤12)}, a heteroarene_{(C≤12)}, or a substituted heteroarene_{(C≤12)};
   or a pharmaceutically acceptable salt thereof.

In some embodiments, X₃ is —S—. In some embodiments, X₄ is —N═. In some embodiments, R₁₃ is dialkylamino_{(C≤8)} or substituted dialkylamino_{(C≤8)}. In further embodiments, R₁₃ is dialkylamino(c s), such as dimethylamino. In some embodiments, R₉ is hydrogen. In some embodiments, R₁₀ is hydrogen. In some embodiments, R₁₁ is hydrogen. In some embodiments, R₁₂ is hydrogen. In some embodiments, R₁₄ is hydrogen. In some embodiments, X₅ is ⁺NR′R″. In further embodiments, R′ is alkyl_{(C≤8)} or substituted alkyl_{(C≤8)}. In some embodiments, R′ is alkyl_{(C≤8)}, such as methyl. In some embodiments, R″ is alkyl_{(C≤8)} or substituted alkyl_{(C≤8)}. In further embodiments, R″ is alkyl_{(C≤8)}, such as methyl.

In some embodiments, R″ is a cell targeting moiety. In some embodiments, the cell targeting moiety further comprises a linker.

In some embodiments, the compound is further defined as:

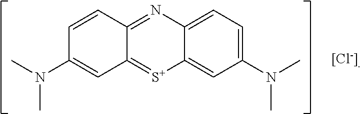

In some embodiments, the energy source is gamma rays, X-rays, ultraviolet (UV) light, visible (Vis) light, near-infrared (NIR) light, infrared light (IR), microwaves, radio waves, electric fields, ionizing radiation, magnetic fields, mechanical forces, ultrasound, or combinations thereof. In some embodiments, wherein the energy source is light. In further embodiments, the energy source is light with a wavelength from about 250 nm to about 2,000 nm. In some embodiments, the wavelength is from about 350 nm to about 1,000 nm. In further embodiments, the wavelength is from about 450 nm to about 900 nm. In some embodiments, the intensity of the energy source is less than 200 mW/cm². In further embodiments, the intensity of the energy is less than 100 mW/cm². In still further embodiments, the intensity of the energy is less than 25 mW/cm².

In another aspect, the present disclosure provides methods of treating a disease or disorder in a patient comprising:
(A) contacting the cell membrane of at least one cell of said patient with a compound, wherein the compound capable of generating a vibronic-driven action and optionally further comprising a cell targeting moiety; and
(B) exposing the compound to an energy source sufficient to generate a vibronic-driven action, wherein the vibronic-driven action is sufficient to disrupt the cell membrane of at least one cell of said patient.

In another aspect, the present disclosure provides compounds for use in the preparation of a medicament for treating a disease or disorder in a patient comprising a compound, wherein the compound capable of generating a vibronic-driven action and optionally further comprising a cell targeting moiety; wherein the vibronic-driven action is sufficient to disrupt the cell membrane of at least one cell of said patient.

In another aspect, the present disclosure provides uses of a compound for treating a disease or disorder in a patient comprising:
(A) contacting the cell membrane of at least one cell of said patient with a compound, wherein the compound capable of generating a vibronic-driven action and optionally further comprising a cell targeting moiety; and
(B) exposing the compound to an energy source sufficient to generate a vibronic-driven action, wherein the vibronic-driven action is sufficient to disrupt the cell membrane of at least one cell of said patient.

In some embodiments, the methods further comprise administering the compound with a therapeutic agent. In some embodiments, the methods comprise administering the compound in combination with the therapeutic agent. In some embodiments, the contacting of step (A) comprises administering the compound. In some embodiments, the compound disrupts the cell membrane allowing the therapeutic agent to enter a cell. In some embodiments, the therapeutic agent is sufficient to treat or prevent the disease or disorder. In some embodiments, the compound is further defined as a compound disclosed in the present disclosure. In some embodiments, the patient is a mammal, such as a human.

In another aspect, the present disclosure provides methods of opening a cell membrane comprising:
(A) contacting the cell membrane with a compound, wherein the compound capable of generating a vibronic-driven action and optionally further comprising a cell targeting moiety; and
(B) exposing the compound to an energy source sufficient to generate a vibronic-driven action, wherein the vibronic-driven action is sufficient to open the cell membrane.

In another aspect, the present disclosure provides compounds for use in opening a cell membrane comprising the compound capable of generating a vibronic-driven action and optionally further comprising a cell targeting moiety; and wherein the vibronic-driven action is sufficient to open the cell membrane.

In another aspect, the present disclosure provides use of a compound for opening a cell membrane comprising:
(A) contacting the cell membrane with a compound, wherein the compound capable of generating a vibronic-driven action and optionally further comprising a cell targeting moiety; and
(B) exposing the compound to an energy source sufficient to generate a vibronic-driven action, wherein the vibronic-driven action is sufficient to open the cell membrane.

In some embodiments, the method comprises treating a disease or disorder. In some embodiments, the method comprises killing one or more cells. In some embodiments, the cell is killed by necrosis. In some embodiments, the cell is a parasitic cell. In further embodiments, the parasitic cell is a bacterial cell, a protozoan cell, a virus, or a fungal cell. In other embodiments, the cell is an abnormal human cell, such as a cancer cell. In some embodiments, the compound is further defined as a compound disclosed in the present disclosure.

In yet another aspect, the present disclosure provides methods of reducing the amount of adipose tissue in a patient comprising contracting the adipose tissue with a compound, wherein the compound capable of generating a vibronic-driven action and optionally further comprising a cell targeting moiety; and exposing the compound to an energy source sufficient to generate a vibronic-driven action, wherein the vibronic-driven action is sufficient to reduce the adipose tissue.

In yet another aspect, the present disclosure provides compounds for use in reducing the amount of adipose tissue in a patient comprising a compound, wherein the compound capable of generating a vibronic-driven action and optionally further comprising a cell targeting moiety; and wherein the vibronic-driven action is sufficient to reduce the adipose tissue.

In yet another aspect, the present disclosure provides uses of a compound for reducing the amount of adipose tissue in a patient comprising contracting the adipose tissue with a compound, wherein the compound capable of generating a vibronic-driven action and optionally further comprising a cell targeting moiety; and exposing the compound to an energy source sufficient to generate a vibronic-driven action, wherein the vibronic-driven action is sufficient to reduce the adipose tissue.

In some embodiments, the adipose tissue is an adipocyte cell. In some embodiments, the adipose tissue is a lipocyte cell. In some embodiments, the adipose tissue is a fat cell. In some embodiments, the method is sufficient to reduce the weight of the patient. In some embodiments, the method is sufficient to reduce the circumference of a part of the body of the patient. In some embodiments, the method further comprises a second exposure to the energy source. In some embodiments, the method further comprises applying the compound a second time, a third time, or more than three times. In further embodiments, the weight of the patient or the circumference of a part of the body of the patient is further reduced.

In still another aspect, the present disclosure provides methods of disrupting a cellular component comprising:
(A) contacting the cellular component with a compound, wherein the compound is capable of generating a vibronic-driven action and optionally further comprising a targeting moiety; and
(B) exposing the compound to an energy source sufficient to generate the vibronic-driven action, wherein the vibronic-driven action is sufficient to disrupt the cellular component.

In still another aspect, the present disclosure provides compounds for use in disrupting a cellular component comprising a compound, wherein the compound is capable of generating a vibronic-driven action and optionally further comprising a targeting moiety; and wherein the vibronic-driven action is sufficient to disrupt the cellular component.

In still another aspect, the present disclosure provides use of a compound for disrupting a cellular component comprising:
(A) contacting the cellular component with a compound, wherein the compound is capable of generating a vibronic-driven action and optionally further comprising a targeting moiety; and
(B) exposing the compound to an energy source sufficient to generate the vibronic-driven action, wherein the vibronic-driven action is sufficient to disrupt the cellular component.

In some embodiments, the cellular component is a carbohydrate or carbohydrate complex. In other embodiments, the cellular component is a protein or protein complex. In still other embodiments, the cellular component is a nucleic acid or nucleic acid complex. In some embodiments, the cellular component is a combination of a nucleic acid, a protein, a carbohydrate, a nucleic acid complex, a protein complex, or a carbohydrate complex. In some embodiments, the cellular component is a cellular component of a prokaryotic cell. In other embodiments, the cellular component is a cellular component of a eukaryotic cell. In some embodiments, the cellular component is a cellular component of a parasitic cell. In further embodiments, the parasitic cell is a bacterial cell, a protozoan cell, a virus, or a fungal cell. In other embodiments, the cellular component is a cellular component of a human cell. In further embodiments, the human cell is an abnormal human cell, such as a cancer cell.

In still yet another aspect, the present disclosure provides intermediate compounds are further defined by the formula:

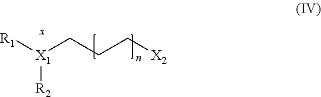

wherein:
x is a positive or negative charge;
n is an integer from 0 to 100;
X₁ is a heteroatom selected from O, N, S, B, P, Ge, As, or Se;
X₂ is hydroxy, amino, or carboxy; or alkylamino_{(C≤12)}, dialkylamino_{(C≤12)}, cycloalkylamino_{(C≤12)}, dicycloalkylamino_{(C≤12)}, alkyl(cycloalkyl)amino_{(C≤12)}, arylamino_{(C≤12)}, diarylamino_{(C≤12)}, alkyl_{(C≤12)}, cycloalkyl_{(C≤12)}, -alkanediyl_{(C≤12)}-cycloalkyl_{(C≤12)}, -alkanediyl(cnig-aralkoxy_{(C≤18)}, heterocycloalkyl_{(C≤12)}, aryl_{(C≤18)}, -arenediyl_{(C≤12)}-alkyl_{(C≤12)}, aralkyl_{(C≤18)}, -arenediyl_{(C≤18)}-heterocycloalkyl_{(C≤12)}, heteroaryl_{(C≤18)}, -heteroarenediyl_{(C≤12)}-alkyl_{(C≤12)}, heteroaralkyl(cis), acyl_{(C≤12)}, alkoxy_{(C≤12)}, or a substituted version of any of these groups;
R₁ and R₂ are each independently alkyl_{(C≤18)}, alkenyl_{(C≤18)}, alkynyl(cis), aryl(cis), aralkyl_{(C≤18)}, heteroaryl_{(C≤18)}, heterocycloalkyl(cis), or a substituted version of any of these groups; or
R₁ and R₂ are taken together to form one, two, three, four, five, or six aliphatic or aromatic rings; comprising at least three carbon atoms and no more than 36 carbon atoms; optionally comprising one, two, three, four, or five nitrogen, sulfur, or oxygen atom.

In some embodiments, X₁ is N. In some embodiments, X₂ is alkyl_{(C≤18)} or substituted alkyl_{(C≤18)}. In other embodiments, X₂ is amino, alkylamino_{(C≤12)}, or substituted alkylamino_{(C≤12)}. In other embodiments, X₂ is carboxy. In some embodiments, R₁ is taken together with R₂ to form one, two, three, four, or five rings. In some embodiments, R₁ is taken together with R₂ to form two, three, or four rings. In some embodiments, R₁ is taken together with R₂ to form three rings. In some embodiments, R₁ is taken together with R₂ to form three rings, wherein one ring is aliphatic and two rings are aromatic. In some embodiments, n is an integer from 1 to 10. In some embodiments, n is an integer selected from 5, 6, or 7. In some embodiments, n is 6.

In some embodiments, the intermediate compounds are further defined as:

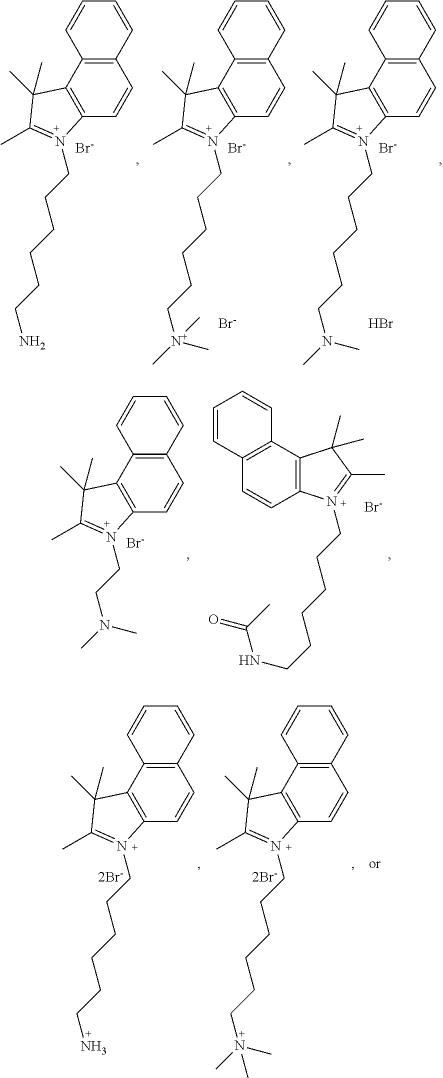

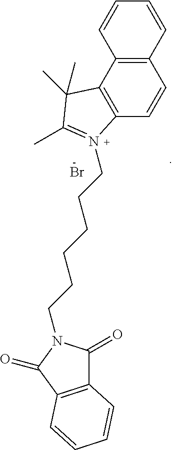

In still yet another aspect, the present disclosure provides compounds of the formula:

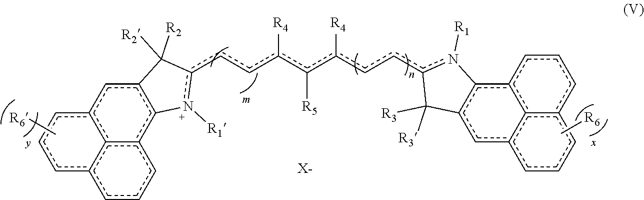

wherein:
R₁ and R₁′ are each independently alkyl_{(C≤8)}, substituted alkyl_{(C≤8)}, or a group of the formula:

-A-NRₐRₐ′Rₐ″
wherein:
   A is an alkanediyl_{(C≤12)} or substituted alkanediyl_{(C≤12)};
   Rₐ and Rₐ′ are each independently hydrogen or alkyl_{(C≤8)}; and
   Rₐ″ is absent, hydrogen, or alkyl_{(C≤8)};
provided at least one of R₁ and R₁′ is a group of the formula: -A-NRₐRₐ′Rₐ″ R₂, R₂′, R₃, and R₃′ are each independently hydrogen, alkyl_{(C≤8)}, or substituted alkyl_{(C≤8)};
R₄ and R₄′ are each independently hydrogen, alkyl_{(C≤8)}, substituted alkyl_{(C≤8)}, or R₄ and R₄′ are taken together as a cycloalkyl group;
R₅ is hydrogen, halo, carboxy, alkyl_{(C≤8)}, substituted alkyl_{(C≤8)}, or —C(O)OR_{b}, wherein R_{b} is alkyl_{(C≤6)} or substituted alkyl_{(C≤6)};
R₆ is hydrogen, amino, halo, hydroxy, or alkyl_{(C≤12)}, alkoxy_{(C≤12)}, alkylamino_{(C≤8)}, dialkylamino_{(C≤8)}, acyl_{(C≤8)}, acyl_{(C≤8)}, acyl_{(C≤8)}, or a substituted version thereof;
m and n are each 0, 1, 2, or 3;
x and y are each independently 0, 1, 2, 3, 4, or 5;
X is a monovalent anion; and
each of the rings is optionally present as either an aromatic or aliphatic ring.

In some embodiments, the compounds are further defined as:

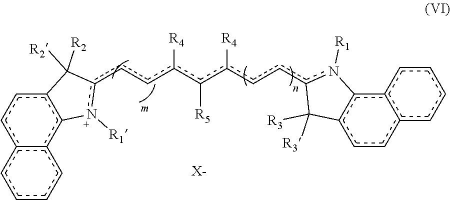

wherein:
R₁ and R₁′ are each independently alkyl_{(C≤8)}, substituted alkyl_{(C≤8)}, or a group of the formula:

-A-NRₐRₐ′Rₐ″
wherein:
   A is an alkanediyl_{(C≤12)} or substituted alkanediyl_{(C≤12)};
   Rₐ and Rₐ′ are each independently hydrogen or alkyl_{(C≤8)}; and
   Rₐ″ is absent, hydrogen, or alkyl_{(C≤8)};
provided at least one of R₁ and R₁′ is a group of the formula: -A-NRₐRₐ′Rₐ″ R₂, R₂′, R₃, and R₃′ are each independently hydrogen, alkyl_{(C≤8)}, or substituted alkyl_{(C≤8)};
R₄ and R₄′ are each independently hydrogen, alkyl_{(C≤8)}, substituted alkyl_{(C≤8)}, or R₄ and R₄′ are taken together as a cycloalkyl group;
R₅ is hydrogen, halo, carboxy, alkyl_{(C≤8)}, substituted alkyl_{(C≤8)}, or —C(O)OR_{b}, wherein R_{b} is alkyl_{(C≤6)} or substituted alkyl_{(C≤6)};
m and n are each 0, 1, 2, or 3;
X is a monovalent anion; and
each of the rings is optionally present as either an aromatic or aliphatic ring.

In some embodiments, the compounds are further defined as:

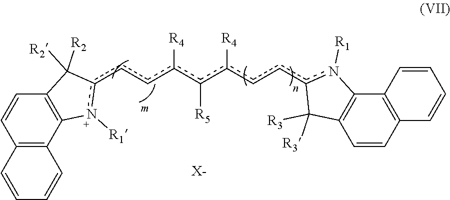

wherein:
R₁ and R₁′ are each independently alkyl_{(C≤8)}, substituted alkyl_{(C≤8)}, or a group of the formula:

-A-NRₐRₐ′Rₐ″
wherein:
   A is an alkanediyl_{(C≤12)} or substituted alkanediyl_{(C≤12)};
   Rₐ and Rₐ′ are each independently hydrogen or alkyl_{(C≤8)}; and
   Rₐ″ is absent, hydrogen, or alkyl_{(C≤8)};
provided at least one of R₁ and R₁′ is a group of the formula: -A-NRₐRₐ′Rₐ″ R₂, R₂′, R₃, and R₃′ are each independently hydrogen, alkyl_{(C≤8)}, or substituted alkyl_{(C≤8)};
R₄ and R₄′ are each independently hydrogen, alkyl_{(C≤8)}, substituted alkyl_{(C≤8)}, or R₄ and R₄′ are taken together as a cycloalkyl group;
R₅ is hydrogen, halo, carboxy, alkyl_{(C≤8)}, substituted alkyl_{(C≤8)}, or —C(O)OR_{b}, wherein R_{b} is alkyl_{(C≤6)} or substituted alkyl_{(C≤6)};
m and n are each 0, 1, 2, or 3; and
X is a monovalent anion.

In some embodiments, the compounds are further defined as:

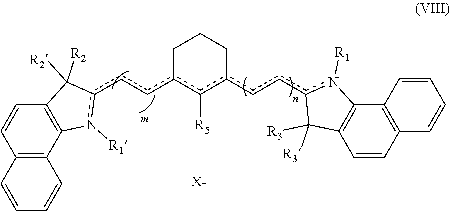

wherein:
R₁ and R₁′ are each independently alkyl_{(C≤8)}, substituted alkyl_{(C≤8)}, or a group of the formula:

-A-NRₐRₐ′Rₐ″
wherein:
   A is an alkanediyl_{(C≤12)} or substituted alkanediyl_{(C≤12)};
   Rₐ and Rₐ′ are each independently hydrogen or alkyl_{(C≤8)}; and
   Rₐ″ is absent, hydrogen, or alkyl_{(C≤8)};
provided at least one of R₁ and R₁′ is a group of the formula: -A-NRₐRₐ′Rₐ″ R₂, R₂′, R₃, and R₃′ are each independently hydrogen, alkyl_{(C≤8)}, or substituted alkyl_{(C≤8)};
R₅ is hydrogen, halo, carboxy, alkyl_{(C≤8)}, substituted alkyl_{(C≤8)}, or —C(O)OR_{b}, wherein R_{b} is alkyl_{(C≤6)} or substituted alkyl_{(C≤6)};
m and n are each 0, 1, 2, or 3; and
X is a monovalent anion.

In some embodiments, the compounds are further defined as:

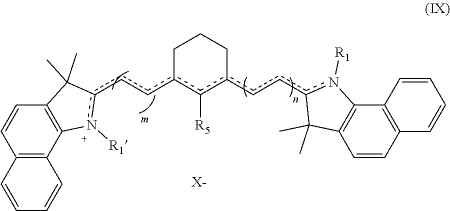

wherein:
R₁ and R₁′ are each independently alkyl_{(C≤8)}, substituted alkyl_{(C≤8)}, or a group of the formula:

-A-NRₐRₐ′Rₐ″
wherein:
   A is an alkanediyl_{(C≤12)} or substituted alkanediyl_{(C≤12)};
   Rₐ and Rₐ′ are each independently hydrogen or alkyl_{(C≤8)}; and
   Rₐ″ is absent, hydrogen, or alkyl_{(C≤8)};
provided at least one of R₁ and R₁′ is a group of the formula: -A-NRₐRₐ′Rₐ″
R₅ is hydrogen, halo, carboxy, alkyl_{(C≤8)}, substituted alkyl_{(C≤8)}, or —C(O)OR_{b}, wherein R_{b} is alkyl_{(C≤6)} or substituted alkyl_{(C≤6)};
m and n are each 0, 1, 2, or 3; and
X is a monovalent anion.

In some embodiments, m is 1. In some embodiments, n is 1. In some embodiments, Rₐ is alkyl_{(C≤8)}. In other embodiments, Rₐ is hydrogen. In some embodiments, Rₐ′ is alkyl_{(C≤8)}. In other embodiments, Rₐ′ is hydrogen.

In some embodiments, the compounds are further defined as:

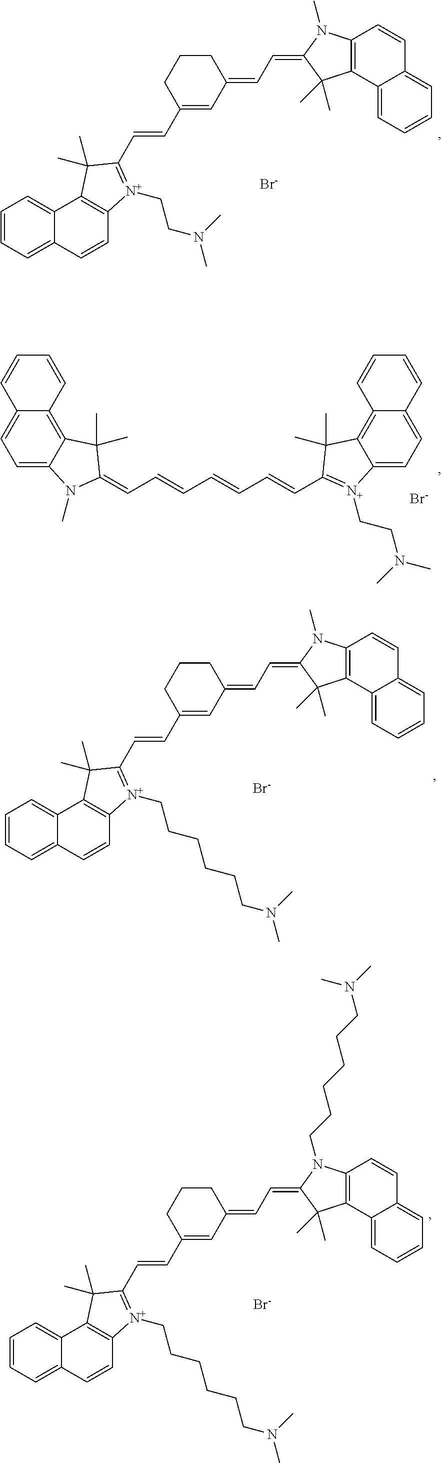

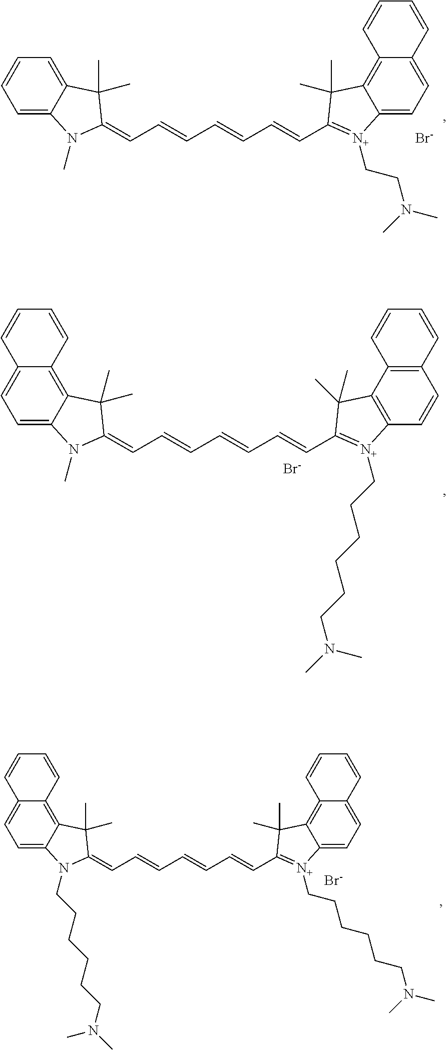 [SMILES: CN(C)CC[N+]1=C(/C=C/C=C/C=C/C=C2/N(C)c3ccccc3C2(C)C)C(C)(C)c2c1ccc1ccccc21 ; [Br-] ; CN(C)CCCCCC[N+]1=C(/C=C/C=C/C=C/C=C2/N(C)c3ccc4ccccc4c3C2(C)C)C(C)(C)c2c1ccc1ccccc21 ; [Br-] ; CN(C)CCCCCCN1/C(=C/C=C/C=C/C=C/C2=[N+](CCCCCCN(C)C)c3ccc4ccccc4c3C2(C)C)C(C)(C)c2c1ccc1ccccc21 ; [Br-]]

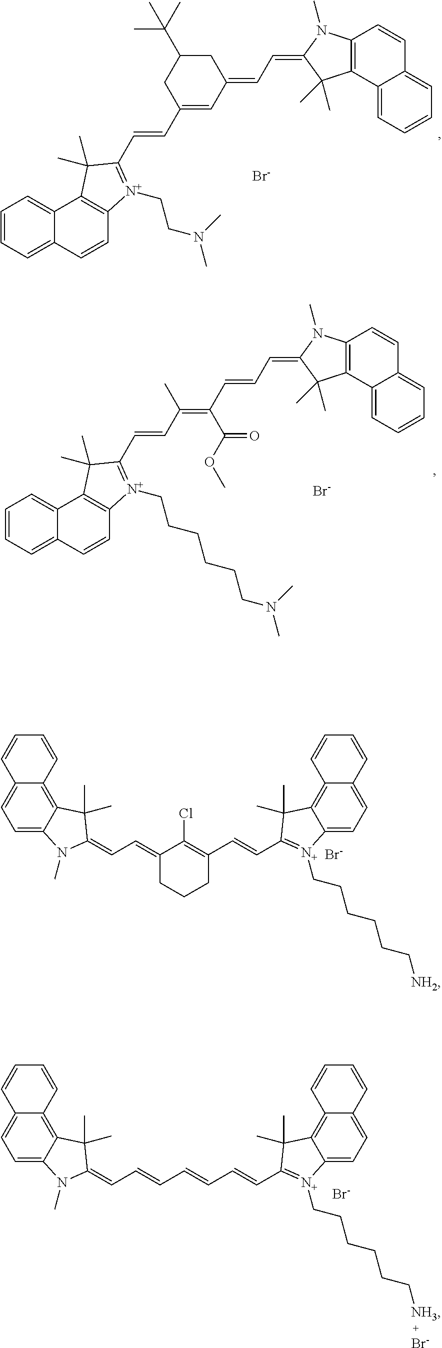

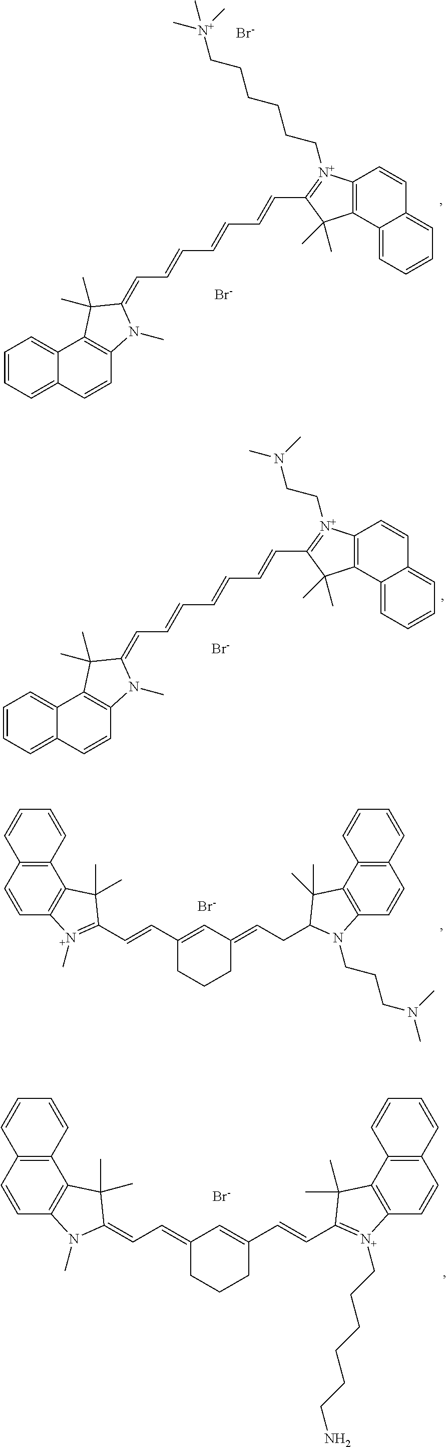

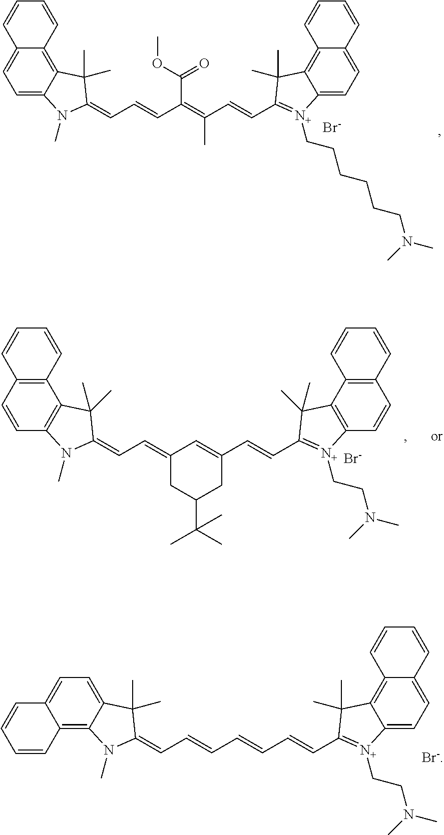

In still yet another aspect, the present disclosure provides methods of disrupting a cell membrane comprising contacting the cell membrane with a compound of formula:

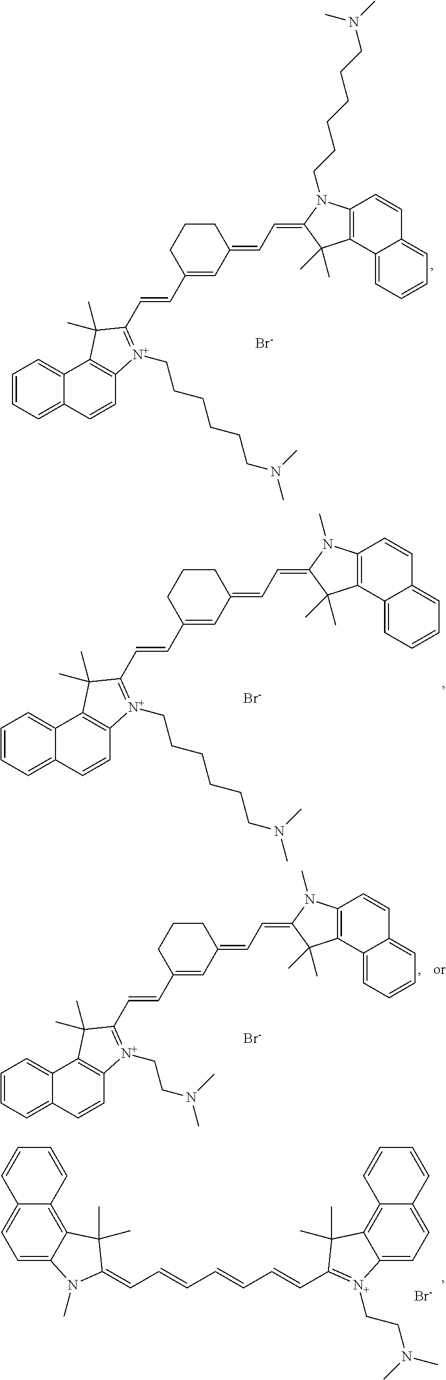

exposing the membrane to an energy source capable of generating vibronic-driven action.

It is contemplated that any method or composition described herein can be implemented with respect to any other method or composition described herein. For example, a compound synthesized by one method may be used in the preparation of a final compound according to a different method.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-C—Schematic representation of bond vibrations represented by arrows using (A) light excitation of a single bond, (B) thermal excitation of multiple bonds and (C) vibronic mode activation (VMA) for whole-molecule excitation with a longitudinal molecular plasmon (LMP, top) or transverse molecular plasmon (TMP, bottom).

FIGS. 2A-B—Cy7.5-amine and Cy7-amine structure and spectra. A) Chemical structure and expected molecular plasmon modes TMP and LMP. B) Absorption spectra of cyanine molecules and assignment of vibrational collective oscillation (vibronic mode), LMP and TMP. The LED light at 730 nm excited mostly the vibrational mode in Cy7.5-amine but not in Cy7-amine.

FIG. 3—Spectral intensity of the LED source and overlapped with the absorption spectrum of cyanine molecules. The spectral intensity of the LED has a λₘₐₓ=734 nm and full width at half maxima (FWHM)=35 nm. Spectral intensity of the LED was provided by the vendor (UHP-F-730 nm, Prizmatix, Israel).

FIGS. 4A-F—Flow cytometry analysis. Fast A375 melanoma cell permeabilization to DAPI (DAPI rapidly enters and stains the membraned-disrupted cells but only slowly on the viable cells) immediately upon treatment with 1 μM Cy7.5-amine or Cy7-amine excited with 730 nm NIR light (80 mW/cm² for 10 min) and analyzed within ˜1 min after the light treatment. A) Control 0.1% DMSO. B) Control 0.1% DMSO+NIR light treatment. C) Cy7-amine. D) Cy7-amine+NIR light treatment. E) Cy7.5-amine. F) Cy7.5 amine+NIR light treatment. The numbers inside the gates (four quadrants) in the flow cytometry plot represent the percentage of cells in each gate: cyanine negative and DAPI negative (left bottom), cyanine positive and DAPI negative (left top), cyanine negative and DAPI positive (right bottom), and cyanine positive and DAPI positive (top right). All the cell suspensions for this study contained 0.1% DMSO which is used to pre-solubilize the cyanine molecule at 8 mM stock solution in 100% DMSO.

FIGS. 5A-B—Flow cytometry analysis. Fast A375 melanoma cell permeabilization to DAPI immediately upon treatment with variable concentration of Cy7.5-amine or Cy7-amine, then excited with 730 nm NIR light (80 mW/cm² for 10 min) and analyzed within ˜1 min after the light treatment. A) Plot showing the permeabilization of cells to DAPI, the red line corresponds to the gate that defines the DAPI impermeable cells (to the left) and the DAPI permeable cells (to the right): a) Control: 0.1% DMSO b) Control: 8 μM Cy7 amine c) 0.5 μM Cy7-amine+NIR light treatment. d) 1 μM Cy7-amine+NIR light treatment. e) 2 μM Cy7-amine+NIR light treatment. f) 4 μM Cy7-amine+NIR light treatment. g) 8 μM Cy7-amine+NIR light treatment. h) 2 μM Cy7.5-amine. i) 0.5 μM Cy7.5-amine+NIR light treatment. j) 1 μM Cy7.5-amine+NIR light treatment. k) 2 μM Cy7.5-amine+NIR light treatment. B) Plot that shows the percentage of DAPI positive cells from flow cytometry analysis. All the cell suspensions contained 0.1% DMSO which is used to pre-solubilize the cyanine molecule at 8 mM stock solution in 100% DMSO.

FIGS. 6A-B—No detection of heat production by Cy7.5-amine under NIR light treatment in the cell suspension (A375 cells). A) Temperature of the cell suspension (A375 cells) with 2 μM Cy7.5-amine and under illumination with 730 nm NIR light (80 mW/cm² for 10 min). The temperature of the media was recorded when the experiment was done at room temperature and also when the cell suspension vessel was submersed in ice bath. The temperature of the cell suspension treated with NIR light and without Cy7.5-amine (DMSO+NIR light) is fully overlapped with temperature profile in the suspension treated with NIR light and containing 2 μM Cy7.5-amine (Cy7.5+NIR light). There was no photothermal effect of Cy7.5-amine beyond the regular and minimal heating caused by the light alone which was only ˜0.5° C. increase. (Averaged temperatures of 3 experiments). B) Percentage of cells permeabilized to DAPI when the treatment was done at room temperature versus when was done placing the cell suspension on ice bath. Experimental groups were: DMSO=0.1% DMSO, DMSO+L=0.1% DMSO+NIR light treatment, Cy7.5=2 μM Cy7.5-amine and Cy7.5+L=2 μM Cy7.5-amine+NIR light treatment. DMSO control contained 0.1% DMSO in the media to pre-solubilize the Cy7.5-amine stock solution at 2 mM and diluted to 1:1000 to achieve a final media concentration of 2 μM Cy7.5-amine with 0.1% DMSO. * p<0.05, ** p<0.01, *** p<0.001

FIGS. 7A-D—ROS scavengers cannot stop the permeabilization of A375 cells to DAPI when treated with 2 μM Cy7.5-amine under illumination with 730 nm NIR light (80 mW/cm² for 10 min). Experimental groups are: DMSO=0.1% DMSO, DMSO+L=0.1% DMSO+NIR light treatment, Cy7.5=2 μM Cy7.5-amine and Cy7.5+L=2 μM Cy7.5-amine+NIR light treatment. A) Effect of 10 mM NAC (N-acetylcysteine). B) Effect of 100 mM TU (thiourea). C) Effect of 2.5 mM SA (sodium azide). D) Effect of ROS scavengers at variable irradiation time of 730 nm NIR light at 80 mW/cm². Five different scavengers were used: TU 100 mM, azide 2.5 mM, NAC 1 mM, Vit C (vitamin C) 5 mM and Met (methionine) 5 mM. DMSO control contains 0.1% DMSO in the media because DMSO is used to pre-solubilize the Cy7.5 amine stock solution at 2 mM and diluted to 1:1000 to get 2 μM Cy7.5 amine in media containing 0.1% DMSO. * p<0.05, ** p<0.01, *** p<0.001 Statistical significance p<0.05.

FIGS. 8A-B—Quantification of cell death by crystal violet assay in A375 cells treated with 2 μM Cy7.5 and 80 mW/cm² of 730 nm NIR light for 10 min. A) Representative microscopy picture of each cell culture well. Experimental groups consist of: Cy7.5+L=2 μM Cy7.5-amine+80 mW/cm² of 730 nm NIR light for 10 min, Cy7.5=2 μM Cy7.5-amine, DMSO+L=0.1% DMSO+80 mW/cm² of 730 nm NIR light for 10 min and, DMSO=0.1% DMSO. B) Plot showing the quantification of the cell viability from the absorbance of crystal violet. * p<0.05, ** p<0.01, *** p<0.001 Statistical significance p<0.05. (Experiment repetitions n=4)

FIGS. 9A-B—Effect of acetic acid on the binding of Cy7.5-amine to A375 melanoma cells and cell permeabilization to DAPI upon NIR light illumination. A) Effect of the concentration of acetic acid in the percentage of cyanine positive cells detected by flow cytometry. B) Effect of the concentration of acetic acid in the percentage of DAPI positive cells detected by flow cytometry. Cells were treated with 2 μM Cy7.5-amine, incubation for 30 min, then illumination with 730 nm light at 80 mW/cm² for 60 s. Method of quantification: Flow cytometry.

FIGS. 10A-F—Effect of indocyanine green (ICG) activated by 730 nm light on A375 melanoma cells. A) Structure of ICG. B) Absorption spectrum of ICG in water. C) Photothermal heating effect of ICG at various concentrations and illuminated by 730 nm light at 80 mW/cm² over time from 0 to 10 min. D) Percentage of permeabilized cells (DAPI positive cells) in the range of ICG concentration from 0 to 16 μM. Cells were illuminated with 730 nm light at 80 mW/cm2 for 10 min. E) Percentage of permeabilized cells (DAPI positive cells) in the range of ICG concentration from 0 to 400 μM. Cells were illuminated with 730 nm light at 80 mW/cm² for 10 min. F) Representative flow cytometry plots showing the DAPI positive gates from where D and E were constructed. The numbers inside the gates (four quadrants) in the flow cytometry plot represent the percentage of cells in each gate: cyanine and DAPI negative (left bottom), cyanine positive and DAPI negative (left top), cyanine negative and DAPI positive (right bottom), and cyanine positive and DAPI positive (top right).

FIGS. 11A-11G—Vibronic Driven Action (VDA) model actuated by plasmon resonance. (A) Absorption spectrum of cyanine-based molecular jackhammer (MJH) and assignment of four major molecular plasmon modes. TMP=transversal molecular plasmon. LMP=longitudinal molecular plasmon. (B) The assignment of the four molecular plasmon modes to the corresponding pictorial model of the electron density distribution in the cyanine molecule. (C) Mechanistic pictorial model of VDA to disassemble lipid bilayers. Step 1: Association of MJH to the lipid bilayer. Step 2: Activation of VDA by NIR light to activate the molecular plasmons and vibrational modes in cyanine molecules. (D) Proposed model of interaction between an aminocyanine and the negatively charged phospholipid cardiolipin (CL). Strategy to build structures of MJH (E) by modifying the side arm, (F) by modifying the position of the fused-benzene on the indole, or (G) by substituting the indole with other resonant structures such as 1-methylquinoline (and the respective resonant form 1-methylquinolinium).

FIG. 12—Chemical structures of cyanine-based MJHs built and utilized in this study. The structures are listed in descending order to least active from left to right and top to bottom.

FIGS. 13A-13C—Vibronic driven action (VDA) to permeabilize human A375 melanoma cells using plasmon-driven MI. (A) Plasmon-driven MJH molecules ordered by the effective concentration needed to permeabilize cells by 50% (VDA IC₅₀). The VDA IC₅₀ of the most active molecule BL-204 is 0.12 μM. The IC₅₀ in the least active molecules BL-206 and ICG is larger than 8 μM. Flow cytometry analysis was used to quantify the percentage of permeabilized cells using DAPI as florescent stain for membrane compromised cells. (B) The molecules are ordered by the plasmonicity index. Here the experimental plasmonicity index was proposed as a parameter that estimates the VDA character in cyanine-based MJH. (C) Correlation plot between the experimental plasmonicity index and the VDA IC₅₀. Light treatment consisted of 730 nm LED light at 80 mW/cm² during 10 min, except for Cy5.5-amine and Cy5-amine a 630 nm LED was used instead to match the absorption of the plasmon resonance of the quadrupole mode in the NIR region.

FIGS. 14A & 14B—Aminocyanine-based MJH targets mitochondria, outer cellular membrane and nuclear membrane in A375 cells. (A) Left: MitroTracker Green, loading concentration C_{loading}=1 μM for 30 min, λₑₓ=488 nm, λₑₘ=500-550 nm. Middle: Cy5.5-amine, C_{loading}=1 μM for 30 min, λₑₓ=640 nm, λₑₘ=663-738 nm. The right panel shows the two (left and middle) overlaid showing in yellow the co-localization of Cy5.5-amine with the mitochondrial stain MitoTracker Green. Notice that Cy5.5-amine stains the outer cellular membrane in red. (B) Left: CellMask Green, loading concentration C_{loading}=5 μg/mL for 30 min, λₑₓ=488 nm, λₑₘ=500-550 nm. Middle: Cy5.5-amine, C_{loading}=1 μM for 30 min, λₑₓ=640 nm, λₑₘ=663-738 nm. The right panel shows the two (left and middle) overlaid showing co-localization of Cy5.5-amine with the cell membrane stain CellMask Green. Notice that Cy5.5-amine stains nuclear membrane in red. The localization of Cy5.5-amine in the mitochondria is likely at the mitochondrial membrane. Scale bar=25 m.

FIGS. 15A & 15B—The effect of plasmon-driven MJH Cy5.5-amine on disassembling cellular membranes and cytoskeleton upon NIR-light activation. (A) Images of A375 cells recorded overtime under treatment with Cy5.5-amine and 640 nm light-exposure. Loading concentration of Cy5.5-amine: C_{loading}=4 μM for 45 min. CellMask Green is added to visualize the plasma membrane in green, loading concentration C_{loading}=5 μg/mL for 30 min, λₑₓ=488 nm, and λₑₘ=500-550 nm. The 640 nm light-exposure times are shown on the top of each image. The numbers on panel at time=0 min is to label the cells under analysis. (B) The area of the cell and the permeabilization of DAPI into A375 cells was recorded overtime. The area of the cells shows that cytoskeleton is shrinking upon activation of the VDA in Cy5.5-amine. The DAPI staining indicates the permeabilization of the plasma membrane. Loading concentration of DAPI: C_{loading}=1 μM, λₑₓ=405 nm, and λₑₘ=425-475 nm. Scale bar=25 m.

FIGS. 16A & 16B—The effect of plasmon-driven MJH Cy5.5-amine on disassembling of GFP-labeled cytoskeleton upon light activation in A375 cells. (A) Images of A375 cells recorded overtime under treatment with Cy5.5-amine and 640 nm light-exposure. Loading concentration of Cy5.5-amine: C_{loading}=4 μM for 45 min. CellLight Actin-GFP, BacMan 2.0 from Invitrogen, was used to label actin with green fluorescent protein (GFP) in A375 cells. GFP imaging at λₑₓ=488 nm, and λₑₘ=500-550 nm. The 640 nm light-exposure times are shown in each image. The numbers on panel at time=0 min is to label the cells under analysis. (B) The area of the cell and the permeabilization of DAPI into A375 cells was recorded overtime. The area of the cells shows that cytoskeleton is shrinking upon activation of the VDA in Cy5.5-amine. The DAPI staining indicates the permeabilization of the plasma membrane. Loading concentration of DAPI: C_{loading}=1 μM, λₑₓ=405 nm, and λₑₘ=425-475 nm. Scale bar=25 m.

FIGS. 17A-17C—Plasmon-driven MJH Cy5.5-amine disassembles and breaks plasma membranes upon light activation. Images of A375 cells recorded before and after 640 nm light-exposure (exposure time=10 min). Loading concentration of Cy5.5-amine: C_{loading}=4 μM for 45 min. CellMask Green is added to visualize the plasma membrane in green, loading concentration C_{loading}=5 μg/mL for 30 min, λₑₓ=488 nm, and λₑₘ=500-550 nm. The DAPI staining indicates the permeabilization of the plasma membrane. Loading concentration of DAPI: C_{loading}=1 μM, λₑₓ=405 nm, and λₑₘ=425-475 nm. (A) Pictures of A375 cells before light treatment. (B) Pictures of A375 cells after 640 nm light-exposure for 10 min. (C) Cells were refocused for better observation of broken cell membranes after the 640 nm light-treatment was completed. Scale bar=10 m.

FIGS. 18A-18D—Lethal concentration of plasmon-driven MJHs at short contact time (50 min) in A375 cells. Clonogenic assay conducted in the presence of (A) molecule BL-204, (B) molecule BL-141-2 and (C) molecule Cy7-amine. (D) Quantification of the surviving cells and estimation of the lethal concentration to kill 50% of the cells (VDA LC₅₀). Letter L stands for light treatment. A375 cells were incubated with the molecules for 50 min, and then treated with light (730 nm light at 80 mW/cm² for 10 min). Immediately after the treatment the molecules in the media were removed by exchanging with clean media and then the cells were culture for colony formation during 7 days. The clonogenic assay was conducted by triplicate (n=3). The error bars are the standard errors.

FIGS. 19A-19D—Predicted octanol-water partition coefficient (log P value) in cyanine-based MI. The molecules are ordered from more VDA active to less VDA active from bottom to top. (A) Octanol-water partition coefficients (log P values) of molecules in the charged state of the side arm (protonated or ionized). (B) Octanol-water partition coefficients (log P values) of molecules in the neutral state at the side arm (unprotonated or unionized). (C) Correlation plot between VDA IC₅₀ and the log P values for molecules in the charged state. (D) Correlation plot between VDA IC₅₀ and the log P values of molecules in the neutral state. Data shows that most VDA active compounds are not necessarily the most lipophilic (affine to lipid membranes) such as GL-308-2 and BL-204. The values were calculated using a log P value predictor software.

FIGS. 20A-20D—Calculated TD-DFT absorption spectrum and induced charge density plots of the molecular plasmons in Cy7.5-amine. (A) Total and partial, by the orientation of the electric field component (Ei), absorption spectra calculated by time-dependent density-functional theory (TDDFT) using the Lanczos approach. The electric field is used to simulate the optical excitation of the Cy7.5-amine. The partial components of the spectrum are oriented along the transversal molecular plasmon resonance (red), longitudinal (blue) and perpendicular (green) axis of Cy7.5-amine. (B) Absorption spectra comparison between the experimental (top) and the TDDFT calculation (bottom). The dashed lines represent the position of the wavelengths at which the induced charge density maps were calculated for molecular plasmon resonances. The experimental shoulder at 730 nm for the vibronic mode in Cy7.5-amine is observed at 750 nm in the theoretical transversal component of the spectrum, but it is less obvious in the total spectrum. (C) Total induced charge densities [Δρ(r)] at 409, 530, 750 and 809 nm wavelengths for molecular plasmon resonance. (D) Induced charge densities [Δρ(r)] by electric field (Ei) components at 409, 530, 750 and 809 nm wavelengths oriented along the transversal, longitudinal and perpendicular axis of Cy7.5-amine. The vectors on the rightmost represent the orientation of the electric filed components. The long alkyl-amine arm in Cy7.5-amine structure was not included in the electronic structure calculation because it has negligible contributions to the conjugation of the core structure. Instead, a methyl group was substituted for the long alkyl-amine in Cy7.5-amine.

FIGS. 21A-21C—Molecular jackhammer (MJH) model and summary of structures used in this study. (A) General proposed working mechanism of MJH opening cell membranes. (B) Important MJH structural elements. LMP=longitudinal molecular plasmon. TMP=transversal molecular plasmon. The strength of the molecular plasmon (VDA) is expected to be proportional to the length of the π-conjugation. The π-conjugation can be increased in two ways: 1) increasing the length of polymethine bridge and 2) increasing the size of the polycyclic aromatic hydrocarbon (PAH) fused to the indole. The purple color is to highlight the polymethine bridge. The cyanines are named by the number of carbons in the polymethine bridge, in the example it is C7. The red color is to highlight the structure of the indole, and the orange color is for the benzoindole. The heptamethine bridge (C7) can be chemically conjugated with indole to form Cy7 or with benzoindole to form Cy7.5. These structural elements, polymethine and indole or benzoindole, hybridize to from a coupled system with the molecular plasmon-dominated longitudinally by the polymethine bridge (LMP in purple) and transversally by the indole or benzoindole (TMP in green). However, these structures are hybridized and the electronic conjugation of the benzoindole influences the polymethine bridge and vice versa. (C) Summary of structures in this study. The observed effect on the cell killing is summarized for each structure, and each lists the common name of the conjugate backbone. The addend function is listed for each.

FIGS. 22A-22C—Binding of MJH into the external cellular membrane and into internal organelle membranes of A375 human melanoma cell line. (A) Fluorescence confocal microscopy imaging of MJH (Cy5.5-amine and Cy5-amine) loaded in A375 cells. C_{loading}=2 μM, incubation time=30 min, λₑₓ=640 nm, λₑₘ=663-738 nm. The arrows are to indicate the position of the external cellular membrane and its staining with cyanine dyes (MJH). An average of 75 cells were analyzed in each condition in the confocal microscope in 5 different locations. Representative images are shown. (B) Effect of the concentration of acetic acid in the binding of Cy7.5-amine to the A375 cells using flow cytometry analysis for quantification. Average of two experiments is shown (n=2). (C) Effect of the concentration of acetic acid in the percentage of DAPI permeabilized cells using flow cytometry analysis for quantification. Cells were treated with 2 μM Cy7.5-amine, incubation for 30 min, then were illuminated with 730 nm light at 80 mWcm⁻² for 60 s. Since acetic acid protonates the phosphates in the phospholipids, the Cy7.5-amine is not able to bind efficiently to lipid membranes. The lower binding of Cy7.5-amine to the cells is reflected in the lower permeabilization of the cells upon NIR light excitation. Average of two experiments is shown (n=2) Scale bars=25 μm.

FIG. 23—Flow cytometry analysis of Cy7.5-amine activity inhibition for permeabilization of cells using Cy5-amine as competitor molecule. Cy5-amine interacts with the cells and competes with Cy7.5-amine. The 730 nm LED excites Cy7.5-amine but does not excites Cy5-amine (the excitation of Cy5-amine using 730 nm LED is almost negligible). The concentration of Cy7.5-amine was 1 μM and Cy5-amine was 8 μM. The illumination was done with 730 nm LED, 80 mWcm⁻² for 10 min. The red line is to indicate the gate level for DAPI positive cells. The black dotted line is to guide the shifting position of the peak in presence of Cy5-amine. Three independent flow cytometry experiments were conducted (n=3), a representative is presented.

FIGS. 24A-24G—Absorption spectrum of MJH and confocal fluorescence microscopy of A375 cells in the presence of MJH. (A) Absorption spectrum of MJH showing the position of the excitation lasers that were used in the confocal microscope (λₑₓ=405 nm with λₑₘ=425-475 nm or λₑₓ=640 nm with λₑₘ=663-738 nm). (B) Expansion of the x and y axis of the absorption spectrum to observe the expected TMP (transversal molecular plasmon). The Cy7.5-amine shows a strong TMP (strong hybridization of longer C7 heptamethine bridge and larger benzoindole). Cy7-amine shows a weaker TMP and slightly shifted to ˜375 nm because the C7 is hybridized to the smaller indole. Cy5.5-amine shows a strong TMP (larger benzoindole) but shifted to ˜360 nm because of the hybridization with a weaker LMP (shorter C5 pentamethine). Cy5-amine shows little TMP because of the poor hybridization of the shorter C5 and smaller indole; this is the weakest combination because of the poor plasmonicity on both components. (C) Cells in the absence of dyes. (D) Cells in the presence of 2 μM Cy7.5-amine, 30 min of incubation. The emission of TMP mode can be observed at λₑₓ=405 nm in blue color. The excitation at 640 nm excites the tail of the LMP and produces a weak emission that is present but is difficult to see in the picture. (E) Cells in the presence of 2 μM Cy7-amine, 30 min of incubation. The emission from the LMP in Cy7-amine, since it is blue-shifted relative Cy7.5-amine, can be observed as red λₑₘ=663-738 nm). (F) Cells in the presence of 2 μM Cy5.5-amine, 30 min of incubation. The emission from the LMP is clearly visible in red. (G) Cells in the presence of 2 μM Cy5-amine, 30 min of incubation. The emission from LPM is clearly visible in red. The emission from TMP (at λₑₓ=405 nm) is not observed or is very weak in signal in E-G panel since the TMP is not present or shifted to other wavelengths on those molecules (Cy7, Cy5.5, and Cy5). An average of 75 cells were analyzed in each condition in the confocal microscope in 5 different locations. Representative images are shown. Scale bars=25 μm.

FIGS. 25A-25F—Excitation of the 680 nm vibronic shoulder in Cy7 improves the MJH effect for opening cell membranes in A375 cells. (A) Absorption spectra of Cy7-amine and overlaid with the spectral output of two LED lights: 730 nm and 680 nm. (B) Structure of Cy7-amine. (C) Flow cytometry analysis to measure the permeabilization of A375 cells in the presence of Cy7-amine without light. (D) Flow cytometry analysis to measure the permeabilization of A375 cells in the presence of Cy7-amine with 730 nm LED activation. (E) Flow cytometry analysis to measure the permeabilization of A375 cells in the presence of Cy7-amine with 680 nm LED activation. In all the flow cytometry analyses the red line represents the gating to discriminate between DAPI negative and positive cells (permeable). In all the cases the incubation with the cyanine was for 30 min and irradiation was with an equal light dose of 80 mWcm⁻² for 10 min. The light dose was calibrated with an Optical Power Meter from Thorlabs, sensor model S302C and console model PM100D. (F) Percentage of permeabilized cells, the numbers are obtained from the flow cytometry. 10,000 cells are analyzed per each concentration.

FIGS. 26A-26E—Cell membrane permeabilization dependence with the expected strength of the MJH. (A) Structures of MJH and classification according to their expected relative strength in the vibronic-driven action (plasmonicity) based on the extension of the indole with polycyclic aromatic hydrocarbons (PAH) and the length of the π-conjugation in the polymethine bridge. (B) The absorption spectra of each MJH overlaid with the specific LED light that was used for illumination in this experiment. (C) Flow cytometry analysis to measure the permeabilization of A375 cells in the presence of each MJH without illumination. The red line represents the gating to discriminate between DAPI negative and positive cells (permeable). (D) Flow cytometry analysis to measure the permeabilization of A375 cells in the presence of each MJH with specific LED illumination for each cyanine. The red line represents the gating to discriminate between DAPI negative and positive cells (permeable). (E) Plots of the percentage of permeabilized cells, the numbers are extracted from the flow cytometry analysis. 10,000 cells were analyzed in each concentration. The incubation of the MJHs with the cells was 30 min. The light-treated samples were illuminated with the same light dose of 80 mWcm⁻² for 10 min. The shifting on the DAPI fluorescence intensity for the Cy7.5-amine before illumination correspond to the fluorescence emission from Cy7.5-amine which is excited with the same laser that excites DAPI (λₑₓ=405 nm). FIG. 22 supports the conclusion that this observation correlates with the confocal microscopy data. This fluorescence level from Cy7.5-amine can be regarded as background fluorescence and not cell membrane permeabilization. This main factor was considered to draw the position of the gating (red line) to discriminate between DAPI positive cells and DAPI negative cells.

FIGS. 27A-27F—Cell membrane permeabilization of A375 cells over time while the cells were irradiated under the confocal microscope. The permeabilization of DAPI into the cells was recorded as a function of time (rightmost column). (A) Cells in the presence of 4 μM Cy5.5-amine without laser irradiation. (B) Cells in the presence of 4 μM Cy5.5-amine with 640 nm laser irradiation. (C) Cells in the presence of 4 μM Cy5-amine without laser irradiation. (D) Cells in the presence of 4 μM Cy5-amine with 640 nm laser irradiation. (E) Cells in the presence of cell-membrane-targeting 4 μM DiD dye without laser irradiation. (F) Cells in the presence of 4 μM DiD dye with 640 nm laser irradiation. The irradiation times are shown in each image. For the activation of the MJH effect, the cells were irradiated at λₑₓ=640 nm, 25% power. The pictures were recorded every 1 min at low photoactivation powers and short exposures (λₑₓ=640 nm, 5% power, 2.1 s per a single frame) to maintain as intact as possible the non-irradiated controls. The incubation of the cyanines with the cells before irradiation was 30 min. Representative confocal images of each condition are shown. The quantification on the rightmost is the average of 14 cells (n=14), the error bars are the standard error. All scale bars=25 μm.

FIGS. 28A-28G—Temperature of the cell suspension while under light treatment. Temperature on the cell killing experiment using Cy7.5-amine. (A-B, D-E) No detection of heat production by Cy7.5-amine under NIR light treatment in the cell suspension (A375 cells) above the control. Temperature of the cell suspension (A375 cells) with 2 μM Cy7.5-amine and under illumination with 730 nm NIR light (80 mWcm⁻² for 10 min). The temperature of the media was recorded when the experiment was done at room temperature (A-C) and when the cell suspension was placed in an ice bath (D-F). A picture of the experimental set up when done at room temperature is shown in C and in ice bath is shown in F. In D “water+ice” is the increase of the temperature because of the melting of ice without irradiation. In E the change of temperature is corrected by subtracting the temperature increase due to the melting of ice without illumination. The temperature of the cell suspension treated with NIR light and without Cy7.5-amine (DMSO+NIR light) correlates well with temperature profile in the suspension treated with NIR light containing 2 μM Cy7.5-amine (Cy7.5+NIR light). There is no photothermal effect of Cy7.5-amine beyond the minimal heating caused by the light alone of ˜0.5° C. (G) Percentage of cells permeabilized to DAPI when the treatment was done at room temperature versus when was done placing the cell suspension on ice bath. In A and B 3 independent samples were processed and measured (n=3). In D and E 4 independent samples were processed and measured (n=4). In G 3 independent samples were processed and measured by flow cytometry (n=3). In all the plots the error bars are the standard deviation. t-test, two-tail, * p<0.05, ** p<0.01, *** p<0.001 Statistical significance p<0.05, ns=not significant.

FIGS. 29A-29D—ROS effects on the cell killing using Cy7.5-amine. ROS scavengers do not retard the permeabilization of A375 cells to DAPI when treated with 2 μM Cy7.5-amine under illumination with 730 nm NIR light (80 mWcm⁻² for 10 min). (A) Effect of 10 mM NAC (N-acetylcysteine). (B) Effect of 100 mM TU (thiourea). (C) Effect of 2.5 mM SA (sodium azide). (D) Effect of ROS scavengers at variable irradiation time of 730 nm NIR light at 80 mWcm⁻². Five different scavengers were used: TU 100 mM, azide 2.5 mM, NAC 1 mM, Vit C (vitamin C) 5 mM and Met (methionine) 5 mM. DMSO control contains 0.1% DMSO in the media because DMSO is used to pre-solubilize the Cy7.5-amine stock solution at 2 mM and diluted to 1:1000 to obtain 2 μM Cy7.5-amine in media containing 0.1% DMSO. Experimental groups are: DMSO=0.1% DMSO, DMSO+L=0.1% DMSO+NIR light treatment, Cy7.5=2 μM Cy7.5-amine and Cy7.5+L=2 μM Cy7.5-amine+NIR light treatment. In all the plots 3 independent samples were processed and analyzed by flow cytometry (n=3). In all the errors bars are the standard deviation. t-test, two-tail, * p<0.05, ** p<0.01, *** p<0.001 Statistical significance p<0.05, ns=not significant.

FIGS. 30A-30F—Quantification of ROS and singlet oxygen (SO) levels and their effect on the cell killing using Cy7.5-amine versus the cell-membrane-targeting DiR dye. (A) Measurement of ROS levels in A375 cell suspensions using 2′,7′-dichlorodihydrofluorescein diacetate as the ROS probe in the presence of 2 μM Cy7.5-amine versus 2 μM DiR and 20 μM DiR with and without light (L). (B) Percentage of DAPI positive cells as a function of the concentration quantified from the flow cytometry analysis. DiR that produced 10-20-fold more ROS than Cy7.5-amine did not permeabilize the A375 cells. This continues to support that the DiR structure is a weaker MJH with poorer VDA (See FIG. 21) And more importantly, that ROS is not responsible for the permeabilization of DAPI into the cells. (C) Quantification of SO levels by the decomposition rate of DPBF (1,3-diphenylisobenzofuran) under light illumination in the presence of four cyanine dyes: 2.6 μM Cy7.5-amine, 2.6 μM Cy7-amine, 2.6 μM DiR and 2.6 μM ICG. DiR and ICG produces more SO than Cy7.5-amine or Cy7-amine yet DiR was unable to permeabilize the cells. (D) ROS levels in cells in the presence of Cy7.5-amine versus Cy7-amine. Cy7.5-amine and Cy7-amine produced approximately the same levels of SO and ROS yet Cy7.5-amine is a much stronger MJH in cell permeabilization (FIG. 4). (E) Shutting down the levels of SO generation by adding thiourea (TU=100 mM) or sodium azide (SA=2.5 mM) or a combination of TU 100 mM and SA 2.5 mM into the 2 μM Cy7.5-amine solution under LED illumination. (F) Effect of ROS scavenger combo (100 mM TU and 2.5 mM SA) on the percentage of DAPI positive cells as quantified from the flow cytometry analysis in the presence of 2 μM Cy7.5-amine with and without illumination: no difference observed in the cell permeabilization to DAPI. Unless otherwise specified, the light irradiations doses were 80 mWcm⁻² for 10 min using a 730 nm LED. Except, in B the LED light (L) was a 740 nm light from Keber Applied Research Inc. at the same dose of 80 mWcm⁻² for 10 min. In panel A, the average is from 4 treated and measured samples (n=4); for this the samples are quadruplicated in one 96 well. In panel B 5,000 cells are analyzed by flow cytometry for each concentration (one sample per each concentration n=1). In panel C the number of samples is n=1. In panel D, 12 samples are processed and analyzed in each condition from 3 independent experiments, and in each experiment each condition is quadruplicated in a 96 well plate (n=3 experiments×4 samples per condition=12). In panel E the number of samples n=1. In panel F, n=3 independent samples. In all the error bars are the standard deviations.

FIGS. 31A-31D—Quantification of cell death by crystal violet assay and clonogenic assay. A375 cells treated with Cy7.5-amine and 80 mWCm⁻² of 730 nm NIR light for 10 min. (A) Representative microscopy picture of each condition in the crystal violet assay (n=4). Experimental groups consist of. Cy7.5-amine+L=2 μM Cy7.5-amine+80 mWCm⁻² of 730 nm NIR light for 10 min; Cy7.5-amine=2 μM Cy7.5-amine; DMSO+L=0.1% DMSO+80 mWcm⁻² of 730 nm NIR light for 10 min; and DMSO=0.1% DMSO. (B) Crystal violet assay. Plot showing the quantification of the cell viability from the absorbance of crystal violet. Error bars are the standard deviations. Sample repetitions n=4 for each condition in a 24 well plate (independent samples). (C) Clonogenic assay. Representative pictures showing the growth of cell colonies in the controls (0.1% DMSO with or without light) and complete eradication of A375 cells when treated with 1 μM Cy7.5-amine+light (80 mWCm⁻² of 730 nm NIR light for 10 min). (D) Clonogenic assay. Quantification of the number cells forming colonies. The survival is the percentage of cells that formed colonies. Error bars are the standard deviation. The results are normalized relative to the DMSO control. Sample repetitions n=3 (independent samples). t-test, two-tail, * p<0.05, ** p<0.01, *** p<0.001 Statistical significance p<0.05.

FIGS. 32A-32H—Therapeutic effect of Cy7.5-amine in the treatment of tumors in mice. (A) Pictures of the set up and the conditions to treat B16-F10 melanoma tumors in C57BL/6 mice. The Cy7.5-amine was applied by intratumoral injection of 50 μL solution containing 0.16 mg mL⁻¹ in PBS solution with 0.1% DMSO. 0.1% DMSO in PBS is use as a control. (B) Recording of the temperature on the mouse skin using a NIR camera while being treated with Cy7.5-amine or DMSO and irradiated with a 730 nm LED at 150 mWCm⁻² for 5 min. The inset in each picture shows the temperature on the tumor area. On the top of the picture is the time of illumination. (C) Temperature of the tumor area as a function of time. There is little difference with respect to the control. Four independent measurements n=4. Error bars are the standard deviation. (D) Effect of the treatment on the size of the B16-F10 tumors as a function of time. Number of mice per group, n=4. Error bars are the standard error. (E) Representative figure of mice with B16-F10 tumors and the response to treatment. The treated tumors become dry with a necrotic spot upon treatment with Cy7.5-amine+150 mWCm⁻² for 5 min of 730 nm LED. (F) Schematics of the conditions and treatment schedule on A375 human melanoma tumors in nude mice. The Cy7.5-amine was applied by intratumoral injection of 50 μL solution containing 0.16 mg mL⁻¹ in PBS solution with 0.1% DMSO. 0.1% DMSO in PBS is use as a control. Three doses of light were applied in combination with the solutions (Cy7.5-amine or DMSO). (G) Effect of the treatment on the size of the A375 tumors as a function of time. Number of mice per group, n=10. We learned in the process that 150-215 mWCm⁻² produced a mild therapeutic effect in A375 tumor models. With 300 mWCm⁻² a strong effect on the regression of the tumor size was observed. The drop at 60 days in the Cy7.5-amine+L group was due to the exclusion of the euthanized mice. Error bars are the standard error. t-test, two-tail, * p<0.05, ** p<0.01, *** p<0.001. Statistical significance p<0.05. (H) Survival of mice with A375 tumors under the various treatments (n=10 mice per group). 60% of the (6 out of 10) mice survived and 50% are tumor free in the Cy7.5-amine+light (L) group. Two mice (20%) in the Cy7.5-amine group survived.

FIGS. 33A-33D—Flow cytometry data processing and gating strategy. Data analysis was done using FlowJo version 10.5.3. (A) Selection of the cell population by plotting forward scattering area (FSC-A) vs side scattering area (SSC-A). (B) Selection of single cells by plotting FSC-A versus FSC-height (FSC-H). (C) Gating of DAPI positive cells in a control sample containing 0.1% DMSO. (D) Application of the same gate conditions as shown in c to a DAPI positive sample which was treated with Cy7.5-amine and 730 nm light. The number in the inset in each gate shows the percentage of positive cells.

### DESCRIPTION OF ILLUSTRATIVE EMBODIMENTS

Provided herein are methods and compounds that have been demonstrated to disrupt membranes. These methods and compounds may be useful to disrupt human cell membranes, bacterial cell membranes, a virus, fungal cell membranes, protozoal cell membranes, cell membranes of parasites, or adipose (also known as adipocyte, lipocyte and fat) cell membranes. These compounds may be used to treat one or more diseases or disorders for which disruption of a cell membrane may be useful. In some embodiments, these diseases or disorders include cancers, bacterial diseases, viral diseases, fungal diseases, protozoan diseases, or diseases carried by parasites. These methods may use vibronic driven action or a similar vibrational energy to achieve these therapeutic effects. Furthermore, the present methods include using very low intensity energy to complete the destruction of the membrane, biomolecule, or cellular component.

These methods may be used to target adipocytes or fat cells. These methods comprise contacting the fat cells with the compound and exposing the cells to an energy source. The energy source and the compounds may be applied once or two or more times over the course of several weeks to reduce the fat deposits. Such light assisted sculpting methods may be used to reduce the size of fat deposits in a patient. After exposure to the energy source, the resultant fat cells may be slowly absorbed over the course of the days or weeks after the energy exposure.

These methods or compounds may additionally be, in some embodiments, useful in selective regulation of the active site in enzymes, modulation of protein channels, or regulation of the structure or function of supramolecular biological assemblies. The compounds described in this application may be used to disrupt protein or protein complexes, nucleic acids or nucleic acid complexes, or carbohydrates or carbohydrate complexes. In these cases, the methods may be used to disrupt or damage these biomolecules and treat or prevent a disease or disorder. Furthermore, these compounds may represent an improvement over those known in the art as the compounds may be more efficacious than, be less toxic than, be longer acting than, be more potent than, produce fewer side effects than, be more easily absorbed than, and/or have a better pharmacokinetic profile (e.g., higher oral bioavailability and/or lower clearance) than, and/or have other useful pharmacological, physical, or chemical properties. These and more details will be discussed in more detail below.

### I. VIBRONIC-DRIVEN ACTION (VDA) AND METHODS OF DISRUPTING MEMBRANES

In some embodiments, the present disclosure provides methods of using vibronic-driven action to disrupt a cell membrane. In some embodiments, the methods use vibronic-driven action. In some embodiments, the method of disrupting a membrane may comprise contacting the membrane with a compound, wherein the compound comprises a moiety that generates a vibronic-driven action and optionally a cell targeting moiety and exposing the compound to an energy source sufficient to generate a vibronic-driven action. In some embodiments, the method of disrupting a membrane may comprise contacting the membrane with a compound, wherein the compound comprises a moiety that absorbs energy of less than 250 mW/cm² and optionally a cell targeting moiety and exposing the compound to an energy source sufficient to destroy the membrane.

Vibronic coupling, also termed “vibronic mode”, refers to an alignment of vibrational and electronic modes, which may also be known as plasmonic modes and phonon modes, respectively. In a molecule, the vibronic mode may also be described as a “molecular plasmon” coupled to a “molecular phonon”. Upon absorbance of energy, vibrational modes of the atoms of a molecule may hybridize with the electronic transitions of the molecule to induce a vibronic mode. In some embodiments, the energy used to induce the vibronic mode is electromagnetic radiation, such as gamma rays, X-rays, ultraviolet (UV) light, visible (Vis) light, near-infrared (NIR) light, infrared light (IR), microwaves, or radio waves. In some embodiments, the energy source used to induce the vibronic mode may be light with a wavelength from about 250 nm to about 2,000 nm. In some embodiments, the wavelength is from about 350 nm to about 1,000 nm or about 450 nm to about 900 nm. In some embodiments, the wavelength of the light may be about 250, 300, 350, 400, 450, 500, 550, 600, 650, 700, 750, 800, 850, 900, 950, 1000, 1100, 1200, 1300, 1400, 1500, 1600, 1700, 1800, 1900, or 2000 nm, or any range derivable therein. In some embodiments, the wavelength of light is about 730 nm. In some embodiments, other types of stimuli including electric fields, ionizing radiation, magnetic fields, mechanical forces, or ultrasound may also be used to induce vibronic coupling.

The present methods contemplate using different intensities or duration of light. The intensity of the light may be proportional to the effectiveness of the vibronic mode coupling at a particular wavelength. These intensities can range from 10 nW/cm² to 10 W/cm², from 100 nW/cm² to 8 W/cm², or from about 10 μW/cm² to about 5 W/cm². The intensity can be from about 10 nW/cm², 50 nW/cm², 100 nW/cm², 250 nW/cm², 500 nW/cm², 750 nW/cm², 1 W/cm², 10 μW/cm², 25 μW/cm², 50 μW/cm², 100 μW/cm², 200 μW/cm², 300 μW/cm², 400 μW/cm², 500 μW/cm², 600 μW/cm², 700 μW/cm², 800 μW/cm², 900 μW/cm², 1 mW/cm², 5 mW/cm², 10 mW/cm², 25 mW/cm², 50 mW/cm², 75 mW/cm², 100 mW/cm², 150 mW/cm², 200 mW/cm², 300 mW/cm², 400 mW/cm², 500 mW/cm², 600 mW/cm², 700 mW/cm², 800 mW/cm², 900 mW/cm², 1 W/cm², 2 W/cm², 4 W/cm², 5 W/cm², 6 W/cm², 8 W/cm², to about 10 W/cm², or any range derivable therein. The intensity of the light may be less than 250 mW/cm², 200 mW/cm², 175 mW/cm², 150 mW/cm², 125 mW/cm², or 100 mW/cm².

When considering depth of NIR light penetration in a patient, as a general rule, there is a loss of one order of magnitude (10×) of NIR photons per centimeter of light penetration through muscle and skin, and loss of two orders of magnitude (100×) of NIR photons per centimeter of light penetration through fat, such as in breast tissue. Fat contains higher water content, and water absorbs the NIR light. Hence, the starting intensity of the light can vary depending on the requisite penetration depth required for the treatment, and this fact accounts, in part, for the large intensity range. The other account depends on the efficiency of activation within the specific molecule.

The present methods may contemplate the use of an energy source with a specific intensity for a given amount of time. The amount of time may be from about 1 second to about 1 hour, from about 3 seconds to about 30 minutes, from about 5 seconds to about 10 minutes, or from about 10 seconds to about 5 minutes. The amount of time may be from about 1 second, 2 seconds, 3 seconds, 5 seconds, 10 seconds, 15 seconds, 30 seconds, 1 minute, 2 minutes, 3 minutes, 5 minutes, 10 minutes, 15 minutes, 20 minutes, 30 minutes, 45 minutes, 1 hour, 2 hours, to about 3 hours, or any range derivable therein. In some embodiments, these times may be the amount of time that the energy source is exposed to the compound in order to achieve necrosis.

In some embodiments, the compound may be an organic molecule. In particular, the organic compound may exhibit either or both of a longitudinal or a transverse molecular plasmon. In certain embodiments, the moiety that generates a vibronic-driven action has a net dipole, has a high degree of symmetry across the longitudinal and/or transverse axis, and has a resonance structure through a pi-bonded system. The net dipole of the moiety, in some embodiments, may be due to a charge, such as a cation, anion, radical cation, or radical anion. In some embodiments, the net dipole is due to a radical. In some embodiments, the moiety that generates a vibronic-driven action may be an organic dye. In particular, the moiety that generates a vibronic-driven action may be a cyanine dye. In other embodiments, the moiety that generates a vibronic-driven action may be a thiazine dye such as methylene blue, a boron containing dye such as 4,4-difluoro-4-bora-3a,4a-diaza-s-indacene (BODIPY), a xanthene dye such as fluorescein or rose bengal, a triarylmethylene such as phenol red, or a dye such as nile red. In some embodiments, the dye is Cy7.5 or derivatives thereof such as a Cy7.5-amine having the structure:

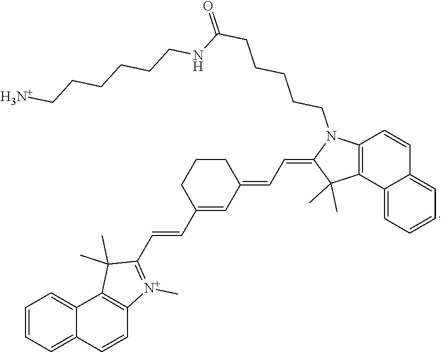 [SMILES: C[N+]1=C(/C=C/C2=CC(=C/C=C3/N(CCCCCC(=O)NCCCCCC[NH3+])c4ccc5ccccc5c4C3(C)C)/CCC2)C(C)(C)c2c1ccc1ccccc21]

In other embodiments, the present disclosure relates to methods that may use a compound of the formula:

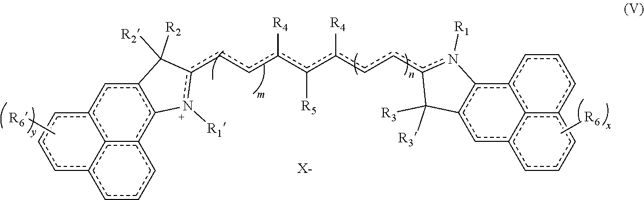

wherein:
R₁ and R₁′ are each independently alkyl_{(C≤8)}, substituted alkyl_{(C≤8)}, or a group of the formula:

-A-NRₐRₐ′Rₐ″
wherein:
   A is an alkanediyl_{(C≤12)} or substituted alkanediyl_{(C≤12)};
   Rₐ and Rₐ′ are each independently hydrogen or alkyl_{(C≤8)}; and
   Rₐ″ is absent, hydrogen, or alkyl_{(C≤8)};
provided at least one of R₁ and R₁′ is a group of the formula: -A-NRₐRₐ′Rₐ″ R₂, R₂′, R₃, and R₃′ are each independently hydrogen, alkyl_{(C≤8)}, or substituted alkyl_{(C≤8)};
R₄ and R₄′ are each independently hydrogen, alkyl_{(C≤8)}, substituted alkyl_{(C≤8)}, or R₄ and R₄′ are taken together as a cycloalkyl group;
R₅ is hydrogen, halo, carboxy, alkyl_{(C≤8)}, substituted alkyl_{(C≤8)}, or —C(O)OR_{b}, wherein R_{b} is alkyl_{(C≤6)} or substituted alkyl_{(C≤6)};
R₆ is hydrogen, amino, halo, hydroxy, or alkyl_{(C≤12)}, alkoxy_{(C≤12)}, alkylamino_{(C≤8)}, dialkylamino_{(C≤8)}, acyl_{(C≤8)}, acyl_{(C≤8)}, acyl_{(C≤8)}, or a substituted version thereof;
m and n are each 0, 1, 2, or 3;
x and y are each independently 0, 1, 2, 3, 4, or 5;
X is a monovalent anion; and
each of the rings is optionally present as either an aromatic or aliphatic ring.

In other embodiments, the methods comprise using a dye that is not Cy7.5-amine. In some embodiments, the methods comprise using methylene blue or a derivative thereof, such as a methylene blue having a formula:

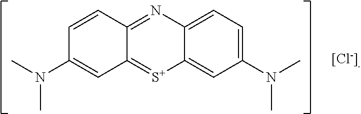

In other aspects, the methods comprising a dye that is not methylene blue. In some aspects, the methods are applicable for at least one compound of Table 2. In some aspects, the methods are applicable for at least one compound denoted as BL-204, GL-308-2, BL-141-2, BL-142 of Table 2.

TABLE 2
Compounds of the Present Disclosure

| ID | Structure |
|---|---|
| BL-204 | 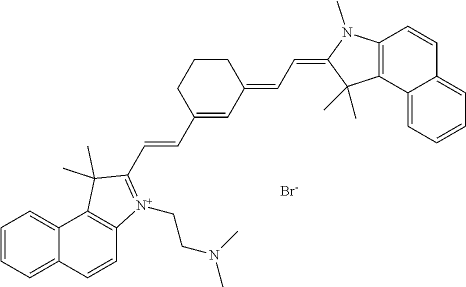 [SMILES: CN(C)CC[N+]1=C(/C=C/C2=CC(=C/C=C3/N(C)c4ccc5ccccc5c4C3(C)C)/CCC2)C(C)(C)c2c1ccc1ccccc21 ; [Br-]] |
| GL-308-2 | 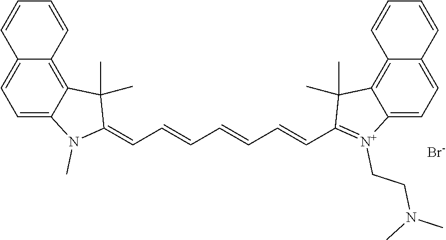 [SMILES: CN(C)CC[N+]1=C(/C=C/C=C/C=C/C=C2/N(C)c3ccc4ccccc4c3C2(C)C)C(C)(C)c2c1ccc1ccccc21 ; [Br-]] |
| BL-141-2 | 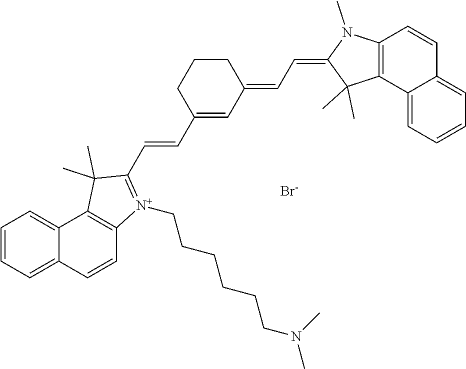 [SMILES: CN(C)CCCCCC[N+]1=C(/C=C/C2=CC(=C/C=C3/N(C)c4ccc5ccccc5c4C3(C)C)/CCC2)C(C)(C)c2c1ccc1ccccc21 ; [Br-]] |
| BL-142 | 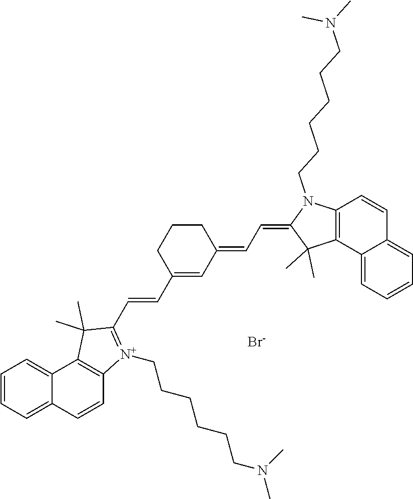 [SMILES: CN(C)CCCCCCN1/C(=C/C=C2C=C(/C=C/C3=[N+](CCCCCCN(C)C)c4ccc5ccccc5c4C3(C)C)CCC/2)C(C)(C)c2c1ccc1ccccc21 ; [Br-]] |
| Cy7.5- amine | 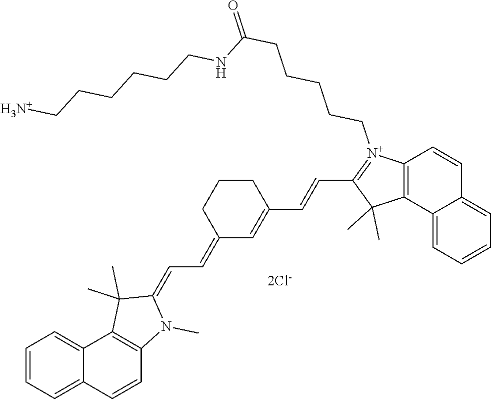 [SMILES: CN1/C(=C\C=C2C=C(/C=C/C3=[N+](CCCCCC(=O)NCCCCCC[NH3+])c4ccc5ccccc5c4C3(C)C)CCC\2)C(C)(C)c2c1ccc1ccccc21] |
| GL-356-2 | 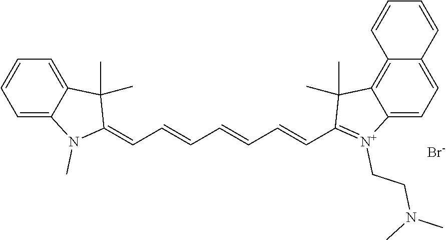 [SMILES: CN(C)CC[N+]1=C(/C=C/C=C/C=C/C=C2/N(C)c3ccccc3C2(C)C)C(C)(C)c2c1ccc1ccccc21 ; [Br-]] |
| GL-291-2 | 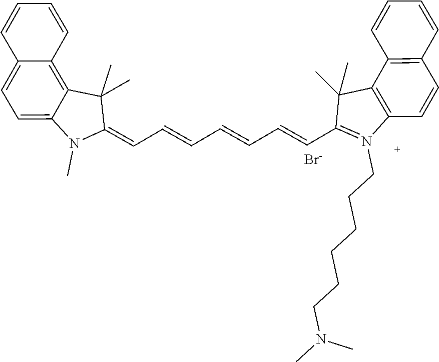 |
| BL-141-1 | 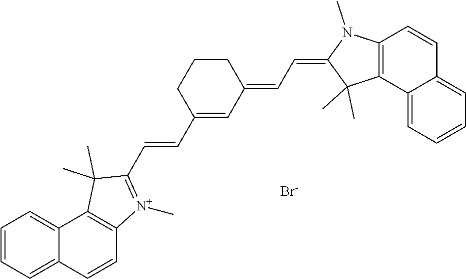 [SMILES: CN1/C(=C/C=C2C=C(/C=C/C3=[N+](C)c4ccc5ccccc5c4C3(C)C)CCC/2)C(C)(C)c2c1ccc1ccccc21 ; [Br-]] |
| BL-205 | 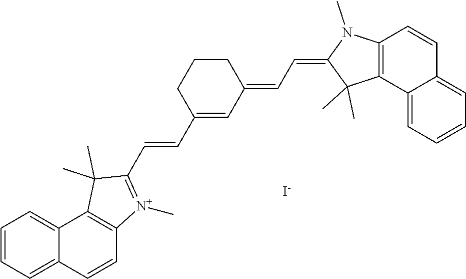 [SMILES: CN1/C(=C/C=C2C=C(/C=C/C3=[N+](C)c4ccc5ccccc5c4C3(C)C)CCC/2)C(C)(C)c2c1ccc1ccccc21 ; [I-]] |
| GL-291-3 | 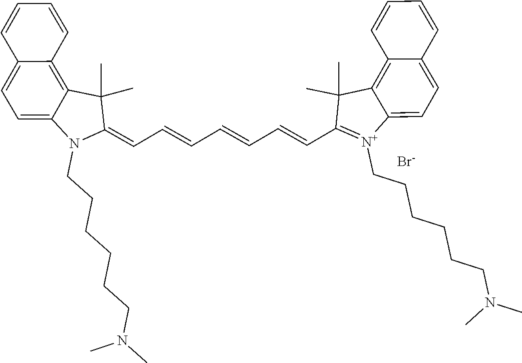 [SMILES: CN(C)CCCCCCN1/C(=C/C=C/C=C/C=C/C2=[N+](CCCCCCN(C)C)c3ccc4ccccc4c3C2(C)C)C(C)(C)c2c1ccc1ccccc21 ; [Br-]] |
| GL-249-2 | 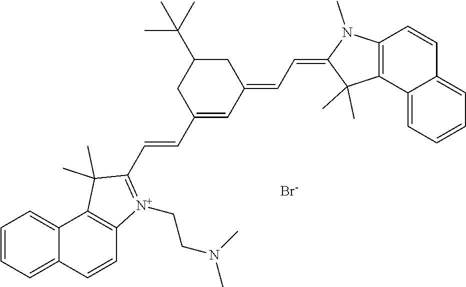 [SMILES: CN(C)CC[N+]1=C(/C=C/C2=CC(=C/C=C3/N(C)c4ccc5ccccc5c4C3(C)C)/CC(C(C)(C)C)C2)C(C)(C)c2c1ccc1ccccc21 ; [Br-]] |
| GL-297-2 | 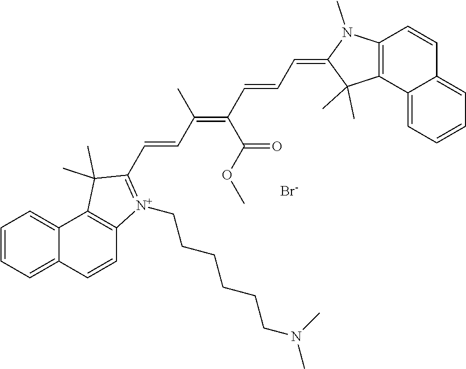 [SMILES: COC(=O)C(/C=C/C=C1/N(C)c2ccc3ccccc3c2C1(C)C)=C(C)\C=C\C1=[N+](CCCCCCN(C)C)c2ccc3ccccc3c2C1(C)C ; [Br-]] |
| GL-161-2 | 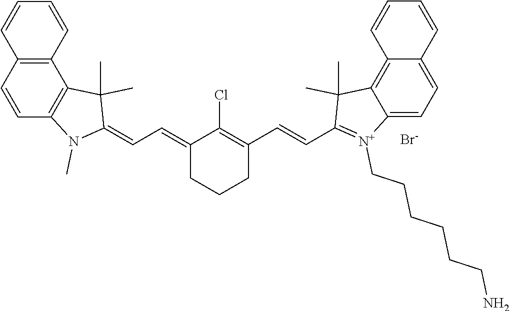 [SMILES: CN1/C(=C/C=C2\CCCC(/C=C/C3=[N+](CCCCCCN)c4ccc5ccccc5c4C3(C)C)=C2Cl)C(C)(C)c2c1ccc1ccccc21 ; [Br-]] |
| GL-286 | 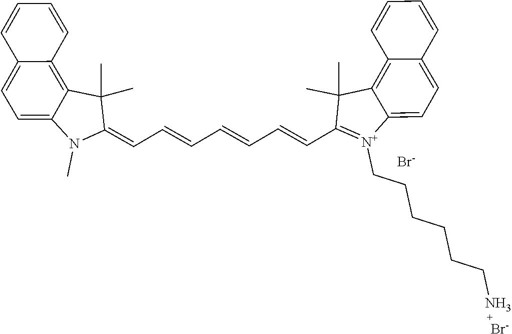 [SMILES: CN1/C(=C/C=C/C=C/C=C/C2=[N+](CCCCCCN)c3ccc4ccccc4c3C2(C)C)C(C)(C)c2c1ccc1ccccc21 ; [Br-] ; [Br-]] |
| GL-176 | 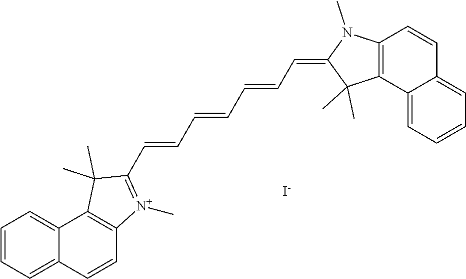 [SMILES: CN1/C(=C/C=C/C=C/C=C/C2=[N+](C)c3ccc4ccccc4c3C2(C)C)C(C)(C)c2c1ccc1ccccc21 ; [I-]] |
| GL-258 | 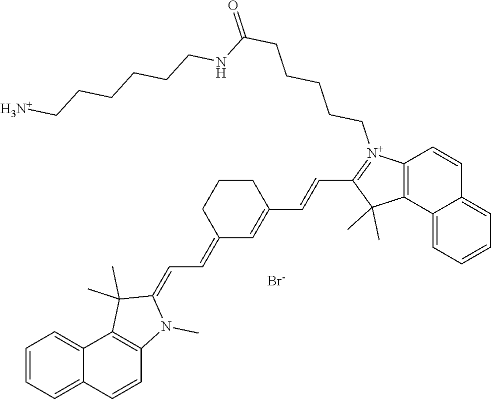 [SMILES: CN1/C(=C\C=C2C=C(/C=C/C3=[N+](CCCCCC(=O)NCCCCCC[NH3+])c4ccc5ccccc5c4C3(C)C)CCC\2)C(C)(C)c2c1ccc1ccccc21 ; [Br-]] |
| Cy5.5 amine | 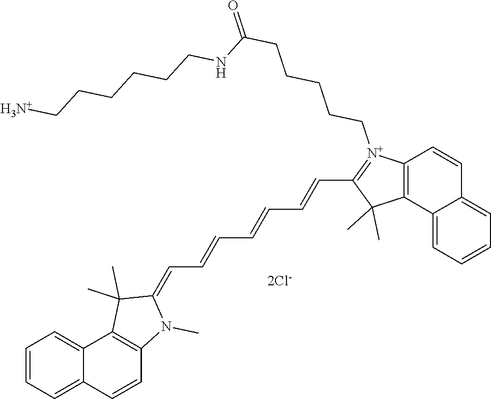 [SMILES: CN1\C(=C/C=C/C=C/C=C/C2=[N+](CCCCCC(=O)NCCCCCC[NH3+])c3ccc4ccccc4c3C2(C)C)C(C)(C)c2c1ccc1ccccc21] |
| GL-261-2 | 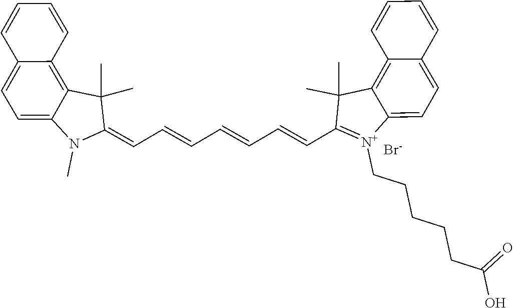 [SMILES: CN1/C(=C/C=C/C=C/C=C/C2=[N+](CCCCCC(=O)O)c3ccc4ccccc4c3C2(C)C)C(C)(C)c2c1ccc1ccccc21 ; [Br-]] |
| Cy7-amine | 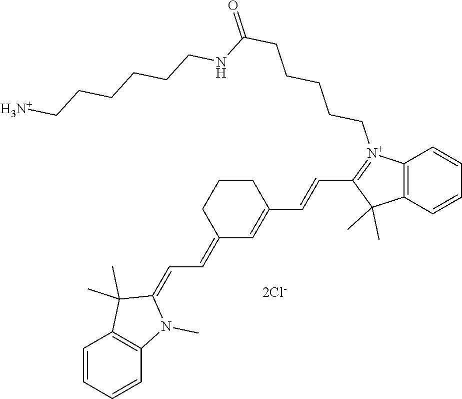 [SMILES: CN1/C(=C\C=C2C=C(/C=C/C3=[N+](CCCCCC(=O)NCCCCCC[NH3+])c4ccccc4C3(C)C)CCC\2)C(C)(C)c2ccccc21] |
| GL-328-2 | 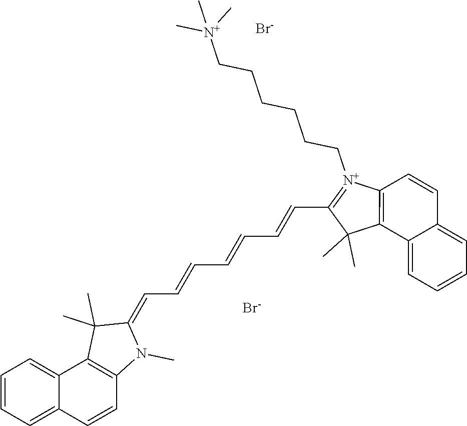 [SMILES: CN1\C(=C/C=C/C=C/C=C/C2=[N+](CCCCCC[N+](C)(C)C)c3ccc4ccccc4c3C2(C)C)C(C)(C)c2c1ccc1ccccc21 ; [Br-] ; [Br-]] |
| GL-254-2 | 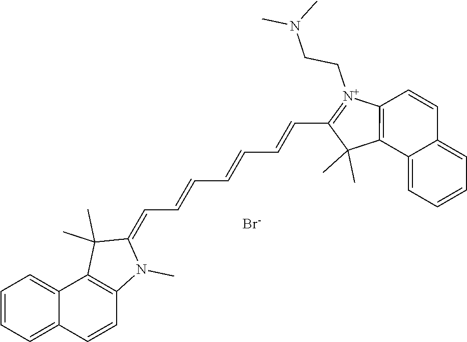 [SMILES: CN(C)CC[N+]1=C(/C=C/C=C/C=C/C=C2\N(C)c3ccc4ccccc4c3C2(C)C)C(C)(C)c2c1ccc1ccccc21 ; [Br-]] |
| Cy5-amine | 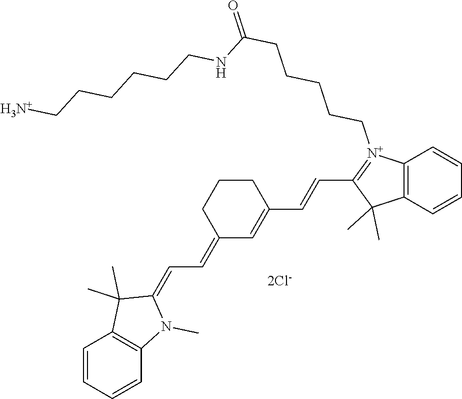 [SMILES: CN1/C(=C\C=C2C=C(/C=C/C3=[N+](CCCCCC(=O)NCCCCCC[NH3+])c4ccccc4C3(C)C)CCC\2)C(C)(C)c2ccccc21] |
| BL-206 | 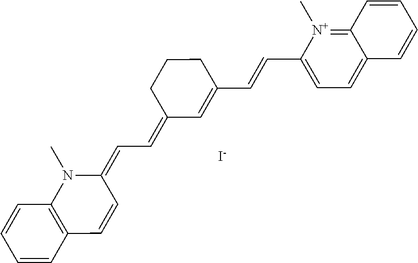 [SMILES: CN1/C(=C/C=C2C=C(/C=C/c3ccc4ccccc4[n+]3C)CCC/2)C=Cc2ccccc21 ; [I-]] |
| BL-242 | 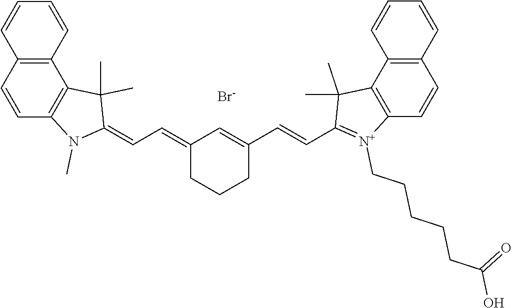 [SMILES: CN1/C(=C/C=C2C=C(/C=C/C3=[N+](CCCCCC(=O)O)c4ccc5ccccc5c4C3(C)C)CCC/2)C(C)(C)c2c1ccc1ccccc21 ; [Br-]] |
| BL-243 | 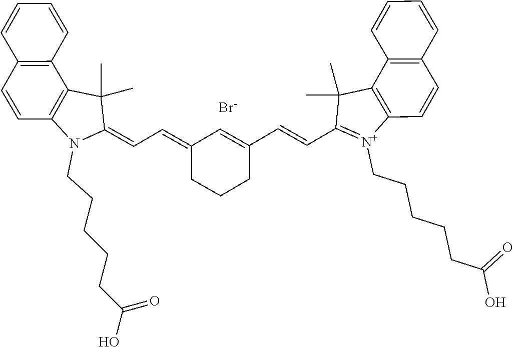 [SMILES: CC1(C)C(/C=C/C2=CC(=C/C=C3/N(CCCCCC(=O)O)c4ccc5ccccc5c4C3(C)C)/CCC2)=[N+](CCCCCC(=O)O)c2ccc3ccccc3c21 ; [Br-]] |
| BL-246-1 | 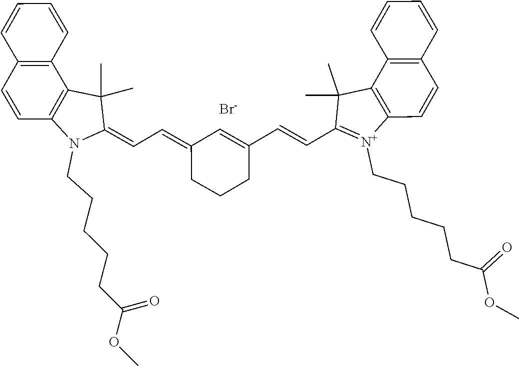 [SMILES: COC(=O)CCCCCN1/C(=C/C=C2C=C(/C=C/C3=[N+](CCCCCC(=O)OC)c4ccc5ccccc5c4C3(C)C)CCC/2)C(C)(C)c2c1ccc1ccccc21 ; [Br-]] |
| BL-246-2 | 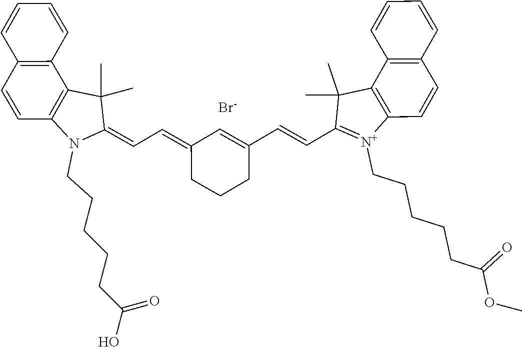 [SMILES: COC(=O)CCCCC[N+]1=C(/C=C/C2=CC(=C/C=C3/N(CCCCCC(=O)O)c4ccc5ccccc5c4C3(C)C)/CCC2)C(C)(C)c2c1ccc1ccccc21 ; [Br-]] |
| BL-248 | 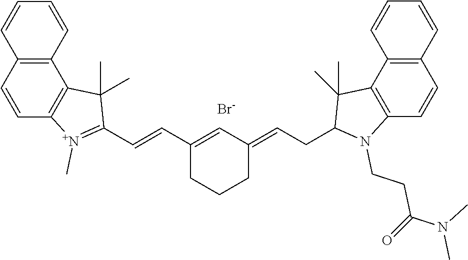 [SMILES: CN(C)C(=O)CCN1c2ccc3ccccc3c2C(C)(C)C1C/C=C1C=C(/C=C/C2=[N+](C)c3ccc4ccccc4c3C2(C)C)CCC/1 ; [Br-]] |
| BL-250 | 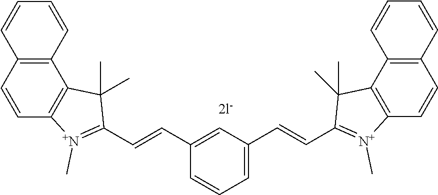 |
| BL-260 | 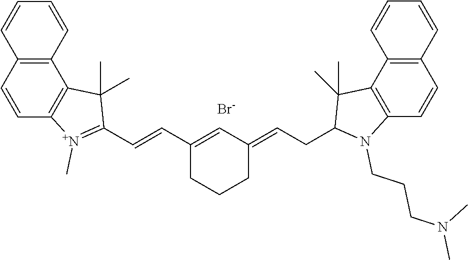 [SMILES: CN(C)CCCN1c2ccc3ccccc3c2C(C)(C)C1C/C=C1C=C(/C=C/C2=[N+](C)c3ccc4ccccc4c3C2(C)C)CCC/1 ; [Br-]] |
| BL-262 | 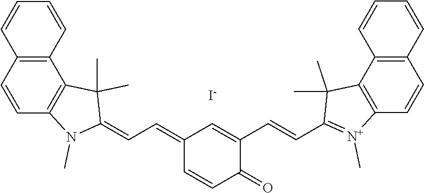 [SMILES: CN1/C(=C/C=C2\C=CC(=O)C(/C=C/C3=[N+](C)c4ccc5ccccc5c4C3(C)C)=C2)C(C)(C)c2c1ccc1ccccc21 ; [I-]] |
| BL-264 | 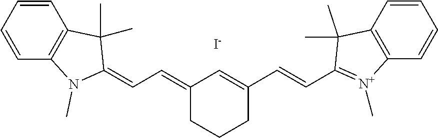 [SMILES: CN1/C(=C/C=C2C=C(/C=C/C3=[N+](C)c4ccccc4C3(C)C)CCC/2)C(C)(C)c2ccccc21 ; [I-]] |
| BL-272 | 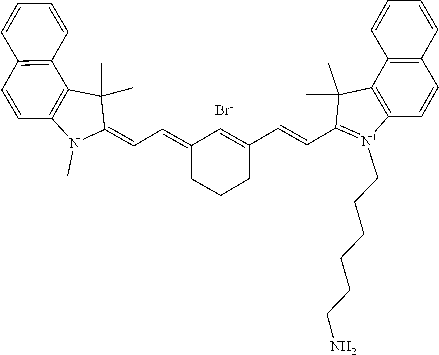 [SMILES: CN1/C(=C/C=C2C=C(/C=C/C3=[N+](CCCCCCN)c4ccc5ccccc5c4C3(C)C)CCC/2)C(C)(C)c2c1ccc1ccccc21 ; [Br-]] |
| BL-273 | 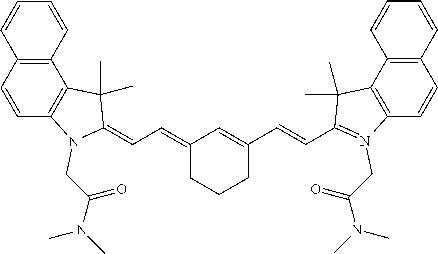 [SMILES: CN(C)C(=O)CN1/C(=C/C=C2C=C(/C=C/C3=[N+](CC(=O)N(C)C)c4ccc5ccccc5c4C3(C)C)CCC/2)C(C)(C)c2c1ccc1ccccc21] |
| GL-258-2 | 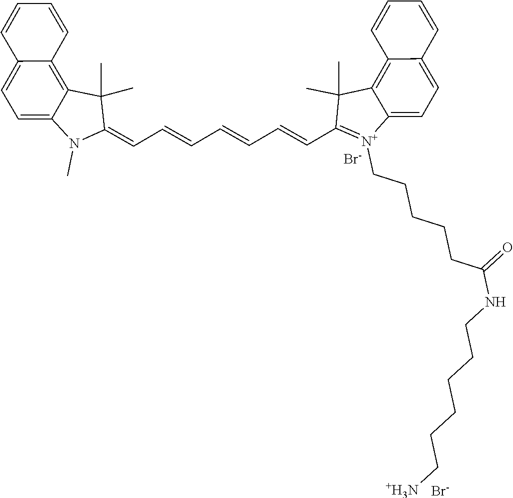 [SMILES: CN1/C(=C/C=C/C=C/C=C/C2=[N+](CCCCCC(=O)NCCCCCC[NH3+])c3ccc4ccccc4c3C2(C)C)C(C)(C)c2c1ccc1ccccc21 ; [Br-] ; [Br-]] |
| GL-368-2 | 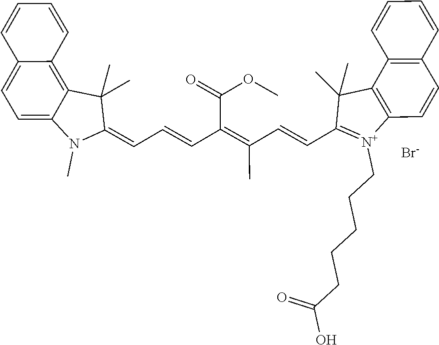 [SMILES: COC(=O)C(/C=C/C=C1/N(C)c2ccc3ccccc3c2C1(C)C)=C(C)\C=C\C1=[N+](CCCCCC(=O)O)c2ccc3ccccc3c2C1(C)C ; [Br-]] |
| GL-297 | 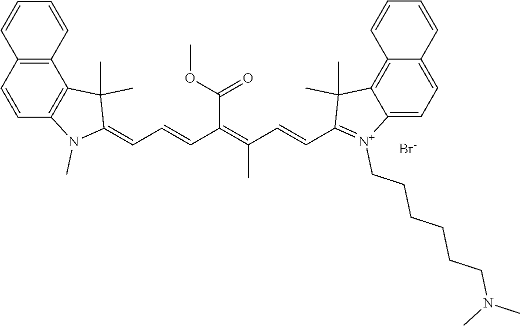 [SMILES: COC(=O)C(/C=C/C=C1/N(C)c2ccc3ccccc3c2C1(C)C)=C(C)\C=C\C1=[N+](CCCCCCN(C)C)c2ccc3ccccc3c2C1(C)C ; [Br-]] |
| GL-264-2 | 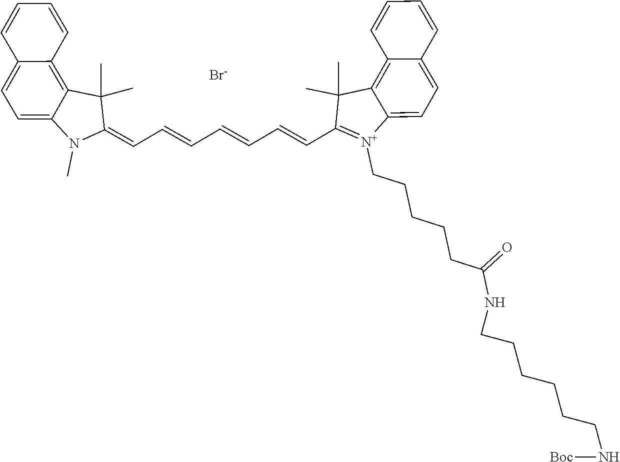 [SMILES: CN1/C(=C/C=C/C=C/C=C/C2=[N+](CCCCCC(=O)NCCCCCCNC(=O)OC(C)(C)C)c3ccc4ccccc4c3C2(C)C)C(C)(C)c2c1ccc1ccccc21 ; [Br-]] |
| GL-349-2 | 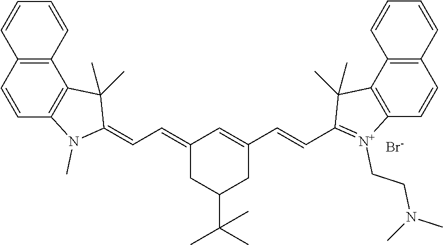 [SMILES: CN(C)CC[N+]1=C(/C=C/C2=CC(=C/C=C3/N(C)c4ccc5ccccc5c4C3(C)C)/CC(C(C)(C)C)C2)C(C)(C)c2c1ccc1ccccc21 ; [Br-]] |
| GL-293-1 | 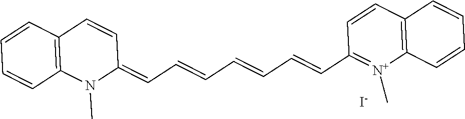 [SMILES: CN1/C(=C/C=C/C=C/C=C/c2ccc3ccccc3[n+]2C)C=Cc2ccccc21 ; [I-]] |
| GL-295-2 | 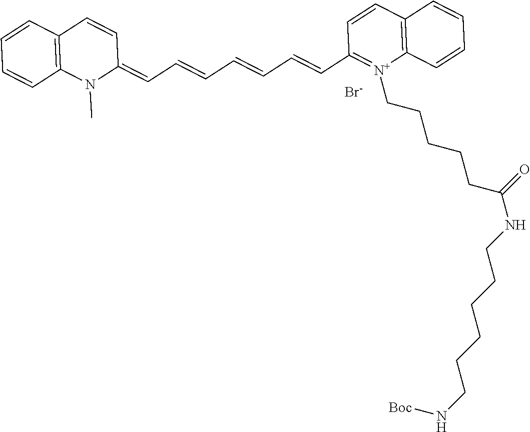 [SMILES: CN1/C(=C/C=C/C=C/C=C/c2ccc3ccccc3[n+]2CCCCCC(=O)NCCCCCCNC(=O)OC(C)(C)C)C=Cc2ccccc21 ; [Br-]] |
| GL-176-2 | 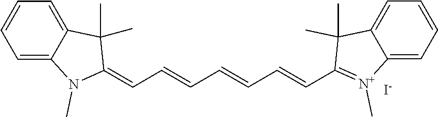 [SMILES: CN1/C(=C/C=C/C=C/C=C/C2=[N+](C)c3ccccc3C2(C)C)C(C)(C)c2ccccc21 ; [I-]] |
| GL-149-2 | 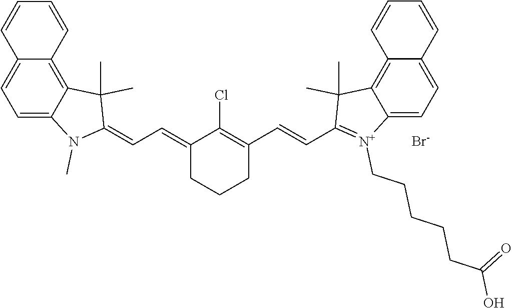 [SMILES: CN1/C(=C/C=C2\CCCC(/C=C/C3=[N+](CCCCCC(=O)O)c4ccc5ccccc5c4C3(C)C)=C2Cl)C(C)(C)c2c1ccc1ccccc21 ; [Br-]] |
| GL-365-2 | 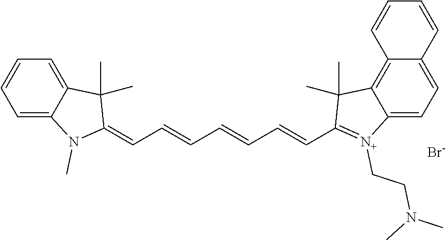 [SMILES: CN(C)CC[N+]1=C(/C=C/C=C/C=C/C=C2/N(C)c3ccccc3C2(C)C)C(C)(C)c2c1ccc1ccccc21 ; [Br-]] |
| GL-293-2 | 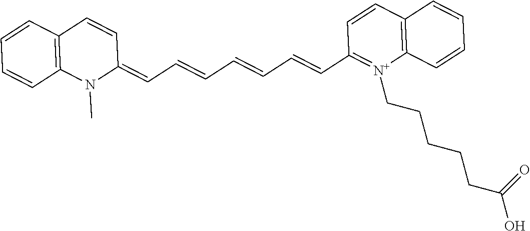 [SMILES: CN1/C(=C/C=C/C=C/C=C/c2ccc3ccccc3[n+]2CCCCCC(=O)O)C=Cc2ccccc21] |
| GL-161-1 | 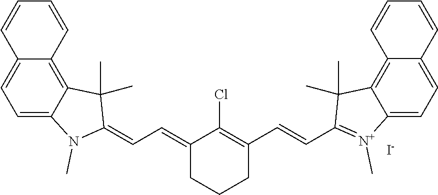 [SMILES: CN1/C(=C/C=C2\CCCC(/C=C/C3=[N+](C)c4ccc5ccccc5c4C3(C)C)=C2Cl)C(C)(C)c2c1ccc1ccccc21 ; [I-]] |
| GL-365-1 | 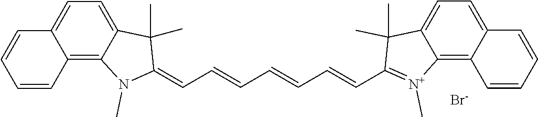 [SMILES: CN1/C(=C/C=C/C=C/C=C/C2=[N+](C)c3c(ccc4ccccc34)C2(C)C)C(C)(C)c2ccc3ccccc3c21 ; [Br-]] |
| GL-354-2 | 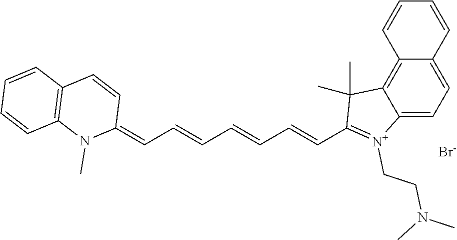 [SMILES: CN(C)CC[N+]1=C(/C=C/C=C/C=C/C=C2\C=Cc3ccccc3N2C)C(C)(C)c2c1ccc1ccccc21 ; [Br-]] |

In other embodiments, the compounds may be an organometallic compound such as an organic ligand bound to one or more metal atoms. The ligands may be bound to one or more metal atoms of the same metal or a different metal. In some aspects, the organometallic compound does not comprise a nanoparticle. In some aspects, the organometallic compound comprises one metal atom. In other aspects, the methods comprise using an organometallic compound with two or more metal atoms. The organometallic compound may comprise two, three, four, or five metal atoms. In particular each of these metal atoms are individually bound to the organic ligand rather than another metal atom. In some embodiments, the metal atoms are not bound together to form some form of metal-metal bond. In some embodiments, the metal atom forms an ionic bond with the organic ligand. In other embodiments, the metal atom forms a covalent bond with the organic ligand.

The compounds may be used in an amount from about 100 nM to about 10 mM, from about 250 nM to about 5 mM, or from about 500 nM to about 2 mM. The amount of the compound used may be from about 50 nM, 100 nM, 200 nM, 250 nM, 500 nM, 750 nM, 1 μM, 10 μM, 25 μM, 50 μM, 75 μM, 100 μM, 200 μM, 250 μM, 300 μM, 400 μM, 500 μM, 600 μM, 700 μM, 750 μM, 800 μM, 900 μM, 1 mM, 2 mM, 3 mM, 4 mM, 5 mM, 7.5 mM, to about 10 mM, or any range derivable therein.

### II. COMPOUNDS AND FORMULATIONS THEREOF

### A. Compounds

The compounds of the present disclosure are shown, for example, above, in the summary of the invention section, the Examples section, and in the claims below. They may be made using the synthetic methods outlined in the Examples section. These methods can be further modified and optimized using the principles and techniques of organic chemistry as applied by a person skilled in the art. Such principles and techniques are taught, for example, in Smith, March's Advanced Organic Chemistry: Reactions, Mechanisms, and Structure, (2013), which is incorporated by reference herein. In addition, the synthetic methods may be further modified and optimized for preparative, pilot- or large-scale production, either batch or continuous, using the principles and techniques of process chemistry as applied by a person skilled in the art. Such principles and techniques are taught, for example, in Anderson, Practical Process Research & Development—A Guide for Organic Chemists (2012), which is incorporated by reference herein.

All the cell membrane disrupting compounds of the present disclosure may in some embodiments be used for the prevention and treatment of one or more diseases or disorders discussed herein or otherwise. In some embodiments, one or more of the compounds characterized or exemplified herein as an intermediate, a metabolite, and/or prodrug, may nevertheless also be useful for the prevention and treatment of one or more diseases or disorders. As such unless explicitly stated to the contrary, all the cell membrane disrupting compounds of the present disclosure are deemed “active compounds” and “therapeutic compounds” that are contemplated for use as active pharmaceutical ingredients (APIs). Actual suitability for human or veterinary use is typically determined using a combination of clinical trial protocols and regulatory procedures, such as those administered by the Food and Drug Administration (FDA). In the United States, the FDA is responsible for protecting the public health by assuring the safety, effectiveness, quality, and security of human and veterinary drugs, vaccines and other biological products, and medical devices.

In some embodiments, the cell membrane disrupting compounds of the present disclosure have the advantage that they may be more efficacious than, be less toxic than, be longer acting than, be more potent than, produce fewer side effects than, be more easily absorbed than, more metabolically stable than, more lipophilic than, more hydrophilic than, and/or have a better pharmacokinetic profile (e.g., higher oral bioavailability and/or lower clearance) than, and/or have other useful pharmacological, physical, or chemical properties over, compounds known in the art, whether for use in the indications stated herein or otherwise.

The cell membrane disrupting compounds of the present disclosure may contain one or more asymmetrically-substituted carbon or nitrogen atom and may be isolated in optically active or racemic form. Thus, all chiral, diastereomeric, racemic form, epimeric form, and all geometric isomeric forms of a chemical formula are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the cell membrane disrupting compounds of the present disclosure can have the S or the R configuration. In some embodiments, the present compounds may contain two or more atoms which have a defined stereochemical orientation.

Chemical formulas used to represent the cell membrane disrupting compounds of the present disclosure will typically only show one of possibly several different tautomers. For example, many types of ketone groups are known to exist in equilibrium with corresponding enol groups. Similarly, many types of imine groups exist in equilibrium with enamine groups. Regardless of which tautomer is depicted for a given compound, and regardless of which one is most prevalent, all tautomers of a given chemical formula are intended.

In addition, atoms making up the cell membrane disrupting compounds of the present disclosure are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

In some embodiments, the cell membrane disrupting compounds of the present disclosure function as prodrugs or can be derivatized to function as prodrugs. Since prodrugs are known to enhance numerous desirable qualities of pharmaceuticals (e.g., solubility, bioavailability, manufacturing, etc.), the compounds employed in some methods of the invention may, if desired, be delivered in prodrug form. Thus, the disclosure contemplates prodrugs of the cell membrane disrupting compounds of the present disclosure as well as methods of delivering prodrugs. Prodrugs of the compounds employed in the disclosure may be prepared by modifying functional groups present in the compound in such a way that the modifications are cleaved, either in routine manipulation or in vivo, to the parent compound. Accordingly, prodrugs include, for example, compounds described herein in which a hydroxy, amino, or carboxy group is bonded to any group that, when the prodrug is administered to a patient, cleaves to form a hydroxy, amino, or carboxylic acid, respectively.

In some embodiments, the cell membrane disrupting compounds of the present disclosure exist in salt or non-salt form. With regard to the salt form(s), in some embodiments the particular anion or cation forming a part of any salt form of a compound provided herein is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

It will be appreciated that many organic compounds can form complexes with solvents in which they are reacted or from which they are precipitated or crystallized. These complexes are known as “solvates.” Where the solvent is water, the complex is known as a “hydrate.” It will also be appreciated that many organic compounds can exist in more than one solid form, including crystalline and amorphous forms. It is contemplated that the present methods include all different polymorphos of the compounds used herein. All solid forms of the cell membrane disrupting compounds provided herein, including any solvates thereof are within the scope of the present invention.

### B. Formulations

In another aspect, for administration to a patient in need of such treatment, pharmaceutical formulations (also referred to as a pharmaceutical preparations, pharmaceutical compositions, pharmaceutical products, medicinal products, medicines, medications, or medicaments) comprise a therapeutically effective amount of a cell membrane disrupting compound disclosed herein formulated with one or more excipients and/or drug carriers appropriate to the indicated route of administration. In some embodiments, the cell membrane disrupting compounds disclosed herein are formulated in a manner amenable for the treatment of human and/or veterinary patients. In some embodiments, formulation comprises admixing or combining one or more of the cell membrane-disrupting compounds disclosed herein with one or more of the following excipients: lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia, sodium alginate, polyvinylpyrrolidone, and/or polyvinyl alcohol. In some embodiments, e.g., for oral administration, the pharmaceutical formulation may be tableted or encapsulated. In some embodiments, the cell membrane disrupting compounds may be dissolved or slurried in water, polyethylene glycol, propylene glycol, ethanol, corn oil, cottonseed oil, peanut oil, sesame oil, benzyl alcohol, sodium chloride, and/or various buffers. In some embodiments, the pharmaceutical formulations may be subjected to pharmaceutical operations, such as sterilization, and/or may contain drug carriers and/or excipients such as preservatives, stabilizers, wetting agents, emulsifiers, encapsulating agents such as lipids, dendrimers, polymers, proteins such as albumin, nucleic acids, and buffers.

Pharmaceutical formulations may be administered by a variety of methods, e.g., orally or by injection (e.g. subcutaneous, intravenous, and intraperitoneal). Depending on the route of administration, the cell membrane disrupting compounds disclosed herein may be coated in a material to protect the compound from the action of acids and other natural conditions which may inactivate the compound. To administer the active compound by other than parenteral administration, it may be necessary to coat the compound with, or co-administer the compound with, a material to prevent its inactivation. In some embodiments, the active compound may be administered to a patient in an appropriate carrier, for example, liposomes, or a diluent. Pharmaceutically acceptable diluents include saline and aqueous buffer solutions. Liposomes include water-in-oil-in-water emulsions as well as conventional liposomes.

The cell membrane disrupting compounds disclosed herein may also be administered parenterally, intraperitoneally, intraspinally, or intracerebrally. Dispersions can be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. The carrier can be a solvent or dispersion medium containing, for example, water, ethanol, polyol (such as, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

The cell membrane disrupting compounds disclosed herein can be administered orally, for example, with an inert diluent or an assimilable edible carrier. The compounds and other ingredients may also be enclosed in a hard or soft-shell gelatin capsule, compressed into tablets, or incorporated directly into the patient's diet. For oral therapeutic administration, the cell membrane disrupting compounds disclosed herein may be incorporated with excipients and used in the form of ingestible tablets, buccal tablets, troches, capsules, elixirs, suspensions, syrups, wafers, and the like. The percentage of the therapeutic compound in the compositions and preparations may, of course, be varied. The amount of the therapeutic compound in such pharmaceutical formulations is such that a suitable dosage will be obtained.

The therapeutic compound may also be administered topically to the skin, eye, ear, or mucosal membranes. Administration of the therapeutic compound topically may include formulations of the compounds as a topical solution, lotion, cream, ointment, gel, foam, transdermal patch, or tincture. When the therapeutic compound is formulated for topical administration, the compound may be combined with one or more agents that increase the permeability of the compound through the tissue to which it is administered. In other embodiments, it is contemplated that the topical administration is administered to the eye. Such administration may be applied to the surface of the cornea, conjunctiva, or sclera. Without wishing to be bound by any theory, it is believed that administration to the surface of the eye allows the therapeutic compound to reach the posterior portion of the eye. Ophthalmic topical administration can be formulated as a solution, suspension, ointment, gel, or emulsion. Finally, topical administration may also include administration to the mucosa membranes such as the inside of the mouth. Such administration can be directly to a particular location within the mucosal membrane such as a tooth, a sore, or an ulcer. Alternatively, if local delivery to the lungs is desired the therapeutic compound may be administered by inhalation in a dry-powder or aerosol formulation.

In some embodiments, it may be advantageous to formulate parenteral compositions in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical carrier. In some embodiments, the specification for the dosage unit forms of the invention are dictated by and directly dependent on (a) the unique characteristics of the therapeutic compound and the particular therapeutic effect to be achieved, and (b) the limitations inherent in the art of compounding such a therapeutic compound for the treatment of a selected condition in a patient. In some embodiments, active compounds are administered at a therapeutically effective dosage sufficient to treat a condition associated with a condition in a patient. For example, the efficacy of a compound can be evaluated in an animal model system that may be predictive of efficacy in treating the disease in a human or another animal.

Single or multiple doses of the agents are contemplated. Desired time intervals for delivery of multiple doses can be determined by one of ordinary skill in the art employing no more than routine experimentation. As an example, patients may be administered two doses daily at approximately 12-hour intervals. In some embodiments, the agent is administered once a day.

The agent(s) may be administered on a routine schedule. As used herein a routine schedule refers to a predetermined designated period of time. The routine schedule may encompass periods of time which are identical, or which differ in length, as long as the schedule is predetermined. For instance, the routine schedule may involve administration twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. In other embodiments, the invention provides that the agent(s) may be taken orally and that the timing of which is or is not dependent upon food intake. Thus, for example, the agent can be taken every morning and/or every evening, regardless of when the patient has eaten or will eat.

### III. INDICATIONS

### A. Cancer and Hyperproliferative Diseases

While hyperproliferative diseases can be associated with any disease which causes a cell to begin to reproduce uncontrollably, the prototypical example is cancer. One of the key elements of cancer is that the cell's normal apoptotic cycle is interrupted and thus agents that interrupt the growth of the cells are important as therapeutic agents for treating these diseases. In some embodiments, the cell membrane that may be disrupted is a human cell, such as a cancer cell. In some embodiments, the compounds of the disclosure may disrupt a human cell, such as an adipose cell. The methods described in the present disclosure contemplate the disruption of either or both a healthy cell or a cancerous cell. In this disclosure, the cell membrane disrupting compounds described herein may be used to lead to decreased cell counts and as such can potentially be used to treat a variety of types of cancer lines. In some embodiments, the cell membrane disrupting compounds described herein are contemplated to open the cell membrane. In further embodiments, the cell membrane disrupting compounds described herein thus allow at least a second therapeutic agent to enter the cell. In some aspects, it is anticipated that the cell membrane disrupting compounds described herein may be used to treat virtually any malignancy.

Cancer cells that may be treated with the compounds of the present disclosure include but are not limited to cells from the skin, bladder, blood, bone, bone marrow, brain, breast, colon, esophagus, gastrointestine, gum, head, kidney, liver, lung, nasopharynx, neck, ovary, prostate, skin, stomach, pancreas, testis, tongue, cervix, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; Leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; Mullerian mixed tumor; nephroblastoma; hepatoblastoma; carcinosarcoma; mesenchymoma, malignant; Brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. In certain aspects, the tumor may comprise an osteosarcoma, angiosarcoma, rhabdosarcoma, leiomyosarcoma, Ewing sarcoma, glioblastoma, neuroblastoma, or leukemia.

### B. Infections

In some embodiments, the cell targeting moiety may target a bacterial cell, a protozoan cell, aa fungal cell, or another type of parasitic cell. In some aspects, the cell targeting moiety may target a virus. In this disclosure, the cell membrane disrupting compounds described herein may be used to lead to decreased cell counts and as such can potentially be used to treat a variety of diseases or conditions associated with or caused by bacteria, protozoa, viruses, a fungi, or other types of parasitic cells. In some embodiments, the cell membrane disrupting compounds described herein are contemplated to open the cell membrane to allow at least a second therapeutic agent to enter a bacterial cell, a protozoan cell, a virus, a fungal cell, or another type of parasitic cell. In some aspects, it is anticipated that the cell membrane disrupting compounds described herein may be used to treat virtually any malignancy associated with or caused by bacteria, protozoa, viruses, a fungi, or other types of parasitic cells.

i. Bacterial Pathogens

There are hundreds of bacterial pathogens in both the Gram-positive and Gram-negative families that cause significant illness and mortality around the word, despite decades of effort developing antibiotic agents. Indeed, antibiotic resistance is a growing problem in bacterial disease.

One of the bacterial diseases with highest disease burden is tuberculosis, caused by the bacterium Mycobacterium tuberculosis, which kills about 2 million people a year, mostly in sub-Saharan Africa. Some non-limiting examples of Mycobacterium tuberculosis antigens include recombinant Ag85A, Ag85B, ESAT6, TB10.4, or fragments thereof including those taught by Ottenhoff and Kaufmann, 2012, which is incorporated herein by reference. Pathogenic bacteria contribute to other globally important diseases, such as pneumonia, which can be caused by bacteria such as Streptococcus and Pseudomonas, and foodborne illnesses, which can be caused by bacteria such as Shigella, Campylobacter, and Salmonella. Pathogenic bacteria also cause infections such as tetanus, typhoid fever, diphtheria, syphilis, and leprosy.

Conditionally pathogenic bacteria are only pathogenic under certain conditions, such as a wound facilitates entry of bacteria into the blood, or a decrease in immune function. For example, Staphylococcus or Streptococcus are also part of the normal human flora and usually exist on the skin or in the nose without causing disease, but can potentially cause skin infections, pneumonia, meningitis, and even overwhelming sepsis, a systemic inflammatory response producing shock, massive vasodilation and death. Some species of bacteria, such as Pseudomonas aeruginosa, Burkholderia cenocepacia, and Mycobacterium avium, are opportunistic pathogens and cause disease mainly in people suffering from immunosuppression or cystic fibrosis.

Other bacterial invariably cause disease in humans, such as obligate intracellular parasites (e.g., Chlamydophila, Ehrlichia, Rickettsia) that are capable of growing and reproducing only within the cells of other organisms. Still, infections with intracellular bacteria may be asymptomatic, such as during the incubation period. An example of intracellular bacteria is Rickettsia. One species of Rickettsia causes typhus, while another causes Rocky Mountain spotted fever. Chlamydia, another phylum of obligate intracellular parasites, contains species that can cause pneumonia or urinary tract infection and may be involved in coronary heart disease. Mycobacterium, Brucella, Francisella, Legionella, and Listeria can exist intracellularly, though they are facultative (not obligate) intracellular parasites. Cell membranes for these bacteria may be disrupted using the methods described herein.

ii. Viral Pathogens

Viral pathogens are important health concerns. These pathogens include respiratory viruses such as Adenoviruses, Avian influenza, Influenza virus type A, Influenza virus type B, Measles, Parainfluenza virus, Respiratory syncytial virus (RSV), Rhinoviruses, SARS-CoV, MERS-CoV, and SARS-CoV-2, gastro-enteric viruses such as Coxsackie viruses, enteroviruses such as Poliovirus and Rotavirus, hepatitis viruses such as Hepatitis B virus, Hepatitis C virus, Bovine viral diarrhea virus (surrogate), herpesviruses such as Herpes simplex 1, Herpes simplex 2, Human cytomegalovirus, and Varicella zoster virus, retroviruses such as Human immunodeficiency virus 1 (HIV-1), and Human immunodeficiency virus 2 (HIV-2), as well as Dengue virus, Hantavirus, Hemorrhagic fever viruses, Lymphocytic choromeningitis virus, Smallpox virus, Ebola virus, Rabies virus, West Nile virus and Yellow fever virus. Some non-limiting viral antigens include hepatitis B virus HBV surface and core antigens, influenza virus haemagglutinin and neuroaminidase antigens, West Nile virus envelop protein (E) and premembrane protein (prM), Dengue virus 80E subunit protein, Ebola virus glycoprotein, HIV envelope protein gp41 and gp120, or fragments thereof. Other HIV antigens can be found in de Taeye, et al., 2016, which is incorporated herein by reference. The cell membranes for any of these viral pathogens may be disrupted using the methods described herein.

iii. Fungal Pathogens

Pathogenic fungi are fungi that cause disease in humans or other organisms. The following are but a few examples.

Candida species are important human pathogens that are best known for causing opportunist infections in immunocompromised hosts (e.g., transplant patients, AIDS sufferers, and cancer patients). Infections are difficult to treat and can be very serious. Aspergillus can and does cause disease in three major ways: through the production of mycotoxins; through induction of allergenic responses; and through localized or systemic infections. With the latter two categories, the immune status of the host is pivotal. The most common pathogenic species are Aspergillus fumigatus and Aspergillus flavus. Cryptococcus neoformans can cause a severe form of meningitis and meningo-encephalitis in patients with HIV infection and AIDS. The majority of Cryptococcus species lives in the soil and do not cause disease in humans. Cryptococcus laurentii and Cryptococcus albidus have been known to occasionally cause moderate-to-severe disease in human patients with compromised immunity. Cryptococcus gattii is endemic to tropical parts of the continent of Africa and Australia and can cause disease in non-immunocompromised people. Histoplasma capsulatum can cause histoplasmosis in humans, dogs and cats. Pneumocystis jirovecii (or Pneumocystis carinii) can cause a form of pneumonia in people with weakened immune systems, such as premature children, the elderly, transplant patients and AIDS patients. Stachybotrys chartarum or “black mold” can cause respiratory damage and severe headaches. It frequently occurs in houses in regions that are chronically damp. Cell membranes from these fungi may be disrupted using the methods described herein. Furthermore, the cell membrane disrupting compounds of the present disclosure may be used to treat onychomycosis.

iv. Parasites

Parasite presents a major health issue, particularly in under-developed countries around the world. Significant pathogenic parasites include Entamoeba histolytica, Giardia lamblia, Trichomonas vaginalis, Plasmodium falciparum, Plasmodium malariae, Plasmodium ovale, Plasmodium vivax, Trypanosoma gambiense, Trypanosoma rhodesiense, Trypanosoma cruzi, Ascaris lumbricoides, Trichinella spiralis, Toxoplasma gondii, Leishmania donovani, Leishmania tropica, Leishmania braziliensis, Schistosoma mansoni, Schistosoma japonicum, Schistosoma haematobium, and Pneumocystis jiroveci. Cell membranes from these parasites may be disrupted using the methods described herein.

### IV. Cell Targeting Moieties

In some aspects, the present disclosure provides compounds conjugated directly or through linkers to a cell targeting moiety. In some embodiments, the conjugation of the compound to a cell targeting moiety increases the efficacy of the compound in treating a disease or disorder. Cell targeting moieties according to the embodiments may be, for example, an antibody, a lipid, a carbohydrate, a polysaccharide, a growth factor, a hormone, a peptide, an aptamer, a small molecule such as a hormone, an imaging agent, acofactor, an amino acid, a natural product, a small organic molecule other than a natural product, or a cytokine. In some embodiments, the cell targeting moiety is a functional group that associates with the cell membrane, a carbohydrate or polysaccharide that binds to one or more markers on the cell membrane, a lipid that binds to one or more markers on the cell membrane, a small molecule that binds to one or more markers on the cell membrane, an aptamer that binds to one or more markers on the cell membrane, or a peptide or an antibody that binds to one or more markers on the cell membrane. In some embodiments, the cell targeting moiety may target a human cell, such as a cancer cell. For instance, a cell targeting moiety according to the embodiments may bind to a liver cancer cell such as a Hep3B cell. It has been demonstrated that the gp240 antigen is expressed in a variety of melanomas but not in normal tissues. Thus, in some embodiments, the compounds of the present disclosure may be used in conjugates with an antibody for a specific antigen that is expressed by a cancer cell but not in normal tissues.

In certain embodiments, the cell targeting group is a functional group such as a positively charged group like an amine. The positively charged group may be used to associate with the negatively charged groups at the surface of the cell membrane. It is contemplated that this group might be used to associate with other negatively charged groups such as negatively charged proteins or nucleic acids.

In certain additional embodiments, it is envisioned that cancer cell targeting moieties bind to multiple types of cancer cells. For example, the 8H9 monoclonal antibody and the single chain antibodies derived therefrom bind to a glycoprotein that is expressed on breast cancers, sarcomas and neuroblastomas (Onda, et al., 2004). Another example is the cell targeting agents described in U.S. Patent Publication No. 2004/005647 and in Winthrop, et al. (2003) that bind to MUC-1, an antigen that is expressed on a variety cancer types. Thus, it will be understood that in certain embodiments, cell targeting constructs according the embodiments may be targeted against a plurality of cancer or tumor types.

Additionally, certain cell surface molecules are highly expressed in tumor cells, including hormone receptors such as human chorionic gonadotropin receptor and gonadotropin releasing hormone receptor (Nechushtan et al., 1997). Therefore, the corresponding hormones may be used as the cell-specific targeting moieties in cancer therapy. Additionally, the cell targeting moiety that may be used include a cofactor, a sugar, a drug molecule, an imaging agent, or a fluorescent dye. Many cancerous cells are known to over express folate receptors and thus folic acid or other folate derivatives may be used as conjugates to trigger cell-specific interaction between the conjugates of the present disclosure and a cell (Campbell, et al., 1991; Weitman, et al., 1992).

Since a large number of cell surface receptors have been identified in hematopoietic cells of various lineages, ligands or antibodies specific for these receptors may be used as cell-specific targeting moieties. IL-2 may also be used as a cell-specific targeting moiety in a chimeric protein to target IL-2R+ cells. Alternatively, other molecules such as B7-1, B7-2 and CD40 may be used to specifically target activated T cells (The Leucocyte Antigen Facts Book, 1993, Barclay, et al. (eds.), Academic Press). Furthermore, B cells express CD19, CD40 and IL-4 receptor and may be targeted by moieties that bind these receptors, such as CD40 ligand, IL-4, IL-5, IL-6 and CD28. The elimination of immune cells such as T cells and B cells is particularly useful in the treatment of lymphoid tumors.

Other cytokines that may be used to target specific cell subsets include the interleukins (IL-1 through IL-15), granulocyte-colony stimulating factor, macrophage-colony stimulating factor, granulocyte-macrophage colony stimulating factor, leukemia inhibitory factor, tumor necrosis factor, transforming growth factor, epidermal growth factor, insulin-like growth factors, and/or fibroblast growth factor (Thompson (ed.), 1994, The Cytokine Handbook, Academic Press, San Diego). In some aspects, the targeting polypeptide is a cytokine that binds to the Fn14 receptor, such as TWEAK (see, e.g., Winkles, 2008; Zhou, et al., 2011 and Burkly, et al., 2007, incorporated herein by reference).

A skilled artisan recognizes that there are a variety of known cytokines, including hematopoietins (four-helix bundles) [such as EPO (erythropoietin), IL-2 (T-cell growth factor), IL-3 (multicolony CSF), IL-4 (BCGF-1, BSF-1), IL-5 (BCGF-2), IL-6 IL-4 (IFN-b2, BSF-2, BCDF), IL-7, IL-8, IL-9, IL-11, IL-13 (P600), G-CSF, IL-15 (T-cell growth factor), GM-CSF (granulocyte macrophage colony stimulating factor), OSM (OM, oncostatin M), and LIF (leukemia inhibitory factor)]; interferons [such as IFN-g, IFN-a, and IFN-b); immunoglobin superfamily (such as B7.1 (CD80), and B7.2 (B70, CD86)]; TNF family [such as TNF-α (cachectin), TNF-b (lymphotoxin, LT, LT-a), LT-b, CD40 ligand (CD40L), Fas ligand (FasL), CD27 ligand (CD27L), CD30 ligand (CD30L), and 4-1BBL)]; and those unassigned to a particular family [such as TGF-b, IL 1a, IL-1b, IL-1 RA, IL-10 (cytokine synthesis inhibitor F), IL-12 (NK cell stimulatory factor), MIF, IL-16, IL-17 (mCTLA-8), and/or IL-18 (IGIF, interferon-g inducing factor)]. Furthermore, the Fc portion of the heavy chain of an antibody may be used to target Fc receptor-expressing cells such as the use of the Fc portion of an IgE antibody to target mast cells and basophils.

Furthermore, in some aspects, the cell-targeting moiety may be a peptide sequence or a cyclic peptide. Examples, cell- and tissue-targeting peptides that may be used according to the embodiments are provided, for instance, in U.S. Pat. Nos. 6,232,287; 6,528,481; 7,452,964; 7,671,010; 7,781,565; 8,507,445; and 8,450,278, each of which is incorporated herein by reference.

Thus, in some embodiments, cell targeting moieties are antibodies or avimers. Antibodies and avimers can be generated against virtually any cell surface marker thus, providing a method for targeted to delivery of GrB to virtually any cell population of interest. Methods for generating antibodies that may be used as cell targeting moieties are detailed below. Methods for generating avimers that bind to a given cell surface marker are detailed in U.S. Patent Publications Nos. 2006/0234299 and 2006/0223114, each incorporated herein by reference.

Additionally, it is contemplated that the compounds described herein may be conjugated to a nanoparticle or other nanomaterial. Some non-limiting examples of nanoparticles include metal nanoparticles such as gold or silver nanoparticles or polymeric nanoparticles such as poly-L-lactic acid or poly(ethylene) glycol polymers. Nanoparticles and nanomaterials which may be conjugated to the instant compounds include those described in U.S. Patent Publications Nos. 2006/0034925, 2006/0115537, 2007/0148095, 2012/0141550, 2013/0138032, and 2014/0024610 and PCT Publication No. 2008/121949, 2011/053435, and 2014/087413, each incorporated herein by reference.

### IV. THERAPIES

### A. Methods of Treatment

In particular, the compositions that may be used in treating a disease or disorder in a subject (e.g., a human subject) are disclosed herein. The compositions described above are preferably administered to a mammal (e.g., rodent, human, non-human primates, canine, bovine, ovine, equine, feline, etc.) in an effective amount, that is, an amount capable of producing a desirable result in a treated subject (e.g., slowing, stopping, reducing or eliminating one or more symptoms or underlying causes of disease). Toxicity and therapeutic efficacy of the compositions utilized in methods of the disclosure can be determined by standard pharmaceutical procedures. As is well known in the medical and veterinary arts, dosage for any one animal depends on many factors, including the subject's size, body surface area, body weight, age, the particular composition to be administered, time and route of administration, general health, the clinical symptoms and other drugs being administered concurrently. In some embodiments, amount of the cell membrane disrupting compounds used is calculated to be from about 0.01 mg to about 10,000 mg/day. In some embodiments, the amount is from about 1 mg to about 1,000 mg/day. In some embodiments, these dosings may be reduced or increased based upon the biological factors of a particular patient such as increased or decreased metabolic breakdown of the drug or decreased uptake by the digestive tract if administered orally. Additionally, the cell membrane disrupting compounds may be more efficacious and thus a smaller dose is required to achieve a similar effect. Such a dose is typically administered once a day for a few weeks or until sufficient achieve clinical benefit.

Also provided herein are uses of a composition in the treatment of a disease or disorder. These compositions may also be used in the preparation of a medicament for the treatment of a disease or disorder. Finally, the present disclosure also contemplates the use of a compound as described herein for the preparation of a medicament.

The therapeutic methods of the disclosure (which include prophylactic treatment) in general include administration of a therapeutically effective amount of the compositions described herein to a subject in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, family history, and the like).

### B. Combination Therapies

It is envisioned that the cell membrane disrupting compounds described herein may be used in combination therapies with one or more additional therapies or a compound which mitigates one or more of the side effects experienced by the patient. It is common in the field of medicine to combine therapeutic modalities. The following is a general discussion of therapies that may be used in conjunction with the therapies of the present disclosure.

To treat diseases or disorders using the methods and compositions of the present disclosure, one would generally contact a cell or a subject with a cell membrane disrupting compound and at least one other therapy. These therapies would be provided in a combined amount effective to achieve a reduction in one or more disease parameter. This process may involve contacting the cells/subjects with both agents/therapies at the same time, e.g., using a single composition or pharmacological formulation that includes both agents, or by contacting the cell/subject with two distinct compositions or formulations, at the same time, wherein one composition includes the compound and the other includes the other agent.

Alternatively, the compounds described herein may precede or follow the other treatment by intervals ranging from minutes to weeks. One would generally ensure that a significant period of time did not expire between the times of each delivery, such that the therapies would still be able to exert an advantageously combined effect on the cell/subject. In such instances, it is contemplated that one would contact the cell with both modalities within about 12-24 hours of each other, within about 6-12 hours of each other, or with a delay time of only about 1-2 hours. In some situations, it may be desirable to extend the time period for treatment significantly; however, where several days (2, 3, 4, 5, 6 or 7) to several weeks (1, 2, 3, 4, 5, 6, 7 or 8) lapse between the respective administrations.

It also is conceivable that more than one administration of either the compound or the other therapy will be desired. Various combinations may be employed, where a compound of the present disclosure is “A,” and the other therapy is “B,” as exemplified below:
A/B/A B/A/B B/B/A A/A/B B/A/A A/B/B B/B/B/A B/B/A/B A/A/B/B A/B/A/B A/B/B/A B/B/A/A B/A/B/A B/A/A/B B/B/B/A A/A/A/B B/A/A/A A/B/A/A A/A/B/A A/B/B/B B/A/B/B B/B/A/B
   Other combinations are also contemplated. A discussion of other potential therapies that may be used combination with the compounds of the present disclosure is presented elsewhere in this document.

### V. CHEMISTRY BACKGROUND

In some aspects, cell membrane disrupting compounds of this disclosure can be synthesized using the methods of organic chemistry as described in this application. These methods can be further modified and optimized using the principles and techniques of organic chemistry as applied by a person skilled in the art. Such principles and techniques are taught, for example, in March's Advanced Organic Chemistry: Reactions, Mechanisms, and Structure (2007), which is incorporated by reference herein.

### A. Process Scale-Up

The synthetic methods described herein can be further modified and optimized for preparative, pilot- or large-scale production, either batch of continuous, using the principles and techniques of process chemistry as applied by a person skilled in the art. Such principles and techniques are taught, for example, in Practical Process Research & Development (2000), which is incorporated by reference herein. The synthetic method described herein may be used to produce preparative scale amounts of the compounds described herein.

### B. Chemical Definitions

When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means ═NH; “cyano” means —CN; “isocyanyl” means —N═C═O; “azido” means —N₃; in a monovalent context “phosphate” means —OP(O)(OH)₂ or a deprotonated form thereof; in a divalent context “phosphate” means —OP(O)(OH)O— or a deprotonated form thereof; “mercapto” means —SH; and “thio” means ═S; “thiocarbonyl” means —C(═S)—; “sulfonyl” means —S(O)₂—; and “sulfinyl” means —S(O)—.

In the context of chemical formulas, the symbol “—” means a single bond, “═” means a double bond, and “≡” means triple bond. The symbol “----” represents an optional bond, which if present is either single or double. The symbol “ [symbol] ” represents a single bond or a double bond. Thus, the formula

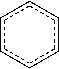

covers, for example,

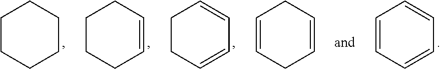

And it is understood that no one such ring atom forms part of more than one double bond. Furthermore, it is noted that the covalent bond symbol “—”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g.,

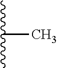

for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol means a single bond where the geometry around a double bond (e.g., either E or Z) is “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a variable is depicted as a “floating group” on a ring system, for example, the group “R” in the formula:

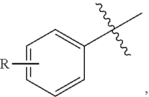

then the variable may replace any hydrogen atom attached to any of the ring atoms, including a depicted, implied, or expressly defined hydrogen, so long as a stable structure is formed. When a variable is depicted as a “floating group” on a fused ring system, as for example the group “R” in the formula:

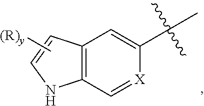

then the variable may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the R enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” or “C=n” defines the exact number (n) of carbon atoms in the group/class. “C n” defines the maximum number (n) of carbon atoms that can be in the group/class, with the minimum number as small as possible for the group/class in question. For example, it is understood that the minimum number of carbon atoms in the groups “alkyl_{(C≤8)}”, “alkanediyl_{(C≤8)}”, “heteroaryl_{(C≤8)}”, and “acyl_{(C≤8)}” is one, the minimum number of carbon atoms in the groups “alkenyl_{(C≤8)}”, “alkynyl_{(C≤8)}”, and “heterocycloalkyl_{(C≤8)}” is two, the minimum number of carbon atoms in the group “cycloalkyl_{(C≤8)}” is three, and the minimum number of carbon atoms in the groups “aryl_{(C≤8)}” and “arenediyl_{(C≤8)}” is six. “Cn-n′” defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C₁₋₄-alkyl”, “C1-4-alkyl”, “alkyl_{(C1-4)}”, and “alkyl_{(C≤4)}” are all synonymous. Except as noted below, every carbon atom is counted to determine whether the group or compound falls with the specified number of carbon atoms. For example, the group dihexylamino is an example of a dialkylamino_{(C12)} group; however, it is not an example of a dialkylamino_{(C6)} group. Likewise, phenylethyl is an example of an aralkyl_{(C≤8)} group. When any of the chemical groups or compound classes defined herein is modified by the term “substituted”, any carbon atom in the moiety replacing the hydrogen atom is not counted. Thus methoxyhexyl, which has a total of seven carbon atoms, is an example of a substituted alkyl_{(C1-6)}. Unless specified otherwise, any chemical group or compound class listed in a claim set without a carbon atom limit has a carbon atom limit of less than or equal to twelve.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” signifies that the compound or chemical group so modified is an acyclic or cyclic, but non-aromatic compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” signifies that the compound or chemical group so modified has a planar unsaturated ring of atoms with 4n+2 electrons in a fully conjugated cyclic R system. An aromatic compound or chemical group may be depicted as a single resonance structure; however, depiction of one resonance structure is taken to also refer to any other resonance structure. For example:

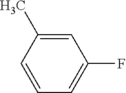

is also taken to refer to

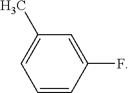

Aromatic compounds may also be depicted using a circle to represent the delocalized nature of the electrons in the fully conjugated cyclic π system, two non-limiting examples of which are shown below:

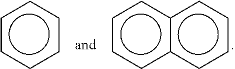

The term “alkyl” refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂ (i-Pr, ⁱPr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tert-butyl, t-butyl, t-Bu or ^{t}Bu), and —CH₂C(CH₃)₃ (neo-pentyl) are non-limiting examples of alkyl groups. The term “alkanediyl” refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)₂CH₂—, and —CH₂CH₂CH₂— are non-limiting examples of alkanediyl groups. The term “alkylidene” refers to the divalent group ═CRR′ in which R and R′ are independently hydrogen or alkyl. Non-limiting examples of alkylidene groups include: =CH₂, =CH(CH₂CH₃), and ═C(CH₃)₂. An “alkane” refers to the class of compounds having the formula H—R, wherein R is alkyl as this term is defined above.

The term “cycloalkyl” refers to a monovalent saturated aliphatic group with a carbon atom as the point of attachment, said carbon atom forming part of one or more non-aromatic ring structures, no carbon-carbon double or triple bonds, and no atoms other than carbon and hydrogen. Non-limiting examples include: —CH(CH₂)₂ (cyclopropyl), cyclobutyl, cyclopentyl, or cyclohexyl (Cy). As used herein, the term does not preclude the presence of one or more alkyl groups (carbon number limitation permitting) attached to a carbon atom of the non-aromatic ring structure. The term “cycloalkanediyl” refers to a divalent saturated aliphatic group with two carbon atoms as points of attachment, no carbon-carbon double or triple bonds, and no atoms other than carbon and hydrogen. The group

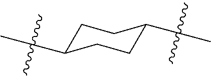 [SMILES: *C1CCC(*)CC1]

is a non-limiting example of cycloalkanediyl group. A “cycloalkane” refers to the class of compounds having the formula H—R, wherein R is cycloalkyl as this term is defined above.

The term “alkenyl” refers to a monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” refers to a divalent unsaturated aliphatic group, with two carbon atoms as points of attachment, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, —CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H—R, wherein R is alkenyl as this term is defined above. Similarly, the terms “terminal alkene” and “α-olefin” are synonymous and refer to an alkene having just one carbon-carbon double bond, wherein that bond is part of a vinyl group at an end of the molecule.

The term “alkynyl” refers to a monovalent unsaturated aliphatic group with a carbon atom as the point of attachment, a linear or branched acyclic structure, at least one carbon-carbon triple bond, and no atoms other than carbon and hydrogen. As used herein, the term alkynyl does not preclude the presence of one or more non-aromatic carbon-carbon double bonds. The groups —C≡CH, —C≡CCH₃, and —CH₂C≡CCH₃ are non-limiting examples of alkynyl groups. An “alkyne” refers to the class of compounds having the formula H—R, wherein R is alkynyl.

The term “aryl” refers to a monovalent unsaturated aromatic group with an aromatic carbon atom as the point of attachment, said carbon atom forming part of a one or more aromatic ring structures, each with six ring atoms that are all carbon, and wherein the group consists of no atoms other than carbon and hydrogen. If more than one ring is present, the rings may be fused or unfused. Unfused rings are connected with a covalent bond. As used herein, the term aryl does not preclude the presence of one or more alkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl (e.g., 4-phenylphenyl). The term “arenediyl” refers to a divalent aromatic group with two aromatic carbon atoms as points of attachment, said carbon atoms forming part of one or more six-membered aromatic ring structures, each with six ring atoms that are all carbon, and wherein the divalent group consists of no atoms other than carbon and hydrogen. As used herein, the term arenediyl does not preclude the presence of one or more alkyl groups (carbon number limitation permitting) attached to the first aromatic ring or any additional aromatic ring present. If more than one ring is present, the rings may be fused or unfused. Unfused rings are connected with a covalent bond. Non-limiting examples of arenediyl groups include:

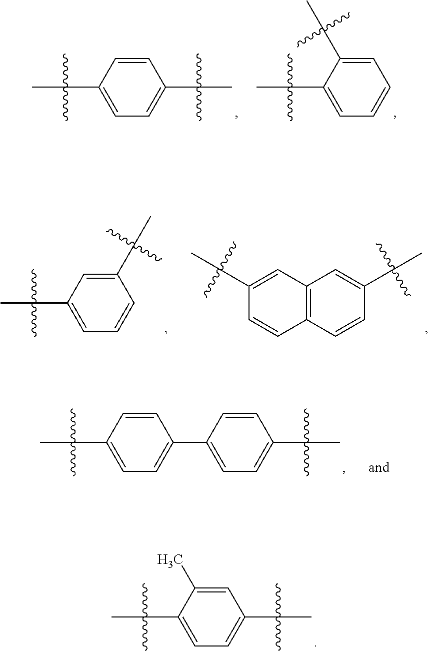

An “arene” refers to the class of compounds having the formula H—R, wherein R is aryl as that term is defined above. Benzene and toluene are non-limiting examples of arenes.

The term “aralkyl” refers to the monovalent group -alkanediyl-aryl, in which the terms alkanediyl and aryl are each used in a manner consistent with the definitions provided above. Non-limiting examples are: phenylmethyl (benzyl, Bn) and 2-phenyl-ethyl.

The term “heteroaryl” refers to a monovalent aromatic group with an aromatic carbon atom or nitrogen atom as the point of attachment, said carbon atom or nitrogen atom forming part of one or more aromatic ring structures, each with three to eight ring atoms, wherein at least one of the ring atoms of the aromatic ring structure(s) is nitrogen, oxygen or sulfur, and wherein the heteroaryl group consists of no atoms other than carbon, hydrogen, aromatic nitrogen, aromatic oxygen and aromatic sulfur. If more than one ring is present, the rings are fused; however, the term heteroaryl does not preclude the presence of one or more alkyl or aryl groups (carbon number limitation permitting) attached to one or more ring atoms. Non-limiting examples of heteroaryl groups include benzoxazolyl, benzimidazolyl, furanyl, imidazolyl (Im), indolyl, indazolyl, isoxazolyl, methylpyridinyl, oxazolyl, oxadiazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. A “heteroarene” refers to the class of compounds having the formula H—R, wherein R is heteroaryl. Pyridine and quinoline are non-limiting examples of heteroarenes.

The term “heteroaralkyl” refers to the monovalent group -alkanediyl-heteroaryl, in which the terms alkanediyl and heteroaryl are each used in a manner consistent with the definitions provided above. Non-limiting examples are: pyridinylmethyl and 2-quinolinyl-ethyl.

The term “heterocycloalkyl” refers to a monovalent non-aromatic group with a carbon atom or nitrogen atom as the point of attachment, said carbon atom or nitrogen atom forming part of one or more non-aromatic ring structures, each with three to eight ring atoms, wherein at least one of the ring atoms of the non-aromatic ring structure(s) is nitrogen, oxygen or sulfur, and wherein the heterocycloalkyl group consists of no atoms other than carbon, hydrogen, nitrogen, oxygen and sulfur. If more than one ring is present, the rings are fused. As used herein, the term does not preclude the presence of one or more alkyl groups (carbon number limitation permitting) attached to one or more ring atoms. Also, the term does not preclude the presence of one or more double bonds in the ring or ring system, provided that the resulting group remains non-aromatic. Non-limiting examples of heterocycloalkyl groups include aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, pyranyl, oxiranyl, and oxetanyl. The term “N-heterocycloalkyl” refers to a heterocycloalkyl group with a nitrogen atom as the point of attachment. N-pyrrolidinyl is an example of such a group.

The term “heterocycloalkalkyl” refers to the monovalent group -alkanediyl-heterocycloalkyl, in which the terms alkanediyl and heterocycloalkyl are each used in a manner consistent with the definitions provided above. Non-limiting examples are: morpholinylmethyl and piperidinylethyl.

The term “acyl” refers to the group —C(O)R, in which R is a hydrogen, alkyl, cycloalkyl, or aryl as those terms are defined above. The groups, —CHO, —C(O)CH₃ (acetyl, Ac), —C(O)CH₂CH₃, —C(O)CH(CH₃)₂, —C(O)CH(CH₂)₂, —C(O)C₆H₅, and —C(O)C₆H₄CH₃ are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkyl group, as defined above, attached to a —CHO group.

The term “alkoxy” refers to the group —OR, in which R is an alkyl, as that term is defined above. Non-limiting examples include: —OCH₃ (methoxy), —OCH₂CH₃ (ethoxy), —OCH₂CH₂CH₃, —OCH(CH₃)₂ (isopropoxy), or —OC(CH₃)₃ (tert-butoxy). The terms “cycloalkoxy”, “alkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as —OR, in which R is cycloalkyl, alkenyl, alkynyl, aryl, aralkyl, heteroaryl, heterocycloalkyl, and acyl, respectively. The term “alkylthio” and “acylthio” refers to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group.

The term “alkylamino” refers to the group —NHR, in which R is an alkyl, as that term is defined above. Non-limiting examples include: —NHCH₃ and —NHCH₂CH₃. The term “dialkylamino” refers to the group —NRR′, in which R and R′ can be the same or different alkyl groups. Non-limiting examples of dialkylamino groups include: —N(CH₃)₂ and —N(CH₃)(CH₂CH₃). The term “amido” (acylamino), when used without the “substituted” modifier, refers to the group —NHR, in which R is acyl, as that term is defined above. A non-limiting example of an amido group is —NHC(O)CH₃.

When a chemical group is used with the “substituted” modifier, one or more hydrogen atom has been replaced, independently at each instance, by —OH, —F, —Cl, —Br, —I, —NH₂, —NO₂, —CO₂H, —CO₂CH₃, —CO₂CH₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. For example, the following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂C1. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂C1 is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups. Non-limiting examples of substituted aralkyls are: (3-chlorophenyl)-methyl, and 2-chloro-2-phenyl-eth-1-yl. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups. The groups —NHC(O)OCH₃ and —NHC(O)NHCH₃ are non-limiting examples of substituted amido groups.

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects or patients. Unless otherwise noted, the term “about” is used to indicate a value of 10% of the reported value, preferably a value of ±5% of the reported value. It is to be understood that, whenever the term “about” is used, a specific reference to the exact numerical value indicated is also included.”

An “active ingredient” (AI) or active pharmaceutical ingredient (API) (also referred to as an active compound, active substance, active agent, pharmaceutical agent, agent, biologically active molecule, or a therapeutic compound) is the ingredient in a pharmaceutical drug that is biologically active.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to the patient or subject, is sufficient to effect such treatment or prevention of the disease as those terms are defined below.

An “excipient” is a pharmaceutically acceptable substance formulated along with the active ingredient(s) of a medication, pharmaceutical composition, formulation, or drug delivery system. Excipients may be used, for example, to stabilize the composition, to bulk up the composition (thus often referred to as “bulking agents,” “fillers,” or “diluents” when used for this purpose), or to confer a therapeutic enhancement on the active ingredient in the final dosage form, such as facilitating drug absorption, reducing viscosity, or enhancing solubility. Excipients include pharmaceutically acceptable versions of antiadherents, binders, coatings, colors, disintegrants, flavors, glidants, lubricants, preservatives, sorbents, sweeteners, and vehicles. The main excipient that serves as a medium for conveying the active ingredient is usually called the vehicle. Excipients may also be used in the manufacturing process, for example, to aid in the handling of the active substance, such as by facilitating powder flowability or non-stick properties, in addition to aiding in vitro stability such as prevention of denaturation or aggregation over the expected shelf life. The suitability of an excipient will typically vary depending on the route of administration, the dosage form, the active ingredient, as well as other factors.

The term “hydrate” when used as a modifier to a compound means that the compound has less than one (e.g., hemihydrate), one (e.g., monohydrate), or more than one (e.g., dihydrate) water molecules associated with each compound molecule, such as in solid forms of the compound.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e. an enzyme, cell, cell receptor or microorganism) by half. The term “EC₅₀” refers to an amount that is an effective concentration to results in a half-maximal response.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human patients are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds disclosed herein which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

A “pharmaceutically acceptable carrier,” “drug carrier,” or simply “carrier” is a pharmaceutically acceptable substance formulated along with the active ingredient medication that is involved in carrying, delivering and/or transporting a chemical agent. Drug carriers may be used to improve the delivery and the effectiveness of drugs, including for example, controlled-release technology to modulate drug bioavailability, decrease drug metabolism, and/or reduce drug toxicity. Some drug carriers may increase the effectiveness of drug delivery to the specific target sites. Examples of carriers include: liposomes, microspheres (e.g., made of poly(lactic-co-glycolic) acid), albumin microspheres, synthetic polymers, nanofibers, protein-DNA complexes, protein conjugates, erythrocytes, virosomes, and dendrimers.

A “pharmaceutical drug” (also referred to as a pharmaceutical, pharmaceutical preparation, pharmaceutical composition, pharmaceutical formulation, pharmaceutical product, medicinal product, medicine, medication, medicament, or simply a drug, agent, or preparation) is a composition used to diagnose, cure, treat, or prevent disease, which comprises an active pharmaceutical ingredient (API) (defined above) and optionally contains one or more inactive ingredients, which are also referred to as excipients (defined above).

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

“Prodrug” means a compound that is convertible in vivo metabolically into an active pharmaceutical ingredient of the present invention. The prodrug itself may or may not have activity with in its prodrug form. For example, a compound comprising a hydroxy group may be administered as an ester that is converted by hydrolysis in vivo to the hydroxy compound. Non-limiting examples of suitable esters that may be converted in vivo into hydroxy compounds include acetates, citrates, lactates, phosphates, tartrates, malonates, oxalates, salicylates, propionates, succinates, fumarates, maleates, methylene-bis-p-hydroxynaphthoate, gentisates, isethionates, di-p-toluoyltartrates, methanesulfonates, ethanesulfonates, benzenesulfonates, p-toluenesulfonates, cyclohexylsulfamates, quinates, and esters of amino acids. Similarly, a compound comprising an amine group may be administered as an amide that is converted by hydrolysis in vivo to the amine compound.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2ⁿ, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease or symptom thereof in a subject or patient that is experiencing or displaying the pathology or symptomatology of the disease.

The term “unit dose” refers to a formulation of the compound or composition such that the formulation is prepared in a manner sufficient to provide a single therapeutically effective dose of the active ingredient to a patient in a single administration. Such unit dose formulations that may be used include but are not limited to a single tablet, capsule, or other oral formulations, or a single vial with a syringeable liquid or other injectable formulations.

The above definitions supersede any conflicting definition in any reference that is incorporated by reference herein. The fact that certain terms are defined, however, should not be considered as indicative that any term that is undefined is indefinite. Rather, all terms used are believed to describe the invention in terms such that one of ordinary skill can appreciate the scope and practice the present invention.

### VI. EMBODIMENTS OF THE DISCLOSURE

The following is a list of the embodiments of the present disclosure.
1. A method of disrupting a membrane comprising:
   (A) contacting the membrane with a compound, wherein the compound is capable of generating a vibronic-driven action and optionally further comprising a targeting moiety; and
   (B) exposing the compound to an energy source sufficient to generate the vibronic-driven action, wherein the vibronic-driven action is sufficient to disrupt the membrane.
2. Use of a compound for disrupting a membrane comprising:
   (A) contacting the membrane with the compound, wherein the compound is capable of generating a vibronic-driven action and optionally further comprising a targeting moiety; and
   (B) exposing the compound to an energy source sufficient to generate the vibronic-driven action, wherein the vibronic-driven action is sufficient to disrupt the membrane.
3. A composition for use in the disrupting of a membrane comprising a compound, wherein the compound is capable of generating a vibronic-driven action and optionally further comprising a targeting moiety, provided that when the compound is exposed to an energy source sufficient to generate the vibronic-driven action, then the vibronic-driven action is sufficient to disrupt the membrane.
4. The method of embodiment 1, wherein the membrane is the outer membrane of a cell.
5. The method of embodiment 1, wherein the membrane is the inner membrane of a cell.
6. The method of either embodiment 1 or embodiment 5, wherein the inner membrane is the membrane of an organelle.
7. The method of embodiment 6, wherein the membrane of the organelle is the membrane of a mitochondria, a nucleus, an endoplasmic reticulum, or a golgi apparatus.
8. The method of embodiment 1, wherein the membrane is the membrane of prokaryotic cell.
9. The method of embodiment 1, wherein the membrane is the membrane of eukaryotic cell.
10. The method according to any one of embodiments 1-7 and 9, wherein the membrane is the membrane of a human cell.
11. The method of embodiment 10, wherein the human cell is a cancer cell.
12. The method of embodiment 10, wherein the human cell is a healthy cell.
13. The method of embodiment 10, wherein the human cell is an adipose cell.
14. The method according to any one of embodiments 1-7, wherein the membrane is a bacterial membrane, a virus, a fungal membrane, or a protozoal membrane.
15. The method of embodiment 14, wherein the membrane is a bacterial membrane.
16. The method of embodiment 14, wherein the membrane is a viral membrane.
17. The method of embodiment 14, wherein the membrane is a fungal membrane.
18. The method of embodiment 14, wherein the membrane is a protozoal membrane.
19. The method according to any one of embodiments 1-7, wherein the membrane is a membrane of a parasite.
20. The method according to any one of embodiments 1-19, wherein the disruption creates a pore in the membrane.
21. The method according to any one of embodiments 1-20, wherein the method results in necrosis of the cell.
22. The method according to any one of embodiments 1-20, wherein the method results in death through the disruption of an organelle in the cell.
23. The method according to any one of embodiments 1-20, wherein the method results in death through the disruption of a nucleus in the cell.
24. The method according to any one of embodiments 1-23, wherein the compound comprises:
   (i) has a net dipole via a charge (cation or anion or radical cation or radical anion) or radical (single unpaired electron);
   (ii) has a high degree of symmetry across the longitudinal and/or transverse axis; and
   (iii) has a resonance structure through a pi-bonded system whereby the charge or radical can oscillate between the near-symmetric two ends via resonance.
25. The method according to any one of embodiments 1-24, wherein the compound is an organomettalic compound.
26. The method of embodiments 25, wherein the organometallic compound is not a nanoparticle.
27. The method of either embodiment 25 or embodiment 26, wherein the organometallic compound is an organic ligand bound individually to one or more metal atoms.
28. The method of embodiment 27, wherein the organic ligand is bound to one metal atom.
29. The method of embodiment 27, wherein the organic ligand is bound to two or more metal atoms.
30. The method according to any one of embodiments 27-29, wherein the metal atom is bound to the organic ligand via a covalent bond.
31. The method according to any one of embodiments 27-29, wherein the metal atom is bound to the organic ligand via an ionic bond.
32. The method according to any one of embodiments 1-24, wherein the compound is an organic molecule.
33. The method of embodiment 32, wherein the organic molecule exhibits either a longitudinal molecular plasmon or a transverse molecular plasmon.
34. The method of either embodiment 32 or embodiment 33, wherein the organic molecule exhibits both a longitudinal molecular plasmon and a transverse molecular plasmon.
35. The method according to any one of embodiments 1-34, wherein the compound is an organic dye.
36. The method according to any one of embodiments 1-35, wherein the compound is further defined by the formula:

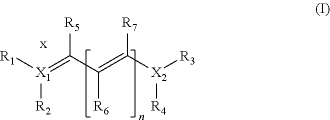

wherein:
   x is a positive or negative charge;
   n is an integer from 0 to 100;
   X₁ and X₂ are each independently a heteroatom selected from O, N, S, B, P, Ge, As, or Se; and
   R₁, R₂, R₃, R₄, R₅, R₆, and R₇ are each independently alkyl_{(C≤18)}, alkenyl_{(C≤18)}, alkynyl_{(C≤18)}, aryl_{(C≤18)}, aralkyl_{(C≤18)}, heteroaryl_{(C≤18)}, heterocycloalkyl_{(C≤18)}, or a substituted version of any of these groups; or
   R₁ and R₂, R₁ and R₅, R₂ and R₅, R₃ and R₄, R₃ and R₇, and R₄ and R₇ are taken together to form one, two, three, four, five, or six aliphatic or aromatic rings; comprising at least three carbon atoms and no more than 36 carbon atoms; optionally comprising one, two, three, four, or five nitrogen, sulfur, or oxygen atom.
37. The method of embodiment 36, wherein the compound is further defined as:

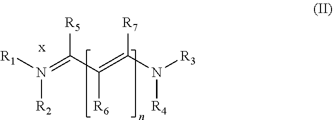

wherein:
   x is a positive charge;
   n is an integer from 0 to 100;
   each R₁, R₂, R₃, R₄, R₅, R₆, and R₇ are each independently hydrogen, alkyl_{(C≤18)}, alkenyl_{(C≤18)}, alkynyl_{(C≤18)}, aryl_{(C≤18)}, aralkyl_{(C≤18)}, heteroaryl_{(C≤18)}, heterocycloalkyl_{(C≤18)}, or a substituted version of any of these groups; or
   each R₁, R₂, R₃, R₄, R₅, R₆, and R₇ are each independently a cell membrane targeting moiety, wherein the cell targeting moiety optionally comprises a linker; or
   each R₁ and R₂, R₁ and R₅, R₂ and R₅, R₃ and R₄, R₃ and R₇, R₄ and R₇, and R₅ and R₇ are taken together and each independently form one, two, three, four, five, or six aliphatic or aromatic rings; comprising at least three carbon atoms and no more than 36 carbon atoms; optionally comprising one, two, three, four, or five nitrogen, sulfur, or oxygen atom.
38. The method of embodiment 36, wherein X₁ and X₂ are identical.
39. The method of either embodiment 36 or embodiment 38, wherein X₁ is N.
40. The method according to any one of embodiments 36, 38, and 39, wherein X₂ is N.
41. The method according to any one of embodiments 36 and 38-40, wherein X₁ and X₂ are N.
42. The method according to any one of embodiments 36-41, wherein R₁ or R₂ are symmetric with R₃ or R₄.
43. The method according to any one of embodiments 36-42, wherein R₁ is taken together with R₅ to form one, two, three, four, or five rings.
44. The method of embodiment 43, wherein R₁ is taken together with R₅ to form two, three, or four rings.
45. The method of either embodiment 43 or embodiment 44, wherein R₁ is taken together with R₅ to form three rings.
46. The method according to any one of embodiments 43-45, wherein R₁ is taken together with R₅ to form three rings, wherein one ring is aliphatic and two rings are aromatic.
47. The method according to any one of embodiments 36-46, wherein R₂ is alkyl_{(C≤18)} or substituted alkyl_{(C≤18)}.
48. The method of embodiment 47, wherein R₂ is alkyl_{(C≤18)}.
49. The method of either embodiment 47 or embodiment 48, wherein R₂ is alkyl_{(C≤8)}.
50. The method according to any one of embodiments 47-49, wherein R₂ is methyl.
51. The method according to any one of embodiments 36-50, wherein R₃ is taken together with R₇ to form one, two, three, four, or five rings.
52. The method of embodiment 51, wherein R₃ is taken together with R₇ to form two, three, or four rings.
53. The method of either embodiment 51 or embodiment 52, wherein R₃ is taken together with R₇ to form three rings.
54. The method according to any one of embodiments 51-53, wherein R₃ is taken together with R₇ to form three rings, wherein one ring is aliphatic and two rings are aromatic.
55. The method according to any one of embodiments 36-54, wherein R₄ is alkyl_{(C≤18)} or substituted alkyl_{(C≤18)}.
56. The method of embodiment 55, wherein R₄ is alkyl_{(C≤18)}.
57. The method of either embodiment 47 or embodiment 48, wherein R₄ is alkyl_{(C≤8)}.
58. The method according to any one of embodiments 55-57, wherein R₄ is methyl.
59. The method according to any one of embodiments 36-58, wherein R₆ is hydrogen.
60. The method according to any one of embodiments 36-59, wherein R₅ and R₇ are taken together and form one, two, or three rings.
61. The method of embodiment 60, wherein R₅ and R₇ are taken together and form a single ring.
62. The method of embodiment 61, wherein the single ring is a five, six, or seven membered ring.
63. The method according to any one of embodiments 36-62, wherein n is an integer from 1 to 10.
64. The method according to any one of embodiments 36-63, wherein n is an integer selected from 2, 3, or 4.
65. The method of embodiment 64, wherein n is 3.
66. The method according to any one of embodiments 36-65, wherein R₄ is a cell targeting moiety with a linker.
67. The method of embodiment 66, wherein the linker is an alkyl chain, an alkenyl chain, an aryl chain, a peptide chain, a polyethylene glycol chain, or a polypropylene chain.
68. The method of embodiment 67, wherein the linker further comprises one or more joining functional group selected from ether, amide, disulfide, ester, amine, or thioether.
69. The method of either embodiment 67 or embodiment 68, wherein the linker is two alkyl chains with an amide joining functional group.
70. The method according to any one of embodiments 36-69, wherein the cell targeting moiety is a functional group that associates with the membrane, a carbohydrate or polysaccharide that binds to one or more markers on the membrane, a lipid that binds to one or more markers on the cell membrane, a small molecule that binds to one or more markers on the cell membrane, an aptamer that binds to one or more markers on the membrane, or a peptide or an antibody that binds to one or more markers on the membrane.
71. The method of embodiment 70, wherein the cell targeting moiety is a functional group that associates with the cell membrane.
72. The method of embodiment 71, wherein the functional group that associates with the cell membrane is an amine.
73. The method of embodiment 72, wherein the amine is protonated.
74. The method according to any one of embodiments 1-73, wherein the compound is further defined as:

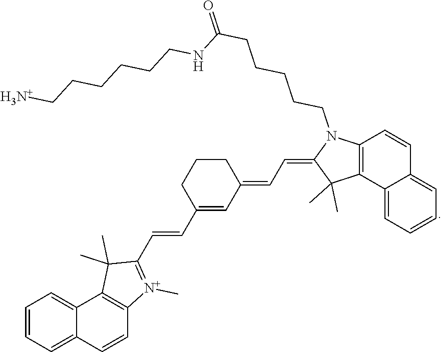 [SMILES: C[N+]1=C(/C=C/C2=CC(=C/C=C3/N(CCCCCC(=O)NCCCCCC[NH3+])c4ccc5ccccc5c4C3(C)C)/CCC2)C(C)(C)c2c1ccc1ccccc21]

75. The method according to any one of embodiments 1-73, wherein the compound is further defined as:

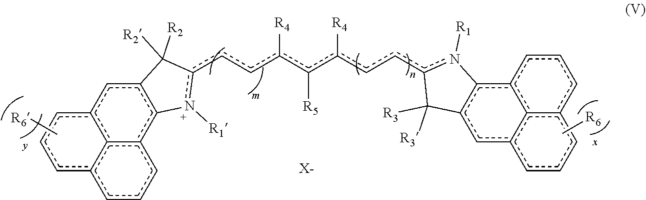

wherein:
   R₁ and R₁′ are each independently alkyl_{(C≤8)}, substituted alkyl_{(C≤8)}, or a group of the formula:

-A-NRₐRₐ′Rₐ″
wherein:
   A is an alkanediyl_{(C≤12)} or substituted alkanediyl_{(C≤12)};
   Rₐ and Rₐ′ are each independently hydrogen or alkyl_{(C≤8)}; and
   Rₐ″ is absent, hydrogen, or alkyl_{(C≤8)};
provided at least one of R₁ and R₁′ is a group of the formula: -A-NRₐRₐ′Rₐ″
R₂, R₂′, R₃, and R₃′ are each independently hydrogen, alkyl_{(C≤8)}, or substituted alkyl_{(C≤8)};
R₄ and R₄′ are each independently hydrogen, alkyl_{(C≤8)}, substituted alkyl_{(C≤8)}, or R₄ and R₄′ are taken together as a cycloalkyl group;
R₅ is hydrogen, halo, carboxy, alkyl_{(C≤8)}, substituted alkyl_{(C≤8)}, or —C(O)OR_{b}, wherein R_{b} is alkyl_{(C≤6)} or substituted alkyl_{(C≤6)};
R₆ is hydrogen, amino, halo, hydroxy, or alkyl_{(C≤12)}, alkoxy_{(C≤12)}, alkylamino_{(C≤8)}, dialkylamino_{(C≤8)}, acyl_{(C≤8)}, acyl_{(C≤8)}, acyl_{(C≤8)}, or a substituted version thereof;
m and n are each 0, 1, 2, or 3;
x and y are each independently 0, 1, 2, 3, 4, or 5;
X is a monovalent anion; and
each of the rings is optionally present as either an aromatic or aliphatic ring.
76. The method according to embodiments 1-75, wherein the compound is at least one compound shown below:

Structure

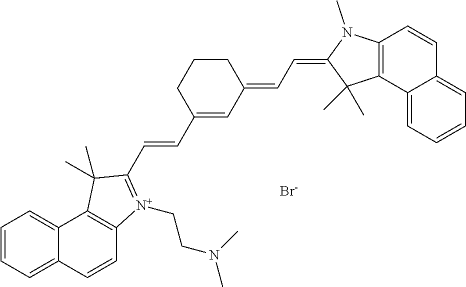 [SMILES: CN(C)CC[N+]1=C(/C=C/C2=CC(=C/C=C3/N(C)c4ccc5ccccc5c4C3(C)C)/CCC2)C(C)(C)c2c1ccc1ccccc21 ; [Br-]]

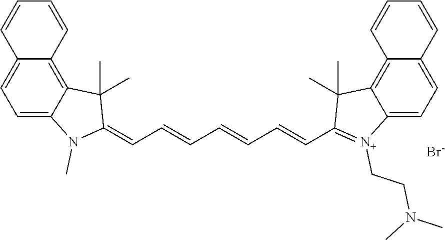 [SMILES: CN(C)CC[N+]1=C(/C=C/C=C/C=C/C=C2/N(C)c3ccc4ccccc4c3C2(C)C)C(C)(C)c2c1ccc1ccccc21 ; [Br-]]

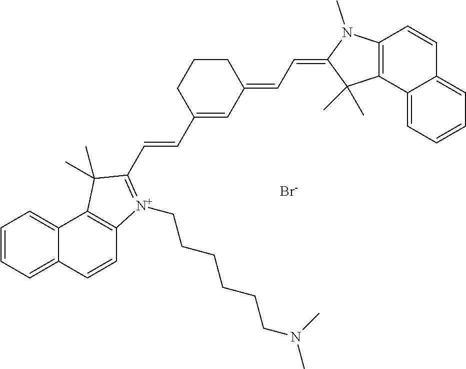 [SMILES: CN(C)CCCCCC[N+]1=C(/C=C/C2=CC(=C/C=C3/N(C)c4ccc5ccccc5c4C3(C)C)/CCC2)C(C)(C)c2c1ccc1ccccc21 ; [Br-]]

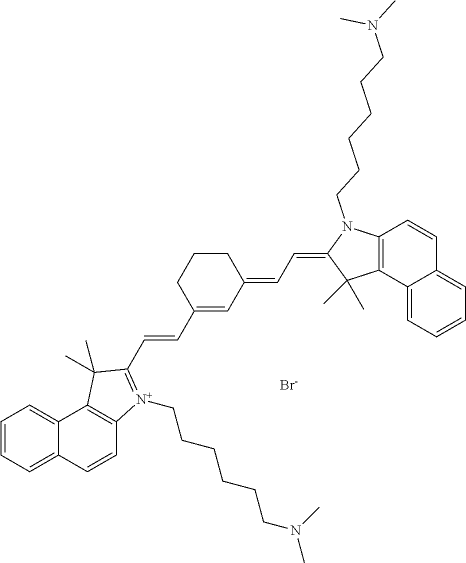 [SMILES: CN(C)CCCCCCN1/C(=C/C=C2C=C(/C=C/C3=[N+](CCCCCCN(C)C)c4ccc5ccccc5c4C3(C)C)CCC/2)C(C)(C)c2c1ccc1ccccc21 ; [Br-]]

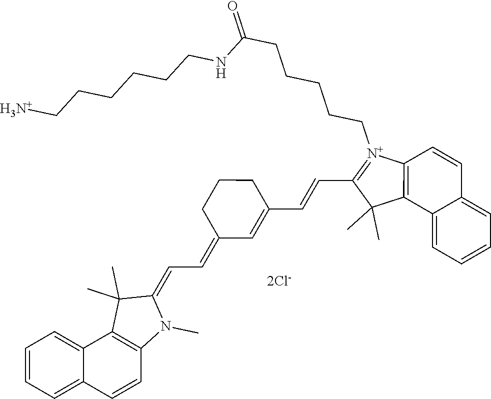 [SMILES: CN1/C(=C\C=C2C=C(/C=C/C3=[N+](CCCCCC(=O)NCCCCCC[NH3+])c4ccc5ccccc5c4C3(C)C)CCC\2)C(C)(C)c2c1ccc1ccccc21]

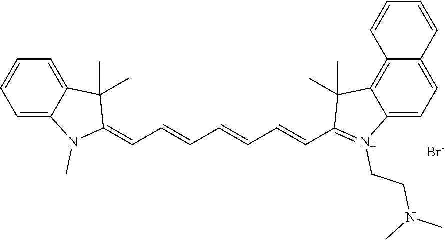 [SMILES: CN(C)CC[N+]1=C(/C=C/C=C/C=C/C=C2/N(C)c3ccccc3C2(C)C)C(C)(C)c2c1ccc1ccccc21 ; [Br-]]

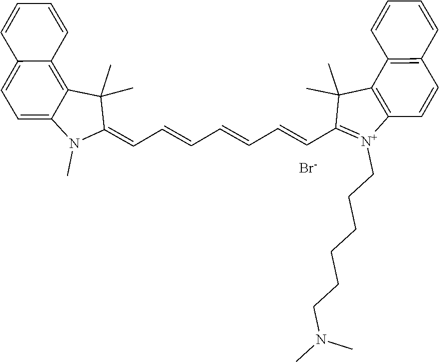 [SMILES: CN(C)CCCCCC[N+]1=C(/C=C/C=C/C=C/C=C2/N(C)c3ccc4ccccc4c3C2(C)C)C(C)(C)c2c1ccc1ccccc21 ; [Br-]]

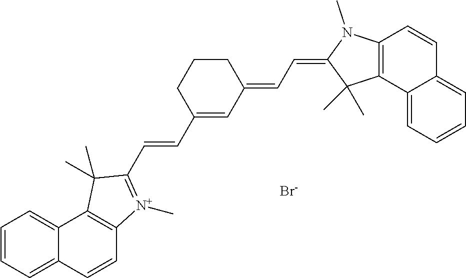 [SMILES: CN1/C(=C/C=C2C=C(/C=C/C3=[N+](C)c4ccc5ccccc5c4C3(C)C)CCC/2)C(C)(C)c2c1ccc1ccccc21 ; [Br-]]

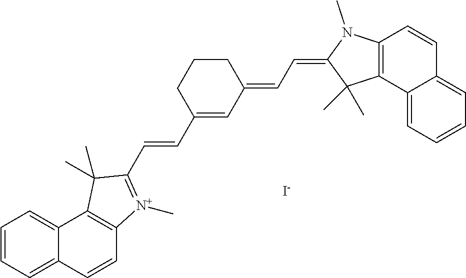 [SMILES: CN1/C(=C/C=C2C=C(/C=C/C3=[N+](C)c4ccc5ccccc5c4C3(C)C)CCC/2)C(C)(C)c2c1ccc1ccccc21 ; [I-]]

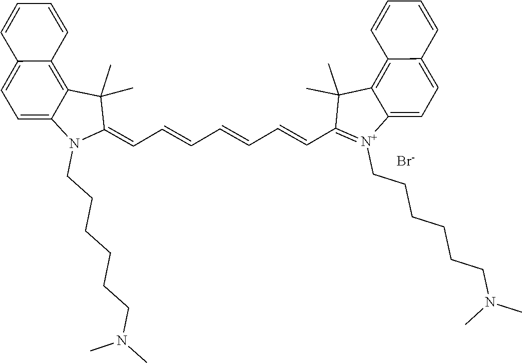 [SMILES: CN(C)CCCCCCN1/C(=C/C=C/C=C/C=C/C2=[N+](CCCCCCN(C)C)c3ccc4ccccc4c3C2(C)C)C(C)(C)c2c1ccc1ccccc21 ; [Br-]]

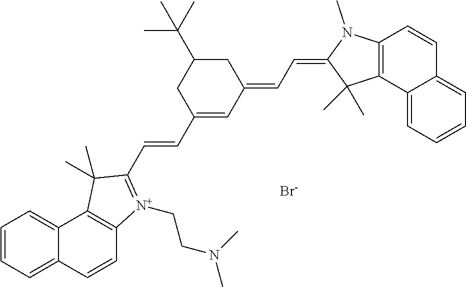 [SMILES: CN(C)CC[N+]1=C(/C=C/C2=CC(=C/C=C3/N(C)c4ccc5ccccc5c4C3(C)C)/CC(C(C)(C)C)C2)C(C)(C)c2c1ccc1ccccc21 ; [Br-]]

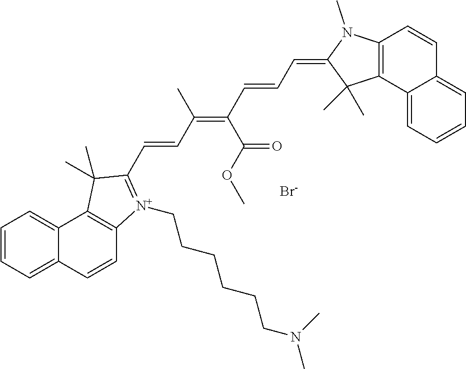 [SMILES: COC(=O)C(/C=C/C=C1/N(C)c2ccc3ccccc3c2C1(C)C)=C(C)\C=C\C1=[N+](CCCCCCN(C)C)c2ccc3ccccc3c2C1(C)C ; [Br-]]

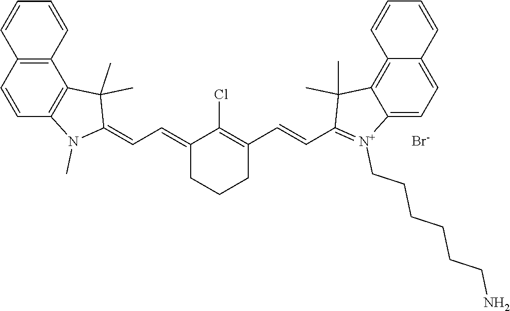 [SMILES: CN1/C(=C/C=C2\CCCC(/C=C/C3=[N+](CCCCCCN)c4ccc5ccccc5c4C3(C)C)=C2Cl)C(C)(C)c2c1ccc1ccccc21 ; [Br-]]

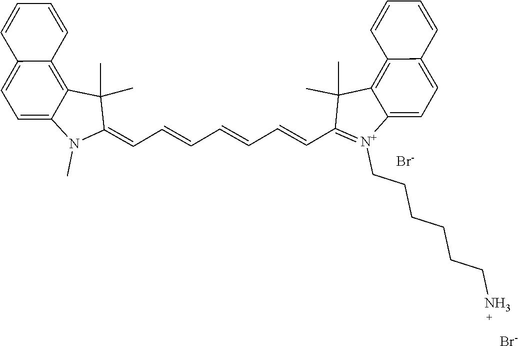 [SMILES: CN1/C(=C/C=C/C=C/C=C/C2=[N+](CCCCCCN)c3ccc4ccccc4c3C2(C)C)C(C)(C)c2c1ccc1ccccc21 ; [Br-] ; [Br-]]

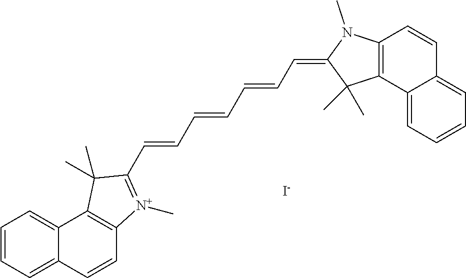 [SMILES: CN1/C(=C/C=C/C=C/C=C/C2=[N+](C)c3ccc4ccccc4c3C2(C)C)C(C)(C)c2c1ccc1ccccc21 ; [I-]]

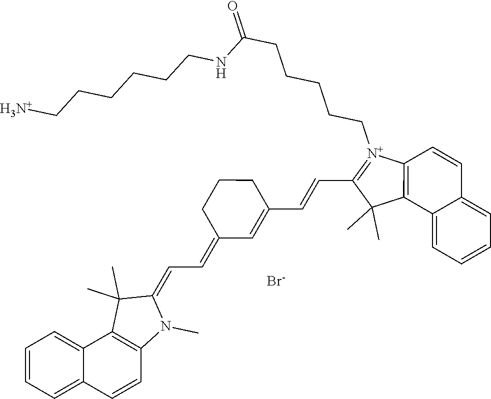 [SMILES: CN1/C(=C\C=C2C=C(/C=C/C3=[N+](CCCCCC(=O)NCCCCCC[NH3+])c4ccc5ccccc5c4C3(C)C)CCC\2)C(C)(C)c2c1ccc1ccccc21 ; [Br-]]

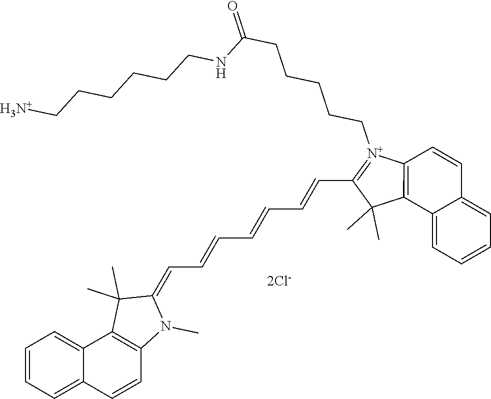 [SMILES: CN1\C(=C/C=C/C=C/C=C/C2=[N+](CCCCCC(=O)NCCCCCC[NH3+])c3ccc4ccccc4c3C2(C)C)C(C)(C)c2c1ccc1ccccc21]

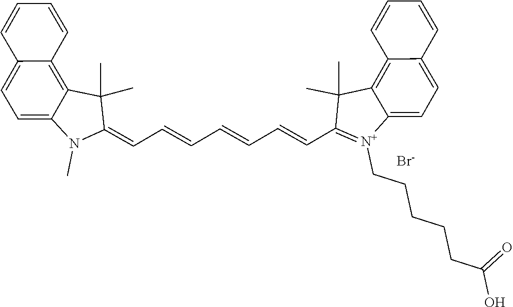 [SMILES: CN1/C(=C/C=C/C=C/C=C/C2=[N+](CCCCCC(=O)O)c3ccc4ccccc4c3C2(C)C)C(C)(C)c2c1ccc1ccccc21 ; [Br-]]

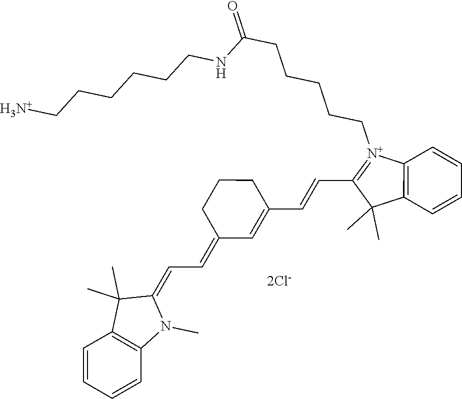 [SMILES: CN1/C(=C\C=C2C=C(/C=C/C3=[N+](CCCCCC(=O)NCCCCCC[NH3+])c4ccccc4C3(C)C)CCC\2)C(C)(C)c2ccccc21]

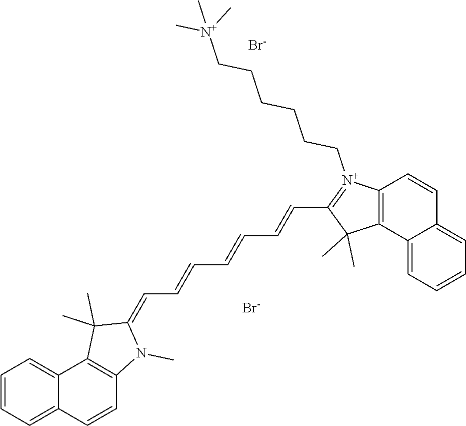 [SMILES: CN1\C(=C/C=C/C=C/C=C/C2=[N+](CCCCCC[N+](C)(C)C)c3ccc4ccccc4c3C2(C)C)C(C)(C)c2c1ccc1ccccc21 ; [Br-] ; [Br-]]

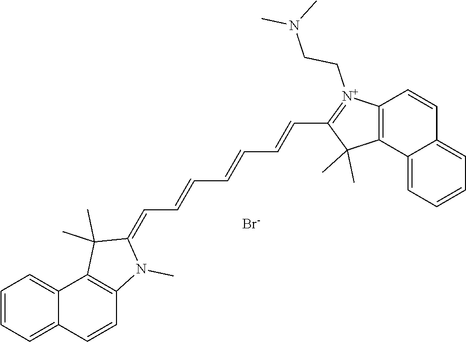 [SMILES: CN(C)CC[N+]1=C(/C=C/C=C/C=C/C=C2\N(C)c3ccc4ccccc4c3C2(C)C)C(C)(C)c2c1ccc1ccccc21 ; [Br-]]

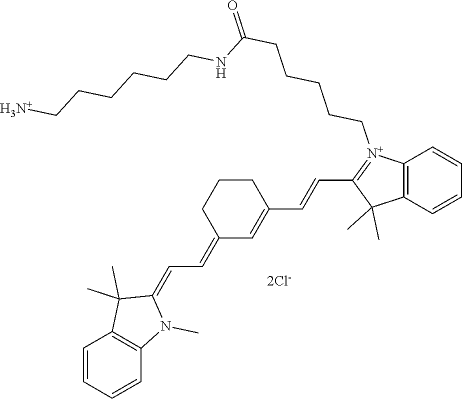 [SMILES: CN1/C(=C\C=C2C=C(/C=C/C3=[N+](CCCCCC(=O)NCCCCCC[NH3+])c4ccccc4C3(C)C)CCC\2)C(C)(C)c2ccccc21]

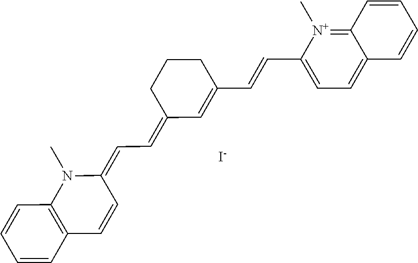 [SMILES: CN1/C(=C/C=C2C=C(/C=C/c3ccc4ccccc4[n+]3C)CCC/2)C=Cc2ccccc21 ; [I-]]

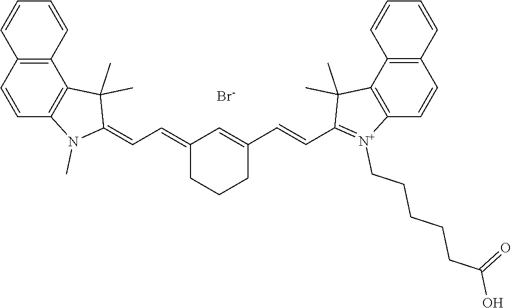 [SMILES: CN1/C(=C/C=C2C=C(/C=C/C3=[N+](CCCCCC(=O)O)c4ccc5ccccc5c4C3(C)C)CCC/2)C(C)(C)c2c1ccc1ccccc21 ; [Br-]]

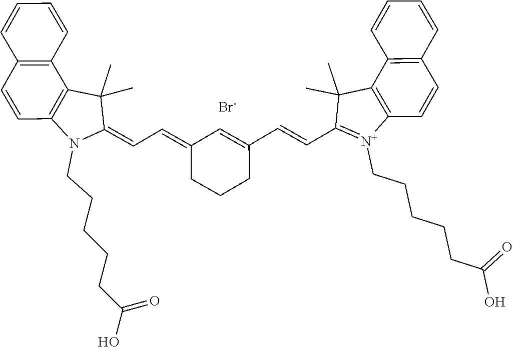 [SMILES: CC1(C)C(/C=C/C2=CC(=C/C=C3/N(CCCCCC(=O)O)c4ccc5ccccc5c4C3(C)C)/CCC2)=[N+](CCCCCC(=O)O)c2ccc3ccccc3c21 ; [Br-]]

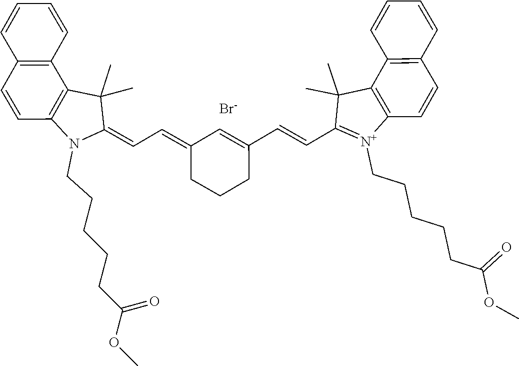 [SMILES: COC(=O)CCCCCN1/C(=C/C=C2C=C(/C=C/C3=[N+](CCCCCC(=O)OC)c4ccc5ccccc5c4C3(C)C)CCC/2)C(C)(C)c2c1ccc1ccccc21 ; [Br-]]

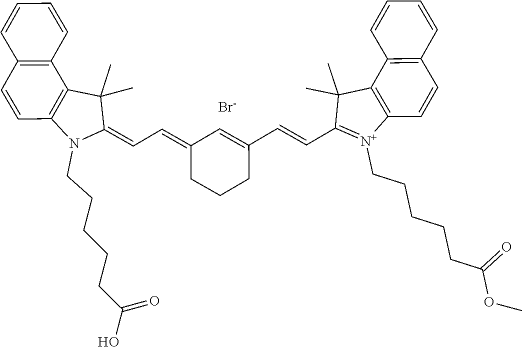 [SMILES: COC(=O)CCCCC[N+]1=C(/C=C/C2=CC(=C/C=C3/N(CCCCCC(=O)O)c4ccc5ccccc5c4C3(C)C)/CCC2)C(C)(C)c2c1ccc1ccccc21 ; [Br-]]

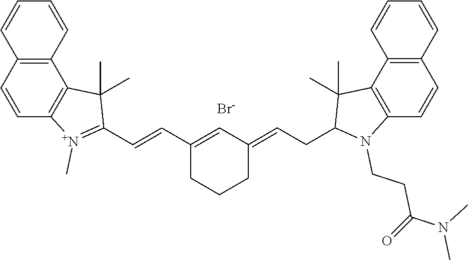 [SMILES: CN(C)C(=O)CCN1c2ccc3ccccc3c2C(C)(C)C1C/C=C1C=C(/C=C/C2=[N+](C)c3ccc4ccccc4c3C2(C)C)CCC/1 ; [Br-]]

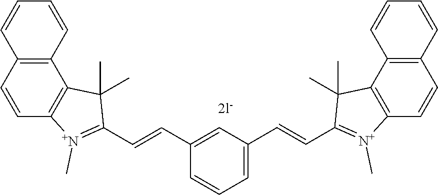

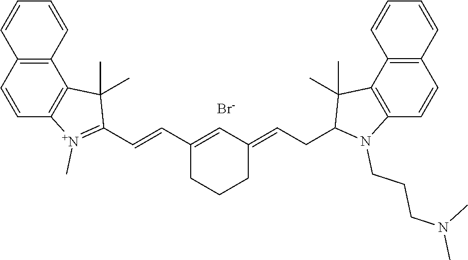 [SMILES: CN(C)CCCN1c2ccc3ccccc3c2C(C)(C)C1C/C=C1C=C(/C=C/C2=[N+](C)c3ccc4ccccc4c3C2(C)C)CCC/1 ; [Br-]]

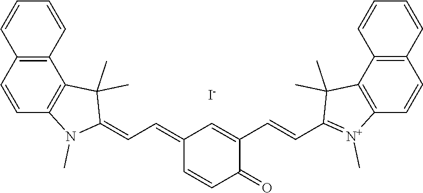 [SMILES: CN1/C(=C/C=C2\C=CC(=O)C(/C=C/C3=[N+](C)c4ccc5ccccc5c4C3(C)C)=C2)C(C)(C)c2c1ccc1ccccc21 ; [I-]]

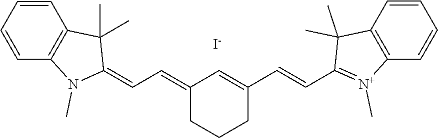 [SMILES: CN1/C(=C/C=C2C=C(/C=C/C3=[N+](C)c4ccccc4C3(C)C)CCC/2)C(C)(C)c2ccccc21 ; [I-]]

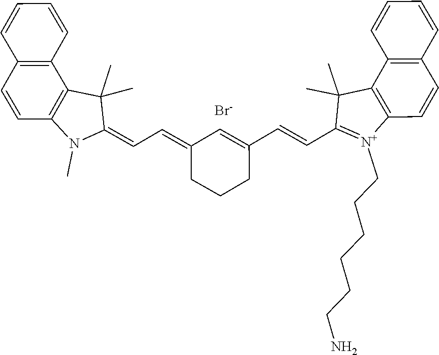 [SMILES: CN1/C(=C/C=C2C=C(/C=C/C3=[N+](CCCCCCN)c4ccc5ccccc5c4C3(C)C)CCC/2)C(C)(C)c2c1ccc1ccccc21 ; [Br-]]

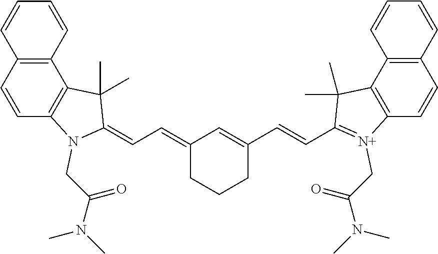 [SMILES: CN(C)C(=O)CN1/C(=C/C=C2C=C(/C=C/C3=[N+](CC(=O)N(C)C)c4ccc5ccccc5c4C3(C)C)CCC/2)C(C)(C)c2c1ccc1ccccc21]

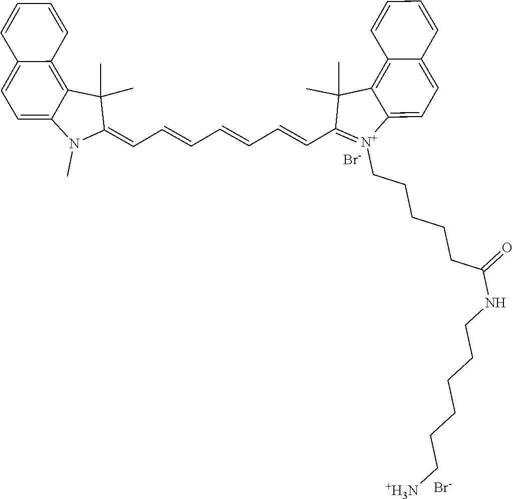 [SMILES: CN1/C(=C/C=C/C=C/C=C/C2=[N+](CCCCCC(=O)NCCCCCC[NH3+])c3ccc4ccccc4c3C2(C)C)C(C)(C)c2c1ccc1ccccc21 ; [Br-] ; [Br-]]

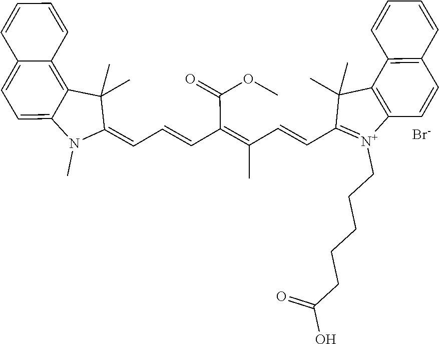 [SMILES: COC(=O)C(/C=C/C=C1/N(C)c2ccc3ccccc3c2C1(C)C)=C(C)\C=C\C1=[N+](CCCCCC(=O)O)c2ccc3ccccc3c2C1(C)C ; [Br-]]

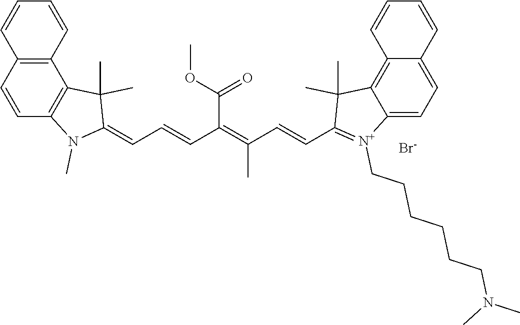 [SMILES: COC(=O)C(/C=C/C=C1/N(C)c2ccc3ccccc3c2C1(C)C)=C(C)\C=C\C1=[N+](CCCCCCN(C)C)c2ccc3ccccc3c2C1(C)C ; [Br-]]

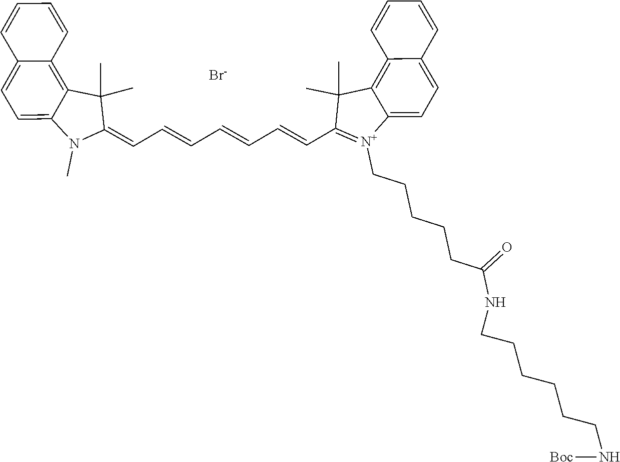 [SMILES: CN1/C(=C/C=C/C=C/C=C/C2=[N+](CCCCCC(=O)NCCCCCCNC(=O)OC(C)(C)C)c3ccc4ccccc4c3C2(C)C)C(C)(C)c2c1ccc1ccccc21 ; [Br-]]

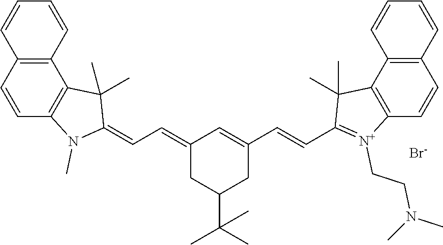 [SMILES: CN(C)CC[N+]1=C(/C=C/C2=CC(=C/C=C3/N(C)c4ccc5ccccc5c4C3(C)C)/CC(C(C)(C)C)C2)C(C)(C)c2c1ccc1ccccc21 ; [Br-]]

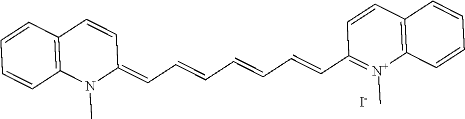 [SMILES: CN1/C(=C/C=C/C=C/C=C/c2ccc3ccccc3[n+]2C)C=Cc2ccccc21 ; [I-]]

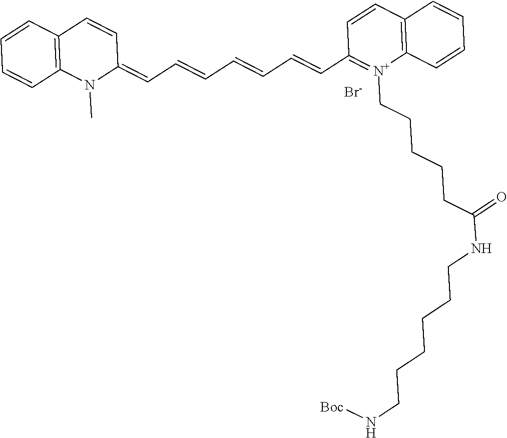 [SMILES: CN1/C(=C/C=C/C=C/C=C/c2ccc3ccccc3[n+]2CCCCCC(=O)NCCCCCCNC(=O)OC(C)(C)C)C=Cc2ccccc21 ; [Br-]]

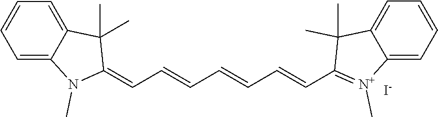 [SMILES: CN1/C(=C/C=C/C=C/C=C/C2=[N+](C)c3ccccc3C2(C)C)C(C)(C)c2ccccc21 ; [I-]]

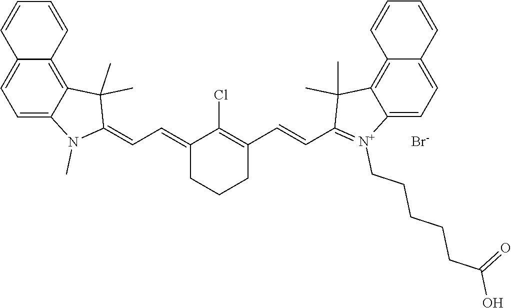 [SMILES: CN1/C(=C/C=C2\CCCC(/C=C/C3=[N+](CCCCCC(=O)O)c4ccc5ccccc5c4C3(C)C)=C2Cl)C(C)(C)c2c1ccc1ccccc21 ; [Br-]]

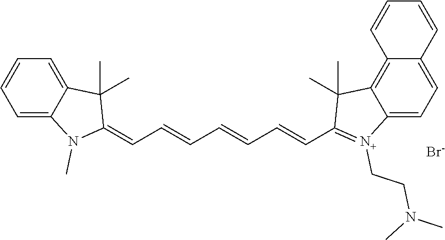 [SMILES: CN(C)CC[N+]1=C(/C=C/C=C/C=C/C=C2/N(C)c3ccccc3C2(C)C)C(C)(C)c2c1ccc1ccccc21 ; [Br-]]

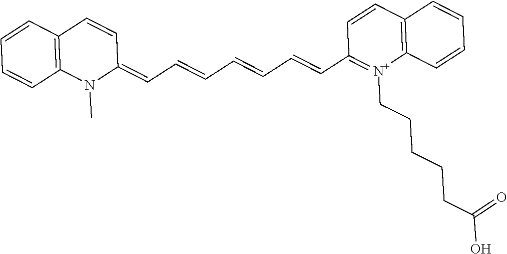 [SMILES: CN1/C(=C/C=C/C=C/C=C/c2ccc3ccccc3[n+]2CCCCCC(=O)O)C=Cc2ccccc21]

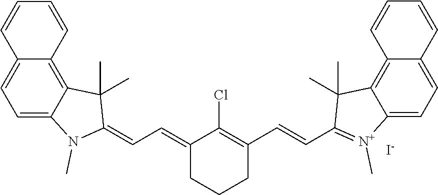 [SMILES: CN1/C(=C/C=C2\CCCC(/C=C/C3=[N+](C)c4ccc5ccccc5c4C3(C)C)=C2Cl)C(C)(C)c2c1ccc1ccccc21 ; [I-]]

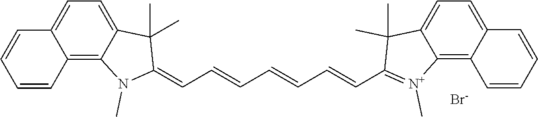 [SMILES: CN1/C(=C/C=C/C=C/C=C/C2=[N+](C)c3c(ccc4ccccc34)C2(C)C)C(C)(C)c2ccc3ccccc3c21 ; [Br-]]

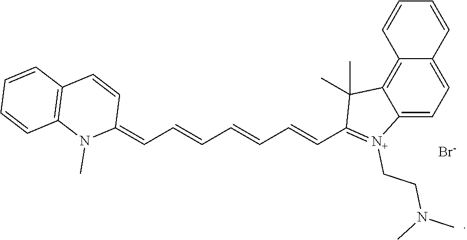 [SMILES: CN(C)CC[N+]1=C(/C=C/C=C/C=C/C=C2\C=Cc3ccccc3N2C)C(C)(C)c2c1ccc1ccccc21 ; [Br-]]

77. The method according to any one of embodiments 1-35, wherein the compound is further defined as:

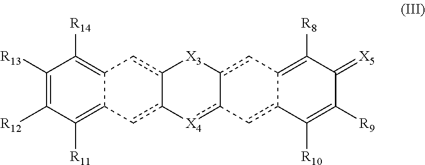

wherein:
   X₃ is —O—, —S—, —C(O)—, —C(S)—, —NRₐ—, or —Si(Rᵤ)(R_{u′})—, wherein:
      Rₐ is hydrogen, alkyl_{(C≤8)}, substituted alkyl_{(C≤8)}, acyl_{(C≤8)}, or substituted acyl_{(C≤8)};
      Rᵤ and R_{u′} are each independently alkyl_{(C≤8)}, substituted alkyl_{(C≤8)}, aryl_{(C≤12)}, or substituted aryl_{(C≤12)};
   X₄ is —N═, —C(R_{b})═, or —C(R_{c})(R_{d})— wherein:
      R_{b} is hydrogen; or
      alkyl_{(C≤8)}, substituted alkyl_{(C≤8)}, aryl_{(C≤12)}, or substituted aryl_{(C≤12)};
      R_{c} and R_{d} are taken together to form a group of the formula:

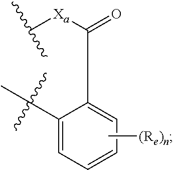

wherein:
n is 0, 1, 2, 3, or 4;
Rₑ at each instance is independently hydrogen, halo, hydroxy, or amino; or
alkyl_{(C≤8)}, alkoxy_{(C≤8)}, alkylamino_{(C≤8)}, dialkylamino_{(C≤8)}, or a substituted version of any of these groups; or
Xₐ is —O— or —NR_{f}—, wherein:
R_{f} is hydrogen; or
alkyl_{(C≤8)}, substituted alkyl_{(C≤8)}, acyl_{(C≤8)}, or substituted acyl_{(C≤8)}; or
—C(O)NHR_{g}, wherein:
R_{g} is alkyl_{(C≤8)}, substituted alkyl_{(C≤8)}, alkenyl_{(C≤8)}, or substituted alkenyl_{(C≤8)};
X₅ is NH, O, or S;
R₈ is hydrogen, halo, hydroxy, amino, nitro, or cyano; or
   alkyl_{(C≤8)}, alkoxy_{(C≤8)}, alkylamino_{(C≤8)}, dialkylamino_{(C≤8)}, amido_{(C≤8)}, or a substituted version of any of these groups; or
   —C(O)Rₕ, wherein;
   Rₕ is hydroxy or amino; or
   alkyl_{(C≤8)}, alkoxy_{(C≤8)}, alkylamino_{(C≤8)}, dialkylamino_{(C≤8)}, or a substituted version of any of these groups; and
R₉, R₁₀, R₁₁, R₁₂, R₁₃, and R₁₄ are each independently hydrogen, halo, hydroxy, amino, nitro, or cyano; or
   alkyl_{(C≤8)}, alkoxy_{(C≤8)}, alkylamino_{(C≤8)}, dialkylamino_{(C≤8)}, amido_{(C≤8)}, or a substituted version of any of these groups; or
   —C(O)Rᵢ, wherein;
   Rᵢ is hydroxy or amino; or
   alkyl_{(C≤8)}, alkoxy_{(C≤8)}, alkylamino_{(C≤8)}, dialkylamino_{(C≤8)}, or a substituted version of any of these groups; or
R₉ and R₁₀, R₁₁ and R₁₂, R₁₂ and R₁₃, or R₁₃ and R₁₄ are taken together to form an arene_{(C≤12)}, a substituted arene_{(C≤12)}, a heteroarene_{(C≤12)}, or a substituted heteroarene_{(C≤12)};
or a pharmaceutically acceptable salt thereof.
78. The method of embodiment 77, wherein the compound is further defined as:

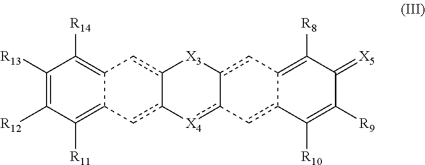

wherein:
   X₃ is —O—, —S—, —C(O)—, —C(S)—, —NRₐ—, or —Si(Rᵤ)(R_{u′})—, wherein:
      Rₐ is hydrogen, alkyl_{(C≤8)}, substituted alkyl_{(C≤8)}, acyl_{(C≤8)}, or substituted acyl_{(C≤8)};
      Rᵤ and R_{u′} are each independently alkyl_{(C≤8)}, substituted alkyl_{(C≤8)}, aryl_{(C≤12)}, or substituted aryl_{(C≤12)};
   X₄ is —N═, —C(R_{b})═, or —C(R_{c})(R_{d})— wherein:
      R_{b} is hydrogen; or
      alkyl_{(C≤8)}, substituted alkyl_{(C≤8)}, aryl_{(C≤12)}, or substituted aryl_{(C≤12)};
      R_{c} and R_{d} are taken together to form a group of the formula:

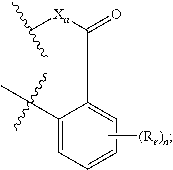

wherein:
n is 0, 1, 2, 3, or 4;
Rₑ at each instance is independently hydrogen, halo, hydroxy, or amino; or
alkyl_{(C≤8)}, alkoxy_{(C≤8)}, alkylamino_{(C≤8)}, dialkylamino_{(C≤8)}, or a substituted version of any of these groups; or
Xₐ is —O— or —NR_{f}—, wherein:
R_{f} is hydrogen; or
alkyl_{(C≤8)}, substituted alkyl_{(C≤8)}, acyl_{(C≤8)}, or substituted acyl_{(C≤8)}; or
—C(O)NHR_{g}, wherein:
R_{g} is alkyl_{(C≤8)}, substituted alkyl_{(C≤8)}, alkenyl_{(C≤8)}, or substituted alkenyl_{(C≤8)};
X₅ is NH, ⁺NR′R″, O, or S;
   wherein:
   R′ and R″ are each independently hydrogen, alkyl_{(C≤8)}, or substituted alkyl_{(C≤8)}; or a cell targeting moiety that optionally comprises a linker;
R₈ is hydrogen, halo, hydroxy, amino, nitro, or cyano; or
   alkyl_{(C≤8)}, alkoxy_{(C≤8)}, alkylamino_{(C≤8)}, dialkylamino_{(C≤8)}, amido_{(C≤8)}, or a substituted version of any of these groups; or
   —C(O)Rₕ, wherein;
   Rₕ is hydroxy or amino; or
   alkyl_{(C≤8)}, alkoxy_{(C≤8)}, alkylamino_{(C≤8)}, dialkylamino_{(C≤8)}, or a substituted version of any of these groups; and
R₉, R₁₀, R₁₁, R₁₂, R₁₃, and R₁₄ are each independently hydrogen, halo, hydroxy, amino, nitro, or cyano; or
   alkyl_{(C≤8)}, alkoxy_{(C≤8)}, alkylamino_{(C≤8)}, dialkylamino_{(C≤8)}, amido_{(C≤8)}, or a substituted version of any of these groups; or
   —C(O)Rᵢ, wherein;
   Rᵢ is hydroxy or amino; or
   alkyl_{(C≤8)}, alkoxy_{(C≤8)}, alkylamino_{(C≤8)}, dialkylamino_{(C≤8)}, or a substituted version of any of these groups; or
R₉ and R₁₀, R₁₁ and R₁₂, R₁₂ and R₁₃, or R₁₃ and R₁₄ are taken together to form an arene_{(C≤12)}, a substituted arene_{(C≤12)}, a heteroarene_{(C≤12)}, or a substituted heteroarene_{(C≤12)};
or a pharmaceutically acceptable salt thereof.
79. The method of either embodiment 77 or embodiment 78, wherein X₃ is —S—.
80. The method according to any one of embodiments 77-79, wherein X₄ is —N═.
81. The method according to any one of embodiments 77-80, wherein R₁₃ is dialkylamino_{(C≤8)} or substituted dialkylamino_{(C≤8)}.
82. The method of embodiment 81, wherein R₁₃ is dialkylamino_{(C≤8)}.
83. The method of either embodiment 81 or embodiment 82, wherein R₁₃ is dimethylamino.
84. The method according to any one of embodiments 77-83, wherein R₉ is hydrogen.
85. The method according to any one of embodiments 77-84, wherein R₁₀ is hydrogen.
86. The method according to any one of embodiments 77-85, wherein R₁₁ is hydrogen.
87. The method according to any one of embodiments 77-86, wherein R₁₂ is hydrogen.
88. The method according to any one of embodiments 77-87, wherein R₁₄ is hydrogen.
89. The method according to any one of embodiments 77-88, wherein X₅ is ⁺NR′R″.
90. The method of embodiment 89, wherein R′ is alkyl_{(C≤8)} or substituted alkyl_{(C≤8)}.
91. The method of embodiment 90, wherein R′ is alkyl_{(C≤8)}.
92. The method of embodiment 91, wherein R′ is methyl.
93. The method according to any one of embodiments 89-92, wherein R″ is alkyl_{(C≤8)} or substituted alkyl_{(C≤8)}.
94. The method of embodiment 93, wherein R″ is alkyl_{(C≤8)}.
95. The method of embodiment 94, wherein R″ is methyl.
96. The method according to any one of embodiments 89-92, wherein R″ is a cell targeting moiety.
97. The method of embodiments 96, wherein the cell targeting moiety further comprises a linker.
98. The method according to any one of embodiments 1-35 and 77-97, wherein the compound is further defined as:

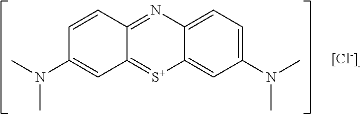

99. The method according to any one of embodiments 1-98, wherein the energy source is gamma rays, X-rays, ultraviolet (UV) light, visible (Vis) light, near-infrared (NIR) light, infrared light (IR), microwaves, radio waves, electric fields, ionizing radiation, magnetic fields, mechanical forces, ultrasound, or combinations thereof.
100. The method according to any one of embodiments 1-99, wherein the energy source is light.
101. The method of embodiment 100, wherein the energy source is light with a wavelength from about 250 nm to about 2,000 nm.
102. The method of embodiment 101, wherein the wavelength is from about 350 nm to about 1,000 nm.
103. The method of embodiment 102, wherein the wavelength is from about 450 nm to about 900 nm.
104. The method according to any one of embodiments 1-103, wherein the intensity of the energy source is less than 200 mW/cm².
105. The method of embodiment 104, wherein the intensity of the energy is less than 100 mW/cm².
106. The method of embodiment 105, wherein the intensity of the energy is less than 25 mW/cm².
107. A method of treating a disease or disorder in a patient comprising:
   (A) contacting the cell membrane of at least one cell of said patient with a compound, wherein the compound capable of generating a vibronic-driven action and optionally further comprising a cell targeting moiety; and
   (B) exposing the compound to an energy source sufficient to generate a vibronic-driven action, wherein the vibronic-driven action is sufficient to disrupt the cell membrane of at least one cell of said patient.
108. The method of embodiment 107, wherein the method further comprises administering the compound with a therapeutic agent.
109. The method of embodiment 108, wherein the method comprises administering the compound in combination with the therapeutic agent.
110. The method according to any one of embodiments 107-109, wherein the contacting of step (A) comprises administering the compound.
111. The method according to any one of embodiments 107-110, wherein the compound disrupts the cell membrane allowing the therapeutic agent to enter a cell.
112. The method according to any one of embodiments 107-111, wherein the therapeutic agent is sufficient to treat or prevent the disease or disorder.
113. The method according to any one of embodiments 107-112, wherein the compound is further defined as the compound in embodiments 32-97.
114. The method according to any one of embodiments 107-113, wherein the patient is a mammal.
115. The method of embodiment 114, wherein the mammal is a human.
116. A method of opening a cell membrane comprising:
   (A) contacting the cell membrane with a compound, wherein the compound capable of generating a vibronic-driven action and optionally further comprising a cell targeting moiety; and
   (B) exposing the compound to an energy source sufficient to generate a vibronic-driven action, wherein the vibronic-driven action is sufficient to open the cell membrane.
117. The method of embodiment 116, wherein the method comprises treating a disease or disorder.
118. The method of either embodiment 116 or embodiment 117, wherein the method comprises killing one or more cells.
119. The method of embodiment 118, wherein the cell is killed by necrosis.
120. The method of either embodiment 118 or embodiment 119, wherein the cell is a parasitic cell.
121. The method of embodiment 120, wherein the parasitic cell is a bacterial cell, a protozoan cell, a virus, or a fungal cell.
122. The method of embodiment 118, wherein the cell is an abnormal human cell.
123. The method of embodiment 122, wherein the cell is a cancer cell.
124. The method according to any one of embodiments 116-123, wherein the compound is further defined as the compound in embodiments 32-97.
125. A method of reducing the amount of adipose tissue in a patient comprising contracting the adipose tissue with a compound, wherein the compound capable of generating a vibronic-driven action and optionally further comprising a cell targeting moiety; and exposing the compound to an energy source sufficient to generate a vibronic-driven action, wherein the vibronic-driven action is sufficient to reduce the adipose tissue.
126. The method of embodiment 125, wherein the adipose tissue is an adipocyte cell.
127. The method of embodiment 125, wherein the adipose tissue is a lipocyte cell.
128. The method of embodiment 125, wherein the adipose tissue is a fat cell.
129. The method according to any one of embodiments 104-107, wherein the method is sufficient to reduce the weight of the patient.
130. The method according to any one of embodiments 125-128, wherein the method is sufficient to reduce the circumference of a part of the body of the patient.
131. The method according to any one of embodiments 125-130, wherein the method further comprises a second exposure to the energy source.
132. The method according to any one of embodiments 125-131, wherein the method further comprises applying the compound a second time.
133. The method according to any one of embodiments 125-132, wherein the weight of the patient or the circumference of a part of the body of the patient is further reduced.
134. A method of disrupting a cellular component comprising:
   (A) contacting the cellular component with a compound, wherein the compound is capable of generating a vibronic-driven action and optionally further comprising a targeting moiety; and
   (B) exposing the compound to an energy source sufficient to generate the vibronic-driven action, wherein the vibronic-driven action is sufficient to disrupt the cellular component.
135. The method of embodiment 134, wherein the cellular component is a carbohydrate or carbohydrate complex.
136. The method of embodiment 134, wherein the cellular component is a protein or protein complex.
137. The method of embodiment 134, wherein the cellular component is a nucleic acid or nucleic acid complex.
138. The method according to any one of embodiments 134-137, wherein the cellular component is a combination of a nucleic acid, a protein, a carbohydrate, a nucleic acid complex, a protein complex, or a carbohydrate complex.
139. The method according to any one of embodiments 134-138, wherein the cellular component is a cellular component of a prokayrotic cell.
140. The method according to any one of embodiments 134-138, wherein the cellar component is a cellular component of a eukaryotic cell.
141. The method according to any one of embodiments 134-140, wherein the cellular component is a cellular component of a parasitic cell.
142. The method of embodiment 141, wherein the parasitic cell is a bacterial cell, a protozoan cell, a virus, or a fungal cell.
143. The method according to any one of embodiments 134-138 and 140, wherein the cellular component is a cellular component of a human cell.
144. The method of embodiment 143, wherein the human cell is an abnormal human cell.
145. The method of embodiment 144, wherein the human cell is a cancer cell.
146. An intermediate compound further defined by the formula:

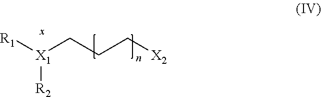

wherein:
   x is a positive or negative charge;
   n is an integer from 0 to 100;
   X₁ is a heteroatom selected from O, N, S, B, P, Ge, As, or Se;
   X₂ is hydroxy, amino, or carboxy; or alkylamino_{(C≤12)}, dialkylamino_{(C≤12)}, cycloalkylamino_{(C≤12)}, dicycloalkylamino_{(C≤12)}, alkyl(cycloalkyl)amino_{(C≤12)}, arylamino_{(C≤12)}, diarylamino_{(C≤12)}, alkyl_{(C≤12)}, cycloalkyl_{(C≤12)}, -alkanediyl_{(C≤12)}-cycloalkyl_{(C≤12)}, -alkanediyl_{(C≤18)}-aralkoxy_{(C≤18)}, heterocycloalkyl_{(C≤12)}, aryl_{(C≤18)}, -arenediyl_{(C≤12)}-alkyl_{(C≤12)}, aralkyl_{(C≤18)}, -arenediyl_{(C≤18)}-heterocycloalkyl_{(C≤12)}, heteroaryl_{(C≤18)}, -heteroarenediyl_{C}≤12)-alkyl_{(C≤12)}, heteroaralkyl_{(C≤18)}, acyl_{(C≤12)}, alkoxy_{(C≤12)}, or a substituted version of any of these groups;
   R₁ and R₂ are each independently alkyl_{(C≤18)}, alkenyl(cis), alkynyl_{(C≤18)}, aryl(cis), aralkyl_{(C≤18)}, heteroaryl_{(C≤18)}, heterocycloalkyl_{(C≤18)}, or a substituted version of any of these groups; or
   R₁ and R₂ are taken together to form one, two, three, four, five, or six aliphatic or aromatic rings; comprising at least three carbon atoms and no more than 36 carbon atoms; optionally comprising one, two, three, four, or five nitrogen, sulfur, or oxygen atom.
147. The method of embodiment 146, wherein X₁ is N.
148. The method of embodiment 146 or embodiment 147, wherein X₂ is alkyl_{(C≤18)} or substituted alkyl_{(C≤18)}.
149. The method of embodiment 146 or embodiment 147, wherein X₂ is amino, alkylamino_{(C≤12)}, or substituted alkylamino_{(C≤12)}.
150. The method of embodiment 146 or embodiment 147, wherein X₂ is carboxy.
151. The method according to any one of embodiments 146-150, wherein R₁ is taken together with R₂ to form one, two, three, four, or five rings.
152. The method according to any one of embodiments 146-151, wherein R₁ is taken together with R₂ to form two, three, or four rings.
153. The method according to any one of embodiments 146-152, wherein R₁ is taken together with R₂ to form three rings.
154. The method according to any one of embodiments 146-153, wherein R₁ is taken together with R₂ to form three rings, wherein one ring is aliphatic and two rings are aromatic.
155. The method according to any one of embodiments 146-154, wherein n is an integer from 1 to 10.
156. The method according to any one of embodiments 146-155, wherein n is an integer selected from 5, 6, or 7.
157. The method of embodiment 155, wherein n is 6.
158. The method of embodiment 146, wherein the intermediate compound is further defined as:

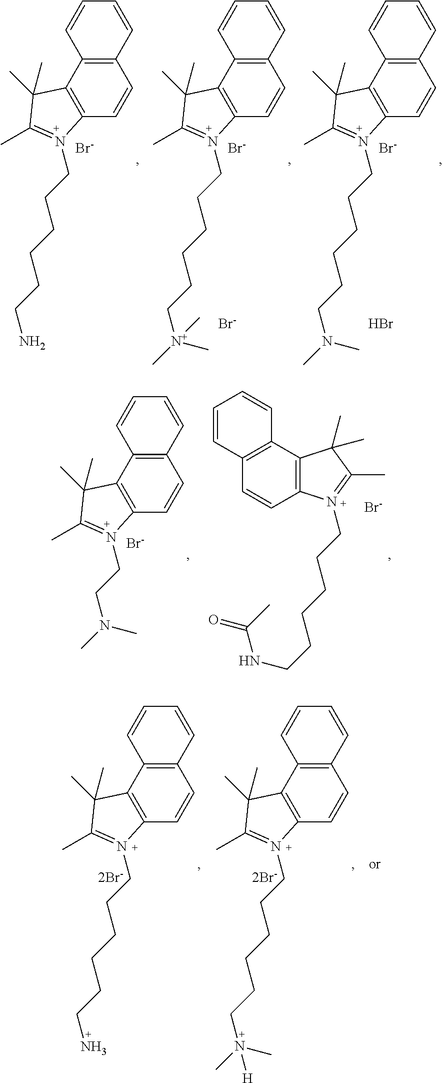

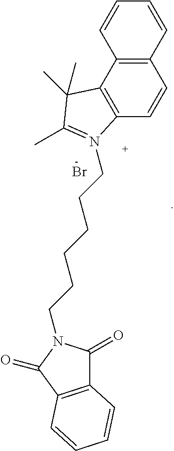

159. A compound of the formula:

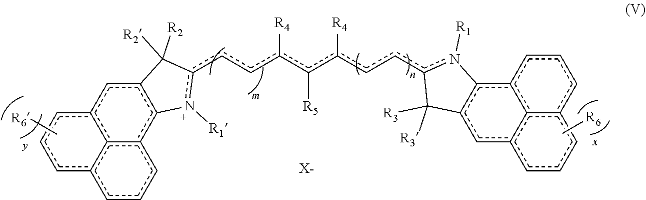

wherein:
   R₁ and R₁′ are each independently alkyl_{(C≤8)}, substituted alkyl_{(C≤8)}, or a group of the formula:

-A-NRₐRₐ′Rₐ″
wherein:
   A is an alkanediyl_{(C≤12)} or substituted alkanediyl_{(C≤12)};
   Rₐ and Rₐ′ are each independently hydrogen or alkyl_{(C≤8)}; and
   Rₐ″ is absent, hydrogen, or alkyl_{(C≤8)};
provided at least one of R₁ and R₁′ is a group of the formula: -A-NRₐRₐ′Rₐ″
R₂, R₂′, R₃, and R₃′ are each independently hydrogen, alkyl_{(C≤8)}, or substituted alkyl_{(C≤8)};
R₄ and R₄′ are each independently hydrogen, alkyl_{(C≤8)}, substituted alkyl_{(C≤8)}, or R₄ and R₄′ are taken together as a cycloalkyl group;
R₅ is hydrogen, halo, carboxy, alkyl_{(C≤8)}, substituted alkyl_{(C≤8)}, or —C(O)OR_{b}, wherein R_{b} is alkyl_{(C≤6)} or substituted alkyl_{(C≤6)};
R₆ is hydrogen, amino, halo, hydroxy, or alkyl_{(C≤12)}, alkoxy_{(C≤12)}, alkylamino_{(C≤8)}, dialkylamino_{(C≤8)}, acyl_{(C≤8)}, acyl_{(C≤8)}, acyl_{(C≤8)}, or a substituted version thereof;
m and n are each 0, 1, 2, or 3;
x and y are each independently 0, 1, 2, 3, 4, or 5;
X is a monovalent anion; and
each of the rings is optionally present as either an aromatic or aliphatic ring.
160. The compound of embodiment 159 further defined as:

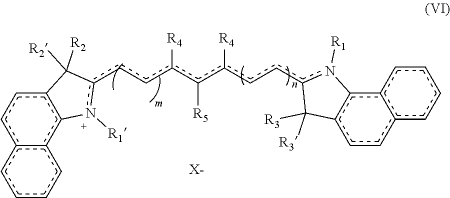

wherein:
   R₁ and R₁′ are each independently alkyl_{(C≤8)}, substituted alkyl_{(C≤8)}, or a group of the formula:

-A-NRₐRₐ′Rₐ″
wherein:
   A is an alkanediyl_{(C≤12)} or substituted alkanediyl_{(C≤12)};
   Rₐ and Rₐ′ are each independently hydrogen or alkyl_{(C≤8)}; and
   Rₐ″ is absent, hydrogen, or alkyl_{(C≤8)};
provided at least one of R₁ and R₁′ is a group of the formula: -A-NRₐRₐ′Rₐ″
R₂, R₂′, R₃, and R₃′ are each independently hydrogen, alkyl_{(C≤8)}, or substituted alkyl_{(C≤8)};
R₄ and R₄′ are each independently hydrogen, alkyl_{(C≤8)}, substituted alkyl_{(C≤8)}, or R₄ and R₄′ are taken together as a cycloalkyl group;
R₅ is hydrogen, halo, carboxy, alkyl_{(C≤8)}, substituted alkyl_{(C≤8)}, or —C(O)OR_{b}, wherein R_{b} is alkyl_{(C≤6)} or substituted alkyl_{(C≤6)};
m and n are each 0, 1, 2, or 3;
X is a monovalent anion; and
each of the rings is optionally present as either an aromatic or aliphatic ring.
161. The compound of either embodiment 159 or embodiment 160 further defined as:

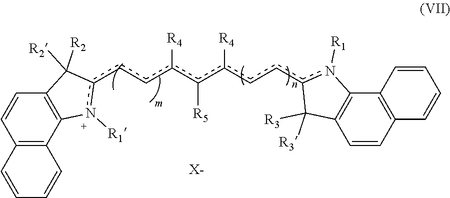

wherein:
   R₁ and R₁′ are each independently alkyl_{(C≤8)}, substituted alkyl_{(C≤8)}, or a group of the formula:

-A-NRₐRₐ′Rₐ″
wherein:
   A is an alkanediyl_{(C≤12)} or substituted alkanediyl_{(C≤12)};
   Rₐ and Rₐ′ are each independently hydrogen or alkyl_{(C≤8)}; and
   Rₐ″ is absent, hydrogen, or alkyl_{(C≤8)};
provided at least one of R₁ and R₁′ is a group of the formula: -A-NRₐRₐ′Rₐ″
R₂, R₂′, R₃, and R₃′ are each independently hydrogen, alkyl_{(C≤8)}, or substituted alkyl_{(C≤8)};
R₄ and R₄′ are each independently hydrogen, alkyl_{(C≤8)}, substituted alkyl_{(C≤8)}, or R₄ and R₄′ are taken together as a cycloalkyl group;
R₅ is hydrogen, halo, carboxy, alkyl_{(C≤8)}, substituted alkyl_{(C≤8)}, or —C(O)OR_{b}, wherein R_{b} is alkyl_{(C≤6)} or substituted alkyl_{(C≤6)};
m and n are each 0, 1, 2, or 3; and
X is a monovalent anion.
162. The compound according to any one of embodiments 159-161 further defined as:

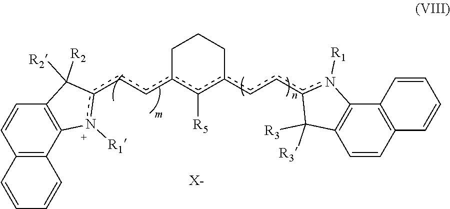

wherein:
   R₁ and R₁′ are each independently alkyl_{(C≤8)}, substituted alkyl_{(C≤8)}, or a group of the formula:

-A-NRₐRₐ′Rₐ″
wherein:
   A is an alkanediyl_{(C≤12)} or substituted alkanediyl_{(C≤12)};
   Rₐ and Rₐ′ are each independently hydrogen or alkyl_{(C≤8)}; and
   Rₐ″ is absent, hydrogen, or alkyl_{(C≤8)};
provided at least one of R₁ and R₁′ is a group of the formula: -A-NRₐRₐ′Rₐ″
R₂, R₂′, R₃, and R₃′ are each independently hydrogen, alkyl_{(C≤8)}, or substituted alkyl_{(C≤8)};
R₅ is hydrogen, halo, carboxy, alkyl_{(C≤8)}, substituted alkyl_{(C≤8)}, or —C(O)OR_{b}, wherein R_{b} is alkyl_{(C≤6)} or substituted alkyl_{(C≤6)};
m and n are each 0, 1, 2, or 3; and
X is a monovalent anion.
163. The compound according to any one of embodiments 159-162 further defined as:

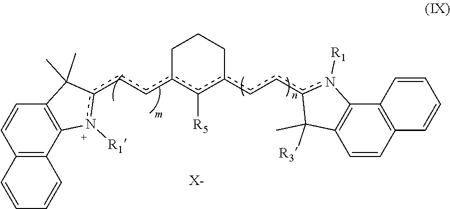

wherein:
   R₁ and R₁′ are each independently alkyl_{(C≤8)}, substituted alkyl_{(C≤8)}, or a group of the formula:

-A-NRₐRₐ′Rₐ″
wherein:
   A is an alkanediyl_{(C≤12)} or substituted alkanediyl_{(C≤12)};
   Rₐ and Rₐ′ are each independently hydrogen or alkyl_{(C≤8)}; and
   Rₐ″ is absent, hydrogen, or alkyl_{(C≤8)};
provided at least one of R₁ and R₁′ is a group of the formula: -A-NRₐRₐ′Rₐ″ R₅ is hydrogen, halo, carboxy, alkyl_{(C≤8)}, substituted alkyl_{(C≤8)}, or —C(O)OR_{b}, wherein R_{b} is alkyl_{(C≤6)} or substituted alkyl_{(C≤6)};
m and n are each 0, 1, 2, or 3; and
X is a monovalent anion.
164. The compound according to any one of embodiments 159-163, wherein m is 1.
165. The compound according to any one of embodiments 159-164, wherein n is 1.
166. The compound according to any one of embodiments 159-165, wherein Rₐ is alkyl_{(C≤8)}.
167. The compound according to any one of embodiments 159-165, wherein Rₐ is hydrogen.
168. The compound according to any one of embodiments 159-167, wherein Rₐ′ is alkyl_{(C≤8)}.
169. The compound according to any one of embodiments 159-167, wherein Rₐ′ is hydrogen.
170. The compound according to any one of embodiments 159-169, wherein the compound is further defined as:

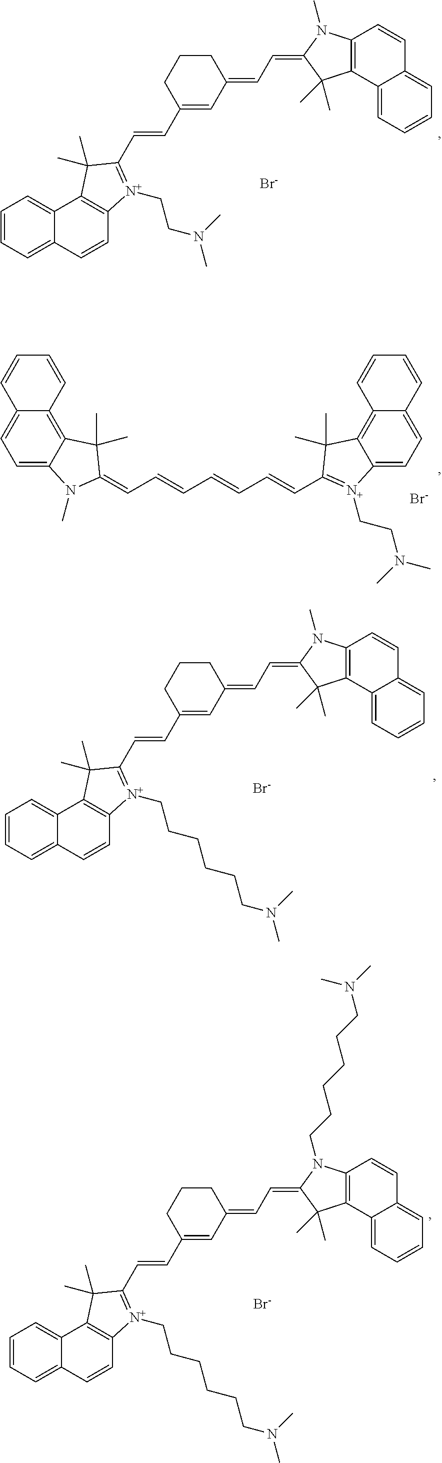

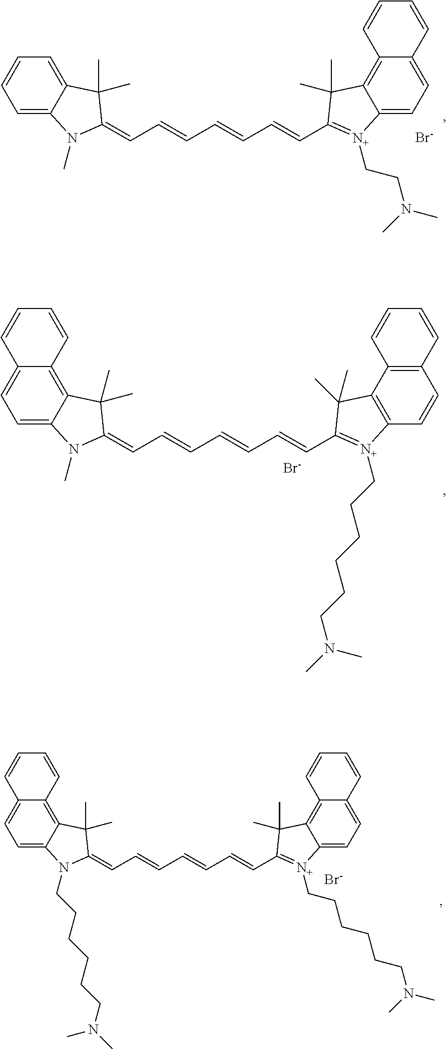 [SMILES: CN(C)CC[N+]1=C(/C=C/C=C/C=C/C=C2/N(C)c3ccccc3C2(C)C)C(C)(C)c2c1ccc1ccccc21 ; [Br-] ; CN(C)CCCCCC[N+]1=C(/C=C/C=C/C=C/C=C2/N(C)c3ccc4ccccc4c3C2(C)C)C(C)(C)c2c1ccc1ccccc21 ; [Br-] ; CN(C)CCCCCCN1/C(=C/C=C/C=C/C=C/C2=[N+](CCCCCCN(C)C)c3ccc4ccccc4c3C2(C)C)C(C)(C)c2c1ccc1ccccc21 ; [Br-]]

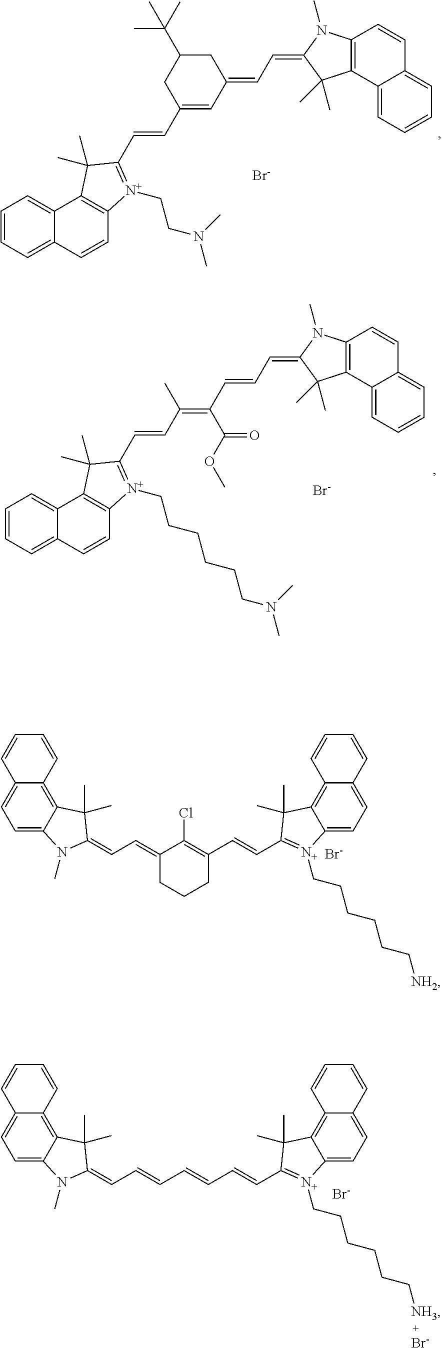

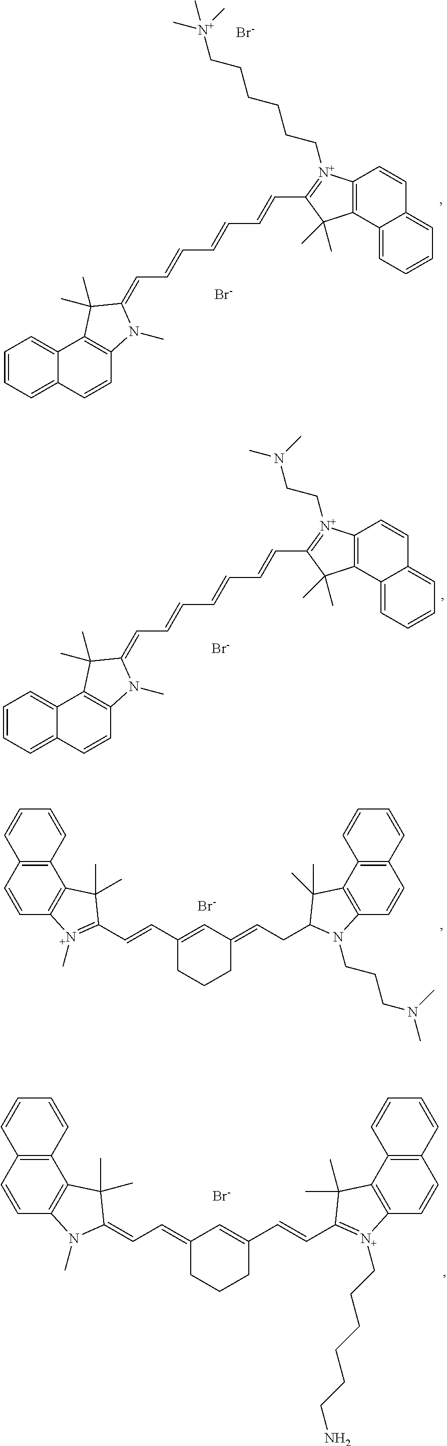

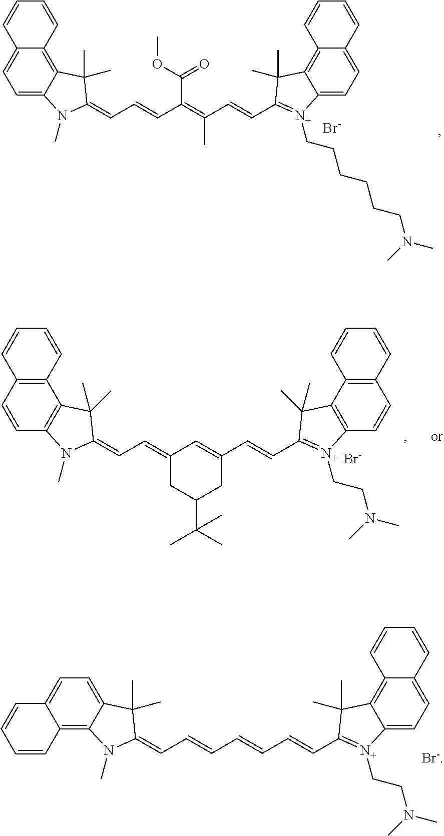

157. A method of disrupting a cell membrane comprising contacting the cell membrane with a compound of formula:

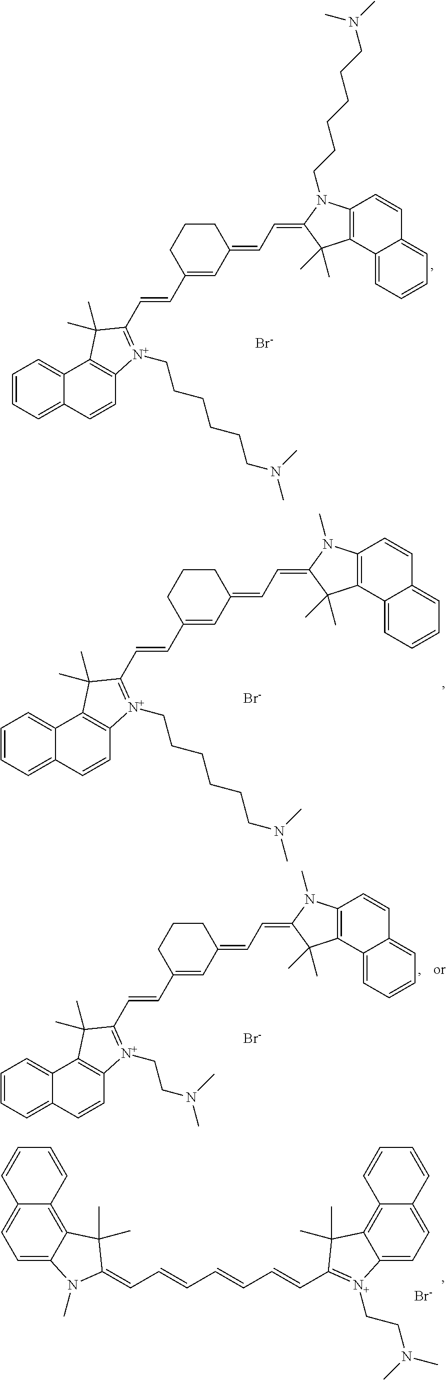

and exposing the membrane to an energy source capable of generating vibronic-driven action.

### VII. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Selective Excitation of Cy7.5-Amine

FIG. 2A shows the chemical structure and absorption spectra of two aminocyanines, Cy7-amine and Cy7.5-amine. Without being bound by theory, the amine moieties, which are protonated at physiological pH, promoted association with the lipid bilayer charged surfaces. Cyanine structures are characterized by an odd-numbered polyene linker connecting two nitrogen-containing heterocycles with unusual photophysical properties. The absorption spectrum of cyanines is dominated by an absorption band in the visible/NIR electromagnetic spectrum with a shoulder located at higher energy (shorter wavelength). The Cy7.5-amine, in contrast to Cy7-amine, has an additional benzene ring that increased the conjugation, causing a red-shifting of the absorption by ˜40 nm relative to Cy7-amine.

The origin of the sub-bands in cyanines have been thoroughly studied, which without being bound by theory concluded that the presence of the shoulder next to the large absorption band is “primarily determined by a dominant vibration associated with its polymethine chain rather than a collection of singly excited vibrations” (Mustroph and Towns, 2018). The vibronic behavior, through the coupling of electronic and vibrational states, is a feature of the conjugated-backbone-near-symmetrical cyanines such as in Cy7-amine and C7.5-amine. In the case of conjugated-backbone-unsymmetrical cyanines, the absorption curves do not exhibit a vibrational fine structure, analogous to most spectra of merocyanines (Mustroph, 2021). Without being bound by theory, the vibronic mode in symmetrical cyanine structures is thought to result from the coupling of a dominant collective oscillation of electronic excitation (molecular plasmon) to a dominant collective vibrational excitation (phonon). The shoulders at ˜730 nm and ˜690 nm in the absorption spectrum of Cy7.5-amine and Cy7-amine, respectively, correspond to this collective vibrational mode.

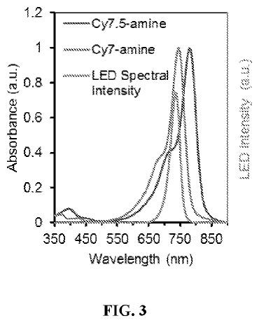

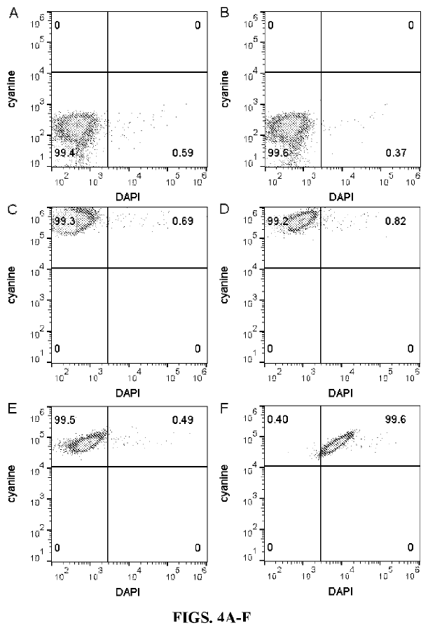

Here the vibronic mode was selectively excited in a cell-membrane-bound Cy7.5-amine using a NIR light-emitting diode (LED) at 730 nm (FIG. 2B and FIG. 3) which resulted in the permeabilization of the cellular membrane to 4′,6-diamidino-2-phenylindole (DAPI), a cell membrane impermeable dye in viable cells that readily stains cellular DNA in membrane-disrupted cells, with induction of rapid necrotic cell death in human A375 melanoma cells (FIG. 4). While 730 nm light did not excite the vibronic shoulder of Cy7-amine, it activated the Cy7.5-amine and permeabilized A375 cells immediately after treatment. The time between sample irradiation and the start of data collection in the flow cytometer was approximately 30 seconds; 10,000 cells were analyzed. In one embodiment, 1 μM Cy7.5-amine, 730 nm LED at 80 mW/cm² for 10 min caused permeabilization to DAPI staining of 99.6% of the A375 cells in a cell suspension containing 2×10⁵ cells in media. In contrast, Cy7-amine was not able to permeabilize the cells under the same conditions. Of the cells with cell-membrane-bound Cy7-amine, 0.8% of the cells were DAPI-positive, which was considered background cell death as observed in the controls without treatment. Without being bound by theory, the difference between the two aminocyanines supported the notion that the 730 nm light can excite the vibronic mode (shoulder) in Cy7.5-amine causing cellular membrane permeabilization and ultimately cell death by necrosis as seen by immediate DAPI staining. Flow cytometry showed both of the aminocyanines were attached efficiently to the cells; they were both loaded into the cell. While Cy7-amine has a larger extinction coefficient (ε=132,000 μM⁻¹ cm⁻¹) at λ=730 nm than Cy7.5-amine (ε=72,000 μM⁻¹ cm⁻¹), Cy7-amine does not permeabilized the cells while Cy7.5-amine readily permeabilizes, consistent with a vibronic-driven action (VDA) on Cy7.5-amine. Without being bound by theory, this data suggested that a photothermal effect is not operating since Cy7-amine had a higher absorption cross-section than Cy7.5 at 730 nm. Likewise, photodynamic therapy was unlikely since Cy7-amine and C7.5-amine have similar yields for singlet oxygen generation (S̆tacková et al., 2020).

FIG. 4-5 compares the Cy7-amine and Cy7.5-amine at various concentrations. It is evident that the Cy7.5-amine is much more efficient at permeabilizing cells upon the VDA with NIR light. Even at much higher concentrations of Cy7-amine (8 μM), the compound still is not able to permeabilize cells as efficiently as the Cy7.5-amine. Without being bound by theory, this was further confirmation that the excitation of the vibronic shoulder in Cy7.5-amine at 730 nm was critical for the activation of the molecular vibrations.

### Example 2—Mechanism of Necrosis and Cell Membrane Binding

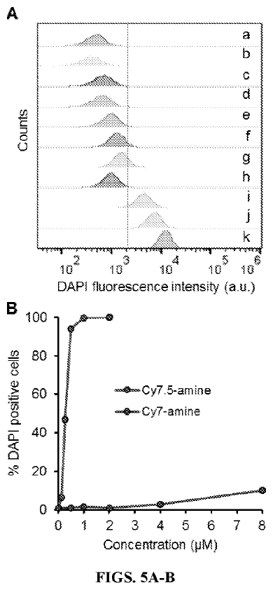

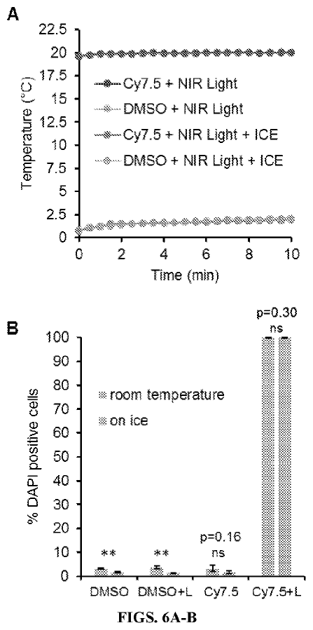

To rule out a photothermal effect, the temperature of the media was measured during the NIR light exposure when the treatment was done at room temperature versus when done on ice bath (FIG. 6). The temperature remained constant at 20° C. and 2° C. when the treatment was done at room temperature and in an ice bath, respectively. In FIG. 6B, the cell permeabilization is not affected by the lower temperature. Thus, again without being bound by theory, the photothermal effect was determined to not be responsible for the necrosis seen in these cells.

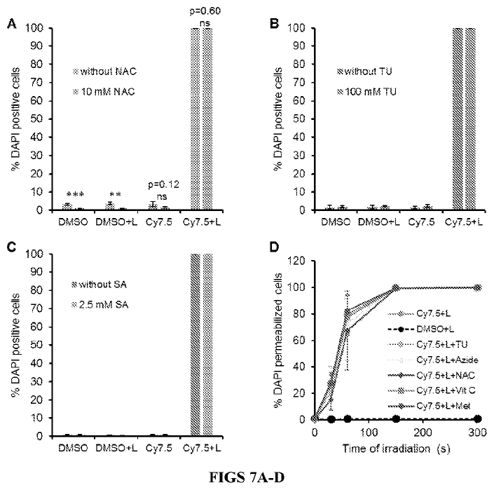

To confirm that a photodynamic ROS generation was not responsible for the necrosis, the permeabilization of A375 melanoma cells was repeated in the presence of ROS scavengers (FIG. 7). Neither N-acetyl-cysteine (NAC, 10 mM), thiourea (TU, 100 mM) nor sodium azide (SA, 2.5 mM) was able to stop the permeabilization of the cells. Further, that ROS is not responsible for the permeabilization of the cells, in FIG. 7D the permeabilization conditions were tuned to lower illumination time where the ROS scavengers potentially could quench ROS more effectively. The results showed that none of the ROS scavengers used (NAC, TU, SA, methionine or vitamin C) were able to stop the permeabilization of the A375 cells. Without being bound by theory, this strongly suggested that ROS is not responsible for the permeabilization of the cells.

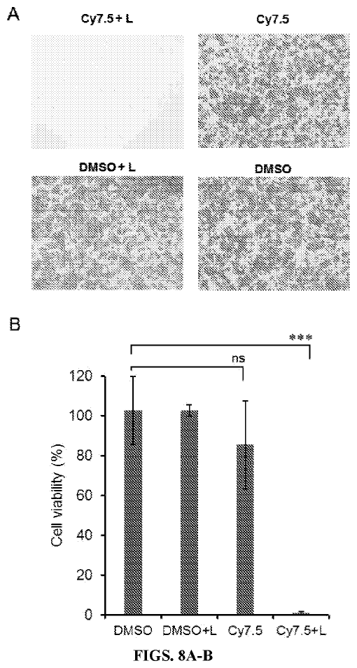

To confirm that the permeabilized cells to DAPI were indeed dead, the cells were cultured after treatment with 2 μM Cy7.5-amine, 30 min incubation, and subsequent illumination for 10 min with 730 nm light at 80 mW/cm² and cell death was quantified by a crystal violet assay. In this test, the viable cells attach and grow in the culture dish while the dead cells detach or are easily detached in the washing step with phosphate buffered saline (PBS). As shown in FIG. 8, the cells permeabilized by light-activated Cy7.5-amine were dead nearly at the same quantity, 99.9%, as was observed in flow cytometry 1 min after treatment.

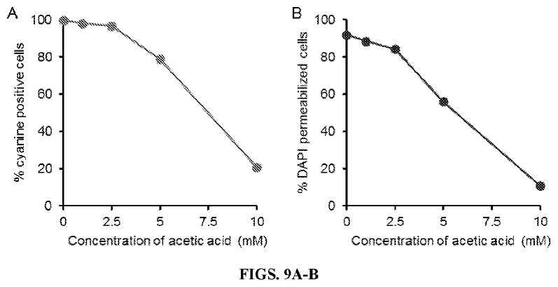

Finally, to confirm that the Cy7.5-amine binding to the cells was likely mediated by the charge on its pendant amine moiety, which without being bound by theory is probably due to interaction with the negatively charged phospholipids, acetic acid was added into the medium (0 mM-10 mM) to protonate the phosphates of the phospholipids. The permeabilization of the cells and flow cytometry analysis was conducted as before. The results, as shown by flow cytometry (FIG. 9), indicated that Cy7.5-amine was not able to bind efficiently to lipid membranes upon addition of acetic acid. The lower binding of Cy7.5-amine to the cells was reflected in the lower permeabilization of the cells upon NIR light excitation.

Another cyanine was also tried that has a vibronic absorption shoulder at 730 nm, indocyanine green (ICG), bearing sulfonate containing addends, as shown in FIG. 10. Without being bound by theory, the sulfonates will not interact with phospholipids in lipid bilayers. ICG was not able to permeabilize the cells under the same conditions in which Cy7.5-amine was tested: 2 μM and illumination for 10 min of 730 nm light at 80 mW/cm² (FIG. 10). The presence of 1% or less of DAPI positive cells was considered normal content of cell death in every cell batch and so was considered the baseline in the control. Furthermore, ICG did not cause a photothermal effect since the temperature of the media remains at the baseline at 20° C. (FIG. 10C). The photothermal effects of ICG were studied at the high concentration range of 8 μM up to 400 μM (FIG. 10C). Below 32 μM, from 0 to 16 μM, there was no observed cell permeabilization to DAPI with or without the light treatment. In the range of 32 μM up to 400 μM, there was observed permeabilization of the cells to DAPI from 2% up to 14%, respectively. This cell permeabilization was caused by the photothermal effect since the medium showed a significant temperature increase in this range of concentrations. The permeabilization of the cells by the photothermal effect with ICG reached its maximum at 200 μM because of the attenuation of the light by ICG; the transmittance of the light was compromised as the ICG concentration increased. Without being bound by theory, when taken together, the results suggested that the effective cell killing of Cy7.5-amine is a combination of the VMA and the amine for rapid cell association. This, again without being bound by theory, causes an energy transfer from these whole-molecule excited structures to the cellular membrane, ultimately disrupting the lipid bilayer, and permitting cell membrane permeabilization.

### Example 3—Evaluation of Additional Cyanine Dyes

### A. Building a Molecular Plasmon Library of Small Organic Molecules

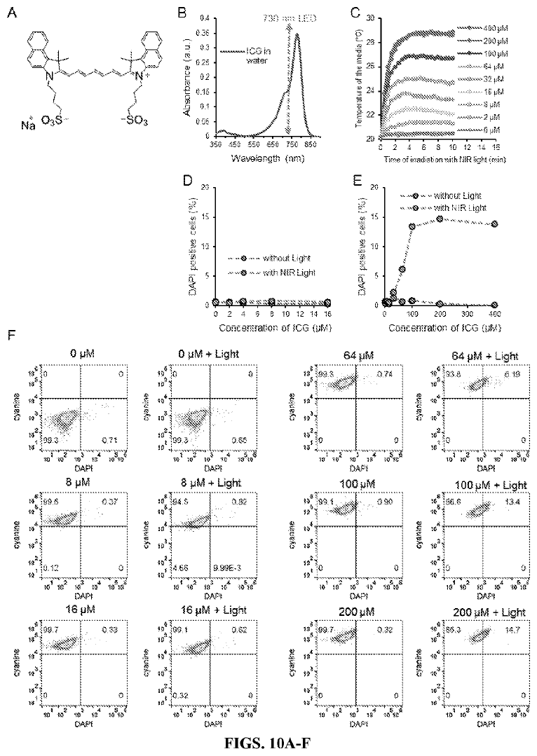

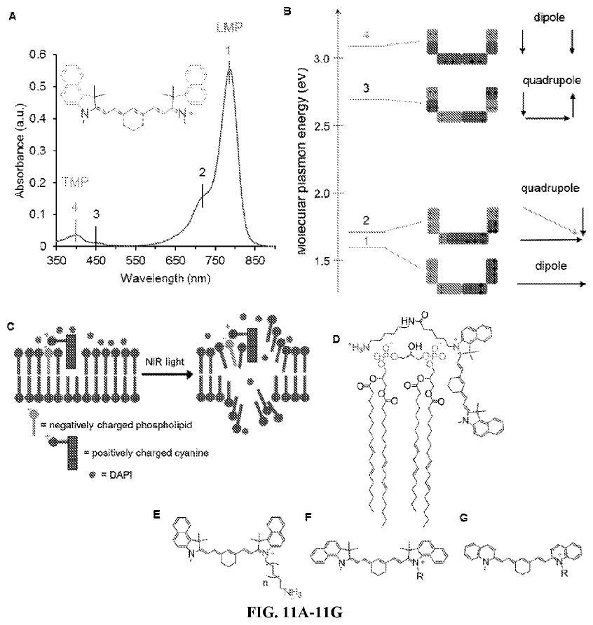

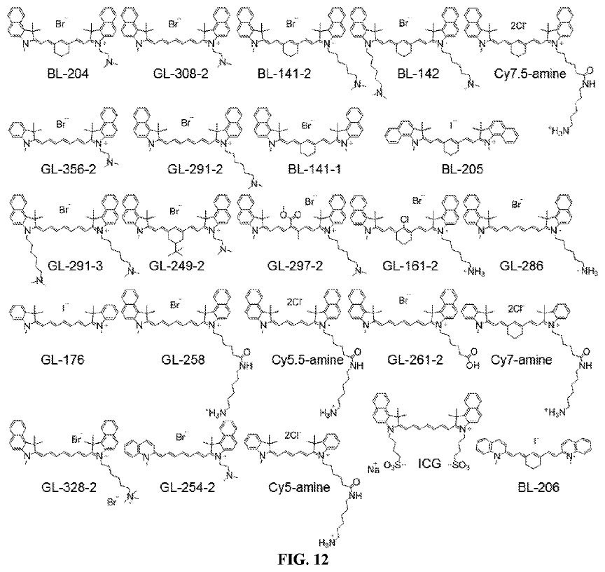

Molecular jackhammers (MJH) are chemical structures that support plasmon resonances in small organic molecules upon optical excitation. Here, four major plasmon resonances have been identified and proposed in cyanine-based molecular plasmons (FIG. 11A). Two plasmons resonances are in the blue region of the visible spectrum and two in the near-infrared (NIR) region, the so-called NIR therapeutic window. These four plasmon resonances correspond to the 1) the dipolar oscillation of the electron density along the longitudinal axis of the molecule which is also called longitudinal molecular plasmon (LMP), 2) the quadrupolar oscillation of the electron density primarily along the longitudinal axis and couple with contributions along the transversal axis, 3) the quadrupolar oscillation of the electron density primarily along the transversal axis and couple with contributions along the longitudinal axis, 4) the dipolar oscillation of the electron density along the transversal axis which is also called transversal molecular plasmon (TMP) as described in FIGS. 11A & 11B. A pictorial representation of the working mechanism of MJHs to open cellular membranes upon NIR-light excitation is proposed in FIGS. 11C & 11D and this is supported by the experimental results. The optical excitation of plasmon resonances in MJHs activates the electron density oscillation and simultaneously this is couple with the nuclear vibrations, giving rise to a vibronic (electronic and vibrational) driven action (VDA) which can disassemble cellular membranes or supramolecular biological assemblies. In particular, the MJH can associate to the lipid bilayers primarily through hydrophobic interactions and through electrostatic interactions between the polar substituents on MJHs and polar heads on phospholipids, such as cardiolipin, as shown in FIG. 11D and further supported by experiments. Following this concept, various chemical structures were synthesized by 1) modifying the side arm that contributes to the binding into the lipid bilayers (FIG. 11E) and 2) modifying the chemical structure of the core that strongly contributes to the plasmon resonance properties (FIGS. 11F-11G). Following this strategy, a comprehensive library of MJH compounds was built (FIG. 12).

B. The Molecular Plasmon Index Correlates with the Activity to Open Cell Membranes.

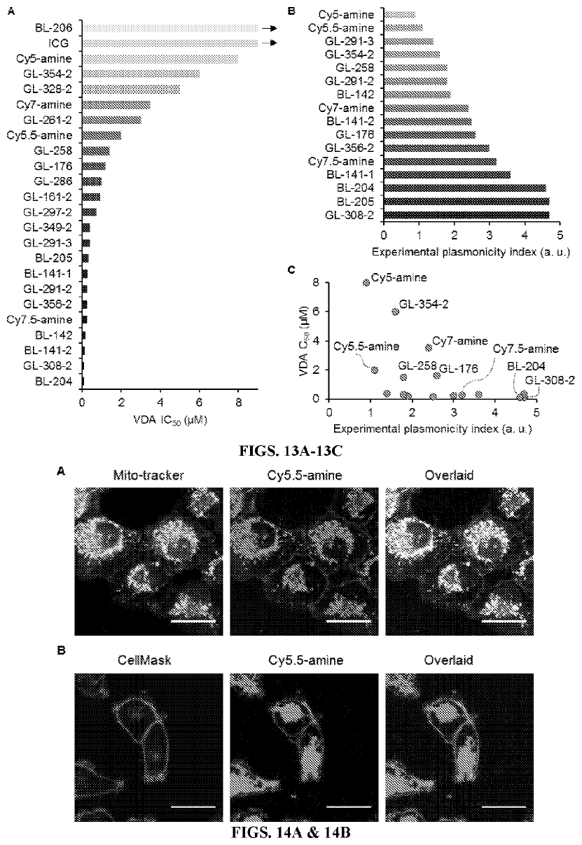

The activity of plasmon-driven MJHs to open cell membranes by VDA upon NIR light-activation was evaluated in human melanoma A375 cells. When the cell membranes were open by VDA, DAPI enters immediately into the cells and stain the nucleus, and flow cytometry analysis was used to quantify the percentage of DAPI positive cells at variable concentrations of MJH. The flow cytometry analysis is conducted immediately after the cells were treated with 730 nm LED light at 80 mW/cm² for 10 min. Typically, it took ˜30 s to start cell counting and observe that DAPI already had entered into the cell membrane-compromised cells but not in the controls without VDA action. This is indicative of a rapid necrotic cell death and cell membrane permeabilization to DAPI by VDA action. The effective concentration needed to permeabilize cells by 50% (VDA IC₅₀) was estimated for each molecule. The library of MJHs compounds was ordered from most active (VDA IC₅₀=0.12 μM) to least active (VDA IC₅₀>>8 μM) in FIG. 13A.

The VDA activity, defined as VDA IC₅₀, correlates with the plasmonicity index, defined here by first time as experimental plasmonicity index (EPI) in FIG. 13B & 13C. The EPI is a semiempirical experimental estimation of the plasmonic character of each molecule. Briefly, the EPI is an estimation of the optical response of a molecular plasmon to the dielectric constant of the solvent which reflects the plasmonic character of the molecule. Interestingly, we observed that the higher the EPI the better the VDA activity to permeabilize cells (FIG. 13C). The correlation between EPI and VDA activity is not perfect for all the cases in FIG. 13C. However, this is expected since the side chain (or arms) in the structures are variable and this strongly influence the amount of the MJH bound to the cell membranes. Therefore, molecules were compared with the same side chains, and found that the correlation is perfect: the higher the EPI the better the VDA activity in all the cases.

### C. MJHs Targets Mitochondria, Outer Cellular Membrane and Nuclear Membrane in A375 Cells.

The specific localization of MJHs in the cell was studied by confocal microscopy since cyanines are standard photostable and high yield fluorescent probes broadly use for live cell, small animal and even human imaging (FIG. 14).(24, 25) In general, the MJH in this study associate with the cellular membranes. The side chain in the cyanine-based MJHs influence the docking into specific cellular membranes. It was observed that Cy5.5-amine binds specifically the outer cell membrane, the nuclear membrane and the mitochondria (FIG. 14) The Cy5.5-amine targeting to the mitochondria is likely through the docking interaction with cardiolipin, a phospholipid exclusively present in the internal lipid bilayer in mitochondria as supported by the flow cytometry analysis (26-28).

### D. Plasmon-Driven MJH Cy5.5-Amine Disassembles Cellular Membranes and Cytoskeleton Upon NIR-Light Activation.

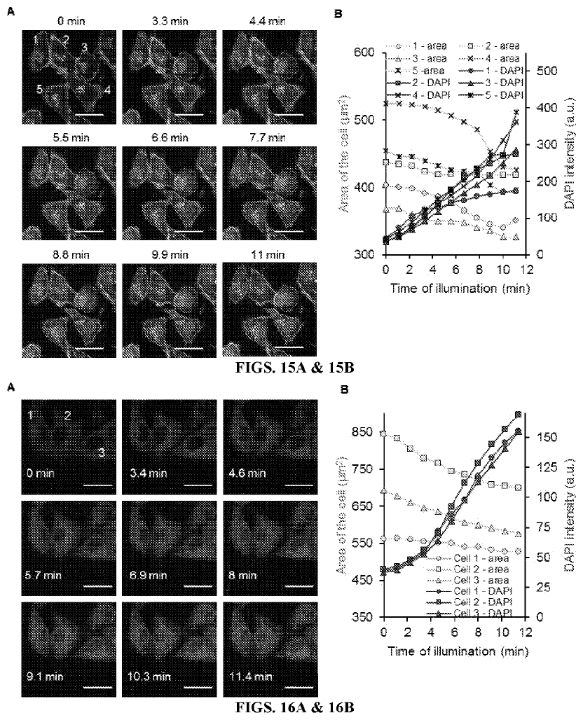

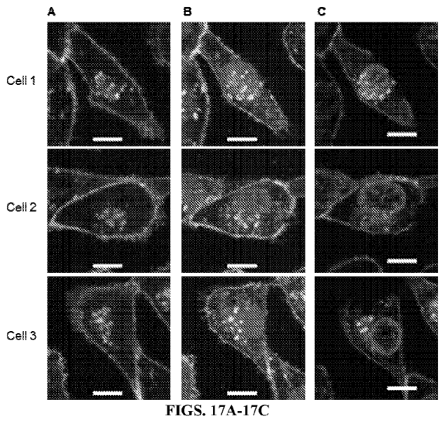

The plasmon resonance in Cy5.5-amine was activated upon laser excitation (λₑₓ=640 nm) under the confocal microscope while imaging real-time opening of the outer cellular membrane, DAPI entering into the cell throughout the holes and staining of nuclear DNA, and cytoskeleton was simultaneously disassembling (FIGS. 15-17). The plasmon-driven MJH disassembled the plasma membranes and pieces of the cell membrane broke apart from the cell in FIG. 17. Simultaneously, on real-time it was observed that the cells were shrinking in size (FIG. 15). This cell shrinkage was quantified as the area defined by the perimeter of the outer cell membrane over the time (FIG. 15). It was confirmed that the shrinkage of the cell is because the cytoskeleton was contracting in a similar experiment but the cytoskeleton was imaged on real-time using GFP-label actin (FIG. 16). This suggest that the cytoskeleton is disassembling simultaneously while the plasmon-driven MJHs are destroying cellular membranes in the cell. The light alone does not cause the shrining of the cell neither DAPI entering into the cell, but instead, the cells responded to the light alone and since they are not being destroyed, still have time to keep control of their movements, cells probably were trying to move away from the light, and we observed instead a slight increase on the area of the cell. This is additional evidence that the VDA in plasmon-driven MJH is conducting a mechanical action different than the action induced by light effects. The MJH without light activation does not affect the cytoskeleton (area of the cell) nor DAPI is entering into the cell within the time window of the experiment. Neither changes are observed in the cells without any treatment, only CellMask Green is added to visualize the cell membrane and DAPI to measure the integrity of the cell membrane.

### E. Lethal Concentration of Plasmon-Driven MJHs at Short Contact Time in A375 Cells.

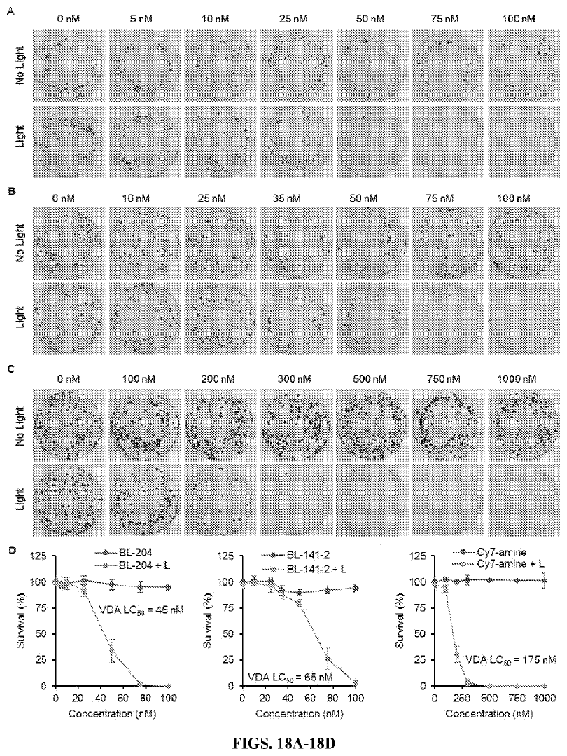

The clonogenic assay was conducted to confirm that cancer cells treated by plasmon-driven MJH are indeed death upon NIR-light activation (FIG. 18). Three molecules were analyzed: the most VDA active BL-204, medium active BL-141-2 and low active Cy7-amine. The lethal concentration to kill the cell population by 50% (VDA IC₅₀) were 45 nM, 65 nM and 175 nM, respectively. This was accomplished by incubating the MJH molecules with the A375 cells for 50 min and immediately activate the plasmon-driven MJH using 730 nm NIR-light at 80 mW/cm² for 10 min. This is considered a short contact time between the MJH and the A375 cells.

### F. Predicted Octanol-Water Partition Coefficient (Log P Value) in Cyanine-Based MJH.

The octanol-water partition coefficient (log P value) was calculated on the MJH structures using online interactive log P calculator. This parameter informs of the lipophilicity or affinity of the MJH to the lipid bilayers.(29, 30) The higher the value the more likely the MJH will bind to the lipid bilayers. The protonation state of the MJH strongly modifies the polarity of the molecules and hence influences the log P values. The log P values were calculated considering the charged state of the arm (FIG. 19A) or in neutral state (FIG. 19B). The charged state is more likely at pH˜7.4 of the medium and the pKa of the alkyl amines ˜9.5-11 and of the carboxylic acid ˜5. It was found that the log P values do not correlate completely with the VDA to permeabilize cells (FIG. 19). There are highly VDA active MJHs such as BL-204, GL-308-2, GL-356-2 with relatively low log P values, then low affinity to lipid bilayers. In contrast, there are molecules (BL-205, BL-141-1, BL-142) with relatively high log P values that should be loaded in higher amounts into the lipid bilayers. However, these last three are not the most VDA active. This supports that the VDA activities that were observed are not simply due to that more MJH is loaded into the lipid bilayers but because there is a plasmon-mediated action that strongly defines the VDA activity.

The lack of full correlation between log P values and VDA activity does not mean that the affinity and loading into the lipid bilayers is not playing a role. Indeed, in a selected group of molecules (ICG, GL-328-2, GL-286, and GL-291-2) the log P values correlates well with the VDA activity. In this case, the correlation exists because the molecules have the same plasmonic core structure and the same length of the alkyl chain, the major variation is coming from a primary amine, secondary amine, quaternary amine and sulfonate. Therefore, the plasmon properties are expected to be similar among these molecules but the affinity to membranes defines a correlation with the VDA activity.

There is another group of molecules in which a correlation between the log P value and VDA activity could be expected because they have similar plasmonic core structure but different side arms. However, this is not the case. The plasmonicity index playing a stronger role specially on BL-204 and GL-308-2. In these two molecules, the protonated secondary amine, is too close to the core structure permitting the amine to act as an electron withdrawing group that enhances the plasmonicity. And second, comparing molecules such as BL-141-1, BL-141-2 and BL-142, suggest that docking by specific and selective interactions into the lipid bilayers could be playing a role which influences strongly the outcome of the VDA activity.

### Example 4—Synthesis of Molecular Jackhammers

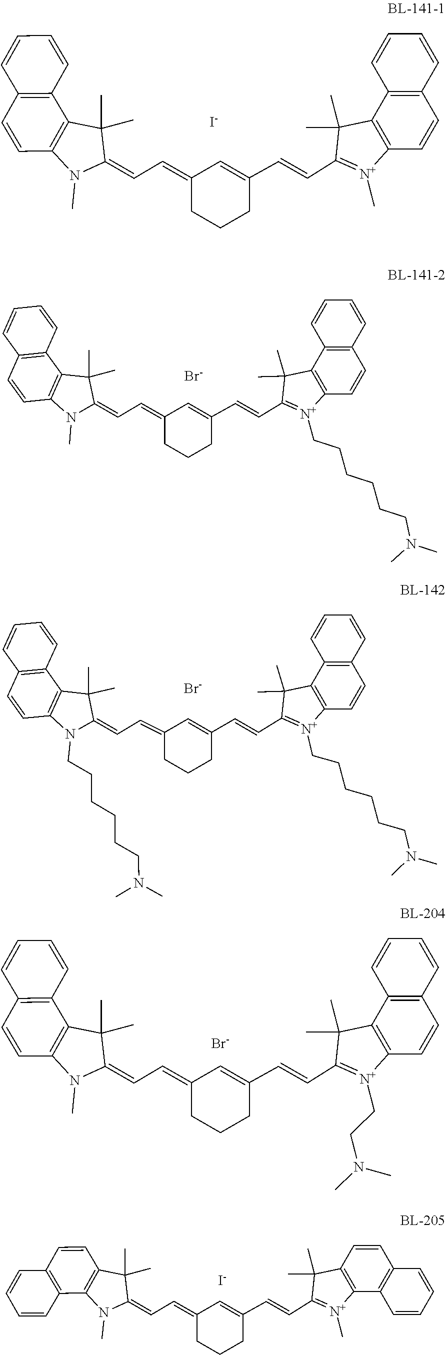

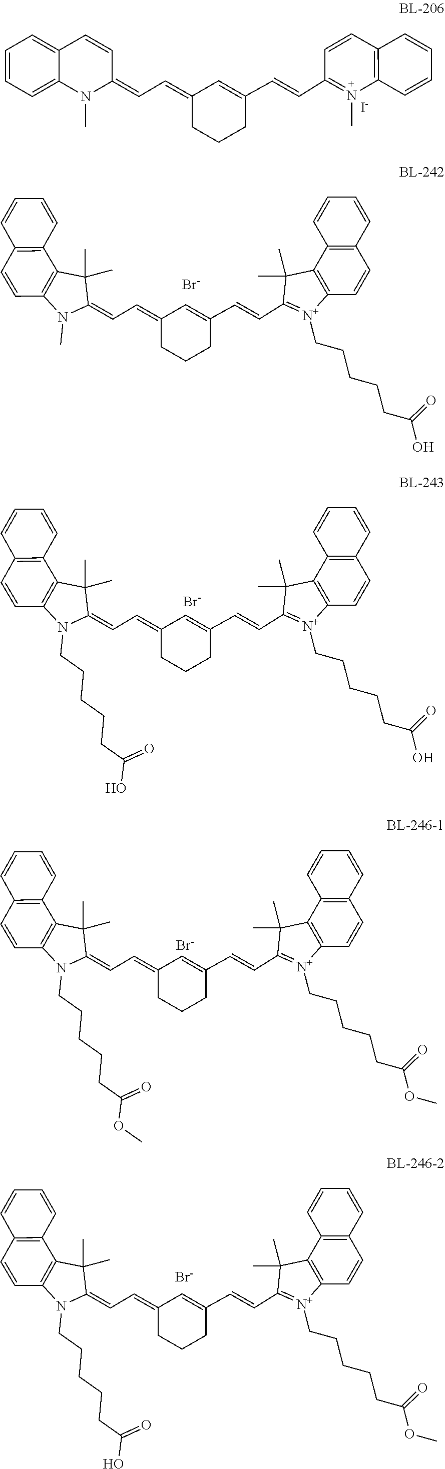

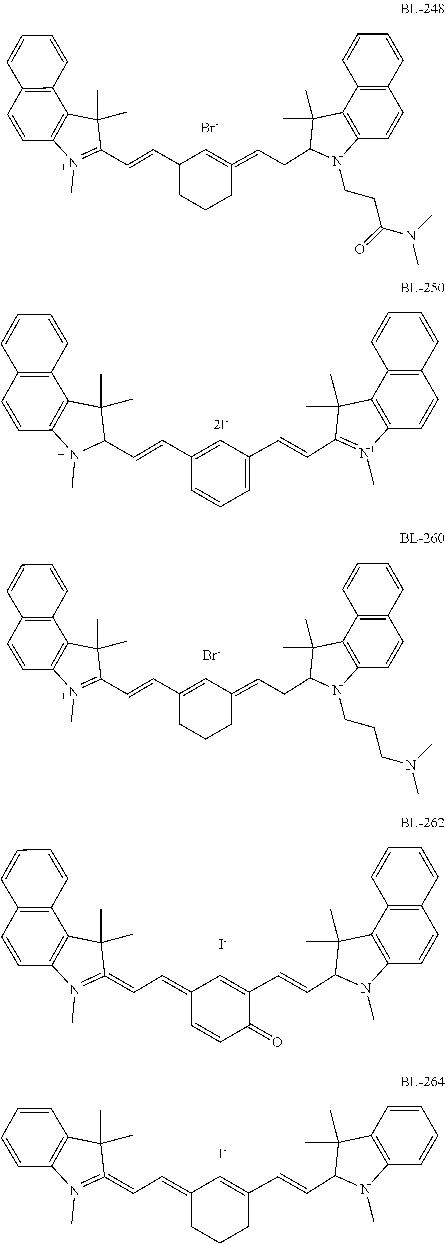

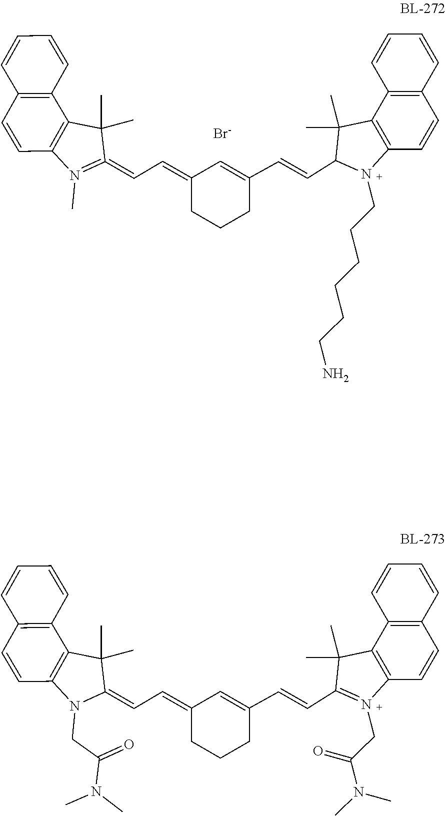

General information: All glassware was oven-dried overnight prior to use. All reactions were carried out under an N₂ atmosphere unless otherwise noted. All other chemicals were purchased from commercial suppliers and used without further purification.

TABLE 1
Condition optimizations of cyanine dyes key intermediate 2

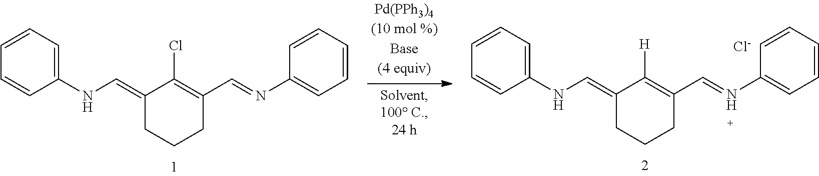 [SMILES: ClC1=C(/C=N/c2ccccc2)CCC/C1=C\Nc1ccccc1 ; C1=C(/C=N/c2ccccc2)CCC/C1=C\Nc1ccccc1 ; [Cl-]]

| Entry^{[a]} | Base | Solvent | Yield (%)^{[b]} |
|---|---|---|---|
| 1 | K₃PO₄ | DMF/H₂O (5:1) | 31 |
| 2 | K₂HPO₄ | DMF/H₂O (5:1) | 30 |
| 3 | KF | DMF/H₂O (5:1) | 25 |
| 4 | K₂CO₃ | DMF/H₂O (5:1) | 32 |
| 5 | NaOtBu | DMF/H₂O (5:1) | 0 |
| 6 | NaOAc | DMF/H₂O (5:1) | 0 |
| 7 | CsF | DMF/H₂O (5:1) | 3 |
| 8 | Cs₂CO₃ | DMF/H₂O (5:1) | 10 |
| 9 | K₂CO₃ | DMF/H₂O/IPA (5:1:1) | 35 |
| 10 | K₂CO₃ | EtOH | 37 |
| 11 | K₂CO₃ | IPA | 66 (61)^{[c]} |

^{[a]}The reactions were carried out with conditions: 1) 1 (0.25 mmol), Pd(PPh₃)₄ (10 mol %), Base (4 equiv) in solvent at 100° C. for 24 h;
^{[b]}Yields were determined by HPLC on a C18 reverse-phase column;
^{[c]}Isolated yield.

General procedure: To a screw-capped vial was added 1 (0.25 mmol), Pd(PPh₃)₄ (0.025 mmol, 10 mol %), Base (1 mmol, 4 equiv). The vial was sealed with a PTFE septum and then evacuated and backfilled with N₂ for three times, followed by addition of solvent via syringe and vigorous stirring. The sealed reaction was heated to 100° C. in oil bath heating for 24 h, then the reaction was cooled to room temperature and concentrated under reduced pressure, followed by 6M HCl at 0° C. and stirred for 10 mins at room temperature. The precipitate was filtered, washed with H₂O, Et₂O and acetone, dried in vacuo to provide compound 2 as a dark red solid.

N-((E)-(3-((E)-(phenylimino)methyl)cyclohex-2-en-1-ylidene)methyl)aniline (2): ¹H-NMR (600 MHz, MD₃OD) δ 8.14 (s, 2H), 7.79 (s, 1H), 7.49-7.39 (m, 8H), 7.28-7.23 (m, 2H), 2.58 (t, J=6.2 Hz, 4H), 1.99-1.93 (m, 2H). ¹³C NMR (151 MHz, MD₃OD) δ 163.58, 153.18, 140.74, 130.98, 127.16, 119.97, 119.18, 22.96, 21.58. HRMS (ESI) for calculated for [M+H, C₂₀H₂₁N₂]⁺: 289.1626, found: 289.1707.

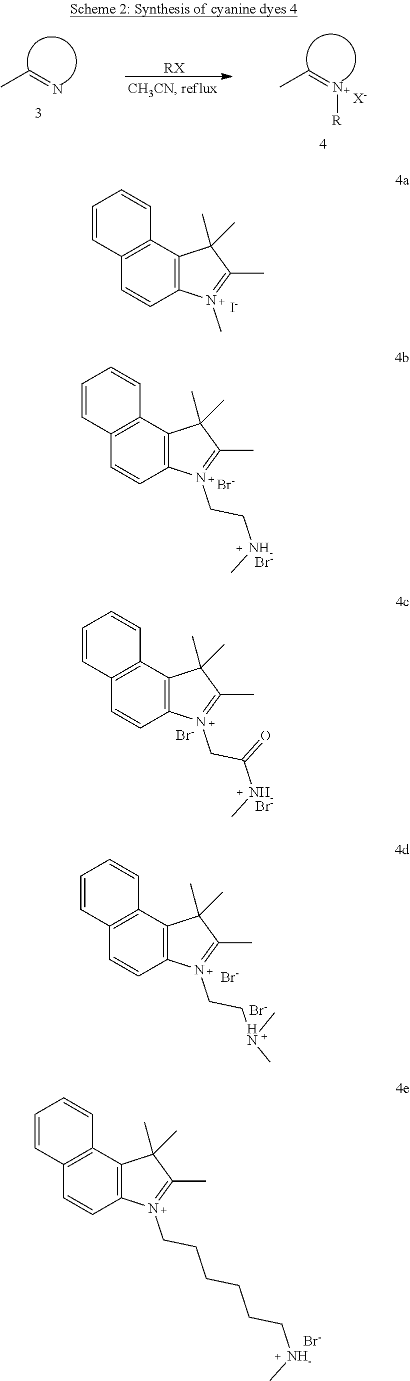

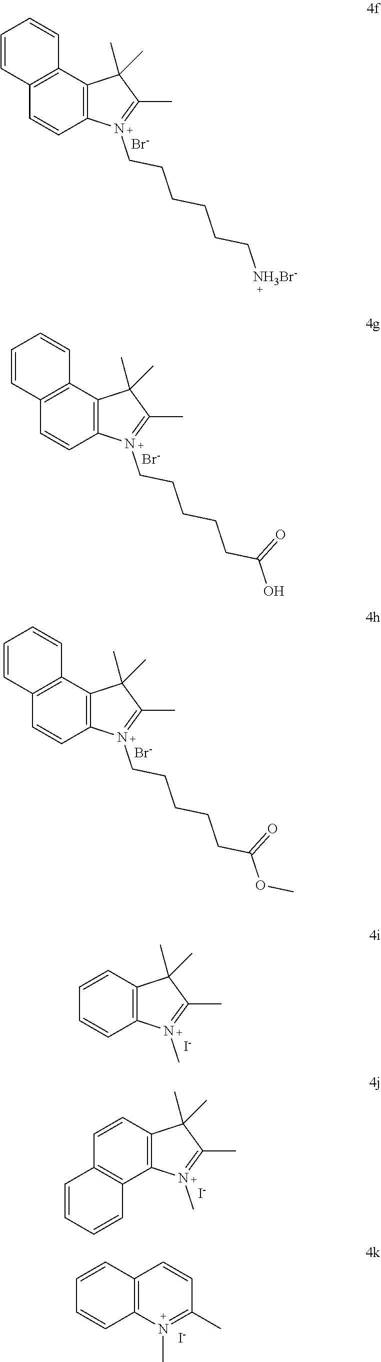

General procedure: To a screwed-capped vial charged with compound 3 (1 equiv) and alkyl halides (1.5 equiv) were heated to reflux in CH₃CN until compound 3 consumed all. Subsequently, the mixture was cooled to room temperature, then diethyl ether was added to precipitate the product. That product was collected by filtration and washed with diethyl ether to obtained compound 4.

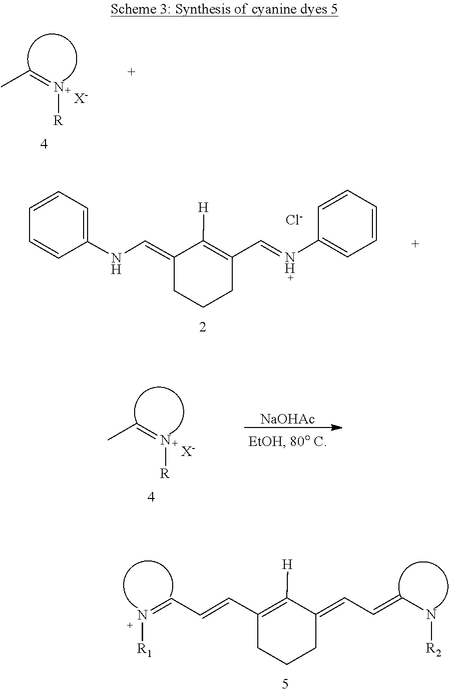

General Synthetic Procedure: All glassware was oven-dried overnight prior to use. All reactions were carried out under an N₂ atmosphere unless otherwise noted. All other chemicals were purchased from commercial suppliers and used without further purification.

General procedure: To a screwed-capped vial charged with compound 3 (1 equiv) and alkyl halides (1.5 equiv) were heated to reflux in CH₃CN until compound 3 consumed all. Subsequently, the mixture was cooled to room temperature, then diethyl ether was added to precipitate the product. That product was collected by filtration and washed with diethyl ether to obtained compound 4.

Procedure b: To a screw-capped vial charged with compound 4 (1 equiv), 2 (1 equiv), 4 (1 equiv) and NaOAc (3 equiv) were dissolved in absolute ethanol. The mixture was heated at 80° C. for overnight under N₂ atmosphere. The final product was purified by silica column chromatography to obtain compound 5.

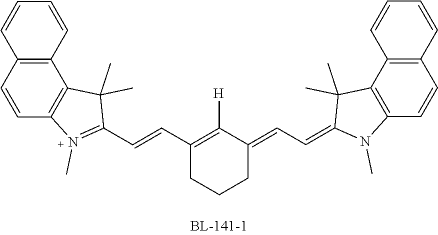

1,1,3-trimethyl-2-((E)-2-((E)-3-((E)-2-(1,1,3-trimethyl-1,3-dihydro-2H-benzo[e]indol-2-ylidene)ethylidene)cyclohex-1-en-1-yl)vinyl)-1H-benzo[e]indol-3-ium (BL-141-1): Prepared according to the general procedure from 4a (176 mg, 0.5 mmol, 1 equiv) and 2 (81 mg, 0.25 mmol, 1 equiv). Yield: 88% yield (149 mg). ¹H-NMR (600 MHz, MD₃OD) δ 8.24-8.21 (m, 2H), 8.00 (d, J=8.8 Hz, 2H), 7.97 (d, J=8.6 Hz, 2H), 7.87 (d, J=14.2 Hz, 2H), 7.64-7.60 (m, 2H), 7.57 (d, J=8.8 Hz, 2H), 7.53 (s, 1H), 7.48-7.44 (m, 2H), 6.19 (d, J=14.1 Hz, 2H), 3.72 (s, 6H), 2.61 (t, J=6.1 Hz, 4H), 2.03-1.95 (m, 14H). HRMS (ESI) calculated for [M, C₄₀H₄₁N₂]⁺: 549.3264, found: 549.3275.

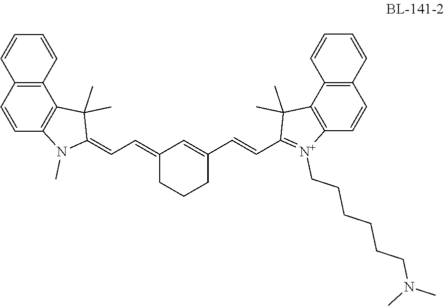 [SMILES: CN(C)CCCCCC[N+]1=C(/C=C/C2=CC(=C/C=C3/N(C)c4ccc5ccccc5c4C3(C)C)/CCC2)C(C)(C)c2c1ccc1ccccc21]

3-(6-(dimethylamino)hexyl)-1,1-dimethyl-2-((E)-2-((E)-3-((E)-2-(1,1,3-trimethyl-1,3-dihydro-2H-benzo[e]indol-2-ylidene)ethylidene)cyclohex-1-en-1-yl)vinyl)-1H-benzo[e]indol-3-ium (BL-141-2): Prepared according to the general procedure from 4a (176 mg, 0.5 mmol, 1 equiv), 2 (162 mg, 0.5 mmol, 1 equiv) and 4e (248 mg, 0.5 mmol, 1 equiv). Yield: 14% yield (45 mg). ¹H-NMR (600 MHz, MD₃OD) δ 8.27-8.21 (m, 2H), 8.05-7.96 (m, 4H), 7.93-7.89 (m, 1H), 7.85 (d, J=14.0 Hz, 1H), 7.67-7.61 (m, 2H), 7.60 (d, J=8.8 Hz, 1H), 7.57 (d, J=8.8 Hz, 1H), 7.52 (s, 1H), 7.50-7.45 (m, 2H), 6.21 (dd, J=23.7, 14.1 Hz, 2H), 4.24 (t, J=7.4 Hz, 2H), 3.75 (s, 3H), 3.04 (t, J=9.0 Hz, 2H), 2.80 (s, 6H), 2.63-2.60 (m, 4H), 2.06-1.90 (m, 14H), 1.75-1.70 (m, 2H), 1.59-1.54 (m, 2H), 1.51-1.47 (m, 2H). HRMS (ESI) calculated for [M, C₄₇H₅₆N₃]⁺: 662.4469, found: 662.4476.

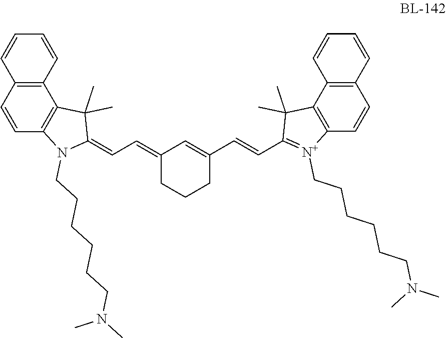 [SMILES: CN(C)CCCCCCN1/C(=C/C=C2C=C(/C=C/C3=[N+](CCCCCCN(C)C)c4ccc5ccccc5c4C3(C)C)CCC/2)C(C)(C)c2c1ccc1ccccc21]

3-(6-(dimethylamino)hexyl)-2-((E)-2-((E)-3-((E)-2-(3-(6-(dimethylamino)hexyl)-1,1-dimethyl-1,3-dihydro-2H-benzo[e]indol-2-ylidene)ethylidene)cyclohex-1-en-1-yl)vinyl)-1,1-dimethyl-1H-benzo[e]indol-3-ium (BL-142): Prepared according to the general procedure from 2 (81 mg, 0.25 mmol, 1 equiv) and 4e (248 mg, 0.5 mmol, 2 equiv). Yield: 20% yield (40 mg). ¹H-NMR (600 MHz, MD₃OD) δ 8.25-8.22 (m, 2H), 8.03-7.96 (m, 4H), 7.89 (d, J=14.1 Hz, 2H), 7.66-7.62 (m, 2H), 7.60 (d, J=8.8 Hz, 2H), 7.56 (s, 1H), 7.50-7.45 (m, 2H), 6.24 (d, J=14.1 Hz, 2H), 4.27 (t, J=7.4 Hz, 4H), 4.27 (t, J=7.4 Hz, 4H), 2.82 (s, 12H), 2.63 (t, J=6.2 Hz, 4H), 2.05-1.96 (m, 12H), 1.95-1.90 (m, 6H), 1.76-1.72 (m, 4H), 1.60-1.55 (m, 4H), 1.52-1.47 (m, 4H). HRMS (ESI) calculated for [M, C₅₄H₇₁N₄]⁺: 775.5673, found: 775.5662.

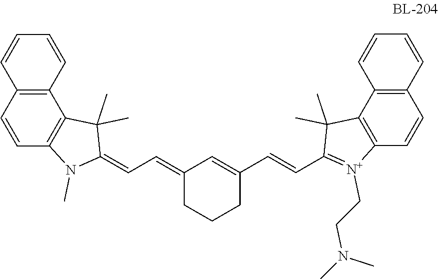 [SMILES: CN(C)CC[N+]1=C(/C=C/C2=CC(=C/C=C3/N(C)c4ccc5ccccc5c4C3(C)C)/CCC2)C(C)(C)c2c1ccc1ccccc21]

3-(2-(dimethylamino)ethyl)-1,1-dimethyl-2-((E)-2-((E)-3-((E)-2-(1,1,3-trimethyl-1,3-dihydro-2H-benzo[e]indol-2-ylidene)ethylidene)cyclohex-1-en-1-yl)vinyl)-1H-benzo[e]indol-3-ium (BL-204): Prepared according to the general procedure from 4a (70 mg, 0.2 mmol, 1 equiv), 2 (65 mg, 0.2 mmol, 1 equiv) and 4b (107 mg, 0.2 mmol, 1 equiv). Yield: 18% yield (26 mg). ¹H-NMR (600 MHz, MD₃OD) δ 8.25 (d, J=8.6 Hz, 1H), 8.22 (d, J=8.7 Hz, 1H), 8.05-7.90 (m, 5H), 7.82 (d, J=13.9 Hz, 1H), 7.67-7.59 (m, 3H), 7.56-7.44 (m, 4H), 6.28 (d, J=14.2 Hz, 1H), 6.21 (d, J=14.1 Hz, 1H), 4.30 (t, J=7.4 Hz, 2H), 3.77 (s, 3H), 2.77 (t, J=7.4 Hz, 2H), 2.66-2.59 (m, 4H), 2.43 (s, 6H), 2.06-1.95 (m, 14H). HRMS (ESI) calculated for [M, C₄₃H₄₈N₃]⁺: 606.3843, found: 606.3847.

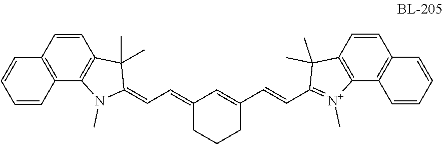 [SMILES: CN1/C(=C/C=C2C=C(/C=C/C3=[N+](C)c4c(ccc5ccccc45)C3(C)C)CCC/2)C(C)(C)c2ccc3ccccc3c21]

1,3,3-trimethyl-2-((E)-2-((E)-3-((E)-2-(1,3,3-trimethyl-1,3-dihydro-2H-benzo[g]indol-2-ylidene)ethylidene)cyclohex-1-en-1-yl)vinyl)-3H-benzo[g]indol-1-ium (BL-205): Prepared according to the general procedure from 4j (70 mg, 0.2 mmol, 2 equiv) and 2 (33 mg, 0.1 mmol, 1 equiv). Yield: 74% yield (50 mg). ¹H-NMR (600 MHz, MD₃OD) δ 8.59 (d, J=8.7 Hz, 2H), 7.99 (d, J=8.2 Hz, 2H), 7.82 (d, J=8.9 Hz, 4H), 7.67-7.59 (m, 4H), 7.58-7.52 (m, 2H), 7.50 (s, 1H), 6.33 (d, J=14.0 Hz, 2H), 4.18 (s, 6H), 2.63 (t, J=6.2 Hz, 4H), 2.02-1.96 (m, 2H), 1.77 (s, 12H). HRMS (ESI) calculated for [M, C₄₀H₄₁N₂]⁺: 549.3264, found: 549.3247.

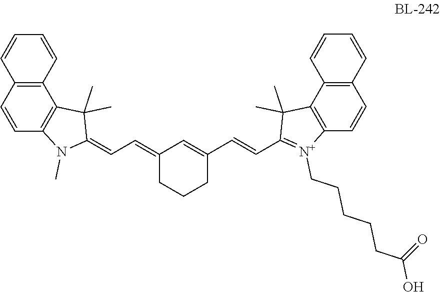 [SMILES: CN1/C(=C/C=C2C=C(/C=C/C3=[N+](CCCCCC(=O)O)c4ccc5ccccc5c4C3(C)C)CCC/2)C(C)(C)c2c1ccc1ccccc21]

3-(5-carboxypentyl)-1,1-dimethyl-2-((E)-2-((E)-3-((E)-2-(1,1,3-trimethyl-1,3-dihydro-2H-benzo[e]indol-2-ylidene)ethylidene)cyclohex-1-en-1-yl)vinyl)-1H-benzo[e]indol-3-ium (BL-242): Prepared according to the general procedure from 4a (117 mg, 0.33 mmol, 1 equiv), 2 (107 mg, 0.33 mmol, 1 equiv) and 4g (134 mg, 0.33 mmol, 1 equiv). Yield: 18% yield (130 mg). ¹H-NMR (600 MHz, MD₃OD) δ 8.12 (d, J=8.5 Hz, 2H), 7.91-7.85 (m, 4H), 7.84-7.69 (m, 2H), 7.55-7.51 (m, 2H), 7.50-7.32 (m, 5H), 6.21-5.95 (m, 2H), 4.20-4.06 (m, 2H), 3.63 (s, 3H), 2.68-2.30 (m, 4H), 2.23 (t, J=7.3 Hz, 2H), 1.99-1.84 (m, 14H), 1.83-1.77 (m, 2H), 1.65-1.60 (m, 2H), 1.48-1.41 (m, 2H); ¹³C-NMR (151 MHz, MD₃OD) δ 177.49, 174.88, 173.98, 163.31, 163.08, 162.84, 149.06, 148.67, 141.83, 141.22, 134.55, 133.35, 133.29, 132.30, 131.66, 131.61, 131.10, 129.68, 129.53, 129.46, 128.66, 125.84, 124.39, 123.33, 113.51, 111.95, 111.83, 100.54, 100.28, 54.81, 52.05, 44.83, 34.81, 31.83, 28.22, 27.60, 27.50, 27.41, 25.78, 25.02, 22.78, 22.07. HRMS (ESI) calculated for [M, C₄₅H₄₉N₂O₂]⁺: 649.3789, found: 649.3793.

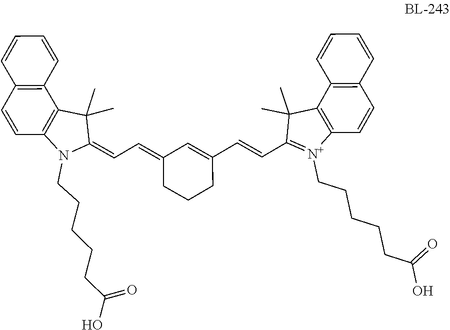 [SMILES: CC1(C)C(/C=C/C2=CC(=C/C=C3/N(CCCCCC(=O)O)c4ccc5ccccc5c4C3(C)C)/CCC2)=[N+](CCCCCC(=O)O)c2ccc3ccccc3c21]

3-(5-carboxypentyl)-2-((E)-2-((E)-3-((E)-2-(3-(5-carboxypentyl)-1,1-dimethyl-1,3-dihydro-2H-benzo[e]indol-2-ylidene)ethylidene)cyclohex-1-en-1-yl)vinyl)-1,1-dimethyl-1H-benzo[e]indol-3-ium (BL-243): Prepared according to the general procedure from 2 (33 mg, 0.1 mmol, 1 equiv) and 4 μg (81 mg, 0.2 mmol, 2 equiv). Yield: 45% yield (39 mg). ¹H-NMR (600 MHz, MD₃OD) δ 8.23 (d, J=8.5 Hz, 2H), 8.00 (d, J=8.9 Hz, 2H), 7.98 (d, J=8.0 Hz, 2H), 7.86 (d, J=14.0 Hz, 2H), 7.65-7.61 (m, 2H), 7.57 (d, J=8.8 Hz, 2H), 7.50-7.44 (m, 3H), 6.22 (d, J=14.1 Hz, 2H), 4.23 (t, J=7.5 Hz, 4H), 2.61 (t, J=6.2 Hz, 4H), 2.25 (t, J=7.4 Hz, 4H), 2.03-1.96 (m, 12H), 1.93-1.87 (m, 4H), 1.75-1.70 (m, 4H), 1.56-1.50 (m, 4H). HRMS (ESI) calculated for [M, C₅₀H₅₇N₂O₄]⁺: 749.4313, found: 749.4312.

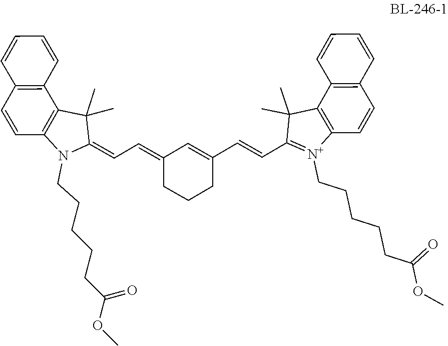 [SMILES: COC(=O)CCCCCN1/C(=C/C=C2C=C(/C=C/C3=[N+](CCCCCC(=O)OC)c4ccc5ccccc5c4C3(C)C)CCC/2)C(C)(C)c2c1ccc1ccccc21]

3-(6-methoxy-6-oxohexyl)-2-((E)-2-((E)-3-((E)-2-(3-(6-methoxy-6-oxohexyl)-1,1-dimethyl-1,3-dihydro-2H-benzo[e]indol-2-ylidene)ethylidene)cyclohex-1-en-1-yl)vinyl)-1,1-dimethyl-1H-benzo[e]indol-3-ium (BL-246-1): Prepared according to the general procedure from 2 (65 mg, 0.2 mmol, 1 equiv) and 4h (167 mg, 0.4 mmol, 2 equiv). Yield: 18% yield (30 mg). ¹H-NMR (600 MHz, MD₃OD) δ 8.23 (d, J=8.5 Hz, 2H), 8.01 (d, J=8.9 Hz, 2H), 7.99 (d, J=8.1 Hz, 2H), 7.87 (d, J=14.1 Hz, 2H), 7.65-7.62 (m, 2H), 7.57 (d, J=8.8 Hz, 2H), 7.51-7.46 (m, 3H), 6.23 (d, J=14.1 Hz, 2H), 4.27-4.19 (m, 4H), 3.69 (s, 3H), 3.62 (s, 3H), 2.62 (t, J=6.2 Hz, 4H), 2.36 (dt, J=11.5, 7.3 Hz, 4H), 2.06-1.96 (m, 12H), 1.93-1.88 (m, 4H), 1.76-1.70 (m, 4H), 1.56-1.50 (m, 4H). HRMS (ESI) calculated for [M, C₅₂H₆₁N₂O₄]⁺: 777.4626, found: 777.4626.

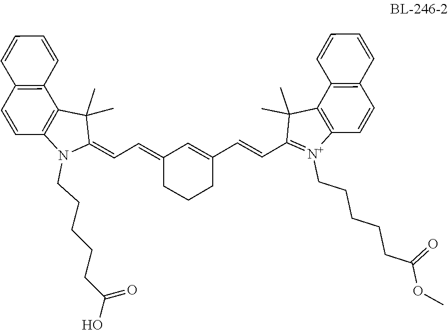 [SMILES: COC(=O)CCCCC[N+]1=C(/C=C/C2=CC(=C/C=C3/N(CCCCCC(=O)O)c4ccc5ccccc5c4C3(C)C)/CCC2)C(C)(C)c2c1ccc1ccccc21]

2-((E)-2-((E)-3-((E)-2-(3-(5-carboxypentyl)-1,1-dimethyl-1,3-dihydro-2H-benzo[e]indol-2-ylidene)ethylidene)cyclohex-1-en-1-yl)vinyl)-3-(6-methoxy-6-oxohexyl)-1,1-dimethyl-1H-benzo[e]indol-3-ium (BL-246-2): Prepared according to the general procedure from 2 (65 mg, 0.2 mmol, 1 equiv) and 4h (167 mg, 0.4 mmol, 2 equiv). Yield: 17% yield (28 mg). ¹H-NMR (600 MHz, MD₃OD) δ 8.23 (d, J=8.5 Hz, 2H), 8.21-7.96 (m, 4H), 7.87 (dd, J=14.1, 9.3 Hz, 2H), 7.65-7.61 (m, 2H), 7.59-7.54 (m, 2H), 7.51-7.45 (m, 3H), 6.22 (t, J=14.0 Hz, 2H), 4.27-4.18 (m, 4H), 3.70-3.61 (m, 3H), 2.61 (t, J=6.4 Hz, 4H), 2.36 (dt, J=11.5, 7.2 Hz, 2H), 2.29 (t, J=7.3 Hz, 2H), 2.03-2.00 (m, 8H), 1.97 (s, 6H), 1.94-1.87 (m, 4H), 1.76-1.68 (m, 4H), 1.57-1.50 (m, 4H); HRMS (ESI) calculated for [M, C₅₁H₅₉N₂O₄]⁺: 763.4469, found: 763.4455.

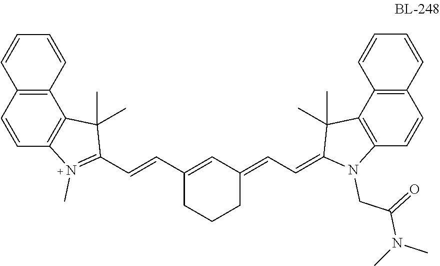

2-((E)-2-((E)-3-(2-(3-(3-(dimethylamino)-3-oxopropyl)-1,1-dimethyl-2,3-dihydro-1H-benzo[e]indol-2-yl)ethylidene)cyclohex-1-en-1-yl)vinyl)-1,1,3-trimethyl-1H-benzo[e]indol-3-ium (BL-248): Prepared according to the general procedure from 4a (70 mg, 0.2 mmol, 1 equiv), 2 (65 mg, 0.2 mmol, 1 equiv) and 4c (75 mg, 0.2 mmol, 1 equiv). Yield: 20% yield (28 mg). ¹H-NMR (600 MHz, MD₃OD) δ 8.16-8.11 (m, 2H), 7.94-7.88 (m, 2H), 7.86-7.81 (m, 3H), 7.69 (d, J=13.8 Hz, 1H), 7.56-7.49 (m, 3H), 7.43-7.38 (m, 2H), 7.35-7.32 (m, 1H), 7.30 (d, J=8.8 Hz, 1H), 6.17 (d, J=14.3 Hz, 1H), 5.88 (d, J=13.7 Hz, 1H), 5.08 (s, 2H), 3.67 (s, 3H), 3.22 (s, 3H), 2.94 (s, 3H), 2.50 (t, J=6.2 Hz, 2H), 2.46 (t, J=6.2 Hz, 2H), 1.96 (s, 6H), 1.91 (s, 6H), 1.88-1.82 (m, 2H). ¹³C-NMR (151 MHz, MD₃OD) δ 174.77, 172.68, 165.98, 154.83, 149.06, 146.27, 140.60, 140.25, 133.71, 132.79, 132.18, 132.13, 131.72, 130.32, 129.93, 129.74, 129.66, 128.19, 127.96, 127.38, 127.08, 124.76, 124.06, 122.02, 121.85, 110.61, 110.18, 100.14, 98.32, 51.01, 50.46, 44.88, 35.61, 34.87, 30.69, 26.28, 26.00, 23.59, 21.37. HRMS (ESI) calculated for [M, C₄₃H₄₆N₃O]⁺: 620.3635, found: 620.3634.

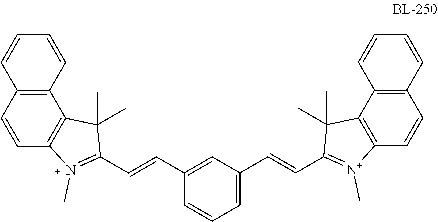

2,2′-((1E,1′E)-1,3-phenylenebis(ethene-2,1-diyl))bis(1,1,3-trimethyl-1H-benzo[e]indol-3-ium) (BL-250): Prepared according to the general procedure from m-Phthalaldehyde (70 mg, 0.52 mmol, 1 equiv) and 4a (365 mg, 1.04 mmol, 2 equiv). Yield: 50% yield (208 mg). ¹H-NMR (600 MHz, MD₃OD) δ 8.64 (d, J=16.5 Hz, 2H), 8.48-8.44 (m, 2H), 8.34-8.30 (m, 2H), 8.27 (d, J=8.9 Hz, 2H), 8.19 (d, J=8.2 Hz, 2H), 8.04 (d, J=8.9 Hz, 2H), 7.96 (d, J=16.5 Hz, 2H), 7.87-7.83 (m, 2H), 7.81 (t, J=7.7 Hz, 2H), 7.76-7.73 (m, 2H), 4.45 (s, 6H), 2.18 (s, 12H). HRMS (ESI) calculated for [M, C₄₀H₃₈N₂]⁺: 546.3024, found: 546.3015.

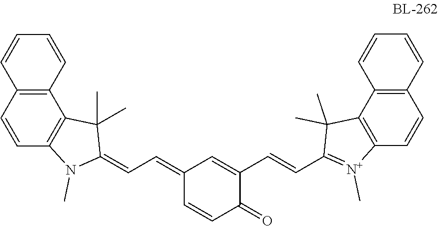 [SMILES: CN1/C(=C/C=C2\C=CC(=O)C(/C=C/C3=[N+](C)c4ccc5ccccc5c4C3(C)C)=C2)C(C)(C)c2c1ccc1ccccc21]

2,2′-((1E,1′E)-1,3-phenylenebis(ethene-2,1-diyl))bis(1,1,3-trimethyl-1H-benzo[e]indol-3-ium) (BL-262): Prepared according to the general procedure from 4-Hydroxyisophthalaldehyde (50 mg, 0.33 mmol, 1 equiv) and 4a (232 mg, 0.66 mmol, 2 equiv). Yield: 80% yield (182 mg). ¹H-NMR (600 MHz, MD₃OD) δ 8.82 (s, 1H), 8.22 (d, J=16.0 Hz, 1H), 8.17 (d, J=8.5 Hz, 1H), 8.09 (d, J=8.8 Hz, 1H), 8.04 (d, J=8.1 Hz, 1H), 7.92 (d, J=8.6 Hz, 1H), 7.85 (d, J=16.0 Hz, 1H), 7.81 (dd, J=8.5, 1.3 Hz, 1H), 7.76 (d, J=8.5 Hz, 1H), 7.74-7.69 (m, 2H), 7.66-7.57 (m, 2H), 7.50 (d, J=10.3 Hz, 1H), 7.39-7.43 (m, 1H), 7.26-7.22 (m, 1H), 6.98 (d, J=8.6 Hz, 1H), 6.73 (d, J=8.4 Hz, 1H), 5.89 (d, J=10.3 Hz, 1H), 4.56 (s, 3H), 2.84 (s, 3H), 2.06 (s, 3H), 2.06 (s, 3H), 1.62 (s, 3H), 1.35 (s, 3H). HRMS (ESI) calculated for [M, C₄₀H₃₇N₂O]⁺: 561.2900, found: 561.2906.

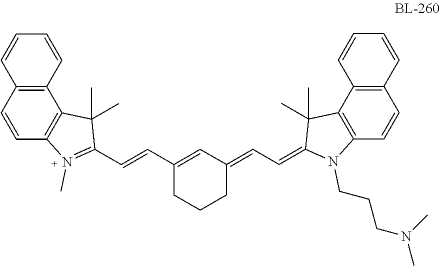

2-((E)-2-((E)-3-(2-(3-(3-(dimethylamino)propyl)-1,1-dimethyl-2,3-dihydro-1H-benzo[e]indol-2-yl)ethylidene)cyclohex-1-en-1-yl)vinyl)-1,1,3-trimethyl-1H-benzo[e]indol-3-ium (BL-260): Prepared according to the general procedure from 4a (70 mg, 0.2 mmol, 1 equiv), 2 (65 mg, 0.2 mmol, 1 equiv) and 4d (91 mg, 0.2 mmol, 1 equiv). Yield: 20% yield (28 mg). ¹H-NMR (600 MHz, MD₃OD) δ 8.24 (d, J=9.1 Hz, 1H), 8.23 (d, J=9.1 Hz, 1H), 8.04-7.95 (m, 4H), 7.91 (d, J=14.2 Hz, 1H), 7.85 (d, J=13.9 Hz, 1H), 7.67-7.56 (m, 4H), 7.54-7.43 (m, 3H), 6.30-6.21 (m, 2H), 4.27 (t, J=7.2 Hz, 2H), 3.76 (s, 3H), 2.65-2.60 (m, 4H), 2.58-2.51 (m, 2H), 2.55 (s, 6H), 2.09-1.96 (m, 14H). HRMS (ESI) calculated for [M, C₄₄H₅₀N₃]⁺: 620.3999, found: 620.4000.

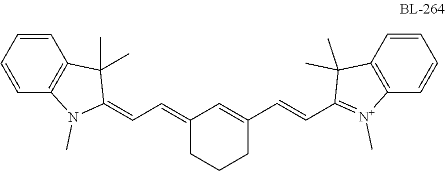 [SMILES: CN1/C(=C/C=C2C=C(/C=C/C3=[N+](C)c4ccccc4C3(C)C)CCC/2)C(C)(C)c2ccccc21]

1,3,3-trimethyl-2-((E)-2-((E)-3-(2-((E)-1,3,3-trimethylindolin-2-ylidene)ethylidene)cyclohex-1-en-1-yl)vinyl)-3H-indol-1-ium (BL-264): Prepared according to the general procedure from 4i (60 mg, 0.2 mmol, 2 equiv) and 2 (33 mg, 0.1 mmol, 1 equiv). Yield: 87% yield (50 mg). ¹H-NMR (600 MHz, MD₃OD) δ 7.76 (d, J=14.0 Hz, 2H), 7.49-7.44 (m, 3H), 7.39 (td, J=7.7, 1.2 Hz, 2H), 7.27-7.21 (m, 4H), 6.15 (d, J=14.0 Hz, 2H), 3.60 (s, 6H), 2.57 (t, J=6.2 Hz, 4H), 1.98-1.92 (m, 2H), 1.71 (s, 12H). ¹³C-NMR (151 MHz, MD₃OD) δ 173.41, 149.81, 144.46, 142.27, 133.79, 129.68, 125.85, 123.24, 111.46, 100.77, 50.15, 31.40, 27.91, 24.96, 22.73. HRMS (ESI) calculated for [M, C₃₂H₃₇N₂]⁺: 449.2951, found: 449.2946.

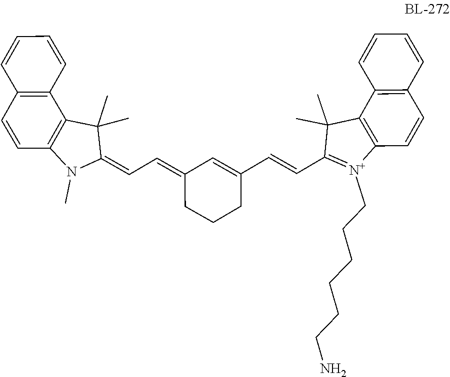 [SMILES: CN1/C(=C/C=C2C=C(/C=C/C3=[N+](CCCCCCN)c4ccc5ccccc5c4C3(C)C)CCC/2)C(C)(C)c2c1ccc1ccccc21]

3-(6-aminohexyl)-1,1-dimethyl-2-((E)-2-((E)-3-((E)-2-(1,1,3-trimethyl-1,3-dihydro-2H-benzo[e]indol-2-ylidene)ethylidene)cyclohex-1-en-1-yl)vinyl)-1H-benzo[e]indol-3-ium (BL-272): Prepared according to the general procedure from 4a (70 mg, 0.2 mmol, 1 equiv), 2 (65 mg, 0.2 mmol, 1 equiv) and 4f (94 mg, 0.2 mmol, 1 equiv). Yield: 10% yield (14 mg). ¹H-NMR (600 MHz, MD₃OD) δ 8.24 (d, J=4.8 Hz, 1H), 8.22 (d, J=4.9 Hz, 1H), 8.03-7.95 (m, 4H), 7.90 (d, J=14.1 Hz, 1H), 7.85 (d, J=14.0 Hz, 1H), 7.65-7.61 (m, 2H), 7.58 (t, J=9.2 Hz, 2H), 7.54 (s, 1H), 7.49-7.44 (m, 2H), 6.25-6.17 (m, 2H), 4.25 (t, J=7.4 Hz, 2H), 3.74 (s, 3H), 2.95-2.90 (m, 2H), 2.64-2.58 (m, 4H), 2.04-1.96 (m, 14H), 1.94-1.88 (m, 2H), 1.72-1.66 (m, 2H), 1.59-1.48 (m, 4H). ¹³C-NMR (151 MHz, MD₃OD) δ 175.21, 173.65, 156.10, 149.40, 148.36, 141.78, 141.29, 134.67, 134.44, 133.83, 133.51, 133.40, 133.22, 131.64, 131.11, 131.09, 129.99, 129.54, 129.42, 128.69, 128.68, 125.93, 125.85, 125.81, 123.35, 123.31, 118.00, 114.13, 111.97, 111.88, 100.80, 100.07, 52.13, 51.97, 44.80, 40.64, 31.90, 28.52, 28.40, 27.62, 27.56, 27.47, 27.29, 25.04, 22.80. HRMS (ESI) calculated for [M, C₄₅H₅₂N₃]⁺: 634.4156, found: 634.4153.

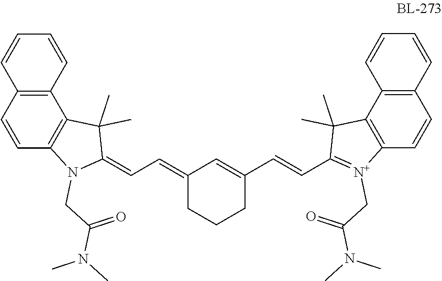 [SMILES: CN(C)C(=O)CN1/C(=C/C=C2C=C(/C=C/C3=[N+](CC(=O)N(C)C)c4ccc5ccccc5c4C3(C)C)CCC/2)C(C)(C)c2c1ccc1ccccc21]

3-(2-(dimethylamino)-2-oxoethyl)-2-((E)-2-((E)-3-((E)-2-(3-(2-(dimethylamino)-2-oxoethyl)-1,1-dimethyl-1,3-dihydro-2H-benzo[e]indol-2-ylidene)ethylidene)cyclohex-1-en-1-yl)vinyl)-1,1-dimethyl-1H-benzo[e]indol-3-ium (BL-273): Prepared according to the general procedure from 2 (33 mg, 0.1 mmol, 1 equiv) and 4c (75 mg, 0.2 mmol, 2 equiv). Yield: 26% yield (20 mg). ¹H-NMR (600 MHz, MD₃OD) δ 8.24 (d, J=8.5 Hz, 2H), 7.98-7.94 (m, 4H), 7.87 (d, J=14.2 Hz, 2H), 7.65-7.61 (m, 2H), 7.51 (s, 1H), 7.47 (t, J=7.6 Hz, 2H), 7.44 (d, J=8.8 Hz, 2H), 6.06 (d, J=14.0 Hz, 2H), 5.24 (s, 4H), 3.32 (s, 6H), 3.04 (s, 6H), 2.55 (t, J=6.2 Hz, 4H), 2.06 (s, 12H), 1.96-1.90 (m, 2H). HRMS (ESI) calculated for [M, C₄₆H₅₁N₄O₂]⁺: 691.4007, found: 691.4002.

### Additional Cyanine Based Dyes

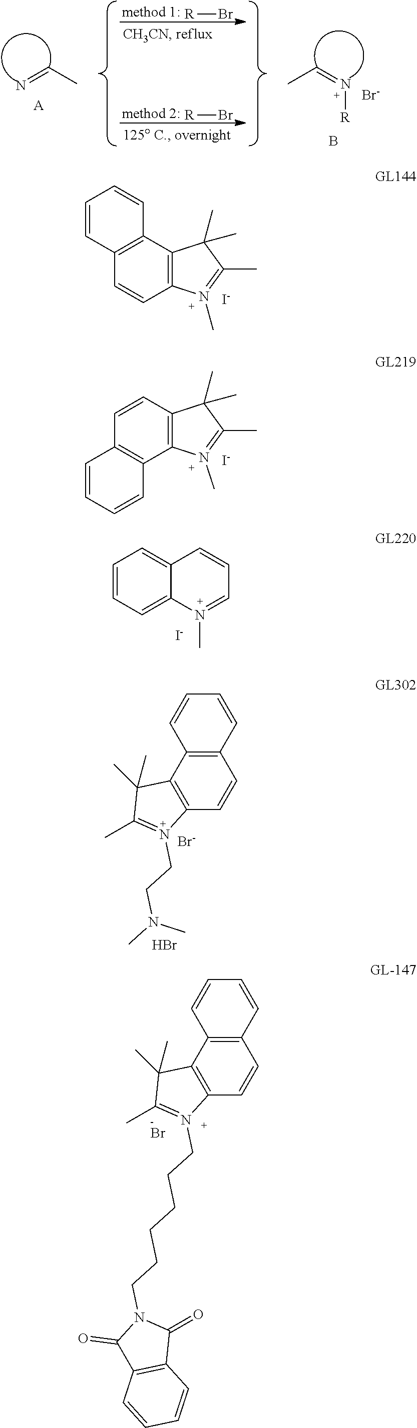

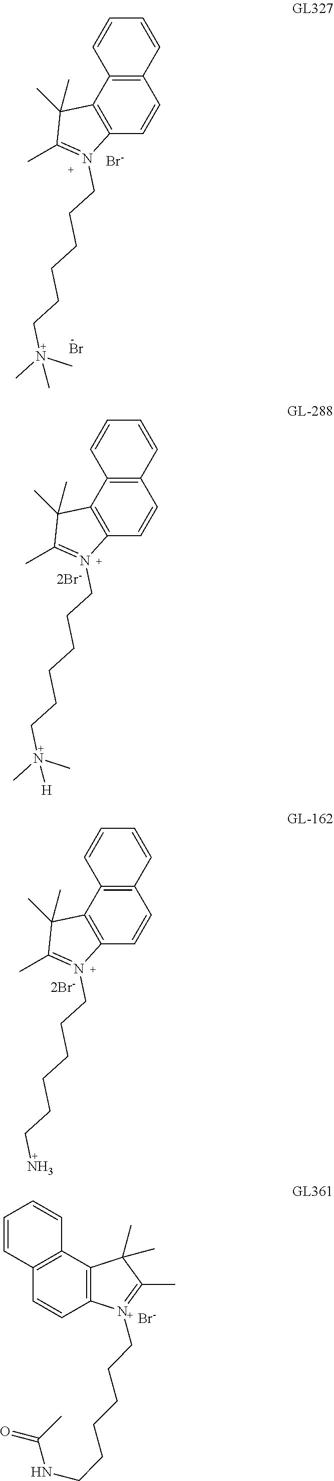

### General Procedure for the Synthesis of Heterocyclic Salt:

Method 1: Compound A (1 equiv.) and R—Br (1.2-1.5 equiv.) were mixed in CH₃CN in a screwed-capped vial. The mixture was stirred and heated to reflux for overnight. Solid was filtered and washed with ether and dried in vacuum.
Method 2: Compound A (1 equiv.) and R—Br (1.0 equiv.) were mixed in an 8 mL of screwed-capped vial. The mixture was stirred and heated to 125° C. for overnight. Blank solid was washed with ethyl acetate ether and recrystallized with methanol/ether.

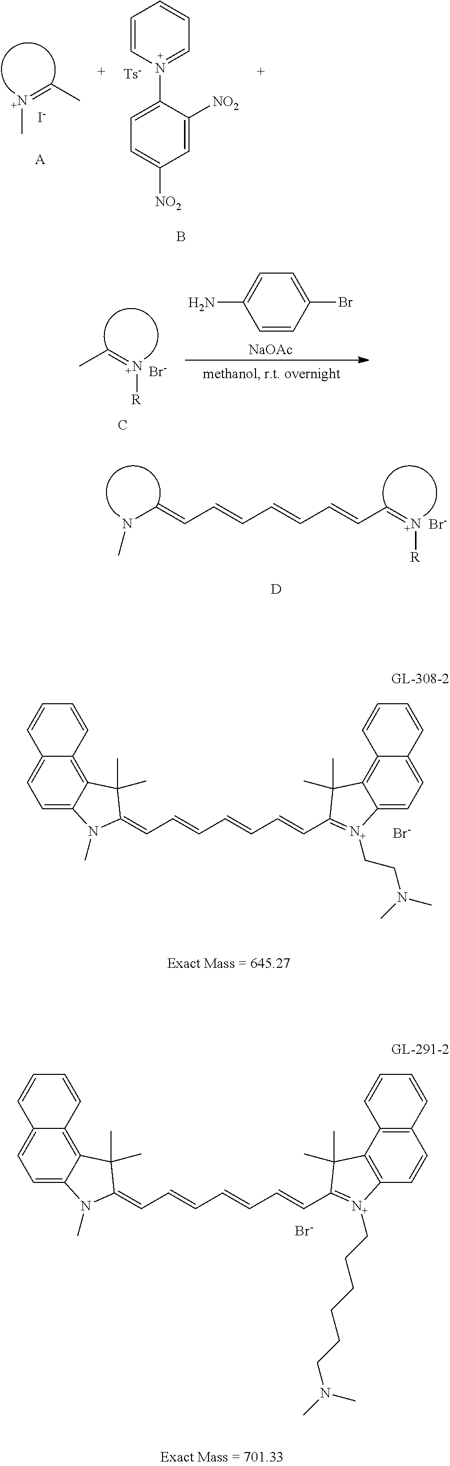

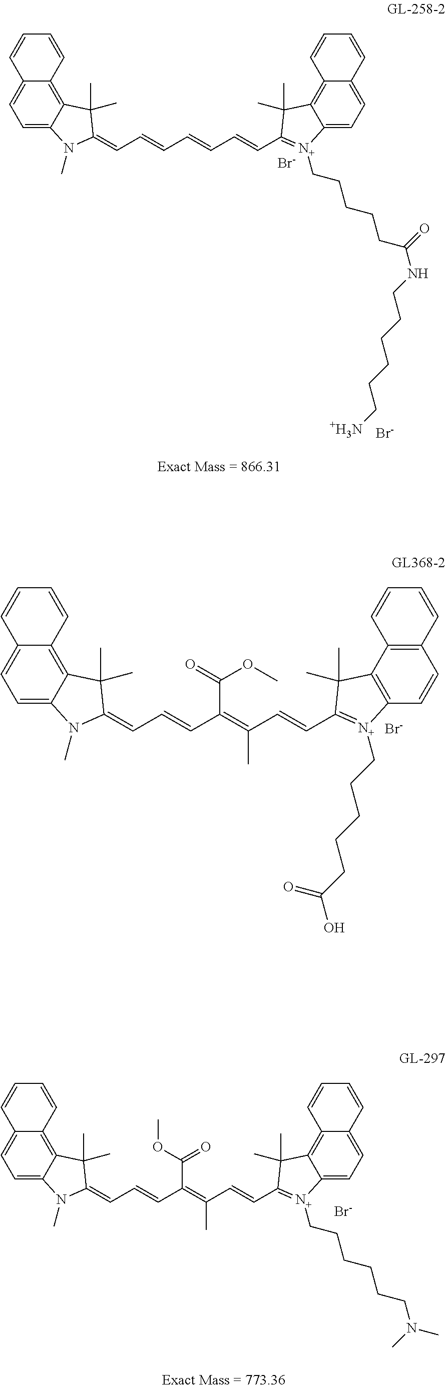

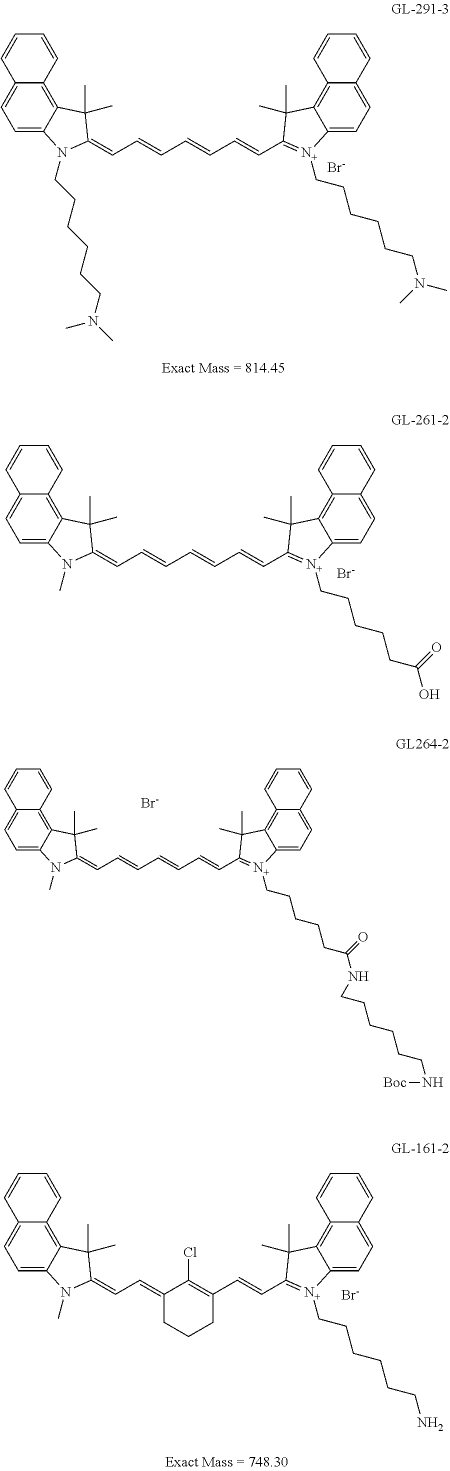

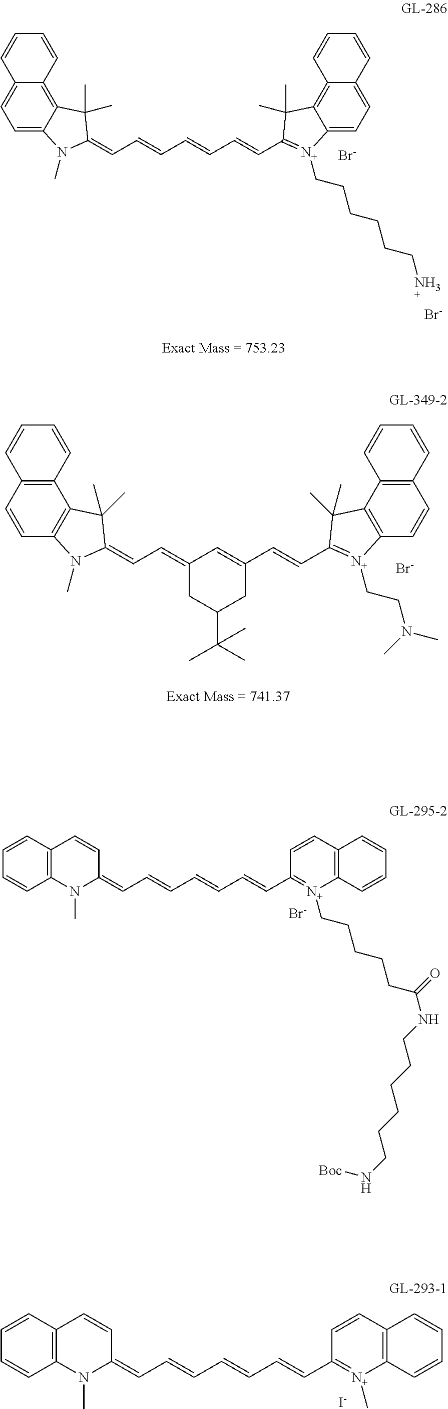

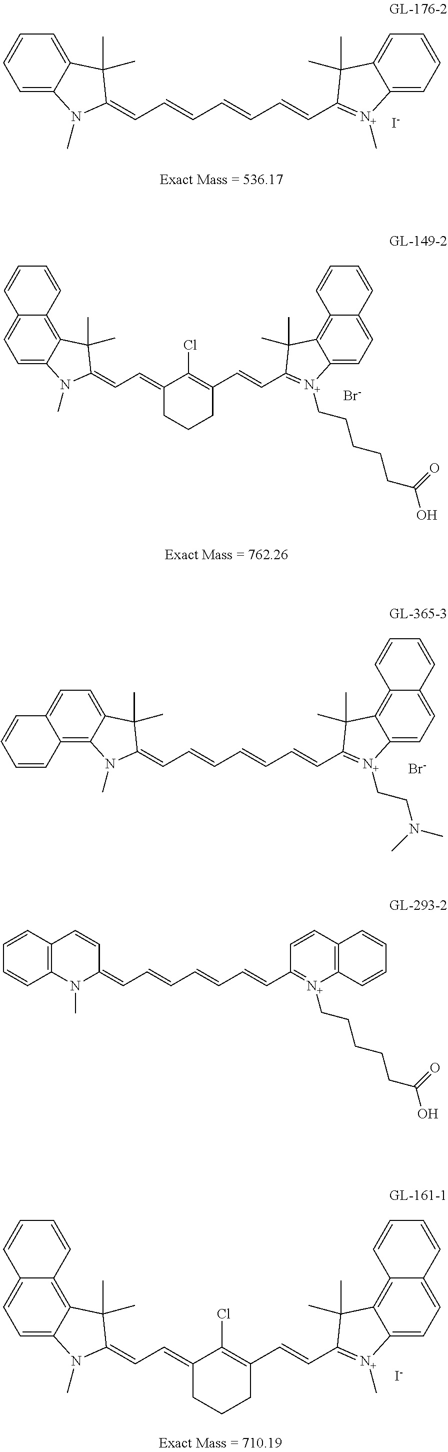

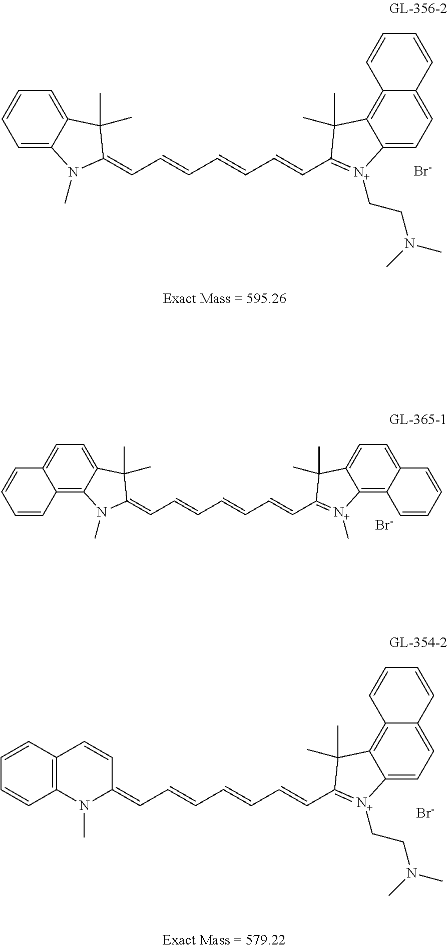 [SMILES: CN(C)CC[N+]1=C(/C=C/C=C/C=C/C=C2/N(C)c3ccccc3C2(C)C)C(C)(C)c2c1ccc1ccccc21 ; [Br-] ; CN1/C(=C/C=C/C=C/C=C/C2=[N+](C)c3c(ccc4ccccc34)C2(C)C)C(C)(C)c2ccc3ccccc3c21 ; [Br-] ; CN(C)CC[N+]1=C(/C=C/C=C/C=C/C=C2\C=Cc3ccccc3N2C)C(C)(C)c2c1ccc1ccccc21 ; [Br-]]

General procedure: The corresponding pyridinium salt B (1 equiv.) and 4-bromoaniline (1 equiv.) were dissolved in methanol (4 mL) in an 8 mL of vial, and the mixture was stirred at room temperature for 30 min. Next, a heterocyclic salt A (1 equiv.), C (1 equiv.) and sodium acetate (3 equiv.) were added. The reaction mixture was stirred for additional overnight at room temperature. The crude product was purified by flash column chromatography (silica gel, dichlormethane/methanol).

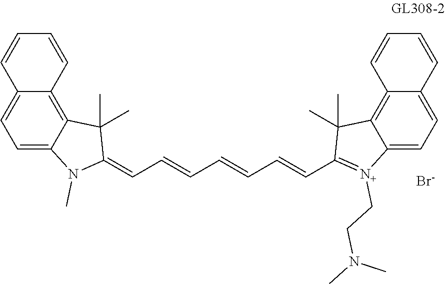 [SMILES: CN(C)CC[N+]1=C(/C=C/C=C/C=C/C=C2/N(C)c3ccc4ccccc4c3C2(C)C)C(C)(C)c2c1ccc1ccccc21 ; [Br-]]

The corresponding pyridinium salt 175 (206 mg, 0.48 mmol) and 4-bromoaniline (103 mg, 0.3 mmol) were dissolved in methanol (4 mL, 7 mL/mmol), and the mixture was stirred at room temperature for 30 min. Next, a heterocyclic salt 144 (176 mg, 0.5 mmol), 302 (286 mg, 0.65 mmol) and sodium acetate (246 mg) were added, and the reaction mixture was stirred for additional 16 h at room temperature. The crude product was purified by flash column chromatography (silica gel, dichlormethane/methanol, 20:1, then 10:1). Affording 58 mg of green solid GL308-2. Yield: 19%. ¹H NMR (600 MHz, Methanol-d4) δ 8.27 (d, J=8.17 Hz, 1H), 8.23 (d, J=8.17 Hz, 1H), 8.11 (t, 1H), 8.06-7.98 (m, 5H), 7.69-7.63 (m, 4H), 7.55 (d, J=8.80 Hz, 1H), 7.52 (t, 1H), 7.48 (t, 1H), 6.67-6.58 (m, 2H), 6.42 (d, J=13.82 Hz, 1H), 6.27 (d, J=13.82 Hz, 1H), 4.29 (t, 2H), 3.79 (s, 3H), 2.77 (t, 2H), 2.43 (s, 6H), 2.02 (d, J=4.66 Hz, 12H). ¹³C NMR (150 MHz, Methanol-d4) δ 174.95, 171.89, 171.50, 149.47, 140.24, 139.83, 133.86, 132.70, 132.20, 131.76, 130.37, 130.24, 129.74, 129.69, 128.20, 127.95, 127.41, 127.24, 125.96, 125.61, 124.85, 124.32, 122.01, 121.84, 110.66, 110.25, 104.22, 102.16, 55.27, 51.09, 50.45, 48.16, 44.52, 41.59, 30.71, 26.27, 25.97. HRMS (ESI) for C₄₀H₄₄N₃⁺ [M-Br]⁺: 566.3530. Found: 566.3534.

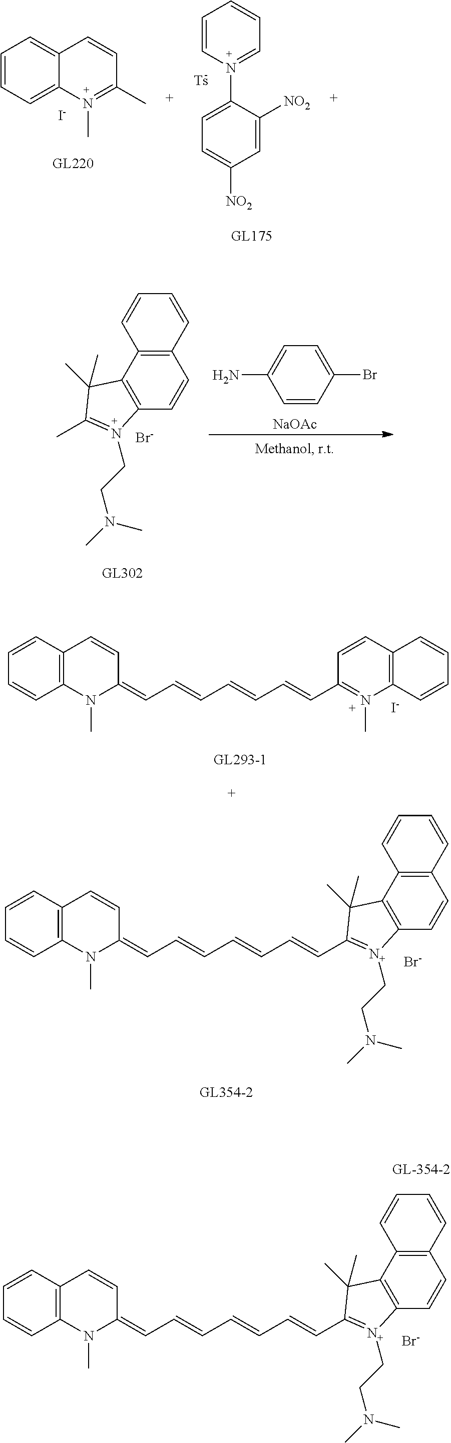

The corresponding pyridinium salt GL175 (209 mg, 0.5 mmol) and 4-bromoaniline (103 mg, 0.65 mmol) were dissolved in pyridine (4 mL, 7 mL/mmol), and the mixture was stirred at room temperature for 30 min. Next, a heterocyclic salt GL220 (143 mg, 0.5 mmol), GL302 (286 mg, 0.65 mmol) and sodium acetate (246 mg) were added, and the reaction mixture was stirred for additional 16 h at room temperature. The crude product was purified by flash column chromatography (silica gel, dichlormethane/methanol, 20:1, 10:1). 80 mg of green solid was obtained. Yield: 27.5%. ¹H NMR (600 MHz, Methanol-d4) δ 8.48 (d, J=9.20 Hz, 1H), 0.8.23 (s, J=9.15 Hz, 2H), 8.13-8.00 (m, 4H), 7.87 (d, J=8.52 Hz, 2H), 7.75 (t, 1H), 7.62 (t, 1H), 7.52 (t, 1H), 7.33 (m, 3H), 6.96 (d, J=13.83 Hz, 1H), 6.69 (t, 1H), 6.42 (t, 1H), 5.84 (d, J=12.51 Hz, 1H), 4.31 (s, 3H), 4.08 (t, 2H), 2.70 (t, 2H), 2.45 (s, 6H), 1.95 (s, 6H), ¹³C NMR (150 MHz, Methanol-d4) δ 164.55, 155.60, 150.45, 141.82, 140.81, 140.41, 139.80, 133.82, 130.69, 129.88, 129.72, 129.58, 129.55, 128.69, 127.33, 126.82, 126.70, 126.28, 124.68, 122.86, 121.44, 119.68, 117.50, 114.24, 109.31, 98.05, 54.70, 48.70, 48.05, 47.88, 44.45, 40.41, 37.20, 26.46. HRMS (ESI) for C₃₅H₃₈N₃⁺ [M-Br]⁺: 500.3060. Found: 500.3087.

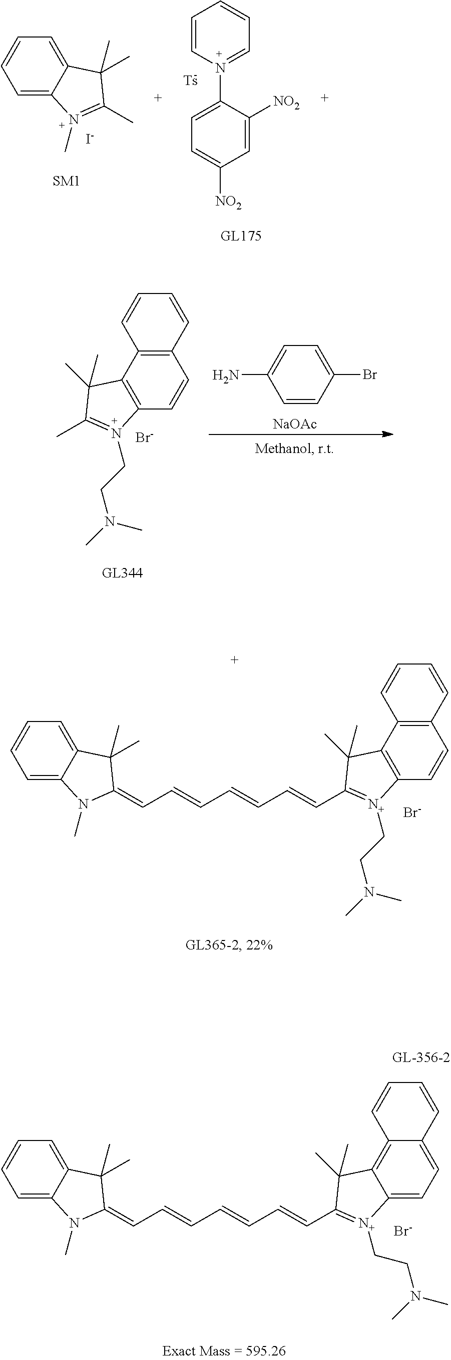

The corresponding pyridinium salt GL175 (237 mg, 0.5 mmol) and 4-bromoaniline (103 mg, 0.6 mmol) were dissolved in methanol (4 mL, 7 mL/mmol), and the mixture was stirred at room temperature for 30 min. Next, a heterocyclic salt GL176-1 (150 mg, 0.5 mmol), GL302 (286 mg, 0.65 mmol) and sodium acetate (246 mg) were added, and the reaction mixture was stirred for additional 16 h at room temperature. The crude product was purified by flash column chromatography (silica gel, dichlormethane/methanol, 20:1, 10:1), affording 54 mg of GL-356-2 as green solid in 18.1% yield. ¹H NMR (600 MHz, Methanol-d4) δ 8.24 (d, J=8.43 Hz, 1H), 8.04-7.99 (m, 4H), 7.66-7.63 (m, 2H), 7.58 (d, J=8.95 Hz, 1H), 7.51 (m, 2H), 7.43 (m, 1H), 7.32-7.27 (m, 2H), 6.61 (t, 2H), 6.33 (m, 2H), 4.33 (t, 2H), 3.64 (s, 3H), 2.80 (t, 2H), 2.45 (s, 6H), 1.99 (s, 6H), 1.73 (s, 6H). ¹³C NMR (150 MHz, Methanol-d4) δ 142.95, 141.05, 139.67, 133.13, 131.97, 131.31, 130.31, 129.70, 128.94, 128.67, 128.36, 128.13, 127.31, 125.79, 124.79, 124.64, 124.53, 121.89, 121.26, 110.48, 110.40, 107.96, 103.83, 102.86, 70.74, 55.28, 50.70, 48.98, 44.48, 41.71, 30.20, 26.51, 26.25. HRMS (ESI) for C₃₆H₄₂N₃⁺ [M-Br]⁺: 516.3373. Found: 516.3380.

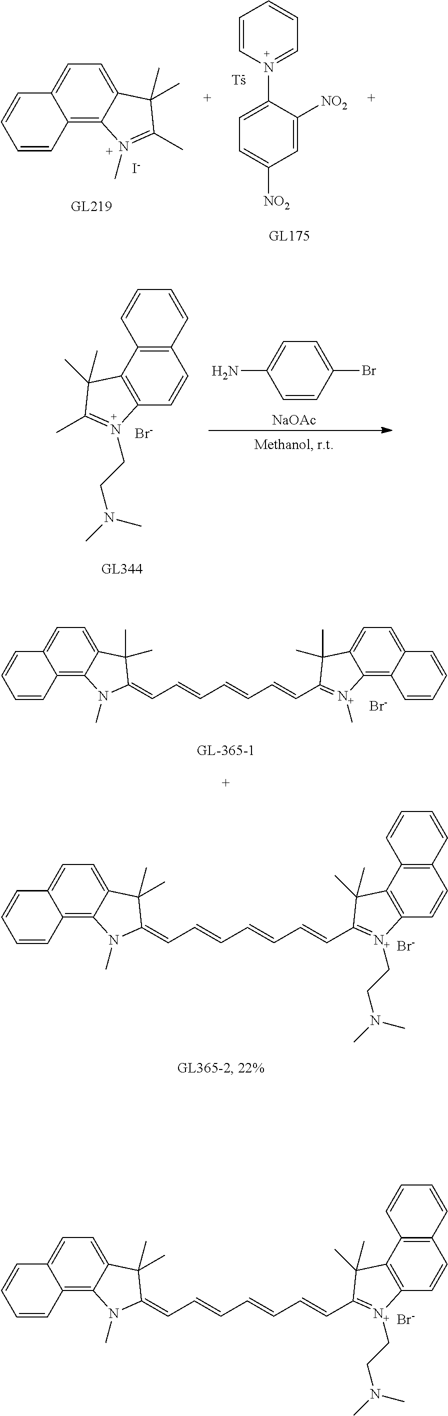

The corresponding pyridinium salt GL175 (103 mg, 0.24 mmol) and 4-bromoaniline (49 mg, 0.29 mmol) were dissolved in methanol (4 mL), and the mixture was stirred at room temperature for 30 min. Next, a heterocyclic salt GL219 (105 mg, 0.3 mmol), GL344 (132 mg, 0.3 mmol) and sodium acetate (116 mg) were added, and the reaction mixture was stirred for additional 16 h at room temperature. The crude product was purified by flash column chromatography (silica gel, dichlormethane/methanol, 20:1, then 10:1), affording GL365-2 as green solid in 22% yield. ¹H NMR (600 MHz, Methanol-d4) δ 8.62 (d, J=8.83 Hz, 1H), 8.23 (d, J=8.83 Hz, 1H), 8.08-7.98 (m, 4H), 7.88-7.82 (m, 2H), 7.71-7.63 (m, 3H), 7.58 (m, 2H), 7.48 (t, 1H), 6.69-6.60 (m, 2H), 6.51 (d, J=13.55 Hz, 1H), 6.31 (d, J=13.55 Hz, 1H), 4.34 (t, 2H), 4.22 (s, 3H), 2.84 (t, 2H), 2.49 (s, 6H), 2.01 (s, 6H), 1.78 (s, 6H). ¹³C NMR (150 MHz, Methanol-d4) δ 174.57, 139.71, 139.02, 137.44, 135.06, 131.85, 130.30, 129.70, 129.40, 128.17, 127.29, 126.73, 126.57, 125.71, 124.44, 121.87, 121.64, 121.30, 118.98, 110.30, 55.13, 53.40, 50.58, 49.00, 44.39, 43.06, 41.49, 36.15, 28.93, 26.38, 26.24, 19.84. HRMS (ESI) for C₃₅H₃₈N₃⁺ [M-Br]⁺: 500.3060. Found: 500.3087.

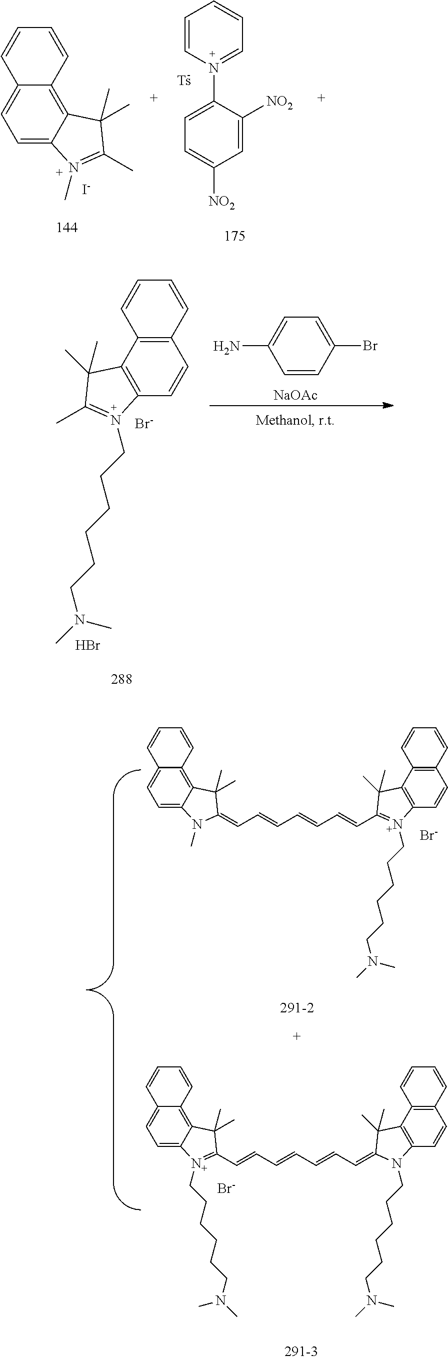

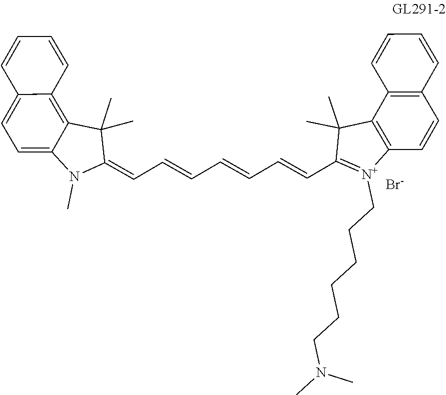 [SMILES: CN(C)CCCCCC[N+]1=C(/C=C/C=C/C=C/C=C2/N(C)c3ccc4ccccc4c3C2(C)C)C(C)(C)c2c1ccc1ccccc21 ; [Br-]]

The corresponding pyridinium salt 175 (206 mg, 0.5 mmol) and 4-bromoaniline (103 mg, 0.6 mmol) were dissolved in methanol (4 mL, 7 mL/mmol), and the mixture was stirred at room temperature for 30 min. Next, a heterocyclic salt 144 (176 mg, 0.5 mmol), 288 (252 mg, 0.65 mmol) and sodium acetate (246 mg) were added, and the reaction mixture was stirred for additional 16 h at room temperature. The crude product was purified by flash column chromatography (silica gel, dichlormethane/methanol, 20:1, 10:1, 8:1). affording GL291-2 as green solid in 19% yield. ¹H NMR (600 MHz, Methanol-d4) δ 8.13 (m, 1H), 7.91 (m, 3H), 7.54-7.48 (m, 2H), 7.37 (m, 1H), 6.52 (m, 1H), 6.24 (m, 1H), 3.38 (m, 2H), 3.02 (m, 2H), 2.79 (m, 12H), 1.89 (m, 6H), 1.81 (m, 2H), 1.64 (m, 2H), 1.42 (m, 2H), 1.33 (m, 2H). ¹³C NMR (150 MHz, Methanol-d4) δ 140.36, 139.80, 136.85, 133.37, 133.30, 131.899, 131.86, 130.91, 130.30, 130.27, 129.71, 129.68, 128.28, 128.13, 128.02, 127.31, 127.21, 124.58, 124.51, 122.99, 121.95, 121.92, 110.68, 110.52, 65.50, 57.55, 53.42, 42.17, 30.60, 27.83, 26.28, 26.12, 24.24, 20.65, 14.05. HRMS (ESI) for C₄₅H₅₂N₃⁺ [M-Br]⁺: 622.4156. Found: 622.4162.

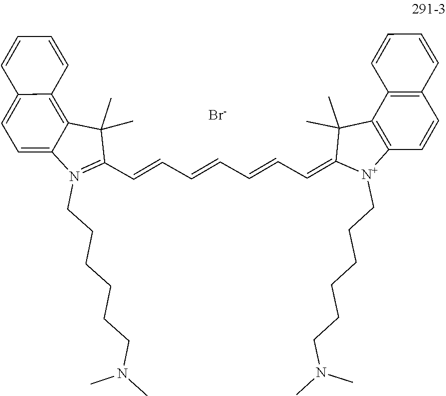

Synthesis affording GL291-3 as green solid in 10% yield. ¹H NMR (600 MHz, Methanol-d4) δ 8.26 (d, J=8.54 Hz, 2H), 8.08 (t, 2H), 8.03-8.00 (m, 3H), 7.97-7.94 (m, 2H), 7.78-7.77 (m, 2H), 7.66 (m, 2H), 7.60 (d, J=8.64 Hz, 2H), 7.51 (t, 2H), 6.64 (t, 2H), 6.37 (d, J=13.71 Hz, 2H), 4.25 (t, 4H), 3.03 (m, 4H), 2.80 (s, 12H), 2.03 (s, 12H), 1.93 (m, 4H), 1.73 (m, 4H), 1.59 (m, 4H), 1.50 (m, 4H). HRMS (ESI) for C₅₁H₆₇N₃⁺ [M-Br]⁺: 735.5360. Found: 735.5368.

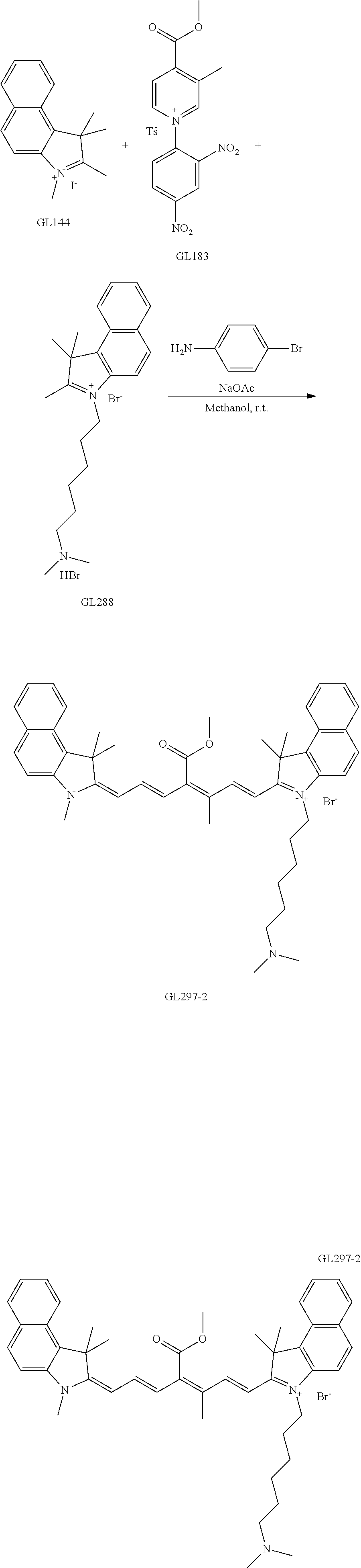

The corresponding pyridinium salt 183 (237 mg, 0.5 mmol) and 4-bromoaniline (103 mg, 0.6 mmol) were dissolved in methanol (4 mL, 7 mL/mmol), and the mixture was stirred at room temperature for 30 min. Next, a heterocyclic salt 144 (176 mg, 0.5 mmol), 288 (322 mg, 0.65 mmol) and sodium acetate (246 mg) were added, and the reaction mixture was stirred for additional 16 h at room temperature. The crude product was purified by flash column chromatography (silica gel, dichlormethane/methanol, 30:1, 20:1, 10:1). affording GL297-2 as green solid in 22% yield. The product was found in LCMS at M:694.4 and [M²⁺]/2: 347.8. ¹H NMR (600 MHz, Methanol-d4) δ 8.28 (m, 2H), 8.08-8.01 (m, 4H), 7.86-7.76 (m, 2H), 7.70-7.62 (m, 4H), 7.55-7.51 (m, 2H), 6.81-6.72 (m, 1H), 6.61-6.57 (m, 1H), 6.41-6.34 (m, 1H), 4.34-4.28 (m, 2H), 4.19 (s, 3H), 3.83 (d, J=6.96 Hz, 3H), 2.90 (m, 2H), 2.68 (m, 6H), 2.21 (d, J=12.22 Hz, 3H), 1.97 (s, 12H), 1.95 (m, 2H), 1.70 (m, 2H), 1.61 (m, 2H), 1.50 (m, 2H).

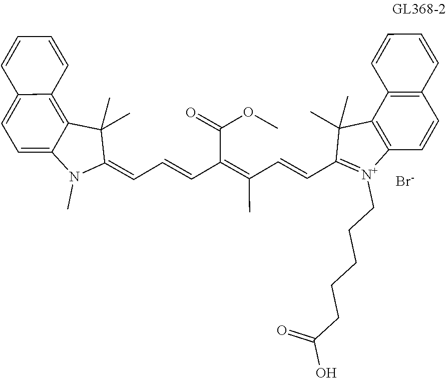 [SMILES: COC(=O)C(/C=C/C=C1/N(C)c2ccc3ccccc3c2C1(C)C)=C(C)\C=C\C1=[N+](CCCCCC(=O)O)c2ccc3ccccc3c2C1(C)C ; [Br-]]

GL368-2 was obtained by use the similar procedure of GL297-2.

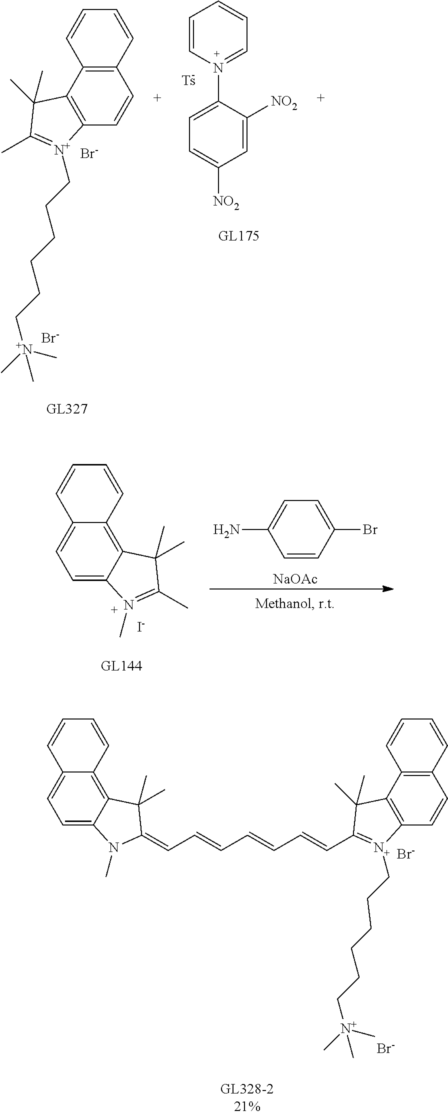

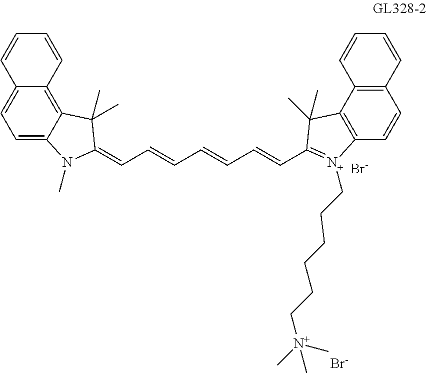 [SMILES: CN1/C(=C/C=C/C=C/C=C/C2=[N+](CCCCCC[N+](C)(C)C)c3ccc4ccccc4c3C2(C)C)C(C)(C)c2c1ccc1ccccc21 ; [Br-] ; [Br-]]

144 (105 mg, 0.3 mmol), 175 (103 mg, 0.24 mmol), 328 (154 mg, 0.3 mmol) and 4-bromoaniline (49 mg), NaOAc (116 mg) and methanol (4 mL) were mixed. The mixture was stirred at room temperature for overnight. After that, the methanol was removed by rotary evaporator. Then, the crude product was purified by flash column chromatography (silica gel, dichlormethane/methanol, 20:1 to 10:1), affording GL328-2 as green solid in 21% yield. ¹H NMR (600 MHz, Methanol-d4) δ 8.26-8.24 (m, 2H), 8.11-7.99 (m, 6H), 7.72-7.63 (m, 3H), 7.61 (t, 2H), 7.49 (m, 2H), 6.65 (m, 2H), 6.36 (d, J=13.67 Hz, 2H), 4.25 (t, 2H), 3.76 (s, 3H), 3.41-3.38 (m, 2H), 3.15 (s, 9H), 2.01 (d, J=1.89 Hz, 12H), 1.96-1.91 (m, 2H), 1.87-1.82 (m, 2H), 1.66-1.61 (m, 2H), 1.54-1.48 (m, 2H). ¹³C NMR (150 MHz, Methanol-d4) δ 140.35, 139.83, 133.45, 133.24, 132.05, 131.85, 130.29, 129.92, 129.72, 129.69, 128.15, 128.02, 127.99, 127.33, 127.32, 124.63, 124.51, 123.99, 121.95, 121.90, 110.61, 110.52, 66.33, 62.91, 52.23, 52.20, 52.18, 50.84, 50.67, 43.46, 30.55, 29.32, 27.21, 26.27, 26.09, 26.05, 25.79, 22.48. LCMS (ESI) for C₄₇H₅₆N₃²⁺ [M-Br]²⁺: 318.7. Found: 318.9.

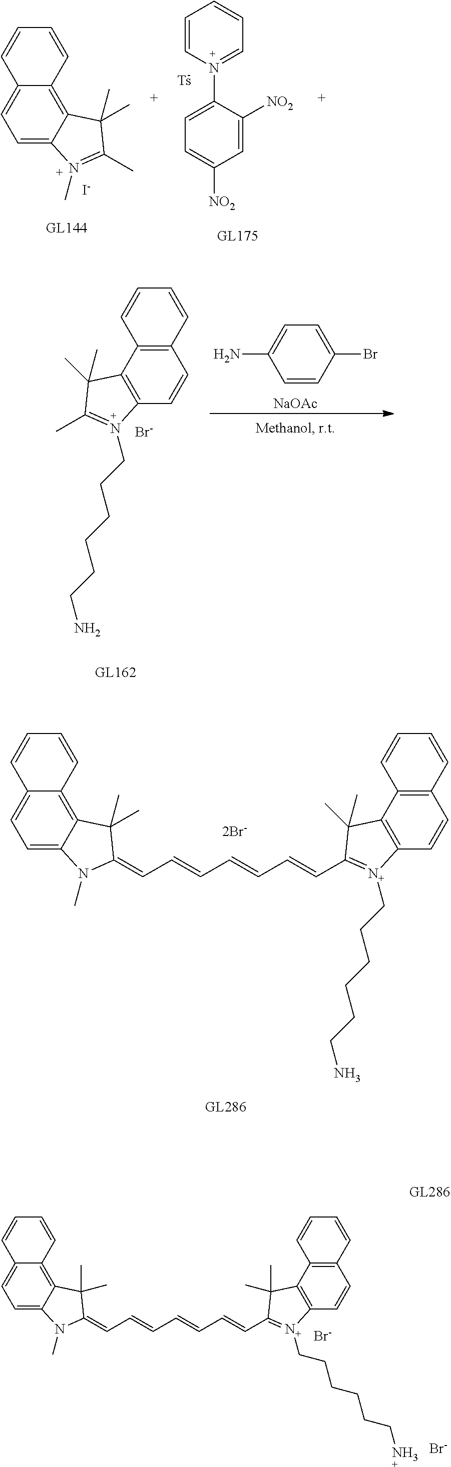

The corresponding pyridinium salt 175 (206 mg, 0.5 mmol) and 4-bromoaniline (103 mg, 0.6 mmol) were dissolved in methanol (4 mL, 7 mL/mmol), and the mixture was stirred at room temperature for 30 min. Next, a heterocyclic salt 144 (176 mg, 0.5 mmol), 162 (252 mg, 0.65 mmol) and sodium acetate (246 mg) were added, and the reaction mixture was stirred for additional 16 h at room temperature. Afterwards, Et₂O (21 mL/mmol) was added, and the mixture was placed to the freezer (−16° C.). The resulting precipitate was filtered, washed with water (2×10 mL/mmol), Et₂O (2×10 mL/mmol) and dried on air. Then, the crude product was purified by flash column chromatography (silica gel, dichlormethane/methanol, 20:1 to 10:1), affording GL286 as green solid in 18% yield. ¹H NMR (600 MHz, Methanol-d4) δ 8.15-8.12 (m, 2H), 7.99-7.87 (m, 6H), 7.56-7.44 (m, 5H), 7.40-7.36 (m, 2H), 6.51 (t, 2H), 6.24-6.20 (m, 2H), 4.11 (t, 2H), 3.64 (s, 3H), 2.82 (t, 2H), 2.26 (s, 2H), 1.90 (s, 12H), 1.81 (m, 2H), 1.58 (m, 2H), 1.43 (m, 2H). HRMS (ESI) for C₄₂H₄₈N₃⁺ [M-Br—HBr]⁺: 594.3843. Found: 594.3846.

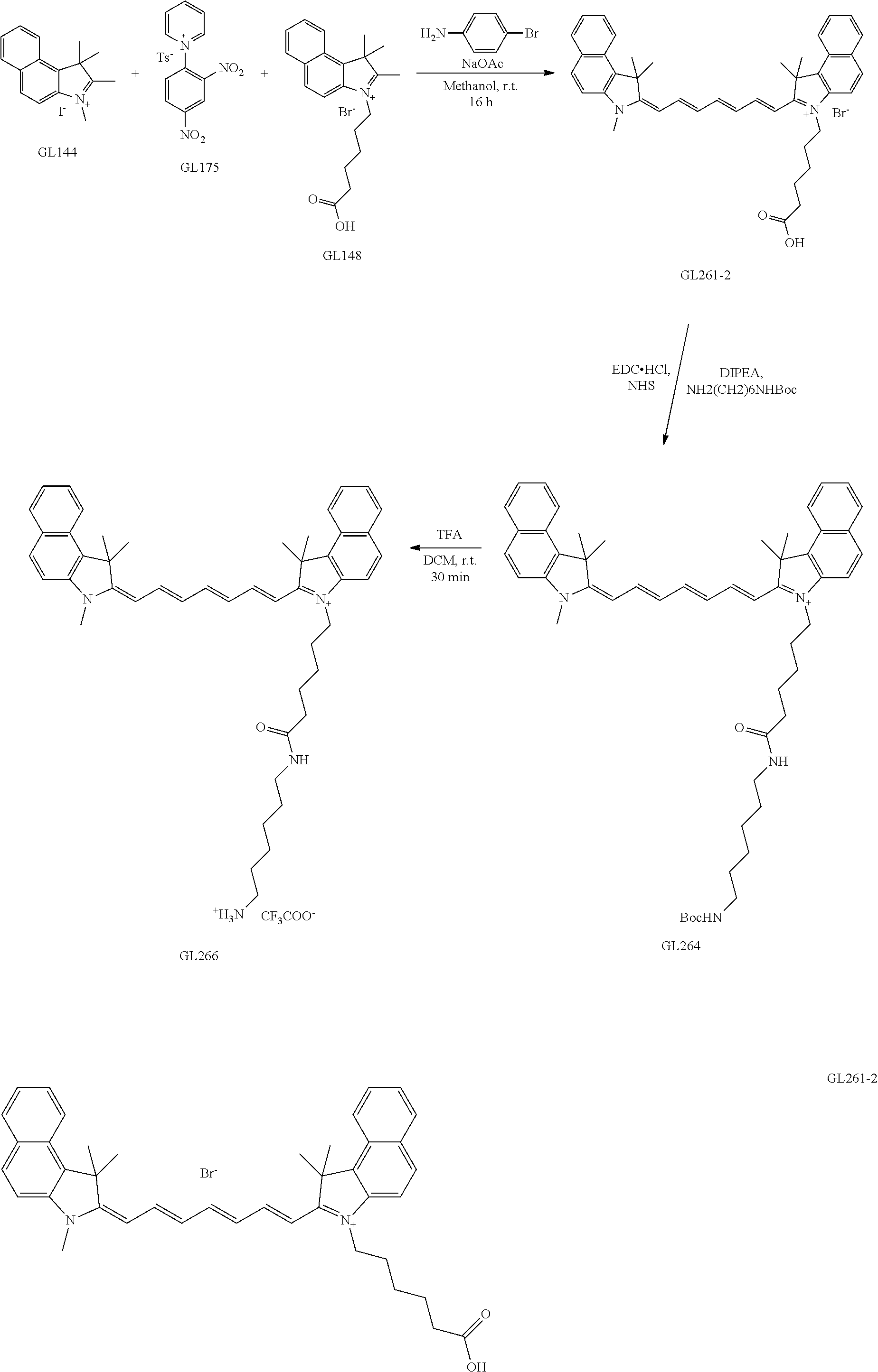

GL144 (210 mg, 0.6 mmol), GL175 (208 mg, 0.5 mmol), GL148 (265 mg, 0.6 mmol) and 4-bromoaniline (100 mg) and NaOAc (232 mg) were dissolved in methanol (4 mL), and the mixture was stirred at room temperature for overnight (16 h). The crude product was purified by flash column chromatography (silica gel, dichlormethane/methanol, 20:1 to 10:1), affording compound GL261 (87 mg, yield: 16%). ¹H NMR (600 MHz, Methanol-d4) δ 8.24 (d, J=8.86 Hz, 2H), 8.09-7.99 (m, 6H), 7.66 (t, 2H), 7.59 (t, 2H), 7.49 (t, 2H), 6.62 (t, 2H), 6.33 (m, 2H), 4.23 (t, 2H), 3.74 (s, 3H), 2.34 (t, 2H), 2.01 (s, 12H), 1.93 (m, 2H), 1.74 (m, 2H), 1.56 (m, 2H). HRMS (ESI) for C₄₂H₄₅N₂O₂⁺ [M-Br]⁺: 609.3476. Found: 609.3481.

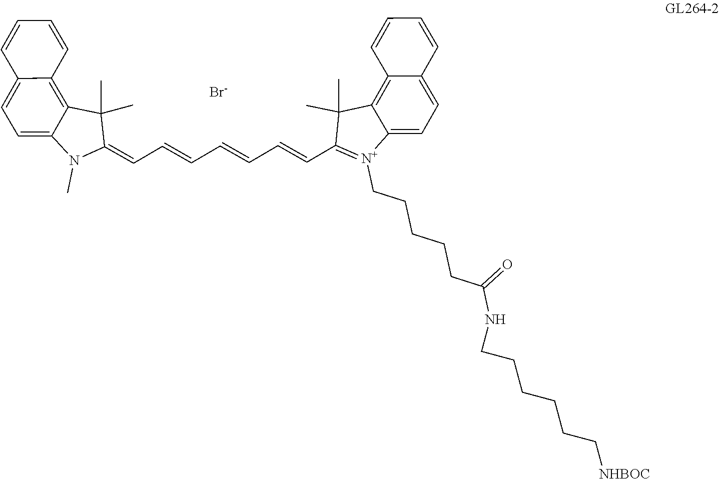

Compound GL261 (87 mg, 0.13 mmol), 1-(3-dimethylaminopropyl)-3-ethylcarbodiimide hydrochloride (EDC HCl, 29 mg, 0.15 mmol) and N-hydroxysuccinimide (NHS, 20 mg, 0.16 mmol) were dissolved in 5 mL DMF and the resulting mixture was stirred at room temperature under nitrogen atmosphere for 24 h in the dark. Then NH₂(CH₂)₆NHBoc (41 mg, 0.19 mmol) and N, N-diisopropylethylamine (DIPEA, 49 mg, 0.38 mmol) were added to the reaction solution and the mixture was stirred for an additional 6 h. The crude product was purified by silica gel column chromatography with gradient elution (dichloromethane/methane of 25:1 to 15:1) to afford GL264-2 as purplish red solid (80 mg, 69%). ¹H NMR (600 MHz, Methanol-d4) δ 8.13 (d, J=7.92 Hz, 2H), 7.98-7.88 (m, 6H), 7.54 (t, 2H), 7.49-7.45 (m, 3H), 7.38 (t, 2H), 6.50 (t, 2H), 6.24 (m, 2H), 4.11 (t, 2H), 3.63 (s, 3H), 2.98 (t, 2H), 2.90 (t, 2H), 2.10 (t, 2H), 1.90 (s, 12H), 1.78 (m, 2H), 1.61 (m, 2H), 1.38 (m, 2H), 1.30 (m, 14H), 1.17 (m, 6H).

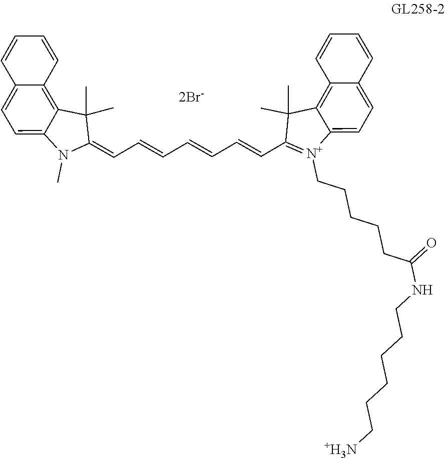 [SMILES: CN1/C(=C/C=C/C=C/C=C/C2=[N+](CCCCCC(=O)NCCCCCC[NH3+])c3ccc4ccccc4c3C2(C)C)C(C)(C)c2c1ccc1ccccc21 ; "2Br-"]

In an 8 ml of vial, 80 mg of Boc compound was dissolved in 10 mL DCM, and 1 ml of TFA added dropwise. The solution was stirred for 30 min, then, the excess of TFA was removed by vacuum, affording GL258-2 as green solid. ¹H NMR (600 MHz, Methanol-d4) δ 8.13 (d, J=8.16 Hz, 2H), 7.98-7.85 (m, 6H), 7.57-7.45 (m, 5H), 7.38 (m, 2H), 6.50 (t, 2H), 6.23 (t, 2H), 4.10 (t, 2H), 3.63 (s, 3H), 3.01 (t, 2H), 2.78 (t, 2H), 2.02 (t, 2H), 1.89 (s, 12H), 1.77 (m, 2H), 1.60 (m, 2H), 1.52 (m, 2H), 1.42-1.32 (m, 4H), 1.29-1.19 (m, 4H).

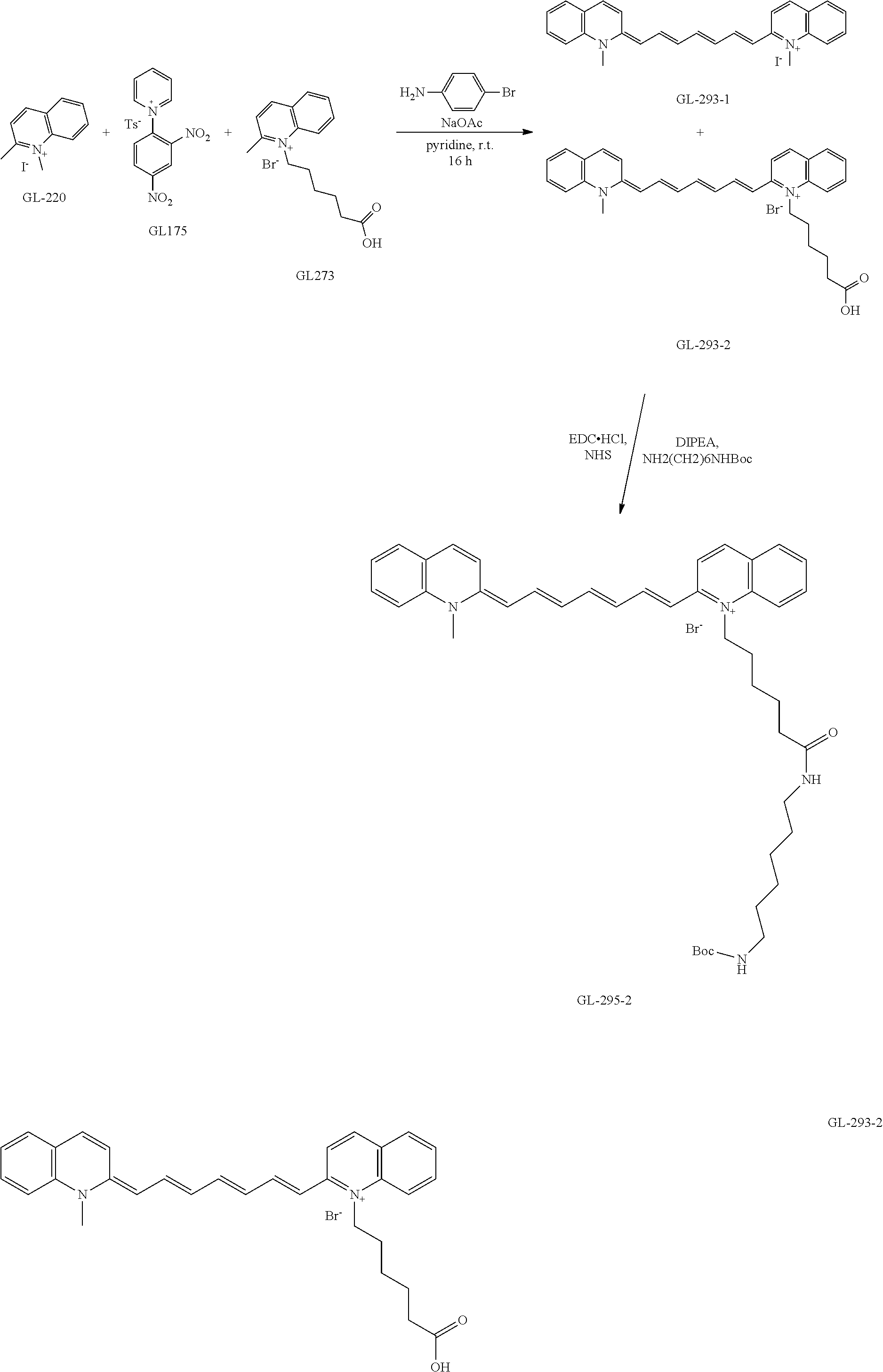

Compound GL293-2 was obtained as green solid in 15% yield by use the similar procedure of GL261-1. ¹H NMR (600 MHz, Methanol-d4) δ 7.84-7.71 (m, 11H), 7.41 (m, 2H), 7.31 (t, 1H), 6.59 (t, 2H), 6.32 (m, 2H), 4.38 (m, 2H), 2.28 (m, 2H), 1.90 (m, 5H), 1.79 (m, 2H), 1.65 (m, 2H). LCMS for C₃₂H₃₃N₂O₂⁺ [M-Br]⁺: 477.25. Found: 477.2.

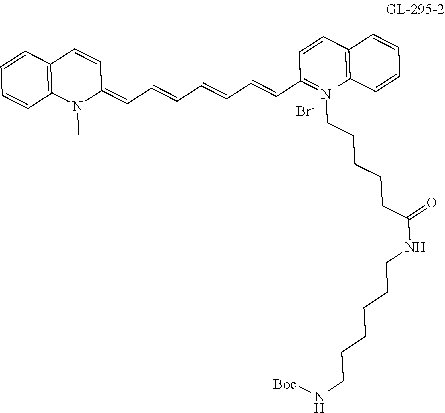 [SMILES: CN1/C(=C/C=C/C=C/C=C/c2ccc3ccccc3[n+]2CCCCCC(=O)NCCCCCCNC(=O)OC(C)(C)C)C=Cc2ccccc21 ; [Br-]]

Compound GL295-2 was afforded as green solid in 71% yield by use the similar procedure of GL264-2. LCMS for C₄₃H₅₅N₄₀₃⁺ [M-Br]⁺: 675.42. Found: 675.4.

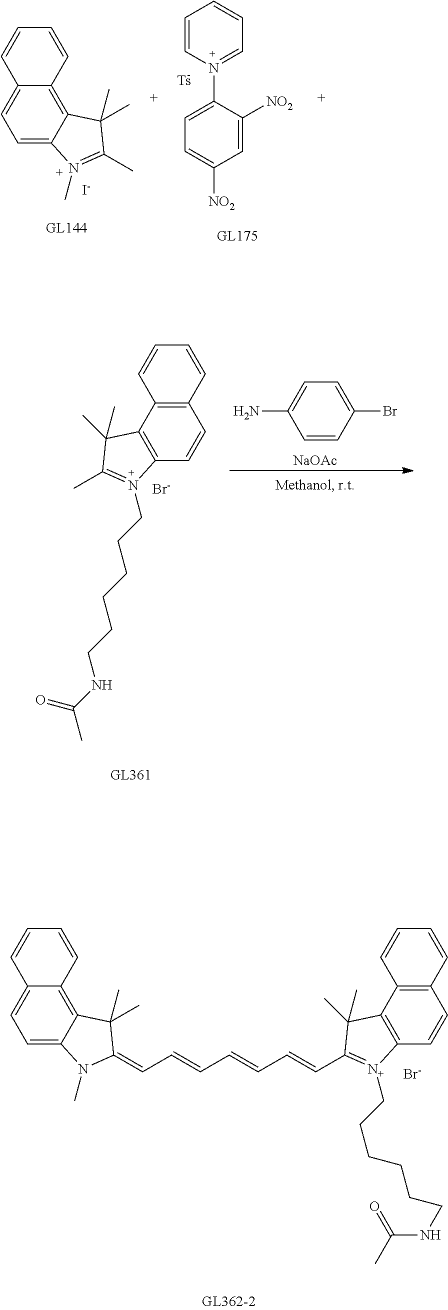

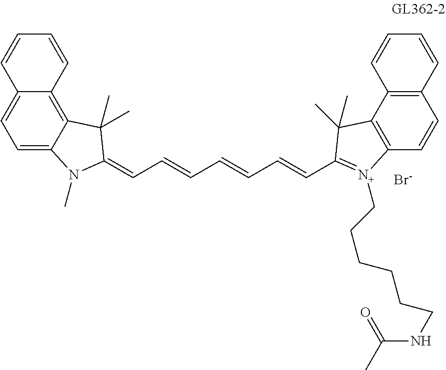 [SMILES: CC(=O)NCCCCCC[N+]1=C(/C=C/C=C/C=C/C=C2/N(C)c3ccc4ccccc4c3C2(C)C)C(C)(C)c2c1ccc1ccccc21 ; [Br-]]

The corresponding pyridinium salt GL175 (103 mg, 0.24 mmol) and 4-bromoaniline (49 mg, 0.29 mmol) were dissolved in methanol (4 mL), and the mixture was stirred at room temperature for 30 min. Next, a heterocyclic salt GL144 (105 mg, 0.3 mmol), GL361 (129 mg, 0.3 mmol) and sodium acetate (116 mg) were added, and the reaction mixture was stirred for additional 16 h at room temperature. The crude product was purified by flash column chromatography (silica gel, dichlormethane/methanol, 20:1, 10:1), affording GL362-2 as green solid in 23% yield. ¹H NMR (600 MHz, Methanol-d4) δ 8.24 (d, J=8.51 Hz, 2H), 8.09-7.99 (m, 6H), 7.70-7.57 (m, 5H), 7.49 (t, 2H), 6.62 (t, 2H), 6.34 (d, J=14.40 Hz, 2H), 4.22 (t, 2H), 3.74 (s, 3H), 3.18 (t, 2H), 2.01 (s, 12H), 1.93 (s, 3H), 1.88 (m, 2H), 1.55 (m, 4H), 1.46 (m, 2H). ¹³C NMR (150 MHz, Methanol-d4) δ 171.84, 140.40, 139.78, 133.39, 133.28, 131.98, 131.92, 130.32, 130.26, 129.71, 128.11, 128.05, 127.31, 125.57, 124.56, 121.92, 110.61, 110.47, 53.40, 50.76, 50.73, 48.47, 48.22, 43.60, 38.88, 30.44, 28.82, 27.28, 26.26, 26.23, 26.11, 26.08, 21.16. HRMS (ESI) for C₄₄H₅₀N₃O⁺ [M-Br]⁺: 636.3948. Found: 636.3954.

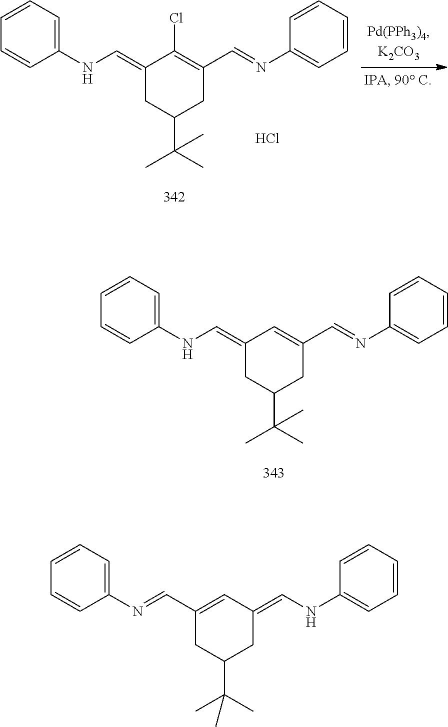 [SMILES: CC(C)(C)C1CC(/C=N/c2ccccc2)=C(Cl)C(=C/Nc2ccccc2)/C1 ; Cl ; CC(C)(C)C1CC(/C=N/c2ccccc2)=CC(=C/Nc2ccccc2)/C1 ; CC(C)(C)C1CC(/C=N/c2ccccc2)=CC(=C/Nc2ccccc2)/C1]

GL342 (830 mg, 2 mmol), Pd(PPh₃)₄ (250 mg, 0.2 mmol), and K₂CO₃ (1.1 mg, 6 mmol) were mixed in 15 mL of isopropanol, and then stirred and heated at 90° C. for overnight. The dark solution was changed to yellow. After cooled down to room temperature, the solvents were removed by rotary evaporate. The crude product was purified by flash silica gel column (Hexane and ethyl acdetate 10:1), affording orange solid GL343. LCMS (ESI) for C₂₄H₂₈N₂[M+H]⁺: 345.23. Found: 345.3.

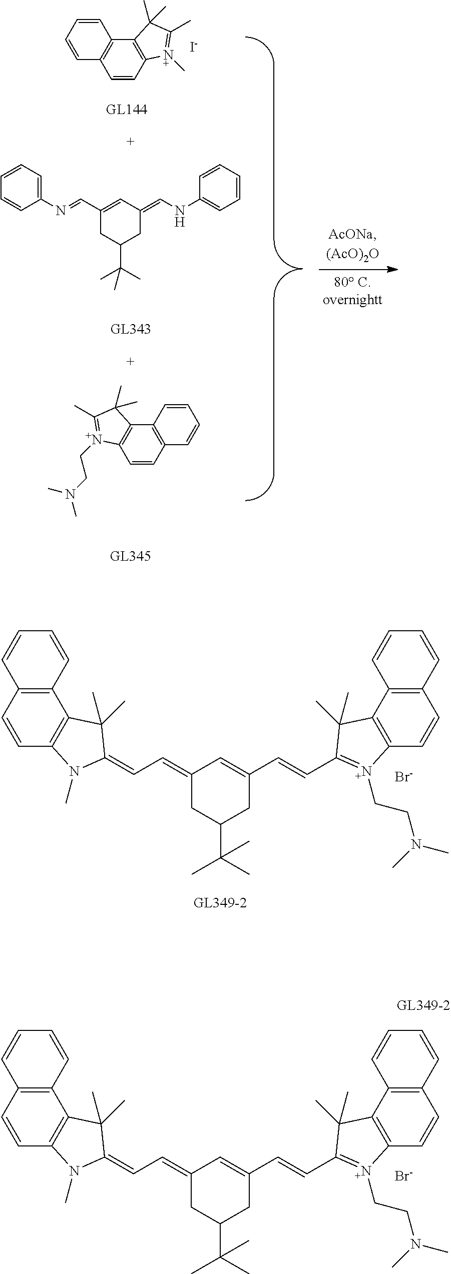

GL144 (176 mg, 0.5 mmol), GL343 (172 mg, 0.5 mmol), compound GL345 (286 mg, 0.65 mmol) and sodium acetate (246 mg, 3 mmol) and Acetic anhydride (4 ml) were stirred and heated at 80° C. for overnight. The dark solution was cooled down and poured into ether. The green solid was filtered and collected. The crude product was purified by silica gel column (DCM and Methanol 10:1). Obtaining GL349-2 as green solid (yield: 10%). ¹H NMR (600 MHz, Methanol-d4) δ 8.27 (d, J=8.82 Hz, 1H), 8.23 (d, J=8.82 Hz, 1H), 8.07-7.98 (m, 5H), 7.85 (d, J=13.24 Hz, 1H), 7.69-7.59 (m, 3H), 7.56-7.46 (m, 4H), 6.32 (d, J=14.42 Hz, 1H), 6.23 (d, J=14.22 Hz, 1H), 4.36 (t, 2H), 3.82 (s, 3H), 2.92 (m, 2H), 2.84 (t, 2H), 2.50 (s, 6H), 2.05 (m, 12H), 2.01 (s, 4H), 1.15 (s, 9H). ¹³C NMR (150 MHz, Methanol-d4) δ 174.35, 149.04, 140.27, 139.76, 132.16, 131.73, 130.34, 130.25, 129.75, 128.24, 127.94, 127.39, 127.22, 124.79, 124.22, 122.02, 121.84, 110.62, 110.07, 100.05, 98.09, 54.94, 51.06, 50.32, 44.40, 43.67, 41.11, 32.06, 30.72, 26.55, 26.29, 26.14, 26.06, 25.92. 19.64. HRMS (ESI) for C₄₇H₅₆N₃⁺ [M-Br]⁺: 662.4469. Found: 662.4474.

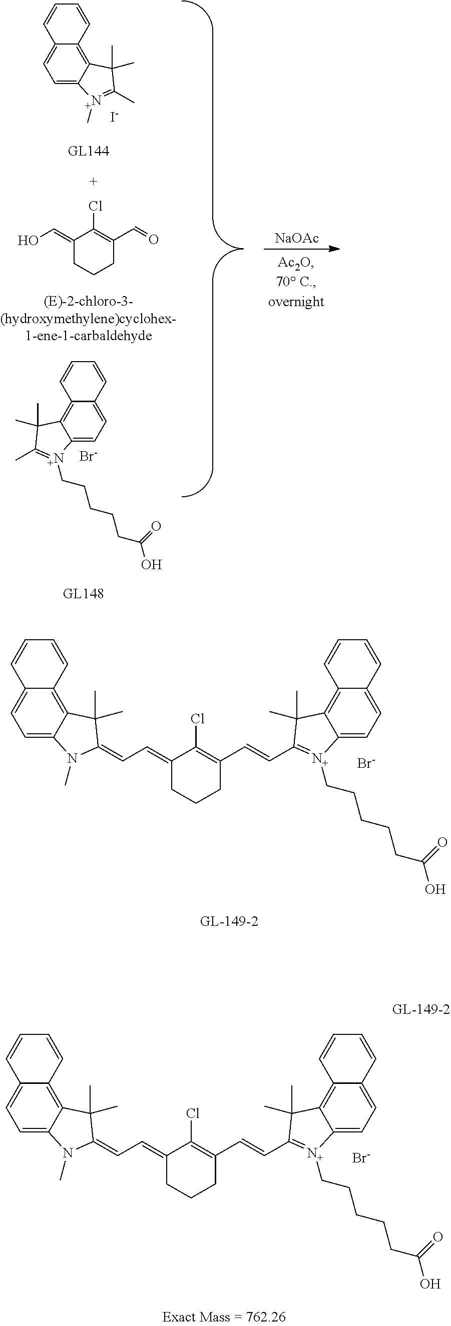

GL144 (176 mg, 0.5 mmol), GL148 (202.2 mg, 0.5 mmol), (E)-2-chloro-3-(hydroxymethylene)cyclohex-1-ene-1-carbaldehyde (86.3 mg, 0.5 mmol) and sodium acetate (86 mg, 1.05 mmol) and Acetic anhydride (5 ml) were stirred and heated at 70° C. for overnight. The dark solution was cooled down and poured into ether. The green solid was filtered and collected. The crude product was purified by silica gel column (DCM and Methanol 10:1). Obtaining GL149-2 as green solid (yield: 26.5%). ¹H NMR (600 MHz, Methanol-d4) δ 8.58 (t, 1H), 8.30 (d, J=7.61 Hz, 1H), 8.07-8.02 (m, 2H), 7.69-7.63 (m, 2H), 7.53 (t, 1H), 6.35 (d, J=12.17 Hz, 1H), 4.33 (t, 1H), 3.83 (s, 3H), 2.80 (s, 2H), 2.34 (t, 1H), 2.12 (s, 6H), 2.04 (m, 2H), 1.96 (m, 2H), 1.75 (m, 2H), 1.57 (m, 2H). ¹³C NMR (150 MHz, Methanol-d4) δ 176.40, 174.90, 174.24, 173.96, 149.19, 149.11, 143.33, 143.18, 143.05, 142.97, 140.32, 139.71, 133.83, 133.81, 132.23, 132.15, 130.49, 130.44, 129.76, 129.34, 128.05, 127.95, 127.44, 127.43, 126.61, 126.57, 126.49, 126.43, 124.92, 124.86, 124.83, 122.04, 110.75, 110.64, 100.80, 100.35, 51.06, 48.16, 43.83, 33.65, 33.07, 30.72, 26.99, 26.48, 26.38, 26.02, 25.96, 25.92, 24.46, 24.24, 20.79. HRMS (ESI) for C₄₅H₄₈ClN₂O₂⁺ [M-Br]⁺: 683.3390. Found: 683.3312.

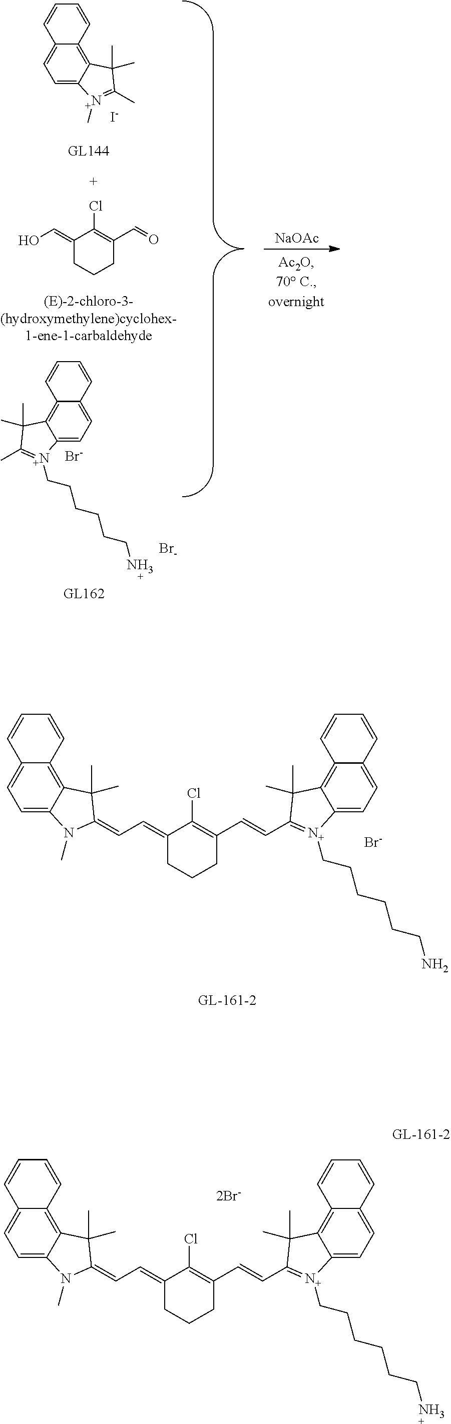

GL144 (176 mg, 0.5 mmol), GL162 (223.5 mg, 0.5 mmol), (E)-2-chloro-3-(hydroxymethylene)cyclohex-1-ene-1-carbaldehyde (86.3 mg, 0.5 mmol) and sodium acetate (86 mg, 1.05 mmol) and Acetic anhydride (3 ml) were stirred and heated at 70° C. for overnight. The dark solution was cooled down and poured into ether. The green solid was filtered and collected. The crude product was purified by silica gel column (DCM and Methanol 10:1). Obtaining GL161-2 as green solid (yield: 26.5%). ¹H NMR (600 MHz, Methanol-d4) 8.62-8.56 (m, 2H), 8.30 (m, 2H), 8.08-8.02 (m, 4H), 7.70-7.63 (m, 4H), 7.53 (m, 2H), 6.35 (m, 2H), 4.32 (t, 2H), 3.83 (s, 3H), 3.18 (t, 2H), 2.80 (m, 4H), 2.06 (d, 12H), 2.03 (m, 2H), 1.95 (m, 2H), 1.55 (m, 4H), 1.48 (m, 2H).

### Example 5—Evaluation of Activity and Anti-Cancer Properties

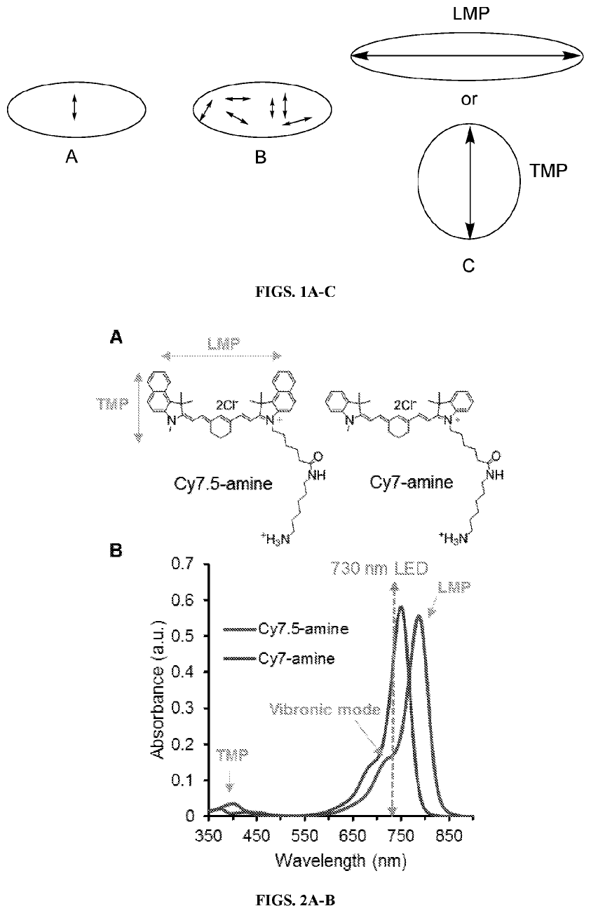

In a typical molecular absorbance of photons, an individual bond or small portion of the molecule starts vibrating (FIG. 1A) or many bonds vibrate in a disconcerted manner (FIG. 1B). But there is another way to excite a molecule wherein a whole-molecule-vibration or collective vibration is achieved that is much longer-range and concerted, spreading through the entire length or width of the molecule (FIG. 1C). When vibrational and electronic modes, sometimes called the phonon and plasmon modes, respectively, are coupled, the two modes together result in vibronic coupling, (Kong et al., 2021; Orlandi et al., 2003) or it can be called a molecular plasmon-phonon coupling. (Cui et al., 2016) More specifically, by absorbance of a suitable energy of light, a molecule's vibrational modes hybridize with the molecule's electronic transitions to induce the vibronic mode. The vibronic mode is analogous to an ultrafast breathing mode of a molecule where the entire molecule is vibrating in unison throughout its length and/or its width because one can have a longitudinal or transverse collective vibration, respectively. (Cui et al., 2016; Chapkin et al., 2018) Here it is shown that when a suitable molecule is cell-membrane-associated, it can rapidly compromise the integrity of the membrane in a manner and rate that no partial molecular vibration (FIG. 1A) or disconcerted vibrations (FIG. 1B) can induce. Heating a molecule through photothermal therapy can cause many vibrations in a molecule, but those vibrations are not coordinated, as shown in FIG. 1B, hence there is no concerted longitudinal or transverse vibration that is sufficient to rapidly open a cell membrane. Hence, high powers and extended times are needed in photothermal therapy to cause slower apoptotic death. Conversely, VDA of a cell-associated molecule results in rapid necrosis even at very low energies. Likewise, VDA is distinct from photodynamic therapy where the latter generates ROS, while VDA in a cell-associated molecule causes cell death that is unaffected by even large doses of ROS-inhibitors. Cyanine dyes have been used in photothermal and photodynamic therapies and they are readily accepted in biological and medicinal studies. (Mishra et al., 2000; Li et al., 2021; Shi et al., 2016; Lange et al., 2021; Bilici et al., 2021) However, here these same classical structures were used in what is generally 10× lower concentration and with 10-50× lower powers than often used (80 mWcm⁻² instead of 1 to 4 Wcm⁻²), exploiting their VDA to kills cells 10-50× faster than with photothermal or photodynamic therapies.

### A. Results

FIGS. 20D-20E show the chemical structure and absorption spectra of two aminocyanines, Cy7.5-amine and Cy7-amine. Cyanine structures are characterized by an odd-numbered polyene linker connecting two nitrogen-containing heterocycles with unusual photophysical properties. The absorption spectrum of cyanines is dominated by an absorption band in the visible/NR electromagnetic spectrum with a shoulder located at higher energy (shorter wavelength). The Cy7.5-amine, in contrast to Cy7-amine, has an additional aryl ring that increase the conjugation which cause a red-shifting of the absorption by ˜40 nm relative to Cy7-amine.

Here, it is proposed that the feature of the higher-energy shoulder next to the large absorption band in symmetrical cyanine structures results because there is coupling of a molecular plasmon (a dominant collective oscillation of electronic excitation) to a dominant collective vibrational excitation, in agreement with the suggestion in the literature that the absorption sub-bands in cyanines are primarily determined by a dominant symmetric vibration rather than a collection of single vibrations. (Mustroph & Towns, 2018) This vibronic behavior, through the coupling of electronic and vibrational states, is a feature of the conjugated-backbone-near-symmetrical cyanines such as in Cy7-amine and Cy7.5-amine. The shoulder (λ˜730 nm) in the absorption spectrum of Cy7.5-amine corresponds to this collective vibrational mode (FIG. 3). The same collective vibrational mode is present in Cy7-amine but at ˜690 nm. The molecular plasmons in cyanines were indeed confirmed by Time-Dependent Density Functional Theory (TDDFT) calculations; these molecules can support longitudinal molecular plasmons (LMP) and transversal molecular plasmons (TMP) (FIGS. 1 and 20). The shoulder band is not the only vibronic mode present in the absorption spectrum, but instead probably the strongest in vibronic character spreading throughout the length and width of the molecule. The larger band at ˜780 nm, dominantly a longitudinal charge density resonance, and the small peak at ˜400 mm, with a transversal charge density resonance, and the shoulder at ˜450 nm, with a short longitudinal charge density resonance, also have vibronic properties with charge density resonances typical of a molecular plasmon, (Cui et al., 2016) as shown in FIG. 20.

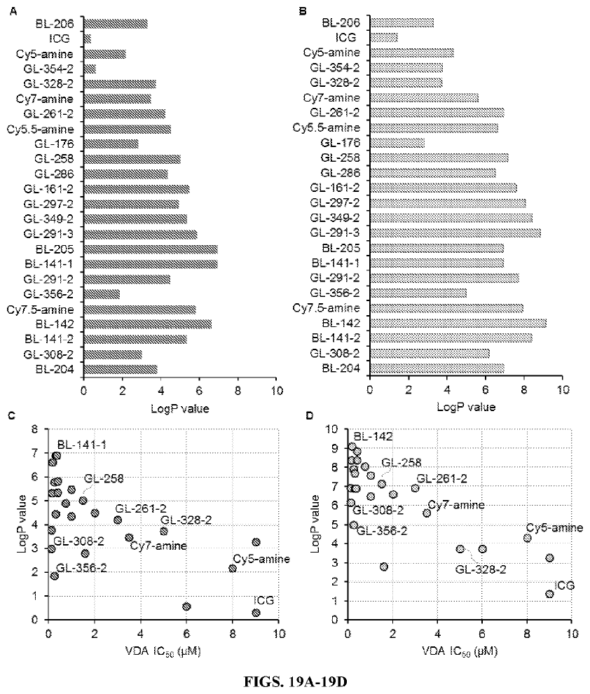

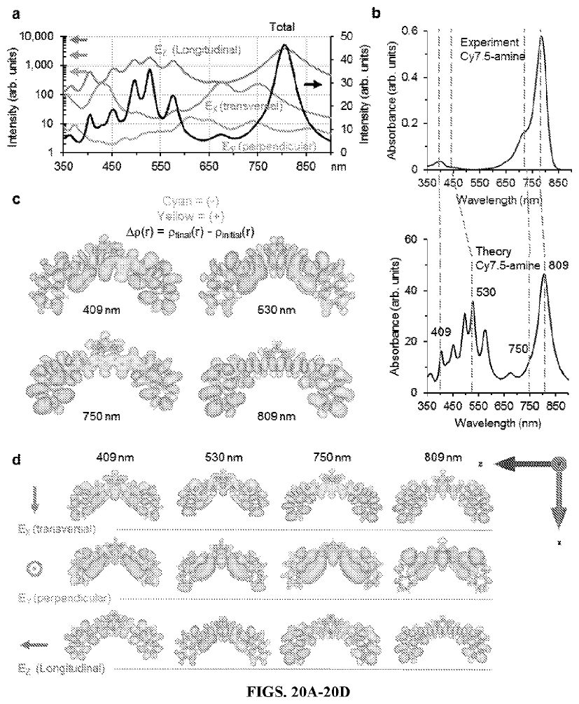

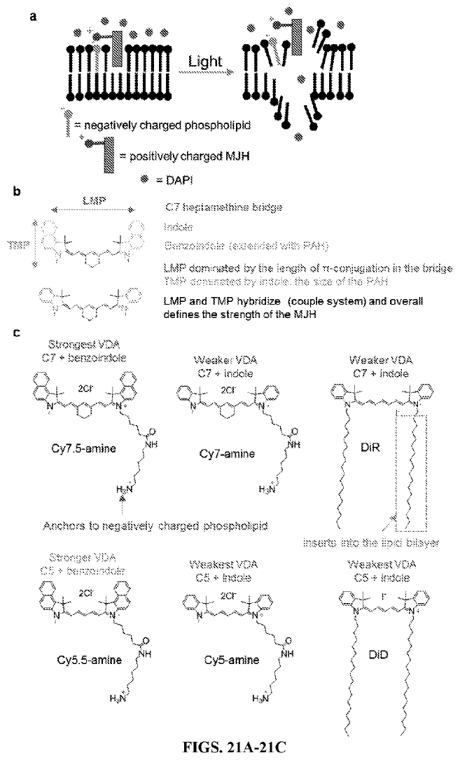

Here the vibronic mode in a cell-membrane-bound Cy7.5-amine was selectively excite using a NIR light-emitting diode (LED) at 730 nm (FIG. 3) which results in the permeabilization of the cellular membrane to 4′,6-diamidino-2-phenylindole (DAPI). DAPI is a cell membrane impermeable dye in viable cells that mainly stains cellular DNA in membrane-disrupted cells, with induction of rapid necrotic cell death in human A375 melanoma cells (FIG. 4). While 730 nm light does not excite the vibronic shoulder of Cy7-amine, it can activate the Cy7.5-amine (FIG. 3) and permeabilize A375 cells immediately after treatment. It took ˜30 s from the time the sample was irradiated to start collecting the data in the flow cytometer. For this experiment, 1 μM Cy7.5-amine, 730 nm LED at 80 mWcm⁻² for 10 min, caused permeabilization to DAPI staining of 99.6% of the A375 cells in a cell suspension containing 2×10⁵ cells. In contrast, Cy7-amine was not able to permeabilize the cells under the same conditions. This difference between the two aminocyanines supports the notion that the 730 nm light can excite the vibronic mode (shoulder) in Cy7.5-amine (FIG. 3) causing cellular membrane permeabilization and ultimately cell death by necrosis as seen by immediate DAPI staining. Both aminocyanines were attached efficiently to the cells as shown by flowcytometry; they were both loaded into the cell (FIG. 4). It is interesting that Cy7-amine has a larger extinction coefficient (ε=132,000 μM⁻¹ cm⁻¹) at λ=730 nm than Cy7.5-amine (ε=72,000 μM⁻¹ cm⁻¹) in water, yet Cy7-amine does not permeabilize the cells at comparable concentrations while Cy7.5-amine permeabilizes readily upon excitation (FIG. 1-4). This also suggests that a photothermal effect is not operating since Cy7-amine has a higher absorption cross-section than Cy7.5 at 730 nm. Likewise, photodynamic therapy is unlikely since Cy7 cyanines and Cy7.5 cyanines have similar yields for singlet oxygen generation. (S̆tacková et al., 2020) A summary of the proposed working mechanism is described in FIG. 21. First, the aminocyanine binding to the cells is possibly mediated by the charge on its pendant amine moiety to the negatively charged phospholipids (FIG. 22) follow by light-activated VDA that open cell membranes. Likewise, the aminocyanine Cy5-amine was added as competitor for cell binding against Cy7.5-amine, the results showed a reduction of permeabilization activity of Cy7.5-amine (FIG. 23). FIG. 24 summarizes the optical spectra of all aminocyanines in this study and the characterization of their binding to the A375 human melanoma cells by confocal microscopy.

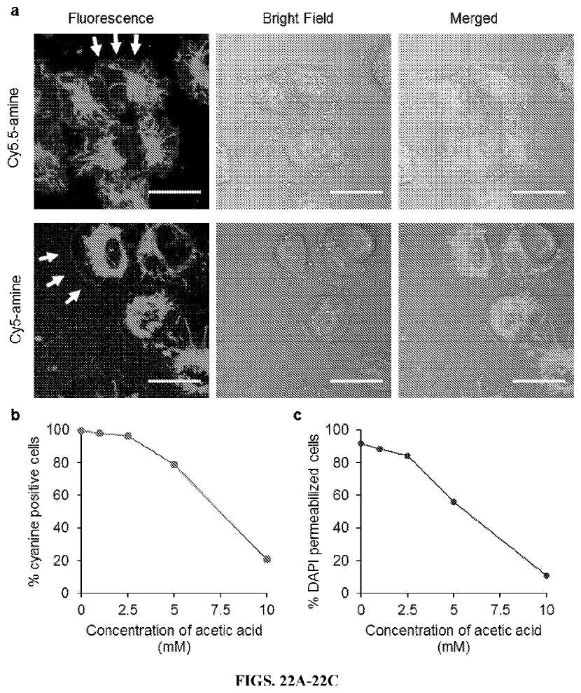

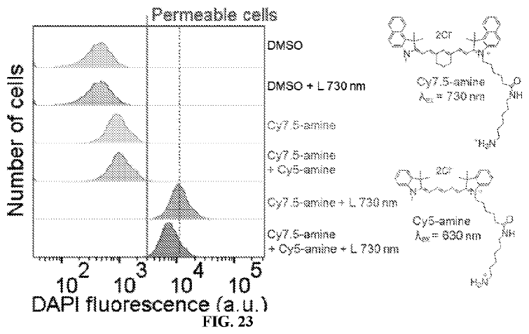

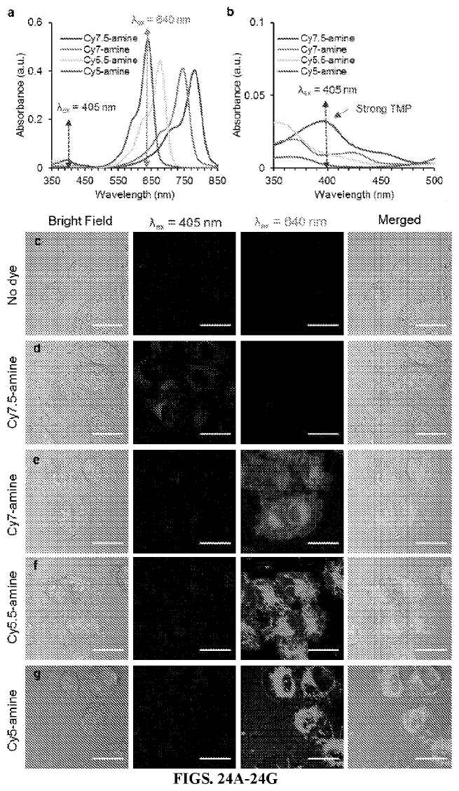

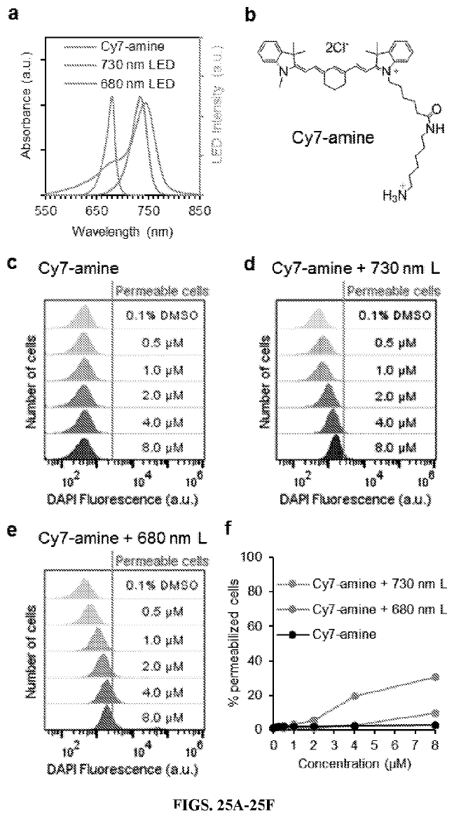

Consistent with the VDA proposed here, excitation of the 680 nm vibronic shoulder in Cy7-amine improves the MJH effect for opening cell membranes in A375 cells (FIG. 25). However, the permeabilization is not as large as for 730 nm excitation of Cy7.5-amine even when exciting the 680 nm shoulder of Cy7-amine using an equal light dose of 80 mWcm⁻² for 10 min. This might result because Cy7-amine lacks the extended aryl ring of Cy7.5-amine, limiting the TMP of the vibronic mode. Hence, Cy7-amine is a weaker MJH than Cy7.5-amine because of the lower plasmonicity of the indole in contrast to the benzoindole (FIG. 1-4).

FIG. 26 compares the Cy7-amine and Cy7.5-amine when exciting at 730 nm at various concentrations. It is evident that the Cy7.5-amine is much more efficient at permeabilizing cells upon the VDA with NIR light. Even at much higher concentrations of Cy7-amine (8 μM), it does not permeabilize cells as efficiently as lower concentrations of Cy7.5-amine. This is further confirmation that the excitation of the vibronic shoulder in Cy7.5-amine at 730 nm and the extension of the conjugation by the aryl rings in the benzoindoles are critical to maximize the VDA. The other example of an aminocyanine family, Cy5.5-amine and Cy5-amine, follows a similar behavior. The results suggest that there is a molecular structure/VDA intensity correlation with the molecular mechanical action (FIG. 26). The VDA increases with the length of the conjugation in the polymethine bridge and the extension of the aromatic ring at the indole.

FIG. 27 shows the confocal microscopic permeabilization of the cells overtime using 630 nm light treatment of Cy5.5-amine and Cy5-amine where the vibronic band in Cy5.5-amine is accessed while only weakly accessed in Cy5-amine (FIG. 26B). The cell permeabilization was done in the presence of the cell-membrane-targeting DiD dye under the confocal microscope. At 4 min of laser excitation, the cells are already permeabilized; the DAPI intensity (cell permeabilization) is 2× relative to the initial and 13× higher at 10 min. The result show that Cy5.5-amine is a stronger MJH than Cy5-amine (2.5×DAPI intensity increase at 10 min) which is consistent with the flow cytometry analysis in FIG. 26C-26E. The DiD dye alone showed a weak effect on cell permeabilization (1.5×DAPI intensity increase at 10 min), as expected because it is analogous to Cy5, a MJH with a weak VDA. In all non-illuminated controls, the DAPI intensity remains at 1× relative to the initial conditions.

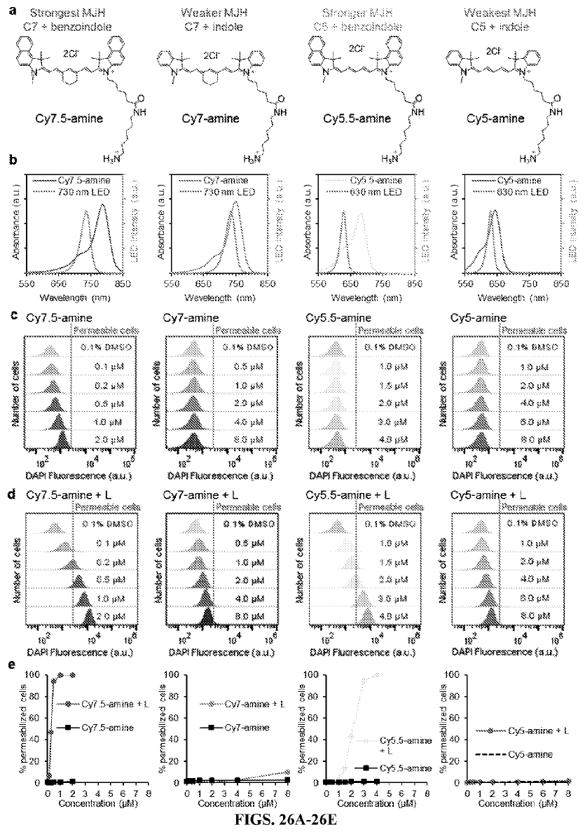

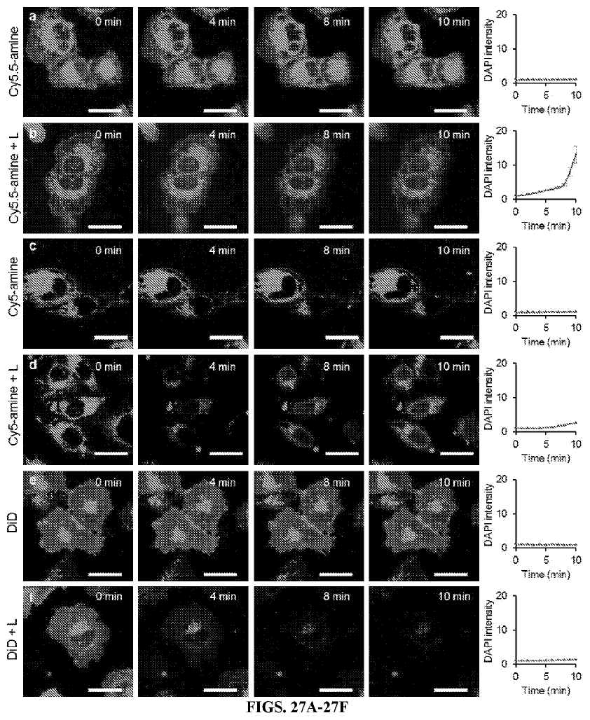

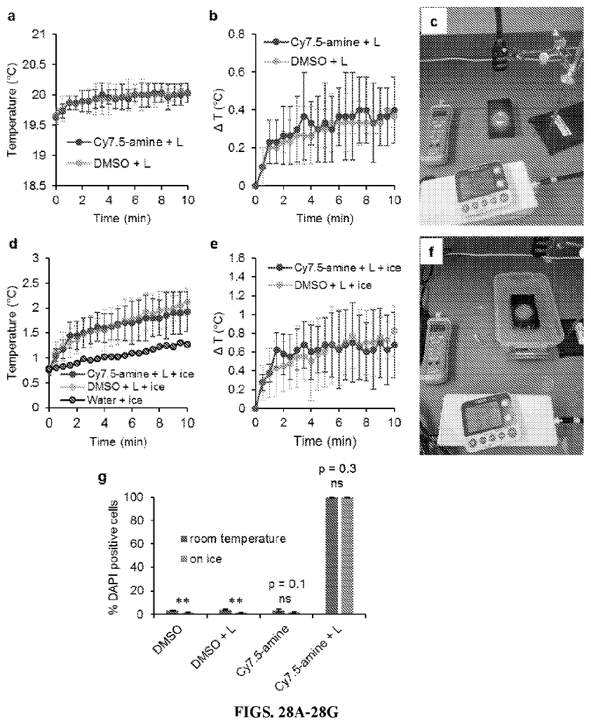

To rule out a photothermal effect, the temperature of the media was measured during the NIR light exposure when the treatment was done at room temperature versus when done in an ice bath (FIG. 28) surrounding the cell culture dish. The temperature remained constant at 20° C. and 2° C. when the treatment was done at room temperature and in an ice bath, respectively. In FIG. 28G, the cell permeabilization is not affected by the lower temperature. Thus, the photothermal effect is not responsible for the necrosis seen in these cells.

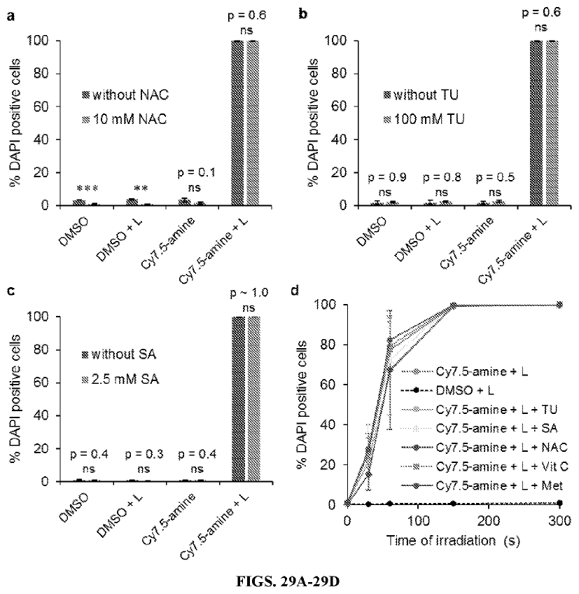

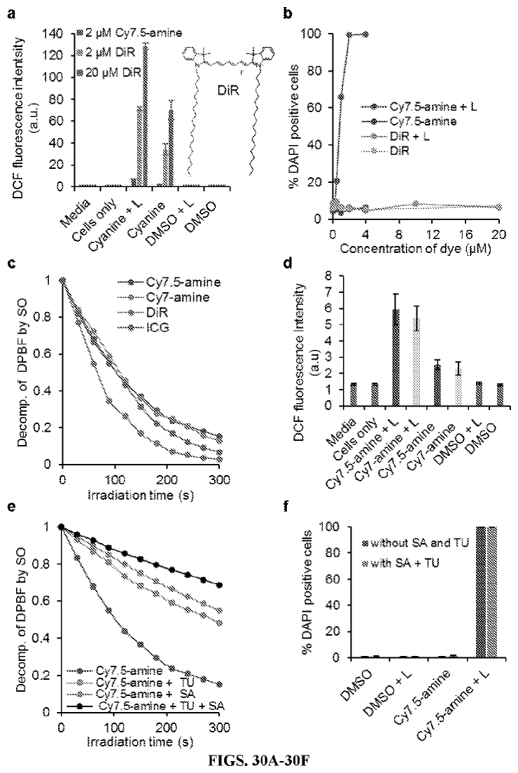

To confirm that a photodynamic ROS generation is not responsible for the necrosis, the permeabilization of A375 melanoma cells was repeated in the presence of ROS scavengers (FIG. 29A-29C). Neither N-acetyl-cysteine (NAC, 10 mM), thiourea (TU, 100 mM) or sodium azide (SA, 2.5 mM) were able to retard the permeabilization of the cells. Further, the ROS is not responsible for the permeabilization of the cells; in FIG. 29B the permeabilization conditions were tuned to lower illumination time where the scavengers might quench ROS more effectively. The results show that none of the ROS scavengers used (NAC, TU, SA, methionine or vitamin C) slowed the permeabilization of the A375 cells. This suggests that ROS is not responsible for the permeabilization of the cells. FIG. 30 shows that singlet oxygen production is also not responsible for the permeabilization of the cells.

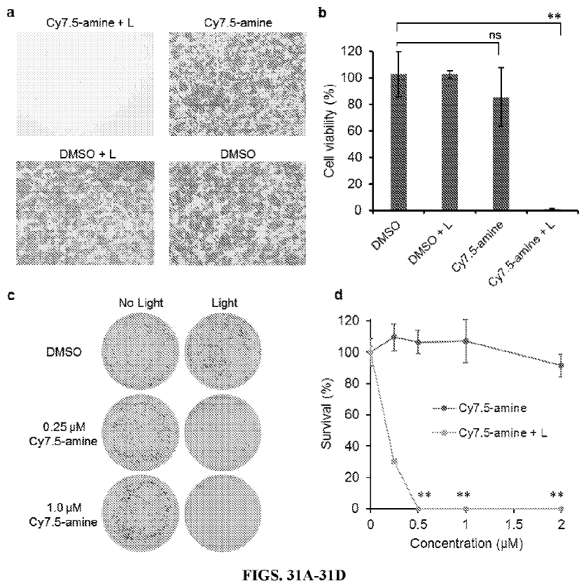

To confirm that the permeabilized cells to DAPI are indeed dead, the cells were cultured after the treatment (with 2 μM Cy7.5-amine and illumination for 10 min with 730 nm light at 80 mWcm⁻²) and quantified cell death by crystal violet and clonogenic assays. FIG. 31 shows that the permeabilized cells by light-activated Cy7.5-amine were dead nearly at the same quantity, 99.9%, as was observed in flow cytometry (FIGS. 1-4). The clonogenic assay shows that 100% of the cells were eradicated when using 0.5 μM Cy7.5-amine and illumination for 10 min with 730 nm light at 80 mWcm⁻².

The FDA-approved indocynine green (ICG) bearing alkylsulfonate addends was also tried; it has a vibronic absorption shoulder at 730 nm, as shown in FIG. 10. The sulfonates will not interact with phospholipids in lipid bilayers. ICG did not permeabilize the cells under the same conditions in which Cy7.5-amine was tested. Taken together, this suggests that the effective cell killing of Cy7.5-amine is a synergy of the amine pendant for rapid cell association followed by VDA. This presumably causes an energy transfer from these whole-molecule excited structures to the cellular membrane, ultimately disrupting the lipid bilayer, permitting cell membrane permeabilization.

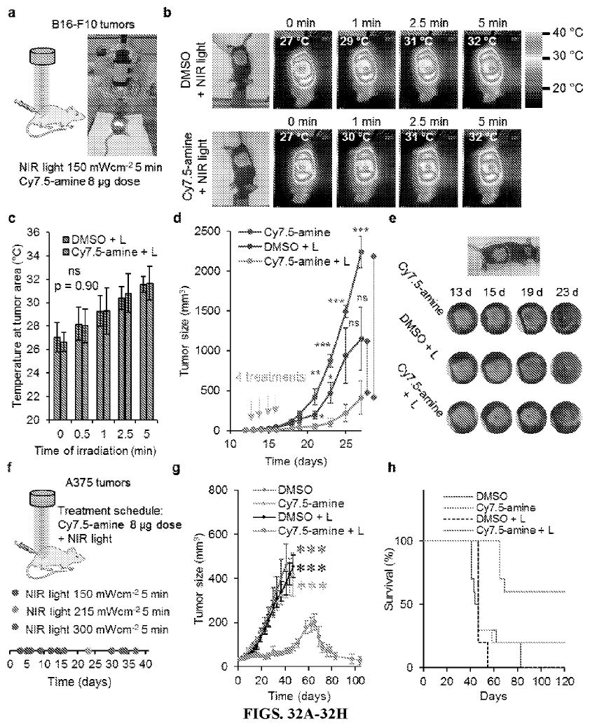

The MJH Cy7.5-amine was applied to treat murine (B16-F10) and human (A375) melanoma tumors in mice (FIG. 32). The temperature of B16-F10 tumors in C57BL/6 mice while under Cy7.5-amine with light treatment (150 mWcm⁻¹ for 5 min) increased ˜5° C. and this was not different than the control with 0.1% DMSO and light (FIG. 32B & 32C). The size of the B16-F10 tumors was significantly reduced using a dose of 8 μg of Cy7.5-amine in 50 μL PBS solution intratumorally and illumination with 730 nm LED at 150 mWcm⁻¹ for 5 min (FIG. 32D) In the treatment of A375 tumors in nude mice, the conditions were optimized, and 300 mWcm⁻² of 730 nm LED for 5 min was found in combination with a intratumoral dose of 8 μg of Cy7.5-amine was sufficient to achieve a survival rate of 60% at day 120 of the study and 50% of the mice became tumor free.

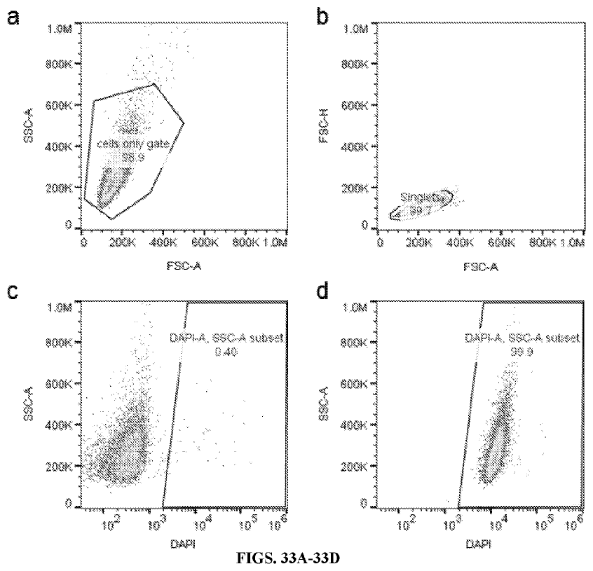

The flow cytometry data was analyzed using FlowJo software version 10.5.3. The results were plot using the forward scattering area (FSC-A) vs side scattering area (SSC-A) as shown in FIG. 33A. Then, a wide polygonal gate was drawn as shown in FIG. 33A since we were interested in detecting any cell death after treatment, we were not only interested in measuring healthy cell populations. Sometimes, we observed that the death cell population upon treatment shifted outside the gate if a narrow gate was used. Then, the singlet cells were selected by plotting the FSC-A vs the forward scattering height (FSC-H) as shown in FIG. 33B. Then, the singlets were selected and the DAPI fluorescence intensity was plotted versus the SSC-A and the gate for DAPI positive cells was drawn as shown in FIG. 33C and the analysis was applied to all samples in the group. In FIG. 33D, a DAPI positive population is observed in the same analysis group. This analysis was applied to calculate the percentage of DAPI positive cells.

### Example 6—Experimental Methods

Cyanine molecules. Cy7.5-amine, Cy7-amine, Cy5.5-amine and Cy5-amine were purchased from Lumiprobe Corp. (Maryland, USA). DiD and DiR cyanines were purchased from Biotium (Fremont, CA).

LED illumination systems. The 730 nm LED (model UHP-F-730) and 630 nm LED (model UHP-F-630) were purchased from Prizmatix, Israel. The 680 nm LED and 740 nm LED were custom made and purchased from Keber Applied Research Inc. (Ontario, Canada).

Culture of human melanoma A375 cells. A375 cells were obtained from ATCC (CRL-1619). Cells were cultured in 10 cm polystyrene tissue culture treated dish (Corning) containing DMEM with L-glutamine, 4.5 g/L glucose, and sodium pyruvate (Corning Inc. 10013CV) and supplemented with 10% FBS (Corning, 35010CV), 2×MEM vitamin solution (Gibco, 11120052), 1×MEM non-essential amino acid solution (Gibco, 11140050) and penicillin/streptomycin. Typically, 0.5-1 million cells were inoculated per dish and cultured for 3-4 days in incubator at 37° C. and 5% CO₂, then transferred to a new dish when confluency reached nearly 95-100%. For the passage step, cells were detached with 0.05% trypsin-EDTA (Gibco, 25-300-054).

Culture of mouse melanoma B16-F10 cells. Mouse melanoma B16-F10 cells were obtained from the ATCC (CRL-6475) and cultured in 10 cm polystyrene tissue culture treated dish (Corning) containing DMEM with 4.5 g/L glucose (Gibco, 11960-044) and supplemented with 10% FBS (SAFC Industries-Sigma-Aldrich, 12303C), 2×(10 mL) MEM vitamin solution (Corning, 25-020-C1), 1×(5 mL) non-essential amino acid (NEAA) mixture (Lonza, 13-114E), 1×(5 mL) of L-glutamine (Lonza, 17-605E), and 1×(5 mL) of penicillin/streptomycin (Hyclone, SV30010). Typically, 0.5-1 million cells were inoculated per dish, and cultured for 2-3 days in incubator at 37° C. and 5% CO₂, then transferred to a new dish when confluency reached nearly 95-100%. For the passage to a new culture dish, cells are detached with 0.05% trypsin-EDTA (Gibco, 25-300-054).

Cell Permeabilization and flow cytometry analysis. A375 cells were cultured as described before. One day before the treatment, cells were inoculated at 5 million cells per dish (10 cm polystyrene tissue culture dish). The cells were harvested using 0.05% trypsin-EDTA (Gibco, 25-300-054), then the cells were counted and were adjusted to a cell density of 2×10⁵ cells/mL in DMEM media with L-glutamine, 4.5 g/L glucose, and sodium pyruvate (Corning Inc. 10013CV) and supplemented with 10% FBS (Corning, 35010CV), 2×MEM vitamin solution (Gibco, 11120052), 1×MEM non-essential amino acid solution (Gibco, 11140050) and penicillin/streptomycin. 1 mL of this cell suspension containing 2×10⁵ cells was used in each treatment. In a 1.5 mL Eppendorf tube, 1 mL of stock solution containing 2 mM Cy7.5-amine (or other cyanine molecule or other concentration) in DMSO (Fisher, 99.7%) was placed in the bottom of the tube, then 1 mL of the cell suspension was added into the tube to get final concentration of 2 μM of Cy7.5-amine containing 0.1% DMSO and 2×10⁵ cells. The mixture was then incubated at 37° C. and 5% CO₂ for 30 min. Then, 1 mM DAPI was added into the cell suspension. Then, the cells suspension was transferred to a 35 mL polystyrene tissue culture dish and immediately the cells were treated under the light beam of NIR light of 730 nm at 80 mW/cm² (or adjusted powers down to 20 mW/cm²) for 10 min (or adjusted illumination times down to 30 s) using LED light source (PRIZMATIX, UHP-F-730, Israel) which covered the entire dish. While the cells were treated, the dish was placed on top of an aluminum block painted black, so that the excess NR light and that was not reflected back into the cell suspension while the aluminum block actED as a heat-sink, maintaining a constant temperature in the dish during the irradiation. The instrument for flow cytometry analysis (SONY, MA900 Multi-Application Cell Sorter) was already set up and calibrated by the time the light treatment was finished. Therefore, as soon as the 10-min light treatment was completed, the cell suspension was rapidly transferred from the 35 mm dish to a flow cytometry tube and the cells were analyzed for DAPI permeabilization and Cy7.5-amine binding. It took ˜30 s to load the sample and to start the analysis. Therefore, the permeabilization of cells was measured as DAPI positive cells and occur immediately due to the membrane permeabilization caused by Cy7.5-amine excitation with the 730 nm NIR light. The light intensity was measured using an Optical Power Meter from Thorlabs, sensor model S302C and console model PM100D.

Temperature measurements. The permeabilization of the cells and flow cytometry analysis was conducted as described above. The temperature of the cell suspension was measured using the temperature probe (Model SC-TT-K-30-36-PP; Omega Engineering, Inc.) immersed in the media. The same was repeated having the cell suspension on top of an ice bath, and the temperature of the cell suspension recorded in the same way during the NIR light illumination. The temperature of the media stayed constant at room temperature of −20° C. upon illumination of the media with the 730 nm LED light at 80 mW/cm² for 10 min. There was only a minor temperature increase of 0.4° C. which was attributed to the light illumination absorption by the media components. Similarly, on ice the temperature of the media only increased 0.6° C. due to the illumination by the 730 nm LED on the media.

ROS scavenger experiments. The permeabilization of the cells and flow cytometry analysis was conducted as described before. But in this case, ROS scavengers were added into the cells suspension and incubated for 1.5-2 h at 37° C. and 5% CO₂ before any treatment to allow the antioxidants interact first and protect the cells. Then the experiments were conducted exactly as described before with and without ROS scavengers present, and results were compared.

Crystal violet cell viability assay. The crystal violet assay was used to measures the cell viability. The principle of this method is that the viable cells adhere to the surface of the cell culture dish and keep growing and remain attached through the standard cell culture conditions during a period of 1-2 days and through the staining conditions in the assay. In contrast dead cells do not adhere to the surface of the cell culture dish, do not grow, and detach easily during the manipulation steps during the assay which includes removal of media and exchange with fresh media and washing steps with PBS buffer. For specific details, A375 cells were harvested and counted, and then 20,000 A375 cells per well were added in 24 cell culture well plate (Corning) and cultured for 1 day at standard incubation conditions of 37° C. and 5% CO₂. The cells were treated in four experimental groups (4 samples per group): group 1) 0.1% DMSO, group 2) 0.1% DMSO+NIR light treatment, group 3) 2 μM Cy7.5-amine, and group 4) 2 μM Cy7.5-amine+NIR light. The treatments with 0.1% DMSO or 2 μM Cy7.5-amine were done by adding those respective concentrations to the cells in the media and then incubated for 60 min. Immediately after the incubation, the cells in the groups with “+NIR light”, were treated with 730 nm light at 80 mW/cm² for 10 min. After the treatment with NIR light, the media in all the groups was removed and fresh media was added. Then, the cells were incubated for 2 days at 37° C. and 5% CO₂. At the end of the incubation, the media was removed and the cells washed with 500 μL of PBS once. Then, the cells were stained with 500 μL of 0.05% w/v crystal violet solution in methanol for 5 min. Then, the crystal violet was removed and the excess of crystal violet was washed with water. The cells contained in the 24 well plate were dried at room temperature. Then, the crystal violet in each well was solubilized in 500 μL of 3.3% v/v acetic acid in water and the total crystal violet recover in this acidic solution. Then the crystal violet was quantified by its absorbance at 570 nm. The cell viability was calculated from the absorbance relative to the absorbance in the cells without any treatment. Cells without treatment were normalized to 100% cell viability.

Clonogenic assay. A375 cells were seeded in 35 mm cell culture dishes at predetermined densities to allow for an approximately equal number of resultant colonies. The next day, cells were treated with Cy7.5-amine at variable concentration and with or without 730 nm light at 80 mWcm⁻² for 10 min. The cells were incubated with Cy7.5-amine for 50 min before the illumination, the media was replaced with fresh media after the illumination and cells were cultured for 6 days to allow for colony formation. Cells were then washed once with PBS and fixed-stained in a 0.5% (w/v) crystal violet in methanol/water solution (1:1) during 10 min. The excess of crystal violet was washed off with water, the plates were dried at room temperature and then the colonies were counted using ImageJ software version 1.52a, and the survival fraction was determined. All treatments were in triplicate.

ROS measurements using H₂DCF-DA. H₂DCF-DA (2′,7′-dichlorodihydrofluorescein diacetate) is a cell permeant reagent. It is deacetylated by cellular esterases to form 2′,7′-dichlorodihydrofluorescein (H₂DCF), a non-fluorescent compound, which is rapidly oxidized in the presence of ROS into 2′,7′-dichlorofluorescein (DCF). DCF is highly fluorescent and is detected with excitation/emission at 488 nm/535 nm. A375 cells in suspension containing 2×10⁵ cells mL⁻¹ were first prepared in DMEM media without phenol red. Then the cells were incubated for 30 min at 37° C. with Cy7.5-amine (or the other cyanines) typically at 2 μM concentration in the media. Then, H₂DCF-DA (Sigma-Aldrich) was added to cells suspension in media to the final concentration of 5 μM (the stock of H₂DCF-DA was at 5 mM in DMSO stored at ˜20° C.). Then transfer the cells to a 96 well plate, 100 μL to each well. Typically, it took 15 min to transfer all the samples and all the controls into the 96 well plate, 6 repetitions per each treatment condition: cyanine+light, cyanine only, DMSO+light, DMSO only, cells only, media only. Then, immediately after the cells were treated with NIR 730 nm light at 80 mWcm⁻² for 10 min. From the time the H₂DCF-DA was added into the cell suspension to the time the cells were treated there was in total a 20 min incubation. After the light treatment, the DCF fluorescence intensity was measured immediately after the light treatment using a 96 well plate reader at λₑₓ=488/9 nm and λₑₘ=535/20 nm. The measurements were normalized with respect to the fluorescence intensity in the media only.

Singlet oxygen measurements. For the measurement of singlet oxygen, the molecular probe DPBF (1,3-diphenylisobenzofuran) was used which decomposes in the presence of singlet oxygen and this was detected by the change in the absorbance of DPBF at 410 nm. DPBF was freshly prepared for every experiment by dissolving DPBF in methanol at 1 mM stock solution. Then dilute the DPBF in methanol and adjust the dilution volume to get an absorbance of ˜1.0 at 410 nm. For this purpose, typically 170 μL of the 1 mM DPBF stock in methanol was diluted in 2830 μL of methanol. Then to this mixture was added 1 μL of cyanine stock solution (8 mM cyanine solution in DMSO stored at ˜20° C.) to get a final concentration of 2.6 μM of cyanine. Then immediately after preparing the mixture, the solution was transferred to a clean spectrophotometer quartz cuvette, and the solution was irradiated with 730 nm LED light at a power intensity of 80 mWcm⁻². The power intensity was calibrated to the distance at the top of the liquid on the quartz cuvette. The sample was irradiated every 30 s, and then absorption spectrum was recorded in between every irradiation interval until a total of 10 min of irradiation was accumulated.

In vivo studies. All animal studies were approved by the Institutional Animal Care and Use Committee (IACUC) of the University of Texas MD Anderson Cancer Center (Houston, TX). These studies used 7-8 weeks old female C57BL/6J mice (Jackson Laboratories, strain #000664) or 7-8 weeks old athymic female nude (nu/nu) mice from Envigo/Harlan labs.

Injection of B16-F10 cells subcutaneously in C57BL/6J mice to generate the melanoma tumors. The B16-F10 cells were cultured as described before. Cells were harvested from sub-confluent plates, ˜90%, and fresh media was added to the cells the day before harvesting. The cells were harvested using 0.05% trypsin-EDTA (Gibco, 25-300-054). The harvested cells were re-dispersed in DMEM media without supplements at 1×10⁶ cells mL⁻¹. The cell suspension was kept in ice. Then 100 μL of cells were injected per mouse (this was 100,000 cells per mouse) subcutaneously in the right flank of a 7-8-week-old female mouse (C57BL/6J), in which the hair in the right flank was previously depilated using a shaver. The tumors were allowed to grow for 12 days counting from the day of cell injection. And at day 12 the hair of the mouse was removed using hair remover cream (Nair Hair Remover Lotion). For this purpose, a drop of the cream was placed on the skin, on top of the area where the tumor was injected. The mice were anesthetized using isoflurane while the hair remover cream was applied. Starting at day 12 the tumors were measured using a caliper. The tumors can be observed as a black spot (due to the melanin present in the B16-F10 cells) under the skin after the cream depilation. The typical volume of the tumors at ˜15 days was ˜25 mm³. The volume of the tumor was calculated as: (½)×length×width×height. When the height was not possible to measure in the case of the tumors which were too small (usually <100 mm³), then the tumor volume was calculated as: (½)×length×width².

Preparation of fresh solution of 200 μM Cy7.5-amine and 2.5% DMSO for in vivo studies. In the day of treatment, fresh solution of 200 μM Cy7.5-amine was prepared by diluting in PBS buffer the 8 mM Cy7.5-amine stock in DMSO, the final dilution contained 2.5% DMSO. As control 2.5% DMSO in PBS buffer was used.

Treatment of B16-F10 tumors with Cy7.5-amine and NIR 730 nm light. The tumors were treated at day 13 counting from the day of cell injection. Mice were divided in 3 groups: 1) Cy7.5-amine only (mice per group, n=4), 2) 2.5% DMSO+Light (n=5) and 3) Cy7.5-amine+light (n=5). The day of treatment, fresh solutions (200 μM of Cy7.5-amine in PBS and controls 2.5% DMSO in PBS) were prepared as described before. The mice were anesthetized with isoflurane using a vaporizer. Then, each mouse was injected with 50 μL of 200 μM Cy7.5-amine solution in PBS or 2.5% DMSO, intratumorally. Then mice were kept for 30 min in the cages to let the Cy7.5-amine solution or DMSO solution interact with the tumors. Then, after the 30 min of incubation, the mice were treated (under anesthesia, using isoflurane) with 730 nm LED light source from Prizmatix applying a power intensity of 150 mWcm⁻² for 5 min. The light intensity was measured using an Optical Power Meter from Thorlabs, sensor model S302C and console model PM100D. While under light treatment the temperature at the tumor area was measured using an IR thermal camera (Model: Compact Seek Thermal for Android. Seek Thermal, Inc. Santa Barbara, CA). When the treatment was finished the mice were put back into the cages and housed in the animal facility. The treatment was repeated once daily for 4 days. The tumor sizes were measured every day starting the day of hair removal with cream. The tumors were measured using a caliper. Then after the 4 treatments, the tumors were measured every other day.

Injection of A375 cells subcutaneously in athymic nude mice to generate the human melanoma tumor model. The A375 cells were culture as described before. Cells were harvested from sub-confluent plates, ˜90%, and fresh media was added to the cells the day before harvesting. The cells were harvested using 0.05% trypsin-EDTA (Gibco, 25-300-054). The harvested cells were re-dispersed in DMEM media without supplements at 50×10⁶ cells mL⁻¹. The cell suspension was kept in ice. Then 100 μL of cells were injected per mouse (this was 5 million of cells per mouse) subcutaneously in the right flank of 7-8 weeks old female athymic nude mouse. Starting at day 2 the tumors were measured using a caliper. The typical volume of the tumors at day 2 was ˜33 mm³. The volume of the tumor was calculated as: (½)×length×width×height. When the height was not possible to measure in the case of the tumors that were too small (usually <100 mm³), then the tumor volume was calculated as: (½)×length×width².

Treatment of A375 tumors with Cy7.5-amine and NIR 730 nm light. The tumors were treated at day 3 (since 5 million initial cells were injected per tumor site) when the average tumors size was 35 mm³. Forty mice were divided in 4 groups (number of mice per group, n=10): 1) Cy7.5-amine only 2) 2.5% DMSO only, 3) 2.5% DMSO+Light and 3) Cy7.5-amine+light. The day of treatment, fresh solutions (200 μM of Cy7.5-amine in PBS and controls 2.5% DMSO in PBS) were prepared as described before. The mice were anesthetized with isoflurane using a vaporizer. Then, each mouse was injected with 50 μL of 200 μM Cy7.5-amine solution in PBS or 2.5% DMSO, intratumorally. Then mice were kept for 25 min in the cages to let the Cy7.5-amine solution or DMSO solution interact with the tumors. Then, after the 25 min of incubation, the mice were treated (under anesthesia, using isoflurane) with 730 nm LED light source from Prizmatix applying a power intensity of 150 mWcm⁻² for 5 min (other power intensities were 210 mWcm⁻² for 5 min and 300 mWcm⁻² for 5 min as described in the treatment schedule in FIG. 27F). The light intensity was measured using an Optical Power Meter from Thorlabs, sensor model S302C and console model PM100D. When the treatment was finished the mice were put back into the cages and housed in the animal facility. The tumors sizes were measured every other day using a caliper in the first 20 days of the study and then in the late stage of the study twice a week.

Time-Dependent Density Functional Theory Analyses (TDDFT). Starting from a ground-state DFT calculation to obtain the energies of all the occupied levels, the absorption spectra were calculated by TDDFT using the Liouville-Lanczos approach as coded in the program Quantum Espresso. (Malcioglu et al., 2011; Giannozzi et al., 2009) The charge density responses were visualized using the software VESTA. (Momma & Izumi, 2011)

All of the compositions and/or methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and/or methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VI. REFERENCES

The following references, to the extent that they provide exemplary procedural or other details supplementary to those set forth herein, are specifically incorporated herein by reference:
Bilici et al., Front. Chem. 9, 2021.
Chapkin et al., Proc. Natl. Acad. Sci., 115:9134-39, 2018.
Cui et al., Nano Lett., 16:6390-95, 2016.
Giannozzi et al., J. Phys.: Condens. Matter, 21:395502, 2009.
Kong et al., Nat. Commun., 12:1280, 2021.
Lange et al., Pharmaceutics, 13, 2021.
Li et al., Bioact. Mater. 6:794-809, 2021.
Liu et al., ACS Nano, 13:6813-23, 2019.
Malcioglu et al., Comput. Phys. Commun. 182:1744-1754, 2011.
Mishra et al., Chem. Rev., 100:1973-2012, 2000.
Momma & Izumi, J. Appl. Cryst., 44:1272-127, 2011.
Mustroph, Phys. Sci. Rev, https://doi.org/10.1515/psr-2020-0145, 2021.
Mustroph & Towns, ChemPhysChem, 19:1016-23, 2018.
Orlandi & Siebrand, J. Chem. Phys., 58:4513, 2003.
Qian et al., J. Phys. Chem. A, 124:9156-65, 2020.
Shi et al., J. Biomed. Opt., 21:050901, 2016.
S̆tacková et al., J. Org. Chem., 85:9776-90, 2020.
Weissleder, Nat. Biotechnol., 19(4):316-17, 2001.

What is claimed:

1. A method of disrupting a membrane comprising:
(A) contacting the membrane with a compound, wherein the compound is capable of generating a vibronic-driven action and optionally further comprising a targeting moiety; and
(B) exposing the compound to an energy source sufficient to generate the vibronic-driven action, wherein the vibronic-driven action is sufficient to disrupt the membrane.

2. Use of a compound for disrupting a membrane comprising:
(A) contacting the membrane with the compound, wherein the compound is capable of generating a vibronic-driven action and optionally further comprising a targeting moiety; and
(B) exposing the compound to an energy source sufficient to generate the vibronic-driven action, wherein the vibronic-driven action is sufficient to disrupt the membrane.

3. A composition for use in the disrupting of a membrane comprising a compound, wherein the compound is capable of generating a vibronic-driven action and optionally further comprising a targeting moiety, provided that when the compound is exposed to an energy source sufficient to generate the vibronic-driven action, then the vibronic-driven action is sufficient to disrupt the membrane.

4. The method according to any one of claims 1-3, wherein the membrane is the membrane of a human cell.

5. The method of claim 4, wherein the human cell is a cancer cell.

6. The method according to any one of claims 1-3, wherein the membrane is a bacterial membrane, a virus, a fungal membrane, or a protozoal membrane.

7. The method according to any one of claims 1-6, wherein the disruption creates a pore in the membrane.

8. The method according to any one of claims 1-7, wherein the method results in necrosis of the cell.

9. The method according to any one of claims 1-8, wherein the compound comprises:
(i) has a net dipole via a charge (cation or anion or radical cation or radical anion) or radical (single unpaired electron);
(ii) has a high degree of symmetry across the longitudinal and/or transverse axis; and
(iii) has a resonance structure through a pi-bonded system whereby the charge or radical can oscillate between the near-symmetric two ends via resonance.

10. The method according to any one of claims 1-8, wherein the compound is an organic molecule.

11. The method of claim 10, wherein the organic molecule exhibits both a longitudinal molecular plasmon and a transverse molecular plasmon.

12. The method according to any one of claims 1-11, wherein the compound is further defined by the formula: 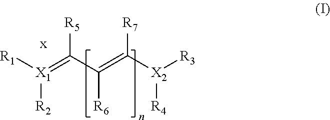
wherein:
x is a positive or negative charge;
n is an integer from 0 to 100;
X₁ and X₂ are each independently a heteroatom selected from O, N, S, B, P, Ge, As, or Se; and
R₁, R₂, R₃, R₄, R₅, R₆, and R₇ are each independently alkyl_{(C≤18)}, alkenyl_{(C≤18)}, alkynyl_{(C≤18)}, aryl_{(C≤18)}, aralkyl_{(C≤18)}, heteroaryl_{(C≤18)}, heterocycloalkyl_{(C≤18)}, or a substituted version of any of these groups; or
R₁ and R₂, R₁ and R₅, R₂ and R₅, R₃ and R₄, R₃ and R₇, and R₄ and R₇ are taken together to form one, two, three, four, five, or six aliphatic or aromatic rings; comprising at least three carbon atoms and no more than 36 carbon atoms; optionally comprising one, two, three, four, or five nitrogen, sulfur, or oxygen atom.

13. The method of claim 12, wherein the compound is further defined as: 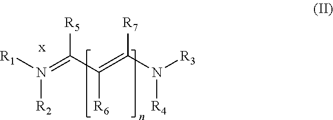
wherein:
x is a positive charge;
n is an integer from 0 to 100;
each R₁, R₂, R₃, R₄, R₅, R₆, and R₇ are each independently hydrogen, alkyl_{(C≤18)}, alkenyl_{(C≤18)}, alkynyl_{(C≤18)}, aryl_{(C≤18)}, aralkyl_{(C≤18)}, heteroaryl_{(C≤18)}, heterocycloalkyl_{(C≤18)}, or a substituted version of any of these groups; or
each R₁, R₂, R₃, R₄, R₅, R₆, and R₇ are each independently a cell membrane targeting moiety, wherein the cell targeting moiety optionally comprises a linker; or
each R₁ and R₂, R₁ and R₅, R₂ and R₅, R₃ and R₄, R₃ and R₇, R₄ and R₇, and R₅ and R₇ are taken together and each independently form one, two, three, four, five, or six aliphatic or aromatic rings; comprising at least three carbon atoms and no more than 36 carbon atoms; optionally comprising one, two, three, four, or five nitrogen, sulfur, or oxygen atom.

14. The method of claim 12, wherein X₁ and X₂ are N.

15. The method according to any one of claims 12-14, wherein R₁ or R₂ are symmetric with R₃ or R₄.

16. The method according to any one of claims 12-15, wherein R₁ is taken together with R₅ to form one, two, three, four, or five rings.

17. The method of claim 16, wherein R₁ is taken together with R₅ to form two, three, or four rings.

18. The method according to any one of claims 12-17, wherein R₃ is taken together with R₇ to form one, two, three, four, or five rings.

19. The method of claim 18, wherein R₃ is taken together with R₇ to form two, three, or four rings.

20. The method of either claim 18 or claim 19, wherein R₃ is taken together with R₇ to form three rings.

21. The method of claim 12, wherein R₅ and R₇ are taken together and form a single ring.

22. The method according to any one of claims 12-21, wherein n is an integer selected from 2, 3, or 4.

23. The method according to any one of claims 12-22, wherein R₄ is a cell targeting moiety with a linker.

24. The method of claim 23, wherein the linker is an alkyl chain, an alkenyl chain, an aryl chain, a peptide chain, a polyethylene glycol chain, or a polypropylene chain.

25. The method of claim 24, wherein the linker further comprises one or more joining functional group selected from ether, amide, disulfide, ester, amine, or thioether.

26. The method according to any one of claims 12-25, wherein the cell targeting moiety is a functional group that associates with the membrane, a carbohydrate or polysaccharide that binds to one or more markers on the membrane, a lipid that binds to one or more markers on the cell membrane, a small molecule that binds to one or more markers on the cell membrane, an aptamer that binds to one or more markers on the membrane, or a peptide or an antibody that binds to one or more markers on the membrane.

27. The method of claim 26, wherein the cell targeting moiety is a functional group that associates with the cell membrane.

28. The method of claim 27, wherein the functional group that associates with the cell membrane is an amine.

29. The method according to any one of claims 1-28, wherein the compound is further defined as:

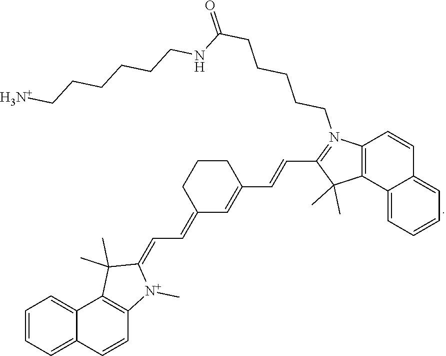

30. The method of claim 1-28, wherein the compound is further defined as: 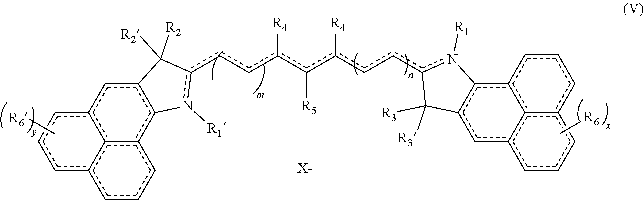
wherein:
R₁ and R₁′ are each independently alkyl_{(C≤8)}, substituted alkyl_{(C≤8)}, or a group of the formula:
A-NRₐRₐ′Rₐ″
wherein:
A is an alkanediyl_{(C≤12)} or substituted alkanediyl_{(C≤12)};
Rₐ and Rₐ′ are each independently hydrogen or alkyl_{(C≤8)}; and
Rₐ″ is absent, hydrogen, or alkyl_{(C≤8)};
provided at least one of R₁ and R₁′ is a group of the formula: -A-NRₐRₐ′Rₐ″ R₂, R₂′, R₃, and R₃′ are each independently hydrogen, alkyl_{(C≤8)}, or substituted alkyl_{(C≤8)};
R₄ and R₄′ are each independently hydrogen, alkyl_{(C≤8)}, substituted alkyl_{(C≤8)}, or R₄ and R₄′ are taken together as a cycloalkyl group;
R₅ is hydrogen, halo, carboxy, alkyl_{(C≤8)}, substituted alkyl_{(C≤8)}, or —C(O)OR_{b}, wherein R_{b} is alkyl_{(C≤6)} or substituted alkyl_{(C≤6)};
R₆ is hydrogen, amino, halo, hydroxy, or alkyl_{(C≤12)}, alkoxy_{(C≤12)}, alkylamino_{(C≤8)}, dialkylamino_{(C≤8)}, acyl_{(C≤8)}, acyl_{(C≤8)}, acyl_{(C≤8)}, or a substituted version thereof;
m and n are each 0, 1, 2, or 3;
x and y are each independently 0, 1, 2, 3, 4, or 5;
X is a monovalent anion; and
each of the rings is optionally present as either an aromatic or aliphatic ring.

31. The method according to claims 1-29, wherein the compound is at least one compound shown below:
Structure

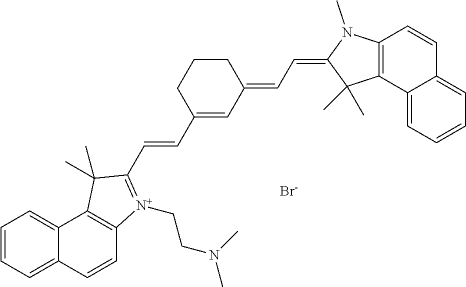 [SMILES: CN(C)CC[N+]1=C(/C=C/C2=CC(=C/C=C3/N(C)c4ccc5ccccc5c4C3(C)C)/CCC2)C(C)(C)c2c1ccc1ccccc21 ; [Br-]]

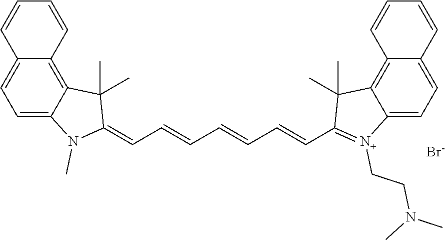 [SMILES: CN(C)CC[N+]1=C(/C=C/C=C/C=C/C=C2/N(C)c3ccc4ccccc4c3C2(C)C)C(C)(C)c2c1ccc1ccccc21 ; [Br-]]

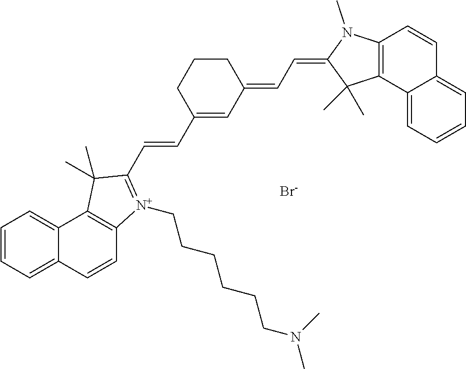 [SMILES: CN(C)CCCCCC[N+]1=C(/C=C/C2=CC(=C/C=C3/N(C)c4ccc5ccccc5c4C3(C)C)/CCC2)C(C)(C)c2c1ccc1ccccc21 ; [Br-]]

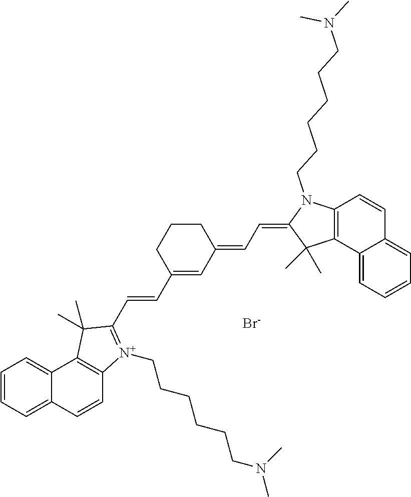 [SMILES: CN(C)CCCCCCN1/C(=C/C=C2C=C(/C=C/C3=[N+](CCCCCCN(C)C)c4ccc5ccccc5c4C3(C)C)CCC/2)C(C)(C)c2c1ccc1ccccc21 ; [Br-]]

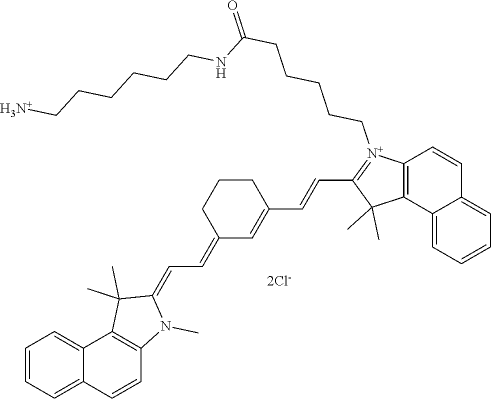 [SMILES: CN1/C(=C\C=C2C=C(/C=C/C3=[N+](CCCCCC(=O)NCCCCCC[NH3+])c4ccc5ccccc5c4C3(C)C)CCC\2)C(C)(C)c2c1ccc1ccccc21]

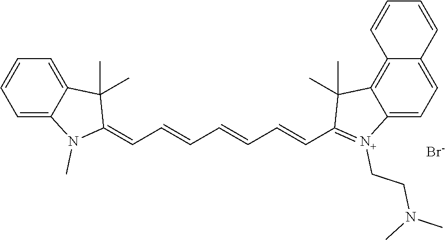 [SMILES: CN(C)CC[N+]1=C(/C=C/C=C/C=C/C=C2/N(C)c3ccccc3C2(C)C)C(C)(C)c2c1ccc1ccccc21 ; [Br-]]

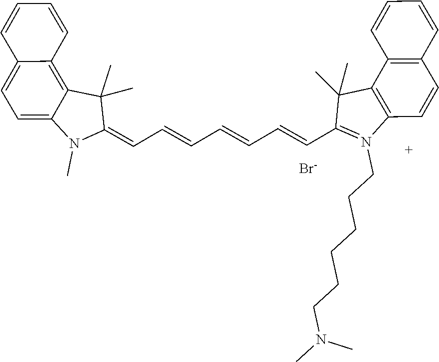

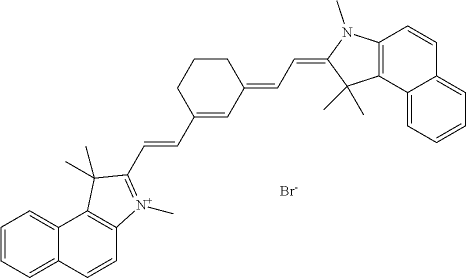 [SMILES: CN1/C(=C/C=C2C=C(/C=C/C3=[N+](C)c4ccc5ccccc5c4C3(C)C)CCC/2)C(C)(C)c2c1ccc1ccccc21 ; [Br-]]

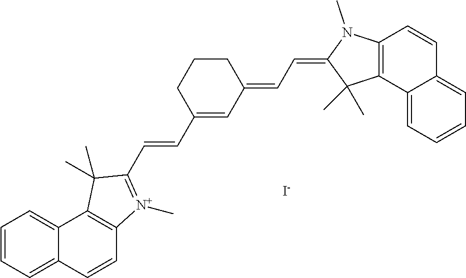 [SMILES: CN1/C(=C/C=C2C=C(/C=C/C3=[N+](C)c4ccc5ccccc5c4C3(C)C)CCC/2)C(C)(C)c2c1ccc1ccccc21 ; [I-]]

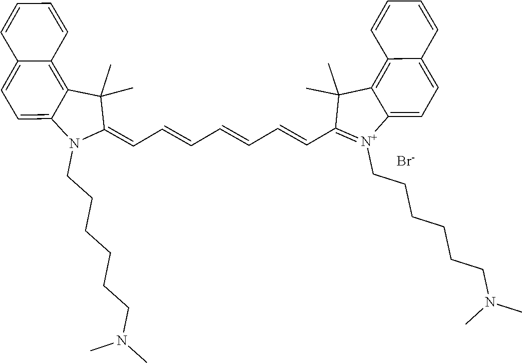 [SMILES: CN(C)CCCCCCN1/C(=C/C=C/C=C/C=C/C2=[N+](CCCCCCN(C)C)c3ccc4ccccc4c3C2(C)C)C(C)(C)c2c1ccc1ccccc21 ; [Br-]]

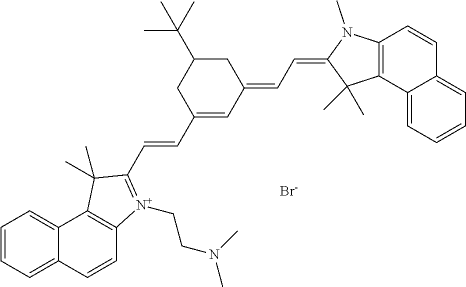 [SMILES: CN(C)CC[N+]1=C(/C=C/C2=CC(=C/C=C3/N(C)c4ccc5ccccc5c4C3(C)C)/CC(C(C)(C)C)C2)C(C)(C)c2c1ccc1ccccc21 ; [Br-]]

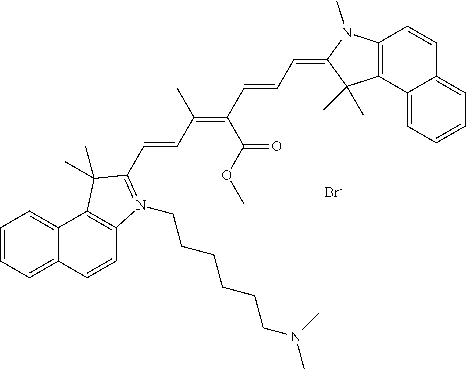 [SMILES: COC(=O)C(/C=C/C=C1/N(C)c2ccc3ccccc3c2C1(C)C)=C(C)\C=C\C1=[N+](CCCCCCN(C)C)c2ccc3ccccc3c2C1(C)C ; [Br-]]

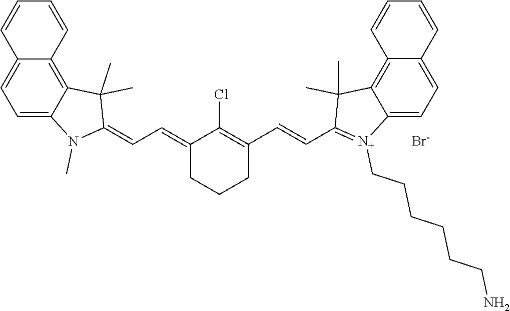 [SMILES: CN1/C(=C/C=C2\CCCC(/C=C/C3=[N+](CCCCCCN)c4ccc5ccccc5c4C3(C)C)=C2Cl)C(C)(C)c2c1ccc1ccccc21 ; [Br-]]

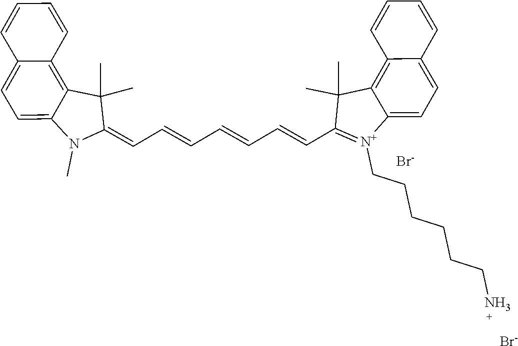 [SMILES: CN1/C(=C/C=C/C=C/C=C/C2=[N+](CCCCCCN)c3ccc4ccccc4c3C2(C)C)C(C)(C)c2c1ccc1ccccc21 ; [Br-] ; [Br-]]

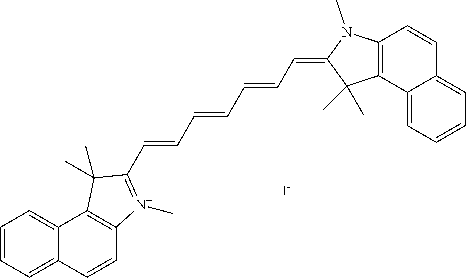 [SMILES: CN1/C(=C/C=C/C=C/C=C/C2=[N+](C)c3ccc4ccccc4c3C2(C)C)C(C)(C)c2c1ccc1ccccc21 ; [I-]]

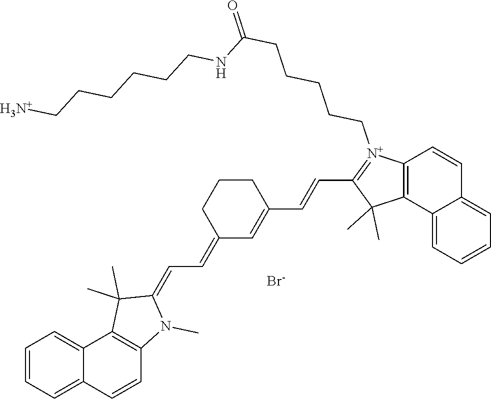 [SMILES: CN1/C(=C\C=C2C=C(/C=C/C3=[N+](CCCCCC(=O)NCCCCCC[NH3+])c4ccc5ccccc5c4C3(C)C)CCC\2)C(C)(C)c2c1ccc1ccccc21 ; [Br-]]

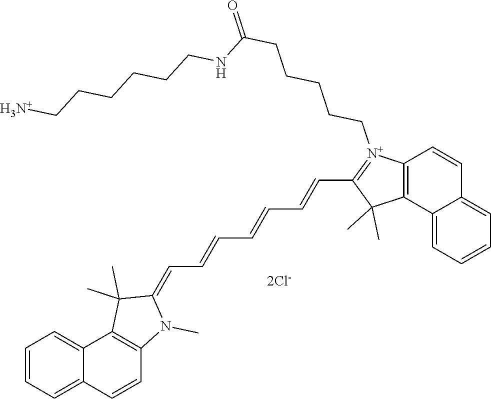 [SMILES: CN1\C(=C/C=C/C=C/C=C/C2=[N+](CCCCCC(=O)NCCCCCC[NH3+])c3ccc4ccccc4c3C2(C)C)C(C)(C)c2c1ccc1ccccc21]

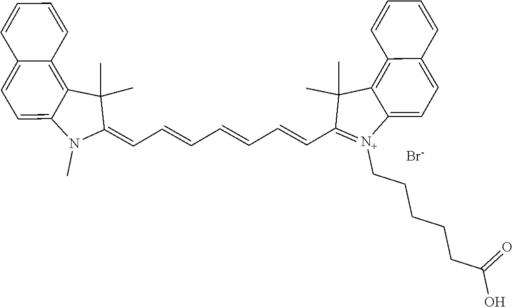 [SMILES: CN1/C(=C/C=C/C=C/C=C/C2=[N+](CCCCCC(=O)O)c3ccc4ccccc4c3C2(C)C)C(C)(C)c2c1ccc1ccccc21 ; [Br-]]

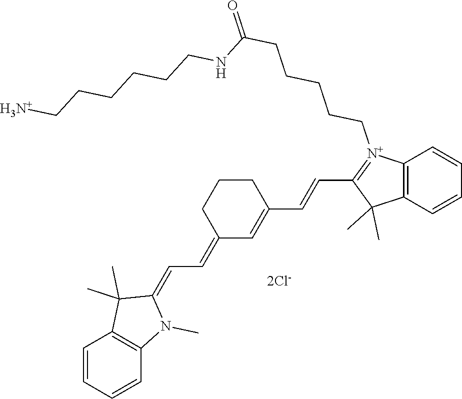 [SMILES: CN1/C(=C\C=C2C=C(/C=C/C3=[N+](CCCCCC(=O)NCCCCCC[NH3+])c4ccccc4C3(C)C)CCC\2)C(C)(C)c2ccccc21]

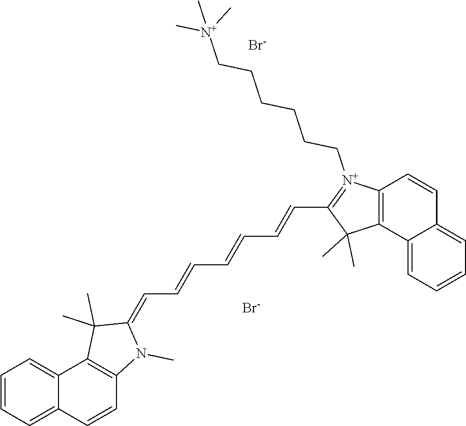 [SMILES: CN1\C(=C/C=C/C=C/C=C/C2=[N+](CCCCCC[N+](C)(C)C)c3ccc4ccccc4c3C2(C)C)C(C)(C)c2c1ccc1ccccc21 ; [Br-] ; [Br-]]

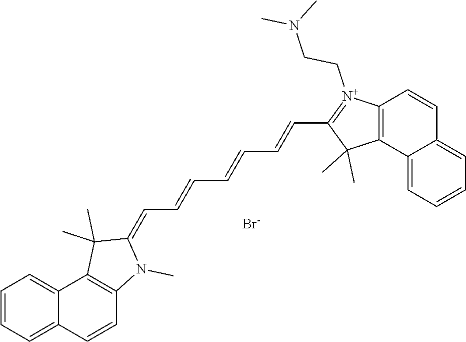 [SMILES: CN(C)CC[N+]1=C(/C=C/C=C/C=C/C=C2\N(C)c3ccc4ccccc4c3C2(C)C)C(C)(C)c2c1ccc1ccccc21 ; [Br-]]

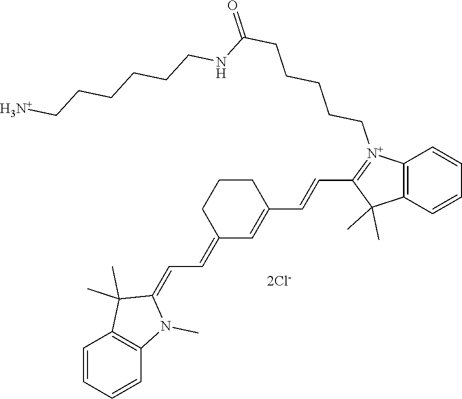 [SMILES: CN1/C(=C\C=C2C=C(/C=C/C3=[N+](CCCCCC(=O)NCCCCCC[NH3+])c4ccccc4C3(C)C)CCC\2)C(C)(C)c2ccccc21]

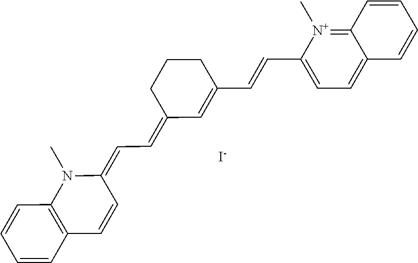 [SMILES: CN1/C(=C/C=C2C=C(/C=C/c3ccc4ccccc4[n+]3C)CCC/2)C=Cc2ccccc21 ; [I-]]

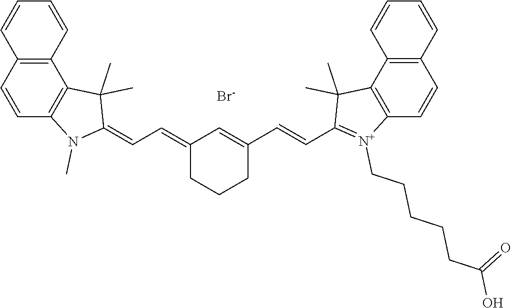 [SMILES: CN1/C(=C/C=C2C=C(/C=C/C3=[N+](CCCCCC(=O)O)c4ccc5ccccc5c4C3(C)C)CCC/2)C(C)(C)c2c1ccc1ccccc21 ; [Br-]]

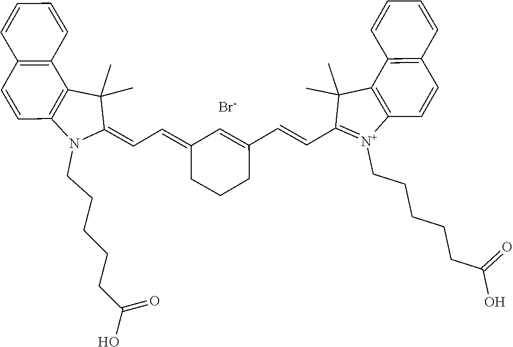 [SMILES: CC1(C)C(/C=C/C2=CC(=C/C=C3/N(CCCCCC(=O)O)c4ccc5ccccc5c4C3(C)C)/CCC2)=[N+](CCCCCC(=O)O)c2ccc3ccccc3c21 ; [Br-]]

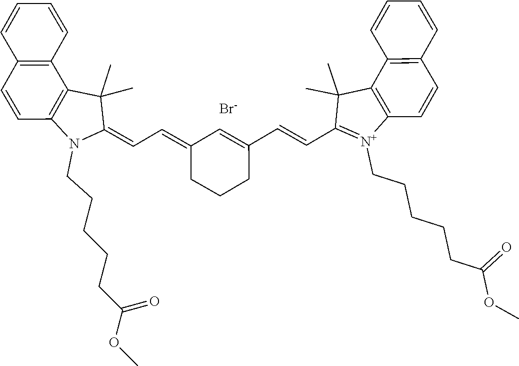 [SMILES: COC(=O)CCCCCN1/C(=C/C=C2C=C(/C=C/C3=[N+](CCCCCC(=O)OC)c4ccc5ccccc5c4C3(C)C)CCC/2)C(C)(C)c2c1ccc1ccccc21 ; [Br-]]

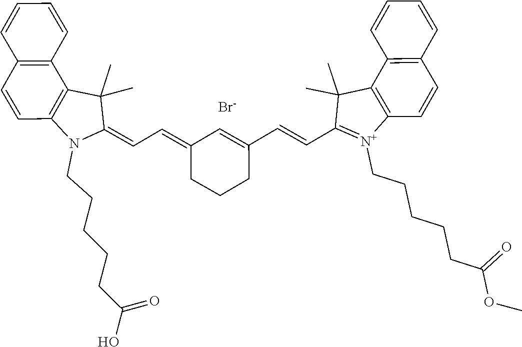 [SMILES: COC(=O)CCCCC[N+]1=C(/C=C/C2=CC(=C/C=C3/N(CCCCCC(=O)O)c4ccc5ccccc5c4C3(C)C)/CCC2)C(C)(C)c2c1ccc1ccccc21 ; [Br-]]

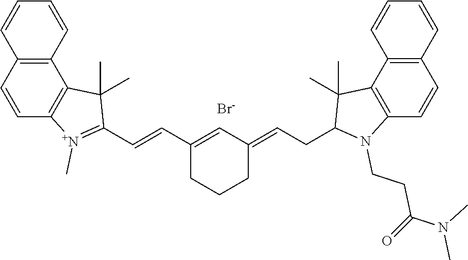 [SMILES: CN(C)C(=O)CCN1c2ccc3ccccc3c2C(C)(C)C1C/C=C1C=C(/C=C/C2=[N+](C)c3ccc4ccccc4c3C2(C)C)CCC/1 ; [Br-]]

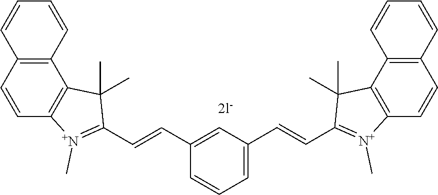

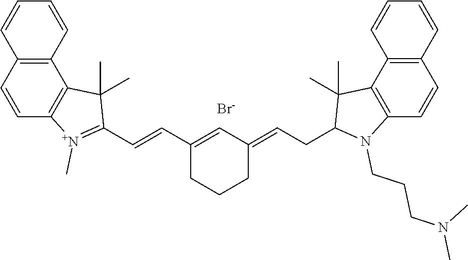 [SMILES: CN(C)CCCN1c2ccc3ccccc3c2C(C)(C)C1C/C=C1C=C(/C=C/C2=[N+](C)c3ccc4ccccc4c3C2(C)C)CCC/1 ; [Br-]]

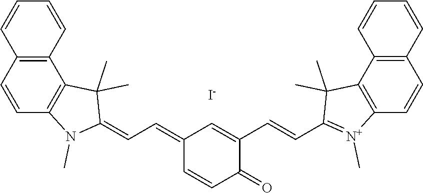 [SMILES: CN1/C(=C/C=C2\C=CC(=O)C(/C=C/C3=[N+](C)c4ccc5ccccc5c4C3(C)C)=C2)C(C)(C)c2c1ccc1ccccc21 ; [I-]]

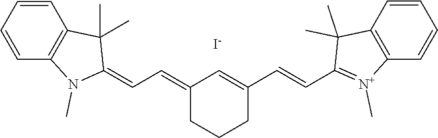 [SMILES: CN1/C(=C/C=C2C=C(/C=C/C3=[N+](C)c4ccccc4C3(C)C)CCC/2)C(C)(C)c2ccccc21 ; [I-]]

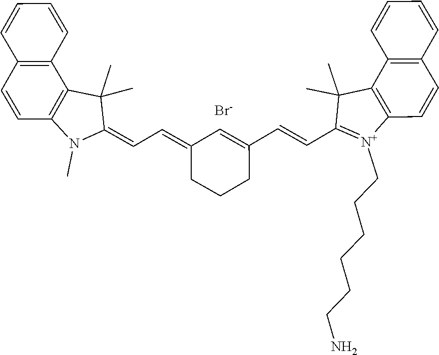 [SMILES: CN1/C(=C/C=C2C=C(/C=C/C3=[N+](CCCCCCN)c4ccc5ccccc5c4C3(C)C)CCC/2)C(C)(C)c2c1ccc1ccccc21 ; [Br-]]

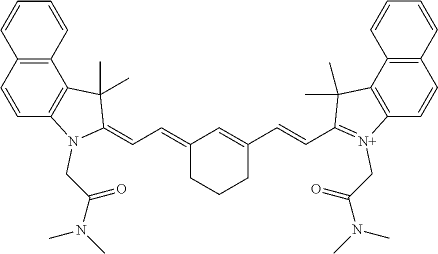 [SMILES: CN(C)C(=O)CN1/C(=C/C=C2C=C(/C=C/C3=[N+](CC(=O)N(C)C)c4ccc5ccccc5c4C3(C)C)CCC/2)C(C)(C)c2c1ccc1ccccc21]

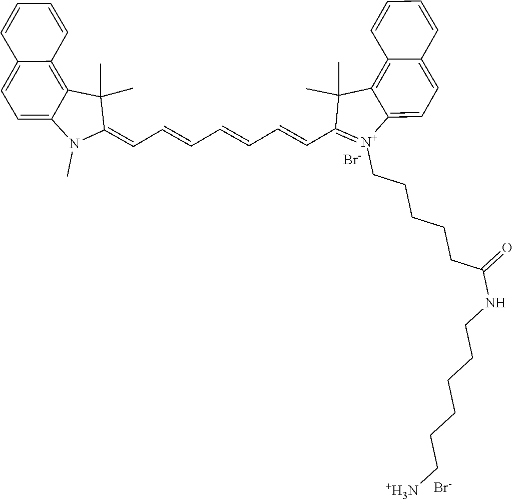 [SMILES: CN1/C(=C/C=C/C=C/C=C/C2=[N+](CCCCCC(=O)NCCCCCC[NH3+])c3ccc4ccccc4c3C2(C)C)C(C)(C)c2c1ccc1ccccc21 ; [Br-] ; [Br-]]

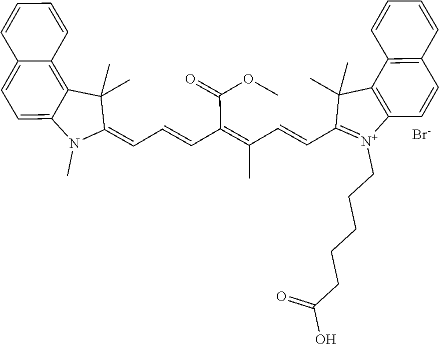 [SMILES: COC(=O)C(/C=C/C=C1/N(C)c2ccc3ccccc3c2C1(C)C)=C(C)\C=C\C1=[N+](CCCCCC(=O)O)c2ccc3ccccc3c2C1(C)C ; [Br-]]

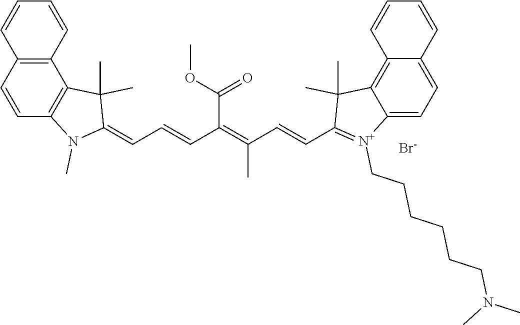 [SMILES: COC(=O)C(/C=C/C=C1/N(C)c2ccc3ccccc3c2C1(C)C)=C(C)\C=C\C1=[N+](CCCCCCN(C)C)c2ccc3ccccc3c2C1(C)C ; [Br-]]

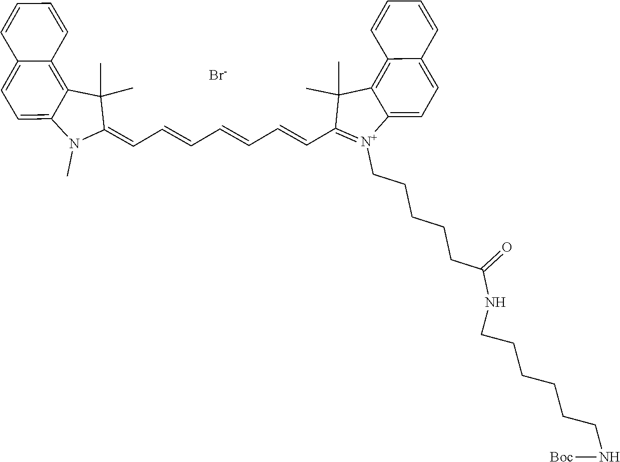 [SMILES: CN1/C(=C/C=C/C=C/C=C/C2=[N+](CCCCCC(=O)NCCCCCCNC(=O)OC(C)(C)C)c3ccc4ccccc4c3C2(C)C)C(C)(C)c2c1ccc1ccccc21 ; [Br-]]

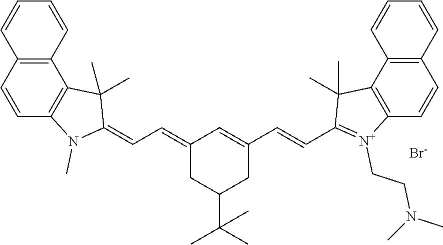 [SMILES: CN(C)CC[N+]1=C(/C=C/C2=CC(=C/C=C3/N(C)c4ccc5ccccc5c4C3(C)C)/CC(C(C)(C)C)C2)C(C)(C)c2c1ccc1ccccc21 ; [Br-]]

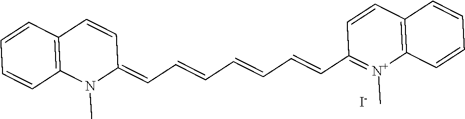 [SMILES: CN1/C(=C/C=C/C=C/C=C/c2ccc3ccccc3[n+]2C)C=Cc2ccccc21 ; [I-]]

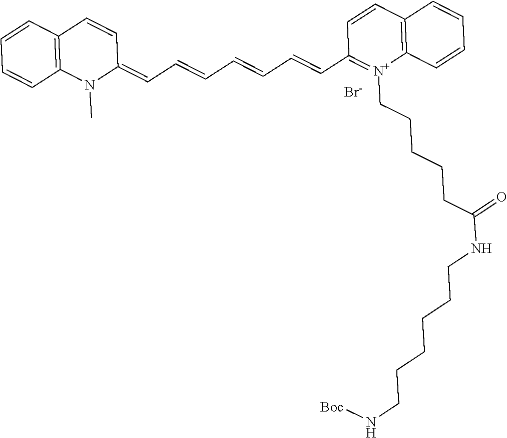 [SMILES: CN1/C(=C/C=C/C=C/C=C/c2ccc3ccccc3[n+]2CCCCCC(=O)NCCCCCCNC(=O)OC(C)(C)C)C=Cc2ccccc21 ; [Br-]]

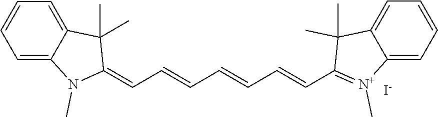 [SMILES: CN1/C(=C/C=C/C=C/C=C/C2=[N+](C)c3ccccc3C2(C)C)C(C)(C)c2ccccc21 ; [I-]]

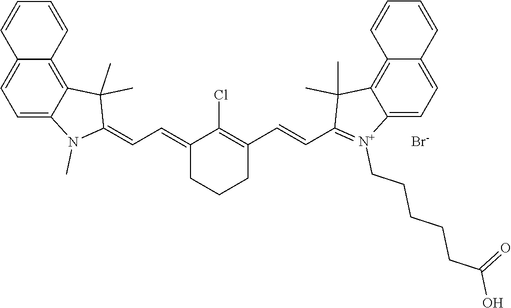 [SMILES: CN1/C(=C/C=C2\CCCC(/C=C/C3=[N+](CCCCCC(=O)O)c4ccc5ccccc5c4C3(C)C)=C2Cl)C(C)(C)c2c1ccc1ccccc21 ; [Br-]]

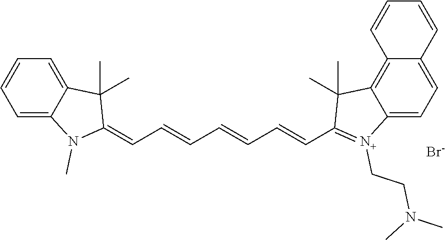 [SMILES: CN(C)CC[N+]1=C(/C=C/C=C/C=C/C=C2/N(C)c3ccccc3C2(C)C)C(C)(C)c2c1ccc1ccccc21 ; [Br-]]

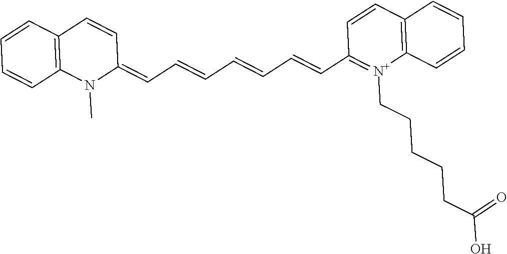 [SMILES: CN1/C(=C/C=C/C=C/C=C/c2ccc3ccccc3[n+]2CCCCCC(=O)O)C=Cc2ccccc21]

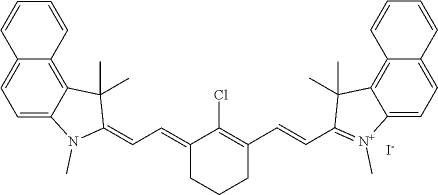 [SMILES: CN1/C(=C/C=C2\CCCC(/C=C/C3=[N+](C)c4ccc5ccccc5c4C3(C)C)=C2Cl)C(C)(C)c2c1ccc1ccccc21 ; [I-]]

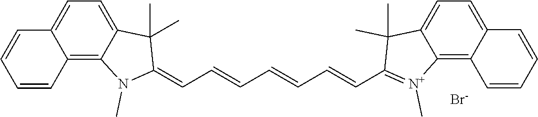 [SMILES: CN1/C(=C/C=C/C=C/C=C/C2=[N+](C)c3c(ccc4ccccc34)C2(C)C)C(C)(C)c2ccc3ccccc3c21 ; [Br-]]

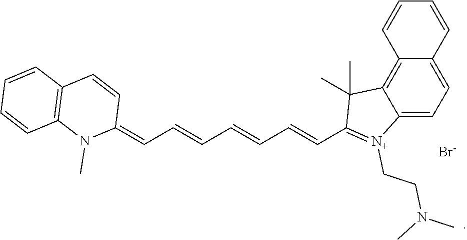 [SMILES: CN(C)CC[N+]1=C(/C=C/C=C/C=C/C=C2\C=Cc3ccccc3N2C)C(C)(C)c2c1ccc1ccccc21 ; [Br-]]

32. The method according to any one of claims 1-31, wherein the energy source is gamma rays, X-rays, ultraviolet (UV) light, visible (Vis) light, near-infrared (NIR) light, infrared light (IR), microwaves, radio waves, electric fields, ionizing radiation, magnetic fields, mechanical forces, ultrasound, or combinations thereof.

33. The method of claim 32, wherein the energy source is light with a wavelength from about 250 nm to about 2,000 nm.

34. The method according to any one of claims 1-33, wherein the intensity of the energy source is less than 200 mW/cm².

35. A method of treating a disease or disorder in a patient comprising:
(A) contacting the cell membrane of at least one cell of said patient with a compound, wherein the compound capable of generating a vibronic-driven action and optionally further comprising a cell targeting moiety; and
(B) exposing the compound to an energy source sufficient to generate a vibronic-driven action, wherein the vibronic-driven action is sufficient to disrupt the cell membrane of at least one cell of said patient.

36. The method of claim 35, wherein the method further comprises administering the compound with a therapeutic agent.

37. The method of claim 35 or claim 36, wherein the contacting of step (A) comprises administering the compound.

38. The method according to any one of claims 35-37, wherein the compound disrupts the cell membrane allowing the therapeutic agent to enter a cell.

39. A method of opening a cell membrane comprising:
(A) contacting the cell membrane with a compound, wherein the compound capable of generating a vibronic-driven action and optionally further comprising a cell targeting moiety; and
(B) exposing the compound to an energy source sufficient to generate a vibronic-driven action, wherein the vibronic-driven action is sufficient to open the cell membrane.

40. The method of claim 39, wherein the method comprises killing one or more cells.

41. The method of claim 40, wherein the cell is killed by necrosis.

42. A method of reducing the amount of adipose tissue in a patient comprising contracting the adipose tissue with a compound, wherein the compound capable of generating a vibronic-driven action and optionally further comprising a cell targeting moiety; and
exposing the compound to an energy source sufficient to generate a vibronic-driven action, wherein the vibronic-driven action is sufficient to reduce the adipose tissue.

43. The method of claim 42, wherein the method is sufficient to reduce the weight of the patient.

44. A method of disrupting a cellular component comprising:
(A) contacting the cellular component with a compound, wherein the compound is capable of generating a vibronic-driven action and optionally further comprising a targeting moiety; and
(B) exposing the compound to an energy source sufficient to generate the vibronic-driven action, wherein the vibronic-driven action is sufficient to disrupt the cellular component.

45. The method of claim 44, wherein the cellular component is a cellular component of a eukaryotic cell.

46. The method of claim 45, wherein the eukaryotic cell is a parasitic cell selected from a bacterial cell, a protozoan cell, a virus, or a fungal cell.

47. The method of claim 44, wherein the cellular component is a cellular component of a human cell.

48. A compound of the formula: 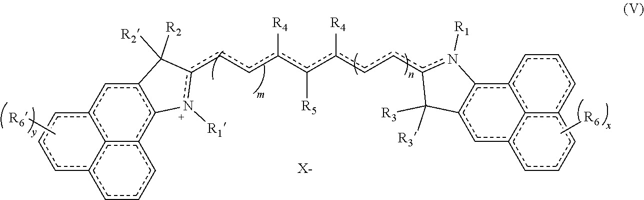
wherein:
R₁ and R₁′ are each independently alkyl_{(C≤8)}, substituted alkyl_{(C≤8)}, or a group of the formula:
-A-NRₐRₐ′Rₐ″
wherein:
A is an alkanediyl_{(C≤12)} or substituted alkanediyl_{(C≤12)};
Rₐ and Rₐ′ are each independently hydrogen or alkyl_{(C≤8)}; and
Rₐ″ is absent, hydrogen, or alkyl_{(C≤8)};
provided at least one of R₁ and R₁′ is a group of the formula: -A-NRₐRₐ′Rₐ″
R₂, R₂′, R₃, and R₃′ are each independently hydrogen, alkyl_{(C≤8)}, or substituted alkyl_{(C≤8)};
R₄ and R₄′ are each independently hydrogen, alkyl_{(C≤8)}, substituted alkyl_{(C≤8)}, or R₄ and R₄′ are taken together as a cycloalkyl group;
R₅ is hydrogen, halo, carboxy, alkyl_{(C≤8)}, substituted alkyl_{(C≤8)}, or —C(O)OR_{b}, wherein R_{b} is alkyl_{(C≤6)} or substituted alkyl_{(C≤6)};
R₆ is hydrogen, amino, halo, hydroxy, or alkyl_{(C≤12)}, alkoxy_{(C≤12)}, alkylamino_{(C≤8)}, dialkylamino_{(C≤8)}, acyl_{(C≤8)}, acyl_{(C≤8)}, acyl_{(C≤8)}, or a substituted version thereof;
m and n are each 0, 1, 2, or 3;
x and y are each independently 0, 1, 2, 3, 4, or 5;
X is a monovalent anion; and
each of the rings is optionally present as either an aromatic or aliphatic ring.

49. The compound of claim 48 further defined as: 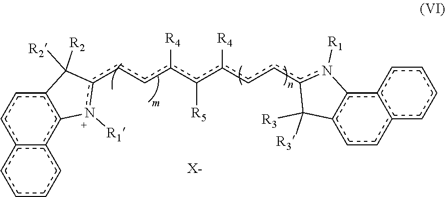
wherein:
R₁ and R₁′ are each independently alkyl_{(C≤8)}, substituted alkyl_{(C≤8)}, or a group of the formula:
-A-NRₐRₐ′Rₐ″
wherein:
A is an alkanediyl_{(C≤12)} or substituted alkanediyl_{(C≤12)};
Rₐ and Rₐ′ are each independently hydrogen or alkyl_{(C≤8)}; and
Rₐ″ is absent, hydrogen, or alkyl_{(C≤8)};
provided at least one of R₁ and R₁′ is a group of the formula: -A-NRₐRₐ′Rₐ″
R₂, R₂′, R₃, and R₃′ are each independently hydrogen, alkyl_{(C≤8)}, or substituted alkyl_{(C≤8)};
R₄ and R₄′ are each independently hydrogen, alkyl_{(C≤8)}, substituted alkyl_{(C≤8)}, or R₄ and R₄′ are taken together as a cycloalkyl group;
R₅ is hydrogen, halo, carboxy, alkyl_{(C≤8)}, substituted alkyl_{(C≤8)}, or —C(O)OR_{b}, wherein R_{b} is alkyl_{(C≤6)} or substituted alkyl_{(C≤6)};
m and n are each 0, 1, 2, or 3;
X is a monovalent anion; and
each of the rings is optionally present as either an aromatic or aliphatic ring.

50. The compound of either claim 48 or claim 49 further defined as: 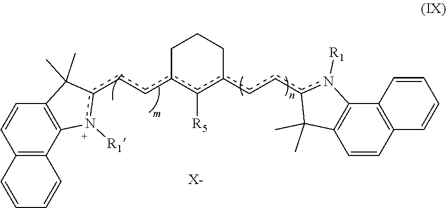
wherein:
R₁ and R₁′ are each independently alkyl_{(C≤8)}, substituted alkyl_{(C≤8)}, or a group of the formula:
-A-NRₐRₐ′Rₐ″
wherein:
A is an alkanediyl_{(C≤12)} or substituted alkanediyl_{(C≤12)};
Rₐ and Rₐ′ are each independently hydrogen or alkyl_{(C≤8)}; and
Rₐ″ is absent, hydrogen, or alkyl_{(C≤8)};
provided at least one of R₁ and R₁′ is a group of the formula: -A-NRₐRₐ′Rₐ″
R₅ is hydrogen, halo, carboxy, alkyl_{(C≤8)}, substituted alkyl_{(C≤8)}, or —C(O)OR_{b}, wherein R_{b} is alkyl_{(C≤6)} or substituted alkyl_{(C≤6)};
m and n are each 0, 1, 2, or 3; and
X is a monovalent anion.

51. The compound according to any one of claims 48-50, wherein the compound is further defined as:

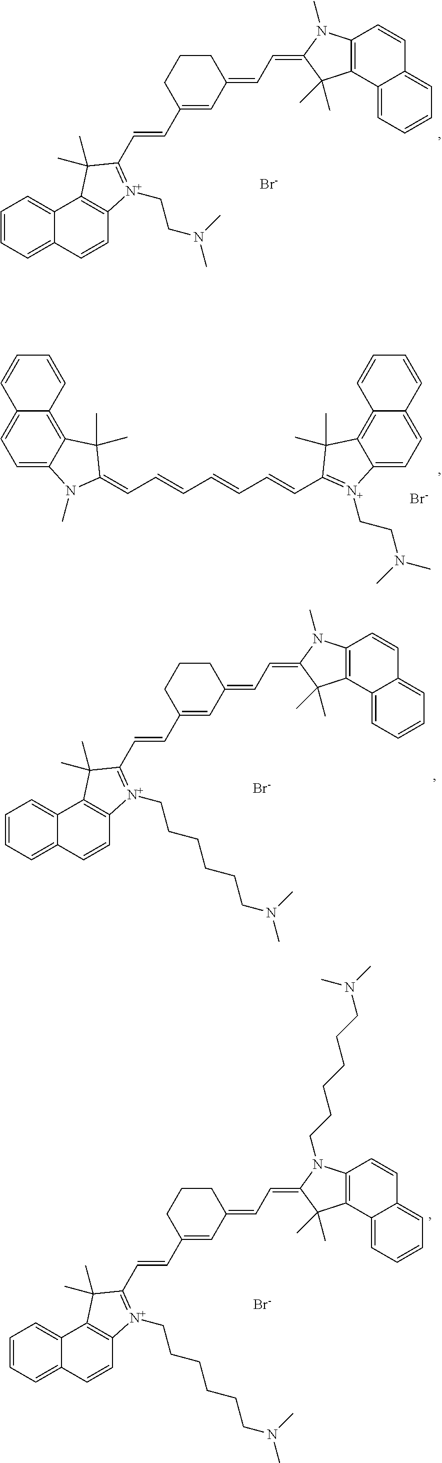

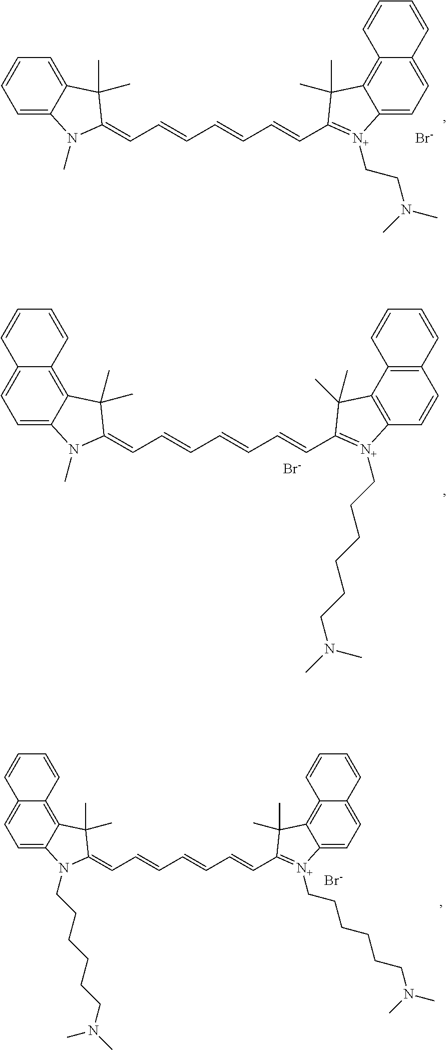 [SMILES: CN(C)CC[N+]1=C(/C=C/C=C/C=C/C=C2/N(C)c3ccccc3C2(C)C)C(C)(C)c2c1ccc1ccccc21 ; [Br-] ; CN(C)CCCCCC[N+]1=C(/C=C/C=C/C=C/C=C2/N(C)c3ccc4ccccc4c3C2(C)C)C(C)(C)c2c1ccc1ccccc21 ; [Br-] ; CN(C)CCCCCCN1/C(=C/C=C/C=C/C=C/C2=[N+](CCCCCCN(C)C)c3ccc4ccccc4c3C2(C)C)C(C)(C)c2c1ccc1ccccc21 ; [Br-]]

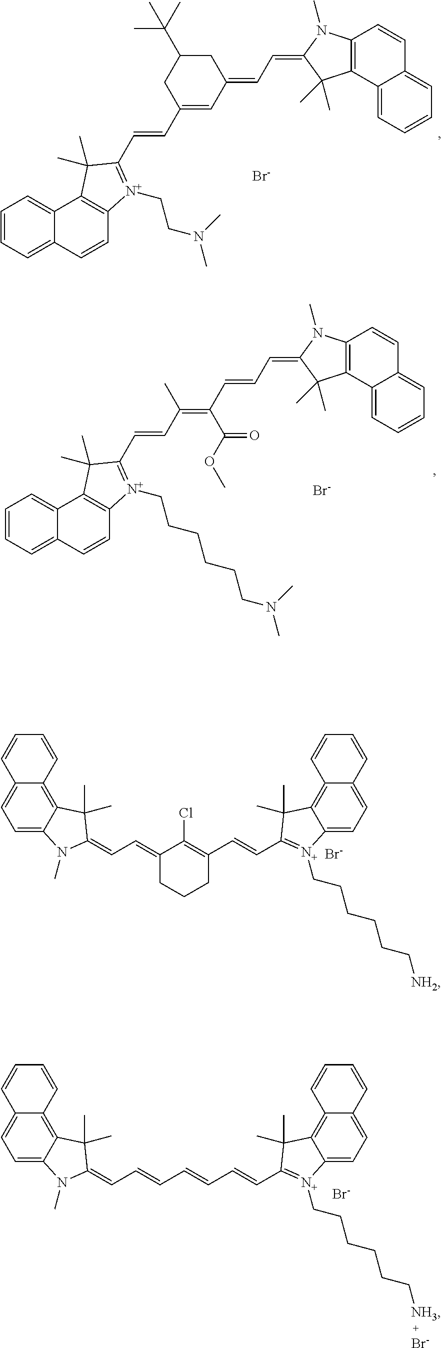

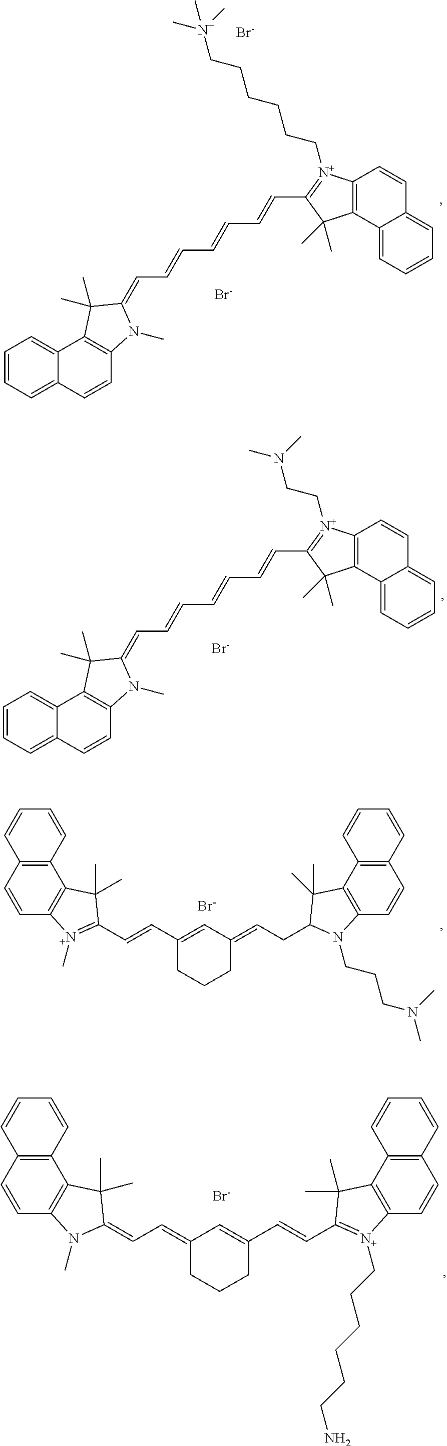

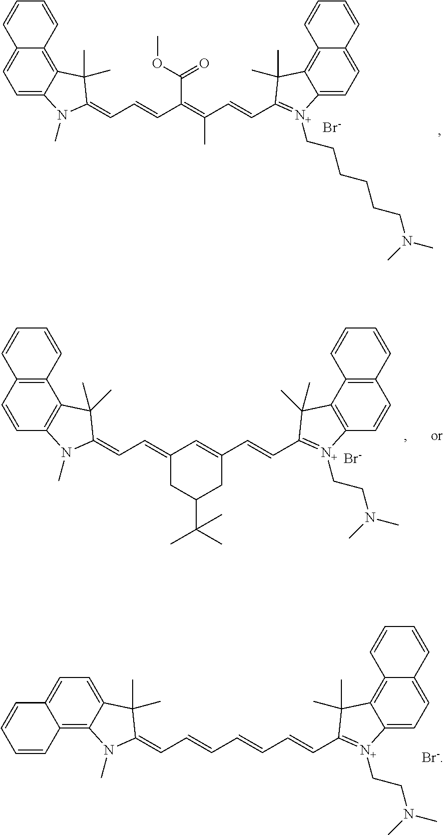

52. A method of disrupting a cell membrane comprising contacting the cell membrane with a compound of formula: 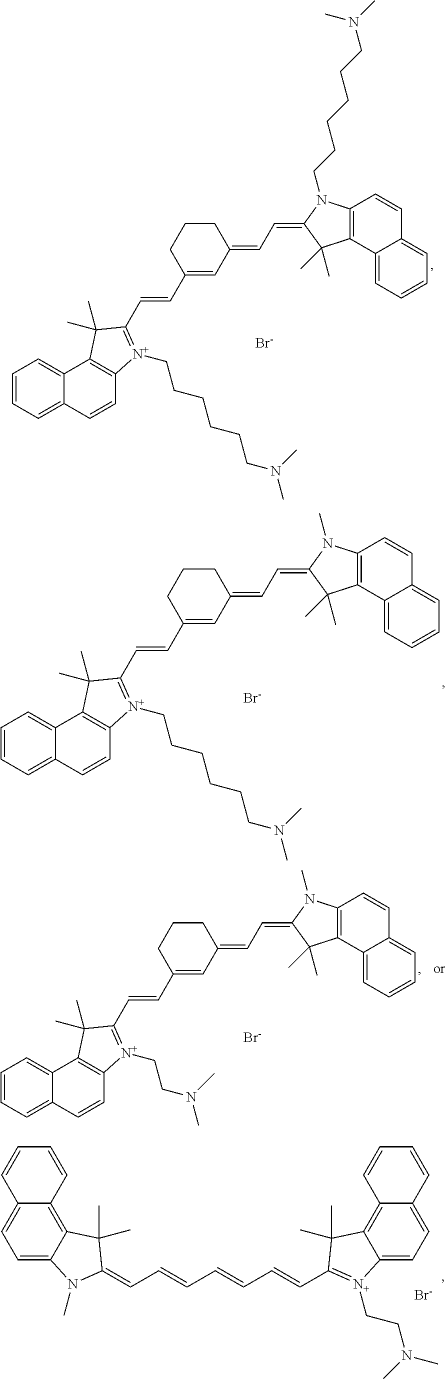
and exposing the membrane to an energy source capable of generating vibronic-driven action.
